SEQUENCE LISTING (46 sequences)

SEQ ID NO: 1 - DNA, 5862 bp, Homo sapiens
  source 1..5862: mol_type = unassigned DNA
gtctaaactttcactttcacagcgcggcggctgcggcggcggcggcggcgggcgagggtgaccggccgagcggcggcggcatggagtagacgcgcggcggcagcggcggcggcggcggacgcgagaggcagcggcgagcgcggcggcggcggcggcagcggcggccccggagccggcggggccgagcttgcgagcggcgagcgcggagcggcgccgggccgagcgcggggccgcgggccgggcgggcgcagcgcggcggaggccggaggagccgagccggagcccgagcccgagcgcggccgccgcctgccgggcctcccctcgccgcggccggccgccgcgctcccgcccgggcgcccagctatgtactccccgtactgcctcacccaggatgagttccacccgttcatcgaggcactgctgcctcacgtccgcgctttctcctacacctggttcaacctgcaggcgcggaagcgcaagtacttcaagaagcatgaaaagcggatgtcgaaggacgaggagcgggcggtgaaggacgagctgctgggcgagaagcccgagatcaagcagaagtgggcatcccggctgctggccaagctgcgcaaggacatccggcccgagttccgcgaggacttcgtgctgaccatcacgggcaagaagcccccctgctgcgtgctctccaaccccgaccagaagggcaagatccggcggattgactgcctgcgccaggctgacaaggtgtggcggctggacctggtcatggtgattttgtttaaggggatccccctggaaagtactgatggggagcggctctacaagtcgcctcagtgctcgaaccccggcctgtgcgtccagccacatcacattggagtcacaatcaaagaactggatctttatctggcttactttgtccacactccggaatccggacaatcagatagttcaaaccagcaaggagatgcggacatcaaaccactgcccaacgggcacttaagtttccaggactgttttgtgacttccggggtctggaatgtgacggagctggtgagagtatcacagactcctgttgcaacagcatcagggcccaacttctccctggcggacctggagagtcccagctactacaacatcaaccaggtgaccctggggcggcggtccatcacctcccctccttccaccagcaccaccaagcgccccaagtccatcgatgacagtgagatggagagccctgttgatgacgtgttctatcccgggacaggccgttccccagcagctggcagcagccagtccagcgggtggcccaacgatgtggatgcaggcccggcttctctaaagaagtcaggaaagctggacttctgcagtgccctctcctctcagggcagctccccgcgcatggctttcacccaccacccgctgcctgtgcttgctggagtcagaccagggagcccccgggccacagcatcagccctgcacttcccctccacgtccatcatccagcagtcgagcccgtatttcacgcacccgaccatccgctaccaccaccaccacgggcaggactcactgaaggagtttgtgcagtttgtgtgctcggatggctcgggccaggccaccggacagcattcgcaacgacaggcgcctcctctgccaaccggtttgtcagcatcggaccccgggacggcaactttctgaacatcccacagcagtctcagtcctggttcctctgataagatcgacaaaagaaacaacaaaatgagaagaagaggttcctcgaaaggggggagaagaaattttgagaatggaaaaatcccccagcccagcccagccccaccgaaaagcaaaaattacacgtcgtcagccactcagcccttctctcctccagcccggggacccccgcgggccccagaagcagcccagttctcagagagcccttggaaggggtctcggtggagctgtgcaccagcagccaagcagaaagaaacacgcgacatggactctgtcaagtagaggacagaaagcaagaaaggatgcagaactgccttcctccccctgaccccgccccggccttctggggaaggaacaaagtccccaaacaaagcaaccagcacaattctgaaggggcctggcctccaccctcaccccttcctaggggaaccccaccctccacacagccggagctgccctagggagcctggagggccagcttgtaaagatgatggggtttagatccctcaggctctcccctccagactccgcccttccctccctccctccctccctccctctctgccaaggctccagcttcttcccccagctgctcccgaccaggagggggagagcagcctccacttaccccaccccacccttgggctaaaagcccccaggcgggcagggggtgacccctggagctagttgcgtgtcccagaatggagggtgttctgacaccccaccctgagccgcaagagcagtcctggggccctggacccctctgtacagtccgtaggaaaaagtcggaatgctctcgacggcctcgtcccagcctgggacaggccccctttcccctctctctgcaggccaggagggcctccttcctgccacgagggaggggagtcgggccccaggtcgcccccgcccccagccctgcatgcaggtgccctcgctccgccccatcagttcctgcccctgcccctcatgcagactgccctgctggggccgggccggagggtggagcagaaaggggaccccggagccgagcgaggaggaccaggcagccgccgctgccgcgctaagccaccacctgcgcttaggtaggcgtcctgctcgccgactttcagttccttgggagggtgttgggtgtcgtccttttcaaaagtgttttggagctttctgtgccccccgactttcccccgcctccccgccccccacgtggccacttttctctggattttagctgtaatgtctttactctttatttaggggtggggcattcattgtttgggtcttttgctgttggaatgggaactcctcctccatttgagcaacttgggaacaatttggtaacacaccacaggaagtagctctcccccccagccccctcctccctcaagggagggttggggggcctgtccagagggtcttcagaagcccccctgggagggaggggaggatgagcacgcccagctcccctccagggtgtgacttggcccctctggcttgtctttctgtgccttactcctcctcctgcgtctcccgttcctggccccttcttgagtccttgtgcctctctctttctctctctttcttaattgtatgaaaacacaaagcacaggtcaggatcctctgagagaaaatcaacattgcaccacgtaggggtgggctatgggctgtatttattgtgaatctagtttgtgaggctgtggccccgagctggcggagggagggaagaggagggagtgacgggaggggaggaggtcagcgacctggggccgtagcggcaggcgaacggtgcctgctacccagctggaagccacaaggtggctggctccaggggcggcttttgttggaagttgagtgaagccctccccctgtcctcagcgtgcagccctagaggaccccagggctgaggggcagtggatcctgcgggagtctcccggggcgtggggagtaaggccccgggggtggggggccgggtgggccgggcgtgacgcgcggtcaaagtgcaatgatttttcagttcggttggctaaacagggtcagagctgagagcgaagcagaaggggctccctgtccggcccacgtgccctttccctcgacgacagtcgagggctcgggctctgtgggactgtgggagctagggtctgcggggcgcctgcccgggcgaggtcggaagctgcaggccagctgggcccgggccggagcgtgcccggcggggctgcccgggcgggcagggggtgggggctgctcctttcccaagtggtgttgtgaggggcaatgagggcaacaggagatgtggggacgtgttaggagagaaaaaaaaaaaaacaaaaatatatatgggggaaattaacttttttttttcattgaaccaagtgcaatgcatcagagagttttcctatctttgtatgttaagagattaagaaaaaaaaattctatttttgttgtaatgtcctcgcggctctggggacgctaaaagaaccgggcctgccccgccctgcgcggggataacgaaagctgagtgtttttcccttttttttgttcgtttttagtttttttttttttaagtcgttttcctgcgttgacgaggatgatctggggtttttatttgtttcgtcgttcgttctgtttcggtgggagggctgaaggaaacgttcacattttagagtttaaaaaaaacacctcgacatttaaaaaatcaaccaacacaagatcaaaaaggaaaaggacgagagaaaaattatttttaagataattaaacataaaaccctggtgcttcttacattataaagtacgttttaaagaacccacaaactattatacataagtttatgaatcaattaaatatcctgcacttgttaggaatacgcatatcccttctttgttgagtttaacggaacgggacagcggcgtgcccccggcggctggactgctccggccgcgggtctccccgggcgcccctccctggggcccagcacccctcctcgccccatccccgtccgggtacgggggcgcggcaggggtccccggcccctcccccgcagaggtcaatgccaacgaacaaacgtcccctccctccctccctctccgccccgagcgcccttctttgagccagacgccaacttgaccctcaccagcattatcaggagcgcgctcagcaagttggtagtttcctcccccctttcccggcgcccctcccgcccccattcaacatctctcatcctatccccgaccccctccggggaacaccgggaaggctcgacgctccaggacaggaccagccacgctgacaggtcgatttgcccaggcccgcgcccgcacgcacgcacgcacacggccccgcacacagccccgccccaccccgcaaccagccctgtcgactgccttatacacccgcccccgcgctggccggccgacctagtgccttgttctcacccccgtgctggcggagcggacgccgcgctctgggtcccagaggggccgggtggctcagacgacccaccactcccccaccctgaccgtgctgaacagacccccccacacgagagaaaataaaggagcaataaagtcacgagaactttcgtcccccaatcgagagcccgaggggcaccccagccccgcctctgctcccccccaccccacccaccctcggggcgcccccctccccccgcaagccagcctgggccagccccgcttcggcccctcccgggagatccgtgcgcccgaccagcaccagcatcgcggaccgcaaaggccgcccgtcccgtcaaacaagtttcttcttaggctaagaaacgcagtatatacgagtatctctatatatagtactaatggatttggtgtgcttcccccttagcgtccccctccctctgctcctcctccttcagcctggtctccccctcttctctgccctccacccccgtctctgcactgagatacataagaaacaagggtagtttactgtctgttttgttttctgggttttcagtgtcctagcggaatgcaagtaggcagccagcccgtctgttccctctccgccccgccccgccccgcccccgtcactgcgcttctgttataccatctttgcctgactctctccggcttctccattgaatggctaatgtgtatgtgaaataaagaaataaagaaaaa

SEQ ID NO: 2 - DNA, 8029 bp, Homo sapiens
  source 1..8029: mol_type = unassigned DNA
agtaagttcagcgcgcccgctccggccggccctgcgcctcccgccgcgcccgggatgtattcgtccccgctctgcctcacccaggatgagttccacccgttcatcgaggccctgctgcctcacgtccgcgccttcgcctacacctggttcaacctgcaggcgcggaagcgcaagtacttcaagaagcacgagaagcggatgtcgaaggacgaggagcgtgcggtcaaggacgagctgctgggcgagaagcccgaggtcaagcagaagtgggcgtcgcggctgctggccaagctgcgcaaggacatccggcccgagtgccgcgaggacttcgtgctgagcatcaccggcaagaaggcgccgggctgcgtgctctccaaccccgaccagaagggcaagatgcggcgcatcgactgtctccggcaggcggacaaggtgtggcggctggacctggtcatggtcatcctgttcaagggcatcccgctggagagcaccgacggcgagcgcctggtcaaggctgcgcagtgcggtcacccggtcctgtgcgtgcagccgcaccacattggcgtggccgtcaaggagctggacctctacctggcctacttcgtgcgtgagcgagatgcagagcaaagcggcagtccccggacagggatgggctctgaccaggaggacagcaagcccatcacgctggacacgaccgacttccaggagagctttgtcacctccggcgtgttcagcgtcactgagctcatccaagtgtcccggacacccgtggtgactggaacaggacccaacttctccctgggggagctgcaggggcacctggcatacgacctgaacccagccagcactggcctcagaagaacgctgcccagcacctcctccagtgggagcaagcggcacaaatcgggctcgatggaggaagacgtggacacgagccctggcggcgattactacacttcgcccagctcgcccacgagtagcagccgcaactggacggaggacatggaaggaggcatctcgtccccggtgaagaagacagagatggacaagtcaccattcaacagcccgtccccccaggactctccccgcctctccagcttcacccagcaccaccggcccgtcatcgccgtgcacagcgggatcgcccggagcccacacccgtcctccgctctgcatttccctacgacgtccatcctaccccagacggcctccacctacttcccccacacggccatccgctacccacctcatctcaacccccaggacccgctcaaagatcttgtctcgctggcctgcgacccagccagccagcaacctggaccgttaaatggaagtggtcagctcaaaatgcccagccactgcctttctgctcagatgctggcacctccgcccccggggctgccacggctggcgctcccccctgccaccaaacccgccaccacctccgagggaggagccacgtcgccgacctcgccttcctactctccgcccgacacgtcccctgcaaaccgttcctttgtgggattaggaccaagggatcctgcgggcatttatcaggcacagtcctggtatctgggatagcaaaggtcttcttccctcgccccttctccatcgtcccaggaatcccagggggcagcacagccggcccccggcccacgttttcggtggaaaattagagtgaacaagaacacccctgccgactcccagcccggccaaaaagacaaaacacatagacgcacacactcaggaggaaaagaaaaaacaaaggcagaagaagaagaagaagaaataaaaacccacccaagcaagaagacaaaaggtaaagacgcaacgtttccaactctcgggacgccaaggccgcaggactggagggccaggccccgccacccccacgggagacccgggacagggcgtcttcctaagttattcatctcctctccgcctgctgctcgggaaggacagacgccggccgcccgcccgcgccccggaggccctggctctgtccggagaccaggtgagcacagcctggagcctgtgcccagggccgacaggcgcgacacccagcaaggccacctctccccgggcccccgcgcctctgccggacacggaccggcccctcagcccccaccgaggacgcagccactggggggaaagggagacacagcggaccccggccgggcagcggagaccgcagaggcgggcagggtggggcaggcgagtggtgtcgcgggggtgcgtggcgcttgcgagccctggccaggggaggaagtgaggcccaggcacctgctgcccctcgagggggccctgcctgccgcggggcctccccacaagcccctcccaaagcgccggccgactcgctgtctcgctggggactctttcagccctcgcgcccgcccgtttgggaggagaagtctctatgcaattggccccggcccctccaccccccacccccggcataggaggcccccccacctcgcccggctcacacccccaaagggagggacccacattgcacacactgtaagaaatgcactttccgaggaaggggatgggggagcccggacacccagagctccccgagttgggggtgcccgtctggagcgcccccgtcagcccctggcggtgggaggtgagagcgagtggtttaagtgcctgattaccaccacccgcccccccctttgtccagctgggacacggaatggccgcgggcctcctccccctcccctccagcctctccaccagcccctccagtcaaccctcatcgccgtgcccccccagagctagagagatggggcccctgcgtggcccgaggggcagagctgggcgtcacttcgcaagcgtcctgccctgccggggcgcgggggtgggctctggggaagccggtgcgccccccacgcctccgctgccagtgccttacattctggagcgacccccctccctggtgcctcccagcgaagggggaccgccgtttgcactttcatcgcctaccccgacgcggggcccagctgcgggacgtgcatcacggctgggcccccagaggagagaggaggccgacgccagcggtccccgctcggaacggggagggttttcggggggttcggcgtcgcaccttggggccccccgcagccgtgtagggggcctcccatctgctaagcgtttttccgttgagccgctccaaaaacactaagctggggacgccaggtgcccccccaccccggctccctggccctatccacacctccacccccaccccaggatcgccatctttaggggaggcctgggagggggtgttaggtgttttagggccaccgagctcaaacacaaggacccctccccggcccacccagcccagccccaactgacctccatgcctagggaaaaactccccccaccactgccccctcccccgacccaggccaaagccagggcaggtctccgggtctcacctgctcctagcctcacccccctgcccccgaaaaccagactctcctcccaaactagcctcaggagcttggcgaacccgctcgctcctaaagagaaagacccaggaccctcccccatcacccccaagagaggttcgccatcctctggcctcgagcccttggtccctccgtccgtctgtcctcggggcccgctcccccggtggcccttggggatcaaagcgtgggccgctctccgggagggcgggcgggggagggggtggtcgggttgtgccattggggtgtccggaagcttctcagccagggtgggggtcgtggagtgggggagggaggccagccgggctccagaggggtcagggcgcgacgagaaccaactctttacctaactttgcatggtgcttagtcaaggactcctgcgacctggctcccgaggtcagctggcggcgctgacacacatgcatggcagactatccctggctctatctccctgttcctcgccccctccaccccccacttcctctttaaaaaaaaaaaaaaaaaaaaaaagatacaagaaaaacctttaaaaaaattccatgtttcctaatttgcacgaaattttctaccacaagatgtgccttgccttccgagaataagtattacctttaaacaatatcagcgcacacacatagctgcatgttctgctcgtgtagtttaaaaaaaaaaagacaaaacagtgacatgaaataaaaaataaaaattgaaaagggatgtatttctatttgtaaaaaaaataaaataaaaaataagaaagtgagaatctaaaaaaaaaaaaaaaaaaaaaaaaggaagaaaaaccacgctaaaaatcaagccactgaaaacaattgcccccaggtctacccagcccctggctgtccttggtcctgtctcccctcctgctgtattcaggggtgccccctggtgctcagcctctaccacccccaaccctgctcttgggtacccagaggggtcatttctgaatcccttgcccagaggacagacctccggggcccatcttggccctgggaaagggctctcctctctgattggtccctaggccacgggccggcccccagacaccattcaccgacccactgcaggctgtcctccaaccatggggtggccactccacccgcagccagactccccgctccccacttttcatgcaggctggcatacccctggctcagggtcaaatgctgttccacacccacctcagaggcaccccctctcccctgccccgtgcatccccacccttcttgccaaaggacctcttttcccctatccagagaccaccccaggtggcattctctcccaccttctcctttgtcccccatcccctgtctctgtcttccagctgtgaatatgaagggtatcctgtatgaaacaaaaacaaaacctgatatatgcaatatctgtctgtctgtctgtacccatgggcctggctcagccattggaggcccagccgagggtccggcagggcacagggacagccaggtggcaccgagtcacaggctgtggtccggtggctgagcatgctgttgtcttgtccttgattttattttcttttgttctttttttttttcttttctttttgtttttaactccagcttcctttgctttttacttgaccaaagctaagacaatagccagatggttagtggggcagccaggcagggaggacccagggctgggattctccaaccttaggccattcctgcagccctcaccacctccagcccctccaagcatctcgtgtagggacccacgcagatggtcccattcattcactattgcccccaaccccgggattttgggtggtctccacagccaccatcatacactcatcccgtgttttcttccaaaaagtcacctcagcagcctccccaggcgatacagagggagagcccagaccaccacagctggccacgacattgcccttaagtaatatgcattggccagagagcccgggctggctgtgcacagcattcatgtagctgatttctagctttttttttttttctgccccactcctgagcaaatctgtcttgccaaggaactaggagcaaccggaggcaaagggagtgggtggccccatcactattgggaccatcgcgtccctgcacagcccacacccgggggcccagagtcctgggctggacgccacccttctcaccccgagcttgcctccttggctcacttggcaccttggctgagtacagcaggcaaaagcccataccaggcagcatgttgtggatggtttagttctccccgcctccctgtttcttggaaaagctacagggtccctgtagggcaaaattcccaggcgccttgctgcagacagagtaagacaaaaacaccaggaagcaggattccgtgcccatctctgcagtttgggttcacaaaagggggtgccgtcatccctgggtggaggagggagtgttggttttttgtttttgtttttttaacatgtatgaaactgacatcttctcaaatcttgttccacccccctctggaagcccccatcacccacccctgctatggacaccacacctatgccaggcccccccccccaccccagtctcattctggggtctgcccatgctgtgggaaagaatagggaggcctcccaaatatatgcaaattgtccccattccgtgggggcacctgacaatgacccgggtggagatggggcatggaggagtaggaagacccagccctatttgactggggagaggaggatctggagtccttcatgcccaggtctggaacccaggttctgaccccagggccccaccctgggctggacaatcagatcccaaaggaatgccaaaggggactcggttgggagagccgcttaggggccagacctgggtccccctgcaggtccccaggcagcagacaattccaccttccctgccccaggaccttgagagacagcagcattccaggcacagacagacttggctgcaccccactgtcccttgcaagacaggttctggagccaggagcaactgtccagccctccagaagagacagcaagcagcccccctacccactctggcctccccaatggtactttgacctccagtgtagggctatactatacatatatatatatatatatatatatatatataattttggaatttgtttctcataatacagaatatatagtggctaccttgtatcttggtctggattctctctctgagaccccggattttactttctctttggagggcgctgggacatacatctctcaatccagcttcctccgcatcctcccatcttgccccatttctgccacgtcagacacttcctgagagtctcaccttcaaaatgacaccgctgcccatccattgctcaatggtacagagtgtggggtcagtccaccacccttgacctcccggcagggcaaggtgaggaggcggacccaaagcagtaccagcaggacttgttgccagtgataccaaaacagacttttcccaagcagtgcctcacatgtctgctggtgtggctttgggattctcctgccccacccccccgtccatggcagccccctccccaaggctttgctcacacctgagacaggaaggaggaaggggatccaataggaatatgggccccggaggggaagtcatgcacccccaagccaccaccccccagccttccacgcacatctcctggctggaagagagccctccaaaaaggggacacaggctgccccggcccctcaactgcatccacaccccatcctctcatcttgggtcccagccaggcccccccaaaaccaaagccccctcaagtcctggggtcccagcctgtgcccccagcttcctgcccacccagccctgagcattctcacacagagaaagaacaagcaagggctccagggggacaggatggggcagggcatacagtggggggtgggggggcagctgggaggagggagggacaaaacaaaacattttcctttgggttttttttttctttcttttttctcccctttactctttgggtggtgttgcttttcctttccttttccctttgagatttttttgttgttgtttcctttttgtattttactgatatcaccaggatagtttactctccttctagctttctgcttaccgcacactggataacacacacatacacacccacaaaaatgctcatgaacccaatccggagaaggttccagcaggtcccccaccctcccctcctcctcctacttctcctcttgacagcgaggacaggagggggacaaggggacacctgggcagacccgccggctctccccccaccccaccccgcccctcacatcatactccaatcataaccttgtatattacgcagtcattttggttttcgcggacgcgcctacctaagtaccatttacagaaagtgactctggctgtcattattttgtttatttgttccctatgcaaaaaaaaaatgaaaatgaaaaaagggggattccataaaagattcaataaaagacaaacaaaaaaaaaagaaaaaagaaaaaaatgtataaaaattaaacaagctatgcttcgactctt

SEQ ID NO: 3 - DNA, 8001 bp, Homo sapiens
  source 1..8001: mol_type = unassigned DNA
ggggaccgagcgcgctcgctccggcgccggcctcgcctcctcgcagcagcgccatggatgagttccacccgttcatcgaggccctgctgcctcacgtccgcgccttcgcctacacctggttcaacctgcaggcgcggaagcgcaagtacttcaagaagcacgagaagcggatgtcgaaggacgaggagcgtgcggtcaaggacgagctgctgggcgagaagcccgaggtcaagcagaagtgggcgtcgcggctgctggccaagctgcgcaaggacatccggcccgagtgccgcgaggacttcgtgctgagcatcaccggcaagaaggcgccgggctgcgtgctctccaaccccgaccagaagggcaagatgcggcgcatcgactgtctccggcaggcggacaaggtgtggcggctggacctggtcatggtcatcctgttcaagggcatcccgctggagagcaccgacggcgagcgcctggtcaaggctgcgcagtgcggtcacccggtcctgtgcgtgcagccgcaccacattggcgtggccgtcaaggagctggacctctacctggcctacttcgtgcgtgagcgagatgcagagcaaagcggcagtccccggacagggatgggctctgaccaggaggacagcaagcccatcacgctggacacgaccgacttccaggagagctttgtcacctccggcgtgttcagcgtcactgagctcatccaagtgtcccggacacccgtggtgactggaacaggacccaacttctccctgggggagctgcaggggcacctggcatacgacctgaacccagccagcactggcctcagaagaacgctgcccagcacctcctccagtgggagcaagcggcacaaatcgggctcgatggaggaagacgtggacacgagccctggcggcgattactacacttcgcccagctcgcccacgagtagcagccgcaactggacggaggacatggaaggaggcatctcgtccccggtgaagaagacagagatggacaagtcaccattcaacagcccgtccccccaggactctccccgcctctccagcttcacccagcaccaccggcccgtcatcgccgtgcacagcgggatcgcccggagcccacacccgtcctccgctctgcatttccctacgacgtccatcctaccccagacggcctccacctacttcccccacacggccatccgctacccacctcatctcaacccccaggacccgctcaaagatcttgtctcgctggcctgcgacccagccagccagcaacctggaccgttaaatggaagtggtcagctcaaaatgcccagccactgcctttctgctcagatgctggcacctccgcccccggggctgccacggctggcgctcccccctgccaccaaacccgccaccacctccgagggaggagccacgtcgccgacctcgccttcctactctccgcccgacacgtcccctgcaaaccgttcctttgtgggattaggaccaagggatcctgcgggcatttatcaggcacagtcctggtatctgggatagcaaaggtcttcttccctcgccccttctccatcgtcccaggaatcccagggggcagcacagccggcccccggcccacgttttcggtggaaaattagagtgaacaagaacacccctgccgactcccagcccggccaaaaagacaaaacacatagacgcacacactcaggaggaaaagaaaaaacaaaggcagaagaagaagaagaagaaataaaaacccacccaagcaagaagacaaaaggtaaagacgcaacgtttccaactctcgggacgccaaggccgcaggactggagggccaggccccgccacccccacgggagacccgggacagggcgtcttcctaagttattcatctcctctccgcctgctgctcgggaaggacagacgccggccgcccgcccgcgccccggaggccctggctctgtccggagaccaggtgagcacagcctggagcctgtgcccagggccgacaggcgcgacacccagcaaggccacctctccccgggcccccgcgcctctgccggacacggaccggcccctcagcccccaccgaggacgcagccactggggggaaagggagacacagcggaccccggccgggcagcggagaccgcagaggcgggcagggtggggcaggcgagtggtgtcgcgggggtgcgtggcgcttgcgagccctggccaggggaggaagtgaggcccaggcacctgctgcccctcgagggggccctgcctgccgcggggcctccccacaagcccctcccaaagcgccggccgactcgctgtctcgctggggactctttcagccctcgcgcccgcccgtttgggaggagaagtctctatgcaattggccccggcccctccaccccccacccccggcataggaggcccccccacctcgcccggctcacacccccaaagggagggacccacattgcacacactgtaagaaatgcactttccgaggaaggggatgggggagcccggacacccagagctccccgagttgggggtgcccgtctggagcgcccccgtcagcccctggcggtgggaggtgagagcgagtggtttaagtgcctgattaccaccacccgcccccccctttgtccagctgggacacggaatggccgcgggcctcctccccctcccctccagcctctccaccagcccctccagtcaaccctcatcgccgtgcccccccagagctagagagatggggcccctgcgtggcccgaggggcagagctgggcgtcacttcgcaagcgtcctgccctgccggggcgcgggggtgggctctggggaagccggtgcgccccccacgcctccgctgccagtgccttacattctggagcgacccccctccctggtgcctcccagcgaagggggaccgccgtttgcactttcatcgcctaccccgacgcggggcccagctgcgggacgtgcatcacggctgggcccccagaggagagaggaggccgacgccagcggtccccgctcggaacggggagggttttcggggggttcggcgtcgcaccttggggccccccgcagccgtgtagggggcctcccatctgctaagcgtttttccgttgagccgctccaaaaacactaagctggggacgccaggtgcccccccaccccggctccctggccctatccacacctccacccccaccccaggatcgccatctttaggggaggcctgggagggggtgttaggtgttttagggccaccgagctcaaacacaaggacccctccccggcccacccagcccagccccaactgacctccatgcctagggaaaaactccccccaccactgccccctcccccgacccaggccaaagccagggcaggtctccgggtctcacctgctcctagcctcacccccctgcccccgaaaaccagactctcctcccaaactagcctcaggagcttggcgaacccgctcgctcctaaagagaaagacccaggaccctcccccatcacccccaagagaggttcgccatcctctggcctcgagcccttggtccctccgtccgtctgtcctcggggcccgctcccccggtggcccttggggatcaaagcgtgggccgctctccgggagggcgggcgggggagggggtggtcgggttgtgccattggggtgtccggaagcttctcagccagggtgggggtcgtggagtgggggagggaggccagccgggctccagaggggtcagggcgcgacgagaaccaactctttacctaactttgcatggtgcttagtcaaggactcctgcgacctggctcccgaggtcagctggcggcgctgacacacatgcatggcagactatccctggctctatctccctgttcctcgccccctccaccccccacttcctctttaaaaaaaaaaaaaaaaaaaaaaagatacaagaaaaacctttaaaaaaattccatgtttcctaatttgcacgaaattttctaccacaagatgtgccttgccttccgagaataagtattacctttaaacaatatcagcgcacacacatagctgcatgttctgctcgtgtagtttaaaaaaaaaaagacaaaacagtgacatgaaataaaaaataaaaattgaaaagggatgtatttctatttgtaaaaaaaataaaataaaaaataagaaagtgagaatctaaaaaaaaaaaaaaaaaaaaaaaaggaagaaaaaccacgctaaaaatcaagccactgaaaacaattgcccccaggtctacccagcccctggctgtccttggtcctgtctcccctcctgctgtattcaggggtgccccctggtgctcagcctctaccacccccaaccctgctcttgggtacccagaggggtcatttctgaatcccttgcccagaggacagacctccggggcccatcttggccctgggaaagggctctcctctctgattggtccctaggccacgggccggcccccagacaccattcaccgacccactgcaggctgtcctccaaccatggggtggccactccacccgcagccagactccccgctccccacttttcatgcaggctggcatacccctggctcagggtcaaatgctgttccacacccacctcagaggcaccccctctcccctgccccgtgcatccccacccttcttgccaaaggacctcttttcccctatccagagaccaccccaggtggcattctctcccaccttctcctttgtcccccatcccctgtctctgtcttccagctgtgaatatgaagggtatcctgtatgaaacaaaaacaaaacctgatatatgcaatatctgtctgtctgtctgtacccatgggcctggctcagccattggaggcccagccgagggtccggcagggcacagggacagccaggtggcaccgagtcacaggctgtggtccggtggctgagcatgctgttgtcttgtccttgattttattttcttttgttctttttttttttcttttctttttgtttttaactccagcttcctttgctttttacttgaccaaagctaagacaatagccagatggttagtggggcagccaggcagggaggacccagggctgggattctccaaccttaggccattcctgcagccctcaccacctccagcccctccaagcatctcgtgtagggacccacgcagatggtcccattcattcactattgcccccaaccccgggattttgggtggtctccacagccaccatcatacactcatcccgtgttttcttccaaaaagtcacctcagcagcctccccaggcgatacagagggagagcccagaccaccacagctggccacgacattgcccttaagtaatatgcattggccagagagcccgggctggctgtgcacagcattcatgtagctgatttctagctttttttttttttctgccccactcctgagcaaatctgtcttgccaaggaactaggagcaaccggaggcaaagggagtgggtggccccatcactattgggaccatcgcgtccctgcacagcccacacccgggggcccagagtcctgggctggacgccacccttctcaccccgagcttgcctccttggctcacttggcaccttggctgagtacagcaggcaaaagcccataccaggcagcatgttgtggatggtttagttctccccgcctccctgtttcttggaaaagctacagggtccctgtagggcaaaattcccaggcgccttgctgcagacagagtaagacaaaaacaccaggaagcaggattccgtgcccatctctgcagtttgggttcacaaaagggggtgccgtcatccctgggtggaggagggagtgttggttttttgtttttgtttttttaacatgtatgaaactgacatcttctcaaatcttgttccacccccctctggaagcccccatcacccacccctgctatggacaccacacctatgccaggcccccccccccaccccagtctcattctggggtctgcccatgctgtgggaaagaatagggaggcctcccaaatatatgcaaattgtccccattccgtgggggcacctgacaatgacccgggtggagatggggcatggaggagtaggaagacccagccctatttgactggggagaggaggatctggagtccttcatgcccaggtctggaacccaggttctgaccccagggccccaccctgggctggacaatcagatcccaaaggaatgccaaaggggactcggttgggagagccgcttaggggccagacctgggtccccctgcaggtccccaggcagcagacaattccaccttccctgccccaggaccttgagagacagcagcattccaggcacagacagacttggctgcaccccactgtcccttgcaagacaggttctggagccaggagcaactgtccagccctccagaagagacagcaagcagcccccctacccactctggcctccccaatggtactttgacctccagtgtagggctatactatacatatatatatatatatatatatatatatataattttggaatttgtttctcataatacagaatatatagtggctaccttgtatcttggtctggattctctctctgagaccccggattttactttctctttggagggcgctgggacatacatctctcaatccagcttcctccgcatcctcccatcttgccccatttctgccacgtcagacacttcctgagagtctcaccttcaaaatgacaccgctgcccatccattgctcaatggtacagagtgtggggtcagtccaccacccttgacctcccggcagggcaaggtgaggaggcggacccaaagcagtaccagcaggacttgttgccagtgataccaaaacagacttttcccaagcagtgcctcacatgtctgctggtgtggctttgggattctcctgccccacccccccgtccatggcagccccctccccaaggctttgctcacacctgagacaggaaggaggaaggggatccaataggaatatgggccccggaggggaagtcatgcacccccaagccaccaccccccagccttccacgcacatctcctggctggaagagagccctccaaaaaggggacacaggctgccccggcccctcaactgcatccacaccccatcctctcatcttgggtcccagccaggcccccccaaaaccaaagccccctcaagtcctggggtcccagcctgtgcccccagcttcctgcccacccagccctgagcattctcacacagagaaagaacaagcaagggctccagggggacaggatggggcagggcatacagtggggggtgggggggcagctgggaggagggagggacaaaacaaaacattttcctttgggttttttttttctttcttttttctcccctttactctttgggtggtgttgcttttcctttccttttccctttgagatttttttgttgttgtttcctttttgtattttactgatatcaccaggatagtttactctccttctagctttctgcttaccgcacactggataacacacacatacacacccacaaaaatgctcatgaacccaatccggagaaggttccagcaggtcccccaccctcccctcctcctcctacttctcctcttgacagcgaggacaggagggggacaaggggacacctgggcagacccgccggctctccccccaccccaccccgcccctcacatcatactccaatcataaccttgtatattacgcagtcattttggttttcgcggacgcgcctacctaagtaccatttacagaaagtgactctggctgtcattattttgtttatttgttccctatgcaaaaaaaaaatgaaaatgaaaaaagggggattccataaaagattcaataaaagacaaacaaaaaaaaaagaaaaaagaaaaaaatgtataaaaattaaacaagctatgcttcgactctt

SEQ ID NO: 4 - DNA, 7875 bp, Homo sapiens
  source 1..7875: mol_type = unassigned DNA
agtaagttcagcgcgcccgctccggccggccctgcgcctcccgccgcgcccgggatgtattcgtccccgctctgcctcacccaggatgagttccacccgttcatcgaggccctgctgcctcacgtccgcgccttcgcctacacctggttcaacctgcaggcgcggaagcgcaagtacttcaagaagcacgagaagcggatgtcgaaggacgaggagcgtgcggtcaaggacgagctgctgggcgagaagcccgaggtcaagcagaagtgggcgtcgcggctgctggccaagctgcgcaaggacatccggcccgagtgccgcgaggacttcgtgctgagcatcaccggcaagaaggcgccgggctgcgtgctctccaaccccgaccagaagggcaagatgcggcgcatcgactgtctccggcaggcggacaaggtgtggcggctggacctggtcatggtcatcctgttcaagggcatcccgctggagagcaccgacggcgagcgcctggtcaaggctgcgcagtgcggtcacccggtcctgtgcgtgcagccgcaccacattggcgtggccgtcaaggagctggacctctacctggcctacttcgtgcgtgagcgagatgcagagcaaagcggcagtccccggacagggatgggctctgaccaggaggacagcaagcccatcacgctggacacgaccgacttccaggagagctttgtcacctccggcgtgttcagcgtcactgagctcatccaagtgtcccggacacccgtggtgactggaacaggacccaacttctccctgggggagctgcaggggcacctggcatacgacctgaacccagccagcactggcctcagaagaacgctgcccagcacctcctccagtgggagcaagcggcacaaatcgggctcgatggaggaagacgtggacacgagccctggcggcgattactacacttcgcccagctcgcccacgagtagcagccgcaactggacggaggacatggaaggaggcatctcgtccccggtgaagaagacagagatggacaagtcaccattcaacagcccgtccccccaggactctccccgcctctccagcttcacccagcaccaccggcccgtcatcgccgtgcacagcgggatcgcccggagcccacacccgtcctccgctctgcatttccctacgacgtccatcctaccccagacggcctccacctacttcccccacacggccatccgctacccacctcatctcaacccccaggacccgctcaaagatcttgtctcgctggcctgcgacccagccagccagcaacctggaccgcctactctccgcccgacacgtcccctgcaaaccgttcctttgtgggattaggaccaagggatcctgcgggcatttatcaggcacagtcctggtatctgggatagcaaaggtcttcttccctcgccccttctccatcgtcccaggaatcccagggggcagcacagccggcccccggcccacgttttcggtggaaaattagagtgaacaagaacacccctgccgactcccagcccggccaaaaagacaaaacacatagacgcacacactcaggaggaaaagaaaaaacaaaggcagaagaagaagaagaagaaataaaaacccacccaagcaagaagacaaaaggtaaagacgcaacgtttccaactctcgggacgccaaggccgcaggactggagggccaggccccgccacccccacgggagacccgggacagggcgtcttcctaagttattcatctcctctccgcctgctgctcgggaaggacagacgccggccgcccgcccgcgccccggaggccctggctctgtccggagaccaggtgagcacagcctggagcctgtgcccagggccgacaggcgcgacacccagcaaggccacctctccccgggcccccgcgcctctgccggacacggaccggcccctcagcccccaccgaggacgcagccactggggggaaagggagacacagcggaccccggccgggcagcggagaccgcagaggcgggcagggtggggcaggcgagtggtgtcgcgggggtgcgtggcgcttgcgagccctggccaggggaggaagtgaggcccaggcacctgctgcccctcgagggggccctgcctgccgcggggcctccccacaagcccctcccaaagcgccggccgactcgctgtctcgctggggactctttcagccctcgcgcccgcccgtttgggaggagaagtctctatgcaattggccccggcccctccaccccccacccccggcataggaggcccccccacctcgcccggctcacacccccaaagggagggacccacattgcacacactgtaagaaatgcactttccgaggaaggggatgggggagcccggacacccagagctccccgagttgggggtgcccgtctggagcgcccccgtcagcccctggcggtgggaggtgagagcgagtggtttaagtgcctgattaccaccacccgcccccccctttgtccagctgggacacggaatggccgcgggcctcctccccctcccctccagcctctccaccagcccctccagtcaaccctcatcgccgtgcccccccagagctagagagatggggcccctgcgtggcccgaggggcagagctgggcgtcacttcgcaagcgtcctgccctgccggggcgcgggggtgggctctggggaagccggtgcgccccccacgcctccgctgccagtgccttacattctggagcgacccccctccctggtgcctcccagcgaagggggaccgccgtttgcactttcatcgcctaccccgacgcggggcccagctgcgggacgtgcatcacggctgggcccccagaggagagaggaggccgacgccagcggtccccgctcggaacggggagggttttcggggggttcggcgtcgcaccttggggccccccgcagccgtgtagggggcctcccatctgctaagcgtttttccgttgagccgctccaaaaacactaagctggggacgccaggtgcccccccaccccggctccctggccctatccacacctccacccccaccccaggatcgccatctttaggggaggcctgggagggggtgttaggtgttttagggccaccgagctcaaacacaaggacccctccccggcccacccagcccagccccaactgacctccatgcctagggaaaaactccccccaccactgccccctcccccgacccaggccaaagccagggcaggtctccgggtctcacctgctcctagcctcacccccctgcccccgaaaaccagactctcctcccaaactagcctcaggagcttggcgaacccgctcgctcctaaagagaaagacccaggaccctcccccatcacccccaagagaggttcgccatcctctggcctcgagcccttggtccctccgtccgtctgtcctcggggcccgctcccccggtggcccttggggatcaaagcgtgggccgctctccgggagggcgggcgggggagggggtggtcgggttgtgccattggggtgtccggaagcttctcagccagggtgggggtcgtggagtgggggagggaggccagccgggctccagaggggtcagggcgcgacgagaaccaactctttacctaactttgcatggtgcttagtcaaggactcctgcgacctggctcccgaggtcagctggcggcgctgacacacatgcatggcagactatccctggctctatctccctgttcctcgccccctccaccccccacttcctctttaaaaaaaaaaaaaaaaaaaaaaagatacaagaaaaacctttaaaaaaattccatgtttcctaatttgcacgaaattttctaccacaagatgtgccttgccttccgagaataagtattacctttaaacaatatcagcgcacacacatagctgcatgttctgctcgtgtagtttaaaaaaaaaaagacaaaacagtgacatgaaataaaaaataaaaattgaaaagggatgtatttctatttgtaaaaaaaataaaataaaaaataagaaagtgagaatctaaaaaaaaaaaaaaaaaaaaaaaaggaagaaaaaccacgctaaaaatcaagccactgaaaacaattgcccccaggtctacccagcccctggctgtccttggtcctgtctcccctcctgctgtattcaggggtgccccctggtgctcagcctctaccacccccaaccctgctcttgggtacccagaggggtcatttctgaatcccttgcccagaggacagacctccggggcccatcttggccctgggaaagggctctcctctctgattggtccctaggccacgggccggcccccagacaccattcaccgacccactgcaggctgtcctccaaccatggggtggccactccacccgcagccagactccccgctccccacttttcatgcaggctggcatacccctggctcagggtcaaatgctgttccacacccacctcagaggcaccccctctcccctgccccgtgcatccccacccttcttgccaaaggacctcttttcccctatccagagaccaccccaggtggcattctctcccaccttctcctttgtcccccatcccctgtctctgtcttccagctgtgaatatgaagggtatcctgtatgaaacaaaaacaaaacctgatatatgcaatatctgtctgtctgtctgtacccatgggcctggctcagccattggaggcccagccgagggtccggcagggcacagggacagccaggtggcaccgagtcacaggctgtggtccggtggctgagcatgctgttgtcttgtccttgattttattttcttttgttctttttttttttcttttctttttgtttttaactccagcttcctttgctttttacttgaccaaagctaagacaatagccagatggttagtggggcagccaggcagggaggacccagggctgggattctccaaccttaggccattcctgcagccctcaccacctccagcccctccaagcatctcgtgtagggacccacgcagatggtcccattcattcactattgcccccaaccccgggattttgggtggtctccacagccaccatcatacactcatcccgtgttttcttccaaaaagtcacctcagcagcctccccaggcgatacagagggagagcccagaccaccacagctggccacgacattgcccttaagtaatatgcattggccagagagcccgggctggctgtgcacagcattcatgtagctgatttctagctttttttttttttctgccccactcctgagcaaatctgtcttgccaaggaactaggagcaaccggaggcaaagggagtgggtggccccatcactattgggaccatcgcgtccctgcacagcccacacccgggggcccagagtcctgggctggacgccacccttctcaccccgagcttgcctccttggctcacttggcaccttggctgagtacagcaggcaaaagcccataccaggcagcatgttgtggatggtttagttctccccgcctccctgtttcttggaaaagctacagggtccctgtagggcaaaattcccaggcgccttgctgcagacagagtaagacaaaaacaccaggaagcaggattccgtgcccatctctgcagtttgggttcacaaaagggggtgccgtcatccctgggtggaggagggagtgttggttttttgtttttgtttttttaacatgtatgaaactgacatcttctcaaatcttgttccacccccctctggaagcccccatcacccacccctgctatggacaccacacctatgccaggcccccccccccaccccagtctcattctggggtctgcccatgctgtgggaaagaatagggaggcctcccaaatatatgcaaattgtccccattccgtgggggcacctgacaatgacccgggtggagatggggcatggaggagtaggaagacccagccctatttgactggggagaggaggatctggagtccttcatgcccaggtctggaacccaggttctgaccccagggccccaccctgggctggacaatcagatcccaaaggaatgccaaaggggactcggttgggagagccgcttaggggccagacctgggtccccctgcaggtccccaggcagcagacaattccaccttccctgccccaggaccttgagagacagcagcattccaggcacagacagacttggctgcaccccactgtcccttgcaagacaggttctggagccaggagcaactgtccagccctccagaagagacagcaagcagcccccctacccactctggcctccccaatggtactttgacctccagtgtagggctatactatacatatatatatatatatatatatatatatataattttggaatttgtttctcataatacagaatatatagtggctaccttgtatcttggtctggattctctctctgagaccccggattttactttctctttggagggcgctgggacatacatctctcaatccagcttcctccgcatcctcccatcttgccccatttctgccacgtcagacacttcctgagagtctcaccttcaaaatgacaccgctgcccatccattgctcaatggtacagagtgtggggtcagtccaccacccttgacctcccggcagggcaaggtgaggaggcggacccaaagcagtaccagcaggacttgttgccagtgataccaaaacagacttttcccaagcagtgcctcacatgtctgctggtgtggctttgggattctcctgccccacccccccgtccatggcagccccctccccaaggctttgctcacacctgagacaggaaggaggaaggggatccaataggaatatgggccccggaggggaagtcatgcacccccaagccaccaccccccagccttccacgcacatctcctggctggaagagagccctccaaaaaggggacacaggctgccccggcccctcaactgcatccacaccccatcctctcatcttgggtcccagccaggcccccccaaaaccaaagccccctcaagtcctggggtcccagcctgtgcccccagcttcctgcccacccagccctgagcattctcacacagagaaagaacaagcaagggctccagggggacaggatggggcagggcatacagtggggggtgggggggcagctgggaggagggagggacaaaacaaaacattttcctttgggttttttttttctttcttttttctcccctttactctttgggtggtgttgcttttcctttccttttccctttgagatttttttgttgttgtttcctttttgtattttactgatatcaccaggatagtttactctccttctagctttctgcttaccgcacactggataacacacacatacacacccacaaaaatgctcatgaacccaatccggagaaggttccagcaggtcccccaccctcccctcctcctcctacttctcctcttgacagcgaggacaggagggggacaaggggacacctgggcagacccgccggctctccccccaccccaccccgcccctcacatcatactccaatcataaccttgtatattacgcagtcattttggttttcgcggacgcgcctacctaagtaccatttacagaaagtgactctggctgtcattattttgtttatttgttccctatgcaaaaaaaaaatgaaaatgaaaaaagggggattccataaaagattcaataaaagacaaacaaaaaaaaaagaaaaaagaaaaaaatgtataaaaattaaacaagctatgcttcgactctt

SEQ ID NO: 5 - DNA, 7847 bp, Homo sapiens
  source 1..7847: mol_type = unassigned DNA
ggggaccgagcgcgctcgctccggcgccggcctcgcctcctcgcagcagcgccatggatgagttccacccgttcatcgaggccctgctgcctcacgtccgcgccttcgcctacacctggttcaacctgcaggcgcggaagcgcaagtacttcaagaagcacgagaagcggatgtcgaaggacgaggagcgtgcggtcaaggacgagctgctgggcgagaagcccgaggtcaagcagaagtgggcgtcgcggctgctggccaagctgcgcaaggacatccggcccgagtgccgcgaggacttcgtgctgagcatcaccggcaagaaggcgccgggctgcgtgctctccaaccccgaccagaagggcaagatgcggcgcatcgactgtctccggcaggcggacaaggtgtggcggctggacctggtcatggtcatcctgttcaagggcatcccgctggagagcaccgacggcgagcgcctggtcaaggctgcgcagtgcggtcacccggtcctgtgcgtgcagccgcaccacattggcgtggccgtcaaggagctggacctctacctggcctacttcgtgcgtgagcgagatgcagagcaaagcggcagtccccggacagggatgggctctgaccaggaggacagcaagcccatcacgctggacacgaccgacttccaggagagctttgtcacctccggcgtgttcagcgtcactgagctcatccaagtgtcccggacacccgtggtgactggaacaggacccaacttctccctgggggagctgcaggggcacctggcatacgacctgaacccagccagcactggcctcagaagaacgctgcccagcacctcctccagtgggagcaagcggcacaaatcgggctcgatggaggaagacgtggacacgagccctggcggcgattactacacttcgcccagctcgcccacgagtagcagccgcaactggacggaggacatggaaggaggcatctcgtccccggtgaagaagacagagatggacaagtcaccattcaacagcccgtccccccaggactctccccgcctctccagcttcacccagcaccaccggcccgtcatcgccgtgcacagcgggatcgcccggagcccacacccgtcctccgctctgcatttccctacgacgtccatcctaccccagacggcctccacctacttcccccacacggccatccgctacccacctcatctcaacccccaggacccgctcaaagatcttgtctcgctggcctgcgacccagccagccagcaacctggaccgcctactctccgcccgacacgtcccctgcaaaccgttcctttgtgggattaggaccaagggatcctgcgggcatttatcaggcacagtcctggtatctgggatagcaaaggtcttcttccctcgccccttctccatcgtcccaggaatcccagggggcagcacagccggcccccggcccacgttttcggtggaaaattagagtgaacaagaacacccctgccgactcccagcccggccaaaaagacaaaacacatagacgcacacactcaggaggaaaagaaaaaacaaaggcagaagaagaagaagaagaaataaaaacccacccaagcaagaagacaaaaggtaaagacgcaacgtttccaactctcgggacgccaaggccgcaggactggagggccaggccccgccacccccacgggagacccgggacagggcgtcttcctaagttattcatctcctctccgcctgctgctcgggaaggacagacgccggccgcccgcccgcgccccggaggccctggctctgtccggagaccaggtgagcacagcctggagcctgtgcccagggccgacaggcgcgacacccagcaaggccacctctccccgggcccccgcgcctctgccggacacggaccggcccctcagcccccaccgaggacgcagccactggggggaaagggagacacagcggaccccggccgggcagcggagaccgcagaggcgggcagggtggggcaggcgagtggtgtcgcgggggtgcgtggcgcttgcgagccctggccaggggaggaagtgaggcccaggcacctgctgcccctcgagggggccctgcctgccgcggggcctccccacaagcccctcccaaagcgccggccgactcgctgtctcgctggggactctttcagccctcgcgcccgcccgtttgggaggagaagtctctatgcaattggccccggcccctccaccccccacccccggcataggaggcccccccacctcgcccggctcacacccccaaagggagggacccacattgcacacactgtaagaaatgcactttccgaggaaggggatgggggagcccggacacccagagctccccgagttgggggtgcccgtctggagcgcccccgtcagcccctggcggtgggaggtgagagcgagtggtttaagtgcctgattaccaccacccgcccccccctttgtccagctgggacacggaatggccgcgggcctcctccccctcccctccagcctctccaccagcccctccagtcaaccctcatcgccgtgcccccccagagctagagagatggggcccctgcgtggcccgaggggcagagctgggcgtcacttcgcaagcgtcctgccctgccggggcgcgggggtgggctctggggaagccggtgcgccccccacgcctccgctgccagtgccttacattctggagcgacccccctccctggtgcctcccagcgaagggggaccgccgtttgcactttcatcgcctaccccgacgcggggcccagctgcgggacgtgcatcacggctgggcccccagaggagagaggaggccgacgccagcggtccccgctcggaacggggagggttttcggggggttcggcgtcgcaccttggggccccccgcagccgtgtagggggcctcccatctgctaagcgtttttccgttgagccgctccaaaaacactaagctggggacgccaggtgcccccccaccccggctccctggccctatccacacctccacccccaccccaggatcgccatctttaggggaggcctgggagggggtgttaggtgttttagggccaccgagctcaaacacaaggacccctccccggcccacccagcccagccccaactgacctccatgcctagggaaaaactccccccaccactgccccctcccccgacccaggccaaagccagggcaggtctccgggtctcacctgctcctagcctcacccccctgcccccgaaaaccagactctcctcccaaactagcctcaggagcttggcgaacccgctcgctcctaaagagaaagacccaggaccctcccccatcacccccaagagaggttcgccatcctctggcctcgagcccttggtccctccgtccgtctgtcctcggggcccgctcccccggtggcccttggggatcaaagcgtgggccgctctccgggagggcgggcgggggagggggtggtcgggttgtgccattggggtgtccggaagcttctcagccagggtgggggtcgtggagtgggggagggaggccagccgggctccagaggggtcagggcgcgacgagaaccaactctttacctaactttgcatggtgcttagtcaaggactcctgcgacctggctcccgaggtcagctggcggcgctgacacacatgcatggcagactatccctggctctatctccctgttcctcgccccctccaccccccacttcctctttaaaaaaaaaaaaaaaaaaaaaaagatacaagaaaaacctttaaaaaaattccatgtttcctaatttgcacgaaattttctaccacaagatgtgccttgccttccgagaataagtattacctttaaacaatatcagcgcacacacatagctgcatgttctgctcgtgtagtttaaaaaaaaaaagacaaaacagtgacatgaaataaaaaataaaaattgaaaagggatgtatttctatttgtaaaaaaaataaaataaaaaataagaaagtgagaatctaaaaaaaaaaaaaaaaaaaaaaaaggaagaaaaaccacgctaaaaatcaagccactgaaaacaattgcccccaggtctacccagcccctggctgtccttggtcctgtctcccctcctgctgtattcaggggtgccccctggtgctcagcctctaccacccccaaccctgctcttgggtacccagaggggtcatttctgaatcccttgcccagaggacagacctccggggcccatcttggccctgggaaagggctctcctctctgattggtccctaggccacgggccggcccccagacaccattcaccgacccactgcaggctgtcctccaaccatggggtggccactccacccgcagccagactccccgctccccacttttcatgcaggctggcatacccctggctcagggtcaaatgctgttccacacccacctcagaggcaccccctctcccctgccccgtgcatccccacccttcttgccaaaggacctcttttcccctatccagagaccaccccaggtggcattctctcccaccttctcctttgtcccccatcccctgtctctgtcttccagctgtgaatatgaagggtatcctgtatgaaacaaaaacaaaacctgatatatgcaatatctgtctgtctgtctgtacccatgggcctggctcagccattggaggcccagccgagggtccggcagggcacagggacagccaggtggcaccgagtcacaggctgtggtccggtggctgagcatgctgttgtcttgtccttgattttattttcttttgttctttttttttttcttttctttttgtttttaactccagcttcctttgctttttacttgaccaaagctaagacaatagccagatggttagtggggcagccaggcagggaggacccagggctgggattctccaaccttaggccattcctgcagccctcaccacctccagcccctccaagcatctcgtgtagggacccacgcagatggtcccattcattcactattgcccccaaccccgggattttgggtggtctccacagccaccatcatacactcatcccgtgttttcttccaaaaagtcacctcagcagcctccccaggcgatacagagggagagcccagaccaccacagctggccacgacattgcccttaagtaatatgcattggccagagagcccgggctggctgtgcacagcattcatgtagctgatttctagctttttttttttttctgccccactcctgagcaaatctgtcttgccaaggaactaggagcaaccggaggcaaagggagtgggtggccccatcactattgggaccatcgcgtccctgcacagcccacacccgggggcccagagtcctgggctggacgccacccttctcaccccgagcttgcctccttggctcacttggcaccttggctgagtacagcaggcaaaagcccataccaggcagcatgttgtggatggtttagttctccccgcctccctgtttcttggaaaagctacagggtccctgtagggcaaaattcccaggcgccttgctgcagacagagtaagacaaaaacaccaggaagcaggattccgtgcccatctctgcagtttgggttcacaaaagggggtgccgtcatccctgggtggaggagggagtgttggttttttgtttttgtttttttaacatgtatgaaactgacatcttctcaaatcttgttccacccccctctggaagcccccatcacccacccctgctatggacaccacacctatgccaggcccccccccccaccccagtctcattctggggtctgcccatgctgtgggaaagaatagggaggcctcccaaatatatgcaaattgtccccattccgtgggggcacctgacaatgacccgggtggagatggggcatggaggagtaggaagacccagccctatttgactggggagaggaggatctggagtccttcatgcccaggtctggaacccaggttctgaccccagggccccaccctgggctggacaatcagatcccaaaggaatgccaaaggggactcggttgggagagccgcttaggggccagacctgggtccccctgcaggtccccaggcagcagacaattccaccttccctgccccaggaccttgagagacagcagcattccaggcacagacagacttggctgcaccccactgtcccttgcaagacaggttctggagccaggagcaactgtccagccctccagaagagacagcaagcagcccccctacccactctggcctccccaatggtactttgacctccagtgtagggctatactatacatatatatatatatatatatatatatatataattttggaatttgtttctcataatacagaatatatagtggctaccttgtatcttggtctggattctctctctgagaccccggattttactttctctttggagggcgctgggacatacatctctcaatccagcttcctccgcatcctcccatcttgccccatttctgccacgtcagacacttcctgagagtctcaccttcaaaatgacaccgctgcccatccattgctcaatggtacagagtgtggggtcagtccaccacccttgacctcccggcagggcaaggtgaggaggcggacccaaagcagtaccagcaggacttgttgccagtgataccaaaacagacttttcccaagcagtgcctcacatgtctgctggtgtggctttgggattctcctgccccacccccccgtccatggcagccccctccccaaggctttgctcacacctgagacaggaaggaggaaggggatccaataggaatatgggccccggaggggaagtcatgcacccccaagccaccaccccccagccttccacgcacatctcctggctggaagagagccctccaaaaaggggacacaggctgccccggcccctcaactgcatccacaccccatcctctcatcttgggtcccagccaggcccccccaaaaccaaagccccctcaagtcctggggtcccagcctgtgcccccagcttcctgcccacccagccctgagcattctcacacagagaaagaacaagcaagggctccagggggacaggatggggcagggcatacagtggggggtgggggggcagctgggaggagggagggacaaaacaaaacattttcctttgggttttttttttctttcttttttctcccctttactctttgggtggtgttgcttttcctttccttttccctttgagatttttttgttgttgtttcctttttgtattttactgatatcaccaggatagtttactctccttctagctttctgcttaccgcacactggataacacacacatacacacccacaaaaatgctcatgaacccaatccggagaaggttccagcaggtcccccaccctcccctcctcctcctacttctcctcttgacagcgaggacaggagggggacaaggggacacctgggcagacccgccggctctccccccaccccaccccgcccctcacatcatactccaatcataaccttgtatattacgcagtcattttggttttcgcggacgcgcctacctaagtaccatttacagaaagtgactctggctgtcattattttgtttatttgttccctatgcaaaaaaaaaatgaaaatgaaaaaagggggattccataaaagattcaataaaagacaaacaaaaaaaaaagaaaaaagaaaaaaatgtataaaaattaaacaagctatgcttcgactctt

SEQ ID NO: 6 - DNA, 7789 bp, Homo sapiens
  source 1..7789: mol_type = unassigned DNA
agtaagttcagcgcgcccgctccggccggccctgcgcctcccgccgcgcccgggatgtattcgtccccgctctgcctcacccaggatgagttccacccgttcatcgaggccctgctgcctcacgtccgcgccttcgcctacacctggttcaacctgcaggcgcggaagcgcaagtacttcaagaagcacgagaagcggatgtcgaaggacgaggagcgtgcggtcaaggacgagctgctgggcgagaagcccgaggtcaagcagaagtgggcgtcgcggctgctggccaagctgcgcaaggacatccggcccgagtgccgcgaggacttcgtgctgagcatcaccggcaagaaggcgccgggctgcgtgctctccaaccccgaccagaagggcaagatgcggcgcatcgactgtctccggcaggcggacaaggtgtggcggctggacctggtcatggtcatcctgttcaagggcatcccgctggagagcaccgacggcgagcgcctggtcaaggctgcgcagtgcggtcacccggtcctgtgcgtgcagccgcaccacattggcgtggccgtcaaggagctggacctctacctggcctacttcgtgcgtgagcgagatgcagagcaaagcggcagtccccggacagggatgggctctgaccaggaggacagcaagcccatcacgctggacacgaccgacttccaggagagctttgtcacctccggcgtgttcagcgtcactgagctcatccaagtgtcccggacacccgtggtgactggaacaggacccaacttctccctgggggagctgcaggggcacctggcatacgacctgaacccagccagcactggcctcagaagaacgctgcccagcacctcctccagtgggagcaagcggcacaaatcgggctcgatggaggaagacgtggacacgagccctggcggcgattactacacttcgcccagctcgcccacgagtagcagccgcaactggacggaggacatggaaggaggcatctcgtccccggtgaagaagacagagatggacaagtcaccattcaacagcccgtccccccaggactctccccgcctctccagcttcacccagcaccaccggcccgtcatcgccgtgcacagcgggatcgcccggagcccacacccgtcctccgctctgcatttccctacgacgtccatcctaccccagacggcctccacctacttcccccacacggccatccgctacccacctcatctcaacccccaggacccgctcaaagatcttgtctcgctggcctgcgacccagccagccagcaacctggaccgtcctggtatctgggatagcaaaggtcttcttccctcgccccttctccatcgtcccaggaatcccagggggcagcacagccggcccccggcccacgttttcggtggaaaattagagtgaacaagaacacccctgccgactcccagcccggccaaaaagacaaaacacatagacgcacacactcaggaggaaaagaaaaaacaaaggcagaagaagaagaagaagaaataaaaacccacccaagcaagaagacaaaaggtaaagacgcaacgtttccaactctcgggacgccaaggccgcaggactggagggccaggccccgccacccccacgggagacccgggacagggcgtcttcctaagttattcatctcctctccgcctgctgctcgggaaggacagacgccggccgcccgcccgcgccccggaggccctggctctgtccggagaccaggtgagcacagcctggagcctgtgcccagggccgacaggcgcgacacccagcaaggccacctctccccgggcccccgcgcctctgccggacacggaccggcccctcagcccccaccgaggacgcagccactggggggaaagggagacacagcggaccccggccgggcagcggagaccgcagaggcgggcagggtggggcaggcgagtggtgtcgcgggggtgcgtggcgcttgcgagccctggccaggggaggaagtgaggcccaggcacctgctgcccctcgagggggccctgcctgccgcggggcctccccacaagcccctcccaaagcgccggccgactcgctgtctcgctggggactctttcagccctcgcgcccgcccgtttgggaggagaagtctctatgcaattggccccggcccctccaccccccacccccggcataggaggcccccccacctcgcccggctcacacccccaaagggagggacccacattgcacacactgtaagaaatgcactttccgaggaaggggatgggggagcccggacacccagagctccccgagttgggggtgcccgtctggagcgcccccgtcagcccctggcggtgggaggtgagagcgagtggtttaagtgcctgattaccaccacccgcccccccctttgtccagctgggacacggaatggccgcgggcctcctccccctcccctccagcctctccaccagcccctccagtcaaccctcatcgccgtgcccccccagagctagagagatggggcccctgcgtggcccgaggggcagagctgggcgtcacttcgcaagcgtcctgccctgccggggcgcgggggtgggctctggggaagccggtgcgccccccacgcctccgctgccagtgccttacattctggagcgacccccctccctggtgcctcccagcgaagggggaccgccgtttgcactttcatcgcctaccccgacgcggggcccagctgcgggacgtgcatcacggctgggcccccagaggagagaggaggccgacgccagcggtccccgctcggaacggggagggttttcggggggttcggcgtcgcaccttggggccccccgcagccgtgtagggggcctcccatctgctaagcgtttttccgttgagccgctccaaaaacactaagctggggacgccaggtgcccccccaccccggctccctggccctatccacacctccacccccaccccaggatcgccatctttaggggaggcctgggagggggtgttaggtgttttagggccaccgagctcaaacacaaggacccctccccggcccacccagcccagccccaactgacctccatgcctagggaaaaactccccccaccactgccccctcccccgacccaggccaaagccagggcaggtctccgggtctcacctgctcctagcctcacccccctgcccccgaaaaccagactctcctcccaaactagcctcaggagcttggcgaacccgctcgctcctaaagagaaagacccaggaccctcccccatcacccccaagagaggttcgccatcctctggcctcgagcccttggtccctccgtccgtctgtcctcggggcccgctcccccggtggcccttggggatcaaagcgtgggccgctctccgggagggcgggcgggggagggggtggtcgggttgtgccattggggtgtccggaagcttctcagccagggtgggggtcgtggagtgggggagggaggccagccgggctccagaggggtcagggcgcgacgagaaccaactctttacctaactttgcatggtgcttagtcaaggactcctgcgacctggctcccgaggtcagctggcggcgctgacacacatgcatggcagactatccctggctctatctccctgttcctcgccccctccaccccccacttcctctttaaaaaaaaaaaaaaaaaaaaaaagatacaagaaaaacctttaaaaaaattccatgtttcctaatttgcacgaaattttctaccacaagatgtgccttgccttccgagaataagtattacctttaaacaatatcagcgcacacacatagctgcatgttctgctcgtgtagtttaaaaaaaaaaagacaaaacagtgacatgaaataaaaaataaaaattgaaaagggatgtatttctatttgtaaaaaaaataaaataaaaaataagaaagtgagaatctaaaaaaaaaaaaaaaaaaaaaaaaggaagaaaaaccacgctaaaaatcaagccactgaaaacaattgcccccaggtctacccagcccctggctgtccttggtcctgtctcccctcctgctgtattcaggggtgccccctggtgctcagcctctaccacccccaaccctgctcttgggtacccagaggggtcatttctgaatcccttgcccagaggacagacctccggggcccatcttggccctgggaaagggctctcctctctgattggtccctaggccacgggccggcccccagacaccattcaccgacccactgcaggctgtcctccaaccatggggtggccactccacccgcagccagactccccgctccccacttttcatgcaggctggcatacccctggctcagggtcaaatgctgttccacacccacctcagaggcaccccctctcccctgccccgtgcatccccacccttcttgccaaaggacctcttttcccctatccagagaccaccccaggtggcattctctcccaccttctcctttgtcccccatcccctgtctctgtcttccagctgtgaatatgaagggtatcctgtatgaaacaaaaacaaaacctgatatatgcaatatctgtctgtctgtctgtacccatgggcctggctcagccattggaggcccagccgagggtccggcagggcacagggacagccaggtggcaccgagtcacaggctgtggtccggtggctgagcatgctgttgtcttgtccttgattttattttcttttgttctttttttttttcttttctttttgtttttaactccagcttcctttgctttttacttgaccaaagctaagacaatagccagatggttagtggggcagccaggcagggaggacccagggctgggattctccaaccttaggccattcctgcagccctcaccacctccagcccctccaagcatctcgtgtagggacccacgcagatggtcccattcattcactattgcccccaaccccgggattttgggtggtctccacagccaccatcatacactcatcccgtgttttcttccaaaaagtcacctcagcagcctccccaggcgatacagagggagagcccagaccaccacagctggccacgacattgcccttaagtaatatgcattggccagagagcccgggctggctgtgcacagcattcatgtagctgatttctagctttttttttttttctgccccactcctgagcaaatctgtcttgccaaggaactaggagcaaccggaggcaaagggagtgggtggccccatcactattgggaccatcgcgtccctgcacagcccacacccgggggcccagagtcctgggctggacgccacccttctcaccccgagcttgcctccttggctcacttggcaccttggctgagtacagcaggcaaaagcccataccaggcagcatgttgtggatggtttagttctccccgcctccctgtttcttggaaaagctacagggtccctgtagggcaaaattcccaggcgccttgctgcagacagagtaagacaaaaacaccaggaagcaggattccgtgcccatctctgcagtttgggttcacaaaagggggtgccgtcatccctgggtggaggagggagtgttggttttttgtttttgtttttttaacatgtatgaaactgacatcttctcaaatcttgttccacccccctctggaagcccccatcacccacccctgctatggacaccacacctatgccaggcccccccccccaccccagtctcattctggggtctgcccatgctgtgggaaagaatagggaggcctcccaaatatatgcaaattgtccccattccgtgggggcacctgacaatgacccgggtggagatggggcatggaggagtaggaagacccagccctatttgactggggagaggaggatctggagtccttcatgcccaggtctggaacccaggttctgaccccagggccccaccctgggctggacaatcagatcccaaaggaatgccaaaggggactcggttgggagagccgcttaggggccagacctgggtccccctgcaggtccccaggcagcagacaattccaccttccctgccccaggaccttgagagacagcagcattccaggcacagacagacttggctgcaccccactgtcccttgcaagacaggttctggagccaggagcaactgtccagccctccagaagagacagcaagcagcccccctacccactctggcctccccaatggtactttgacctccagtgtagggctatactatacatatatatatatatatatatatatatatataattttggaatttgtttctcataatacagaatatatagtggctaccttgtatcttggtctggattctctctctgagaccccggattttactttctctttggagggcgctgggacatacatctctcaatccagcttcctccgcatcctcccatcttgccccatttctgccacgtcagacacttcctgagagtctcaccttcaaaatgacaccgctgcccatccattgctcaatggtacagagtgtggggtcagtccaccacccttgacctcccggcagggcaaggtgaggaggcggacccaaagcagtaccagcaggacttgttgccagtgataccaaaacagacttttcccaagcagtgcctcacatgtctgctggtgtggctttgggattctcctgccccacccccccgtccatggcagccccctccccaaggctttgctcacacctgagacaggaaggaggaaggggatccaataggaatatgggccccggaggggaagtcatgcacccccaagccaccaccccccagccttccacgcacatctcctggctggaagagagccctccaaaaaggggacacaggctgccccggcccctcaactgcatccacaccccatcctctcatcttgggtcccagccaggcccccccaaaaccaaagccccctcaagtcctggggtcccagcctgtgcccccagcttcctgcccacccagccctgagcattctcacacagagaaagaacaagcaagggctccagggggacaggatggggcagggcatacagtggggggtgggggggcagctgggaggagggagggacaaaacaaaacattttcctttgggttttttttttctttcttttttctcccctttactctttgggtggtgttgcttttcctttccttttccctttgagatttttttgttgttgtttcctttttgtattttactgatatcaccaggatagtttactctccttctagctttctgcttaccgcacactggataacacacacatacacacccacaaaaatgctcatgaacccaatccggagaaggttccagcaggtcccccaccctcccctcctcctcctacttctcctcttgacagcgaggacaggagggggacaaggggacacctgggcagacccgccggctctccccccaccccaccccgcccctcacatcatactccaatcataaccttgtatattacgcagtcattttggttttcgcggacgcgcctacctaagtaccatttacagaaagtgactctggctgtcattattttgtttatttgttccctatgcaaaaaaaaaatgaaaatgaaaaaagggggattccataaaagattcaataaaagacaaacaaaaaaaaaagaaaaaagaaaaaaatgtataaaaattaaacaagctatgcttcgactctt

SEQ ID NO: 7 - DNA, 3084 bp, Homo sapiens
  source 1..3084: mol_type = unassigned DNA
gccaggtgctcccgccttccaccctccgccctcctccctcccctgggccctgctccctgccctcctgggcagccagggcagccaggacggcaccaagggagctgccccatggacagggccccacagagacagcaccgagcctcacgggagctgctggctgcaaagaagacccacacctcacaaattgaagtgatcccttgcaaaatctgtggggacaagtcgtctgggatccactacggggttatcacctgtgaggggtgcaagggcttcttccgccggagccagcgctgtaacgcggcctactcctgcacccgtcagcagaactgccccatcgaccgcaccagccgaaaccgatgccagcactgccgcctgcagaaatgcctggcgctgggcatgtcccgagatgctgtcaagttcggccgcatgtccaagaagcagagggacagcctgcatgcagaagtgcagaaacagctgcagcagcggcaacagcagcaacaggaaccagtggtcaagacccctccagcaggggcccaaggagcagataccctcacctacaccttggggctcccagacgggcagctgcccctgggctcctcgcctgacctgcctgaggcttctgcctgtccccctggcctcctgaaagcctcaggctctgggccctcatattccaacaacttggccaaggcagggctcaatggggcctcatgccaccttgaatacagccctgagcggggcaaggctgagggcagagagagcttctatagcacaggcagccagctgacccctgaccgatgtggacttcgttttgaggaacacaggcatcctgggcttggggaactgggacagggcccagacagctacggcagccccagtttccgcagcacaccggaggcaccctatgcctccctgacagagatagagcacctggtgcagagcgtctgcaagtcctacagggagacatgccagctgcggctggaggacctgctgcggcagcgctccaacatcttctcccgggaggaagtgactggctaccagaggaagtccatgtgggagatgtgggaacggtgtgcccaccacctcaccgaggccattcagtacgtggtggagttcgccaagaggctctcaggctttatggagctctgccagaatgaccagattgtgcttctcaaagcaggagcaatggaagtggtgctggttaggatgtgccgggcctacaatgctgacaaccgcacggtcttttttgaaggcaaatacggtggcatggagctgttccgagccttgggctgcagcgagctcatcagctccatctttgacttctcccactccctaagtgccttgcacttttccgaggatgagattgccctctacacagcccttgttctcatcaatgcccatcggccagggctccaagagaaaaggaaagtagaacagctgcagtacaatctggagctggcctttcatcatcatctctgcaagactcatcgccaaagcatcctggcaaagctgccacccaaggggaagcttcggagcctgtgtagccagcatgtggaaaggctgcagatcttccagcacctccaccccatcgtggtccaagccgctttccctccactctacaaggagctcttcagcactgaaaccgagtcacctgtggggctgtccaagtgacctggaagagggactccttgcctctccctatggcctgctggcccacctccctggaccccgttccaccctcacccttttcctttcccatgaaccctggagggtggtccccaccagctctttggaagtgagcagatgctgcggctggctttctgtcagcaggccggcctggcagtgggacaatcgccagagggtggggctggcagaacaccatctccagcctcagctttgacctgtctcatttcccatattccttcacacccagcttctggaaggcatggggtggctgggatttaaggacttctgggggaccaagacatcctcaagaaaacaggggcatccagggctccctggatgaatagaatgcaattcattcagaagctcagaagctaagaataagcctttgaaatacctcattgcatttccctttgggcttcggcttggggagatggatcaagctcagagactggcagtgagagcccagaaggacctgtataaaatgaatctggagctttacattttctgcctctgccttcctcccagctcagcaaggaagtatttgggcaccctaccctttacctggggtctaaccaaaaatggatgggatgaggatgagaggctggagataattgttttatgggatttgggtgtgggactagggtacaatgaaggccaagagcatctcagacatagagttaaaactcaaacctcttatgtgcactttaaagatagactttaggggctggcacaaatctgatcagagacacatatccatacacaggtgaaacacatacagactcaacagcaatcatgcagttccagagacacatgaacctgacacaatctctcttatccttgaggccacagcttggaggagcctagaggcctcaggggaaagtcccaatcctgagggaccctcccaaacatttccatggtgctccagtccactgatcttgggtctggggtgatccaaataccaccccagctccagctgtcttctaccactagaagacccaagagaagcagaagtcgctcgcactggtcagtcggaaggcaagatcagatcctggaggactttcctggcctgcccgccagccctgctcttgttgtggagaaggaagcagatgtgatcacatcaccccgtcattgggcaccgctgactccagcatggaggacaccagggagcagggcctgggcctgtttccccagctgtgatcttgcccagaacctctcttggcttcataaacagctgtgaaccctcccctgagggattaacagcaatgatgggcagtcgtggagttgggggggttgggggtgggattgtgtcctctaaggggacgggttcatctgagtaaacataaaccccaacttgtgccattctttataaaatgattttaaaggcaaaaaaaaaaaaaaaaaaaa

SEQ ID NO: 8 - DNA, 1277 bp, Homo sapiens
  source 1..1277: mol_type = unassigned DNA
tttttttcaatgaacatgacttctggagtcaaggttgttgggccattccccccgttccactcactgggaatataaatagcacccacagcgcagaacacagagccagagagctggaagtgagagcagatccctaaccatgagcaccagccaaccaggggcctgcccatgccagggagctgcaagccgccccgccattctctacgcacttctgagctccagcctcaaggctgtcccccgaccccgtagccgctgcctatgtaggcagcaccggcccgtccagctatgtgcacctcatcgcacctgccgggaggccttggatgttctggccaagacagtggccttcctcaggaacctgccatccttctggcagctgcctccccaggaccagcggcggctgctgcagggttgctggggccccctcttcctgcttgggttggcccaagatgctgtgacctttgaggtggctgaggccccggtgcccagcatactcaagaagattctgctggaggagcccagcagcagtggaggcagtggccaactgccagacagaccccagccctccctggctgcggtgcagtggcttcaatgctgtctggagtccttctggagcctggagcttagccccaaggaatatgcctgcctgaaagggaccatcctcttcaaccccgatgtgccaggcctccaagccgcctcccacattgggcacctgcagcaggaggctcactgggtgctgtgtgaagtcctggaaccctggtgcccagcagcccaaggccgcctgacccgtgtcctcctcacggcctccaccctcaagtccattccgaccagcctgcttggggacctcttctttcgccctatcattggagatgttgacatcgctggccttcttggggacatgcttttgctcaggtgacctgttccagcccaggcagagatcaggtgggcagaggctggcagtgctgattcagcctggccatccccagaggtgacccaatgctcctggagggggcaagcctgtatagacagcacttggctccttaggaacagctcttcactcagccacaccccacattggacttccttggtttggacacagtgttccagctgcctgggaggcttttggtggtccccacagcctctgggccaagactcctgtcccttcttgggatgagaatgaaagcttaggctgcttattggaccagaagtcctatcgactttatacagaactgaattaagttattgatttttgtaataaaaggtatgaaacacttggaaaaaaa

SEQ ID NO: 9 - DNA, 6320 bp, Homo sapiens
  source 1..6320: mol_type = unassigned DNA
aaacacatccacacactctctctgcctagttcacacactgagccactcgcacatgcgagcacattccttccttccttctcactctctcggcccttgacttctacaagcccatggaacatttctggaaagacgttcttgatccagcagggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggtaatagacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcattccccctataggaatacaaggggcacacagggaaggcagatcccctagttggcaagactattttaacttgatacactgcagattcagatgtgctgaaagctctgcctctggctttccggtcatgggttccagttaattcatgcctcccatggacctatggagagcagcaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgaggcacagccagacttgctcagggtggccctgccacaggctgcagctacctaggaacattccttgcagaccccgcattgccctttgggggtgccctgggatccctggggtagtccagctcttcttcatttcccagcgtggccctggttggaagaagcagctgtcacagctgctgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtgagctgctcgggcttctcttggtatgtcttgtttggaaaagtggatttcattcatttctgattgtccagttaagtgatcaccaaaggactgagaatctgggagggcaaaaaaaaaaaaaaagtttttatgtgcacttaaatttggggacaattttatgtatctgtgttaaggatatgtttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatactgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccagggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaatagagtcatacagtagctcaaaaggcaaccataattctctttggtgcaggtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcttttttttgcatccaattgtgcctgaacttttaaaatatgtaaatgctgccatgttccaaacccatcgtcagtgtgtgtgtttagagctgtgcaccctagaaacaacatattgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaactaaa

SEQ ID NO: 10 - DNA, 1182 bp, Homo sapiens
  source 1..1182: mol_type = unassigned DNA
attgtgtcagaggaagcaaccatgcaggtgctaaccaagcgttaccccaagaactgcctgctgaccgtcatggaccggtatgcagccgaggtgcacaacatggagcaggtggtgatgatccccagccttctgcgggacgtgcagctgagtgggcctgggggccaggcccaggctgaggcccctgatctctacacctacttcaccatgctcaaggccatctgtgtggatgtggaccatgggctgctgccgcgggaggagtggcaggccaaggtggcaggcagcgaagagaatggaaccgcagagacagaggaagtcgaggacgagagtgcctcaggagagctggacctggaagcccagttccacctgcacttctccagcctccatcacatcctcatgcacctcaccgagaaagcccaggaggtgacaaggaaataccaggaaatgacgggacaagtttggtagaccttggacactagggaagatcccttcacatgatagaagacagactctttgatgaggtcggcggagcagttcactagccaatgatgagagcagaaaggcctagacctgcagccagaagtgaaggcggctcagttctccgggatgcttctctacctcctgagcaccaattcctggattccagtcactggctcacctttagaatgtctgttgctattcactgctcccctcgctcctcttaacagcttggggaggtgaccagtggttcaggagggactagacaattacctgtccagtgtggtatggtaggaagagtgtaggtgttggcacgtgaccaaaattcacatccctcctcatggcagtcattcagtatgtgtacttgtacaagttatttaacccattggagcctaaattccctcatctataaaatggggataatattatctacctcacaagcttatgaaaactaaacatgatgaatcaaaagcacttggcatgtgagggctattaaaatagcctgattttttttttctccccctctccccaatgtatttgctctggcccttgctttttaccctccagagctaagaggtagcagagtctcttgggatgagtgattcaccctcttacttggcgaccactgatgagatcaacaacaggtgaactataaacctattatttattgcagaactaataaaaaatccaaagccttgtatttgtaaa

SEQ ID NO: 11 - DNA, 3445 bp, Homo sapiens
  source 1..3445: mol_type = unassigned DNA
actaggactggaagatcgggctgtgtctaggccgctgtccgcgaaatccgagacgttttttcagcttggctaggaccgacttcgctgccggtttgagctttctctgcactcgggggtctcctgccgtcctcgaccggtggcgtaacttgggaagagattctgagcagagcactggttcagattctgaggtcctcactgagcggacttcctgctccttcagtactcacactgacctggcctctggtgctgcaggccctgtgcctgctgccatgtcttccatggaggagattcaggtggagctgcaatgtgctgacctctggaagcggttccatgatattggaactgaaatgatcatcaccaaagcaggcaggaggatgtttcctgccatgagagtgaaaatcactggcctagatccacatcagcagtactacatagcaatggacattgtgcctgtggacaataaaagatacagatatgtgtatcatagctccaagtggatggtggctggcaatgctgattcccctgtgcccccaagagtttatatacaccctgattctctagcttctggagacacctggatgagacaggtggtcagttttgacaaactcaagcttaccaacaatgagttggatgatcaaggacatatcattctgcactctatgcacaaataccagcctcgagttcatgtgattcgcaaagacttcagcagtgacctttcacccactaagcctgttcctgttggggatggggtgaaaacgttcaactttcctgagactgtgttcaccacagttacggcctatcagaatcagcagattaccagattaaaaattgaccgaaacccttttgctaaaggattcagagattctgggagaaacagaactggacttgaagccatcatggagacatatgcattctggagacctcctgtgcgcacactcaccttcgaagacttcaccaccatgcagaagcagcaaggaggcagcacaggcacttccccaaccacctccagcactgggacaccatccccttcggcttcttctcatcttttatctccatcctgttctcctccaacttttcatctggcccccaacactttcaatgtgggctgccgagaaagccagctgtgtaatctaaacctctctgattatccaccatgtgcccgaagcaacatggctgccttgcagagctacccagggctgagtgacagtggctacaacaggcttcagagtggcaccacttcagccactcagccctctgaaaccttcatgcctcagaggactccatccctgatctcaggaataccaactcctccctcgttgcctggcaacagcaagatggaagcctacggtggccagctggggtcctttcccacttcccagtttcagtatgtcatgcaggcaggcaatgctgcctccagctcctcatcaccacacatgttcgggggcagccacatgcagcagagctcctacaatgccttctcccttcacaacccttacaacctgtatggatacaatttccccacttcccctaggctagctgcaagcccggaaaaactgagcgcctctcaaagcactttactctgttcttctccttccaacggggcctttggagagaggcagtacctgccgtcagggatggagcacagcatgcacatgattagcccttcacccaataaccaacaggcaaccaacacttgtgatggccggcagtatggggcagttccaggctcctcctcccagatgtccgtgcacatggtttaaaggccagtccaaacaccacggagcatttggcaatcaaggccccagagtctccgtggtcagatcctcctctttgggagtccagtgtctttgaaaaacaggaaccgtgttttttttttttttttttttctggccgaagacatatacccaagaacaagagatacctttaagccagtgaaggatacttgcgatagaatcatccgcaactcagtggccattcttctgccttcccagaccttagttttataaagcattgtctgttccagagtggcctttgaagagactgaataatcacttcgtcataatgttaagggagatgctagtgtgtggcagccatgaaaagttacacatacacacccacatacagacagacctacctatacatacgtgcacacacacatacatattcatacacaattcatacacatgcaatcatacatgcacactgactctgaactgggtgaactctgtggagggaggcccagaatgggtgctttcaccaagaatttgtctgtgtacaactctagatggagtgggccagcagtagctgccagtctttctcccctgcagcttcctctgcttctggaatgaaccatgtatcctggagaccctcccaatggatgagagtggaaagacatcagtacaactggacttggcttccggaaaaagattgcttttgaactttggctctcttcacttgtatgctatcattgatattcccagtggtgcccgtggaaagagggagaaagagaagctgaacaggagaaagacaaacagaaagaatagagaacaggaacgaggtggagagcaagactgacagagaaagtgtgagcaatgatgagaattttaattcaccaaggagacgtgtttttggtttgtccccccaaaccccgcccgccccactacaggttatggaaagaatcatggcattactgaggagtaaacctctctggcacactgagcatggtcagggcattggtcagagggacagagcaaggaatgcatcctgagcccacagctttgaccactgtgatccagaagagaggtgcactacgtgggaagtgctgattccacagcatgcagcctggtaggggaaggaaaataaaagggtgtgaagaaggaatagttttataatctcggaagatgataccaagagcagaggcaacaaatagaggcctggcctccaggtgccggatccagacacctgacctagaatgcctgcccgctatccctgtggcaggaaatatcccctcatgtcccagggaattgcagatgggtcttctatacccttctacctgcccttagatctccatttttatcaaatagtacattgcattttgaagttttgggttttgtccttcatctttccctttcccttcaaatcttttaatggtaagaaagcaagtgaagcttggtgcaagctaaaatttttaaatggtgtggaaatgcaaataataccaagtaaaataatacagatattattaaagtttctggttttgaggtgttgtagataaatgtatttatgtgcctagtggggaatccaatattatgaatatgaaaaagggggcaataaaagggtatgtaaaatatgtatgaagaaaaggtgtacaaaaatttgcccttatgcacggaactctgtttctaagtgccaagcacagaaagccgctaaataaaatctttgcaattgt

SEQ ID NO: 12 - DNA, 5607 bp, Homo sapiens
  source 1..5607: mol_type = unassigned DNA
actcttgtcagggccgcggcacatgggcggccggatgcgctgagcccggcgctgcggggccgcggagcgctggggagcagcggccgccggcgcggggaggggggtggggtgggacggcgcaccgcctccggtgctggcactaggggctggggtcggcgcggtgtcttctgcccttctgcagccgtcgacatttttttttctttctttttttcaattttgaacattttgcaaaacgaggggttcgaggcaggtgagagcatcctgcacgtcgccggggagcccgcgggcacttggcgcgctctcctgggaccgtctgcactggaaacccgaaagtttttttttaatatatatttttatgcagatgtatttataaagatataagtaatttttttcttcccttttctccaccgccttgagagcgagtacttttggcaaaggacggaggaaaagctcagcaacattttagggggcggttgtttctttcttatttctttttttaaggggaaaaaatttgagtgcatcgcgatggagaaaatgtcccgaccgctccccctgaatcccacctttatcccgcctccctacggcgtgctcaggtccctgctggagaacccgctgaagctcccccttcaccacgaagacgcatttagtaaagataaagacaaggaaaagaagctggatgatgagagtaacagcccgacggtcccccagtcggcattcctggggcctaccttatgggacaaaacccttccctatgacggagatactttccagttggaatacatggacctggaggagtttttgtcagaaaatggcattccccccagcccatctcagcatgaccacagccctcaccctcctgggctgcagccagcttcctcggctgccccctcggtcatggacctcagcagccgggcctctgcaccccttcaccctggcatcccatctccgaactgtatgcagagccccatcagaccaggtcagctgttgccagcaaaccgcaatacaccaagtcccattgatcctgacaccatccaggtcccagtgggttatgagccagacccagcagatcttgccctttccagcatccctggccaggaaatgtttgaccctcgcaaacgcaagttctctgaggaagaactgaagccacagcccatgatcaagaaagctcgcaaagtcttcatccctgatgacctgaaggatgacaagtactgggcaaggcgcagaaagaacaacatggcagccaagcgctcccgcgacgcccggaggctgaaagagaaccagatcgccatccgggcctcgttcctggagaaggagaactcggccctccgccaggaggtggctgacttgaggaaggagctgggcaaatgcaagaacatacttgccaagtatgaggccaggcacgggcccctgtaggatggcatttttgcaggctggctttggaatagatggacagtttgtttcctgtctgatagcaccacacgcaaaccaacctttctgacatcagcactttaccagaggcataaacacaactgactcccattttggtgtgcatctgtgtgtgtgtgcgtgtatatgtgcttgtgctcatgtgtgtggtcagcggtatgtgcgtgtgcgtgttcctttgctcttgccattttaaggtagccctctcatcgtcttttagttccaacaaagaaaggtgccatgtctttactagactgaggagccctctcgcgggtctcccatcccctccctccttcactcctgcctcctcagctttgcttcatgttcgagcttacctactcttccaggactctctgcttggattcactaaaaagggccctggtaaaatagtggatctcagtttttaagagtacaagctcttgtttctgtttagtccgtaagttaccatgctaatgaggtgcacacaataacttagcactactccgcagctctagtcctttataagttgctttcctcttactttcagttttggtgataatcgtcttcaaattaaagtgctgtttagatttattagatcccatatttacttactgctatctactaagtttccttttaattctaccaaccccagataagtaagagtactattaatagaacacagagtgtgtttttgcactgtctgtacctaaagcaataatcctattgtacgctagagcatgctgcctgagtattactagtggacgtaggatattttccctacctaagaatttcactgtcttttaaaaaacaaaaagtaaagtaatgcatttgagcatggccagactattccctaggacaaggaagcagagggaaatgggaggtctaaggatgaggggttaatttatcagtacatgagccaaaaactgcgtcttggattagcctttgacattgatgtgttcggttttgttgttccccttccctcacaccctgcctcgcccccacttttctagttaactttttccatatccctcttgacattcaaaacagttacttaagattcagttttcccactttttggtaatatatatatttttgtgaattatactttgttgtttttaaaaagaaaatcagttgattaagttaataagttgatgttttctaaggccctttttcctagtggtgtcatttttgaatgcctcataaattaatgattctgaagcttatgtttcttattctctgtttgcttttgaacgtatgtgctcttataaagtggacttctgaaaaatgaatgtaaaagacactggtgtatctcagaaggggatggtgttgtcacaaactgtggttaatccaatcaatttaaatgtttactatagaccaaaaggagagattattaaatcgtttaatgtttatacagagtaattataggaagttcttttttgtacagtatttttcagatataaatactgacaatgtattttggaagacatatattatatatagaaaagaggagaggaaaactattccatgttttaaaattatatagcaaagatatatattcaccaatgttgtacagagaagaagtgcttgggggtttttgaagtctttaatattttaagccctatcactgacacatcagcatgttttctgctttaaattaaaattttatgacagtatcgaggcttgtgatgacgaatcctgctctaaaatacacaaggagctttcttgtttcttattaggcctcagaaagaagtcagttaacgtcacccaaaagcacaaaatggattttagtcaaatatttattggatgatacagtgttttttaggaaaagcatctgccacaaaaatgttcacttcgaaattctgagttcctggaatggcacgttgctgccagtgccccagacagttcttttctaccctgcgggcccgcacgttttatgaggttgatatcggtgctatgtgtttggtttataatttgatagatgtttgactttaaagatgattgttcttttgtttcattaagttgtaaaatgtcaagaaattctgctgttacgacaaagaaacattttacgctagattaaaatatcctttcatcaatgggattttctagtttcctgccttcagagtatctaatcctttaatgatctggtggtctcctcgtcaatccatcagcaatgcttctctcatagtgtcatagacttgggaaacccaaccagtaggatatttctacaaggtgttcattttgtcacaagctgtagataacagcaagagatgggggtgtattggaattgcaatacattgttcaggtgaataataaaatcaaaaacttttgcaatcttaagcagagataaataaaagatagcaatatgagacacaggtggacgtagagttggcctttttacaggcaaagaggcgaattgtagaattgttagatggcaatagtcattaaaaacatagaaaaatgatgtctttaagtggagaattgtggaaggattgtaacatggaccatccaaatttatggccgtatcaaatggtagctgaaaaaactatatttgagcactggtctctcttggaattagatgtttatatcaaatgagcatctcaaatgttttctgcagaaaaaaataaaaagattctaataaaatgtattctcttgtgtgccaggagaggtttcagaaacctacctcgtcttacaaatttaaacactttggagtctgtacaggtgccttatatgtaggtcattgtcacgatacacacacacgaacactccctctggactggctgcctctccatccagggcagttaactagcaaacaaggcagatctgcttcatggagcgggaggccatggcttgactctgagtgatttgggtcaaccggagtcagacgcatgtctgcacgctgcagctattatgagagtccctttgtcatttttcaccttttcatcctaagcatctttcagagattaattatttggccattaacaatgaatccaaatcatatcatactgacatcatctagacatgatttggaaggaacagcttaggacctcctgatgaggtcacattgttgtttcttttaactagacttggcaaagaaaggcaaaaattgaccagcctatctttctgctggtgctgccttaaggaggtagtttgttgaggggagggctgtagatcattacttctttctcttcaggaagtggccactttgaaccattcaaataccacattaggcaagactgtgataggccttttgtcttcaaatacaacaggcctccactgacccatccctcaaagcagaaggaccctttgaggagagtacagatgggattccacagtggggtgggtggaatggaaacctgtactagaccacccagaggttccttctaacccactggtttggtggggaactcacagtaattccaaatgtacaatcagatgtctagggtctgttttcggaagaagcaagaattatcagtggcaccctccccactgcccccagtgtaaaacaatagacattctgtgaaatgcaaagctattctttggtttttctagtagtttatctcattttaccctattcttcctttaaggaaaactcaatctttatcacagtcaattagagcgatcccaaggcatgggaccaggcctgcttgcctatgtgtgatggcaattggagatctggatttagcactggggtctcagcaccctgcaggtgtctgagactaagtgatctgccctccaggtggcgatcaccttctgctcctaggtacccccactggcaaggccaaggtctcctccacgttttttctgcaattaataatgtcatttaaaaaatgagcaaagccttatccgaatcggatatagcaactaaagtcaatacattttgcaggaggctaagtgtaagagtgtgtgtgtgtgtgtgtgcgtgcatgtgtgtgtgtgtgtatgtgtgtgaataagtcgacataaagtctttaattttgagcaccttaccaaacataacaataatccattatccttttggcaacaccacaaagatcgcatctgttaaacaggtacaagttgacatgaggttagtttaattgtacaccatgatattggtggtatttatgctgttaagtccaaacctttatctgtctgttattcttaatgttgaataaactttgaattttttcctttcaaaaaaaa

SEQ ID NO: 13 - DNA, 5658 bp, Homo sapiens
  source 1..5658: mol_type = unassigned DNA
agatgacactctgagcgctccgggaacggacagcccggcggcttcccgaagccggcggcgcagctgcccggggcgagggggagaaagggagagagggagggggagggcgggcgaagcgggagagccagagactcctcggcgctgagcgcggcggcggcccgggcagccccacgcccctgcctcgcgcgccgcccgcgccatgaagcacatcccggtcctcgaggacgggccgtggaagaccgtgtgcgtgaaggagctgaacggccttaagaagctcaagcggaaaggcaaggagccggcgcggcgcgcgaacggctataaaactttccgactggacttggaagcgcccgagccccgcgccgtagccaccaacgggctgcgggacaggacccatcggctgcagccggtcccggtaccggtgccggtgccagtcccagtggcgccggccgttcccccaagagggggcacggacacagccggggagcgcgggggctctcgggcgcccgaggtctccgacgcgcggaaacgctgcttcgccctaggcgcagtggggccaggactccccacgccgccgccgccgccgcctcctgcgccccagagccaggcacctgggggcccagaggcacagcctttccgggagccgggtctgcgtcctcgcatcttgctgtgcgcaccgcccgcgcgccccgcgccgtcagcacccccagcaccgccagcgcccccggagtccactgtgcgccctgcgcccccgacgcgccccggggaaagttcctactcgtcaatttcacacgtaatttacaataaccaccaggattcctccgcgtcgcctaggaaacgaccgggcgaagcgactgccgcctcctccgagatcaaagccctgcagcagacccggaggctcctggcgaacgccagggagcggacgcgggtgcacaccatcagcgcagccttcgaggcgctcaggaagcaggtgccgtgctactcatatgggcagaagctgtccaaactggccatcctgaggatcgcctgtaactacatcctgtccctggcgcggctggctgaccttgactacagtgccgaccacagcaacctcagcttctccgagtgtgtgcagcgctgcacccgcaccctgcaggccgagggacgtgccaagaagcgcaaggagtgactggctgcaggcaagaccaaggccaccactgtgggccctccttccagtcaggcctgaggacaaggtgagctcgctgagtccagcctcgtggtcttctccaagatgccgccagatgcccagcctacagcctctcagggtcggatcggagcacgcctgcctccctctcccctccgccctcacccagccaatccgaggctgcttcgcactttgccctctgcctggtggggaggggagagctcagcccccgactcactcagaccccaaggcccactgtccagctgcagaaattcgttgccaaagattggacagagacaccgaaggaaatggggtggtgaaaccccacagcgaaaagccacaccgttgctctgtgacttttgctcctcctgttgcctgagccccatctcaagccaaagatgagtcagtggttctgctaggaactcatggaatggatgggcatttgatgacccctgggggtcatcttggccctctgacctggtgctctctctccactgggccttgtgctggctgagtgcaagacaagccttaggggctgtgagagggaggctggggtgcctgggcggggctgggagtgggacctgagatccctgcccactctctccccttcattggctgcccaggccactggccccagttctcagtgtcccttgggtccaggctccttgggccctaagcatcaccagaagggagtaagcagggagagaagcaatattactccctcccctacaccagggacttgccccagggcagctacctatgggtctttgcttccccagccagcctctcctcactgtgacccacccccatgggcccccgtcccaggcagccagcaccatgggcaggccctgccatggacagaaaaagagtttttctcttgttcagcctgcacgtggcctgaggaaggagtagaggctgggttggctggagccgtcctactgggcaagatggcgccccacttggagggcggtggtctgttacagggtgtgcaggggcagagaaggaagggaccaggggactgggccagtatgtggaggatggggcctgcgtgttcaaagccaaggcccgccccttccttgtgctcaaatggccaaagctgttcacgtctgtgctcaaccatctgcttcaaattgaagtaaaagccccaaaatgtcaagaaaatacttgtgttgagtggactctgtgggtgaccaggactttggccggtcatcagctggggagtgtgagggagggggttggtttctacctacaggttgagagcccttcaggatcaggcgctgtccgagtgagagtgtgtgtgtctgtgtgtggaagggggtggagggcggttcccacagtagtctcagcctggactagtgaccaggaggcctggtcaggaacacatgaggagccctctctgtccgcactgcactcaatctgtaccatggatttatgagataggggcccctattattaaccccgtttcacagatggggtaactgaggcctcaagtagacagggtcagtcggtgacagagccagtcatcgaatcaggatgggctcacttcaaatcctgtgctctcaaaccttttccagccccatcaccagtcccagcccaaagtctcttgtgtggccttgtcacattgcttcacctcagcgggcctaaggtagggacaataaaggcccattgggactgggggaaggggtgataagataaaaaataggagagcactgtcaaggcagaagggacagggctggccaaggaaagggggataggaggggaccggaggctgcagccatacaggacacagtttgtcccttggtttcaccagtgtcactttctcgtctctgctgctcagactcctgggctgggctggggctggctgcagggagccccccttgcagtagcgtttctcaggctggccctttaccaaggaccacagtgtccatgctgtcttggatccctaggctggcacagaaacaggggacccaggtggccctgagcactcctcagagcaaaggtgctctggaagcagactggacagagtgggcatggaatggggccaggagggtctgttaggaaggttcagccaccctgtgaagctggcacagataacagcactgctctgttgtccctcggagcctctgagtaaccctgatggcacttcctaaggcagcaggacatgtggactgaccagcatcaaactgttgacatagaagaccatttctattaccaaagggagtgtaccccattctgctgccaagggagcaaacccatggccttaccacccagaaagagcccatcctccacctcccatccccctcctgcatacatacttcattacatgtttccctttcattctgaagcatcattgatgaccagctgcctgtcagacactaagataggcagtgggaatgaagagatggatcttgtgtcatgcatggcatcacggagctctgggttctgtacggagggtgggacagacaggtagacaagcaaataattatgattatagcagatgactaaggtgttgtcgggagcttcaggaaaggaagaactaactcttggggaggttctcaggaaggatttccctggaaagtagccatgggacttgcgtcttaaatggtgagtaaaagctttctgagcaggggagtaggaaaagggctttctatgcagaggagcactcagcgctggcaggaaattggaatcacccaaggagattattaaatattaaatattgatatgaagtattgatgcccaatttcatctccagaaattctgatgtattggtctagggtgttgcctggtcattgggatttttacaagctcctcaagtgatcttaatgtgcaggcaaggttgaagccgctggtctaagtggggtctggtctacgataagaaagtgactttgagccatcgatttgggagacaggctctgggtggatgtgtgtgtgtgcacacatatgtatgtatgtggatgactaaaagtgcatgctctcctctcctttcccagcttcctctccagcacagcaacttgtgttcgtatgcacacacatgcatactctctctcatgggcacatgcatacccacacacacactcgtgtacatttccagaaaatggaattacatttcagatagattcagattccaacggcagtcttctaaacacttttatgcaagcagccattcaaggagaccctcagcaaaatataaatgacgaggagctgccctcatggggccctgtgaaagcactttgcagtccagccttgggtttgtggtcacagagtcacctgtggatgtttgtagcacactctccttgtcttgtctgctctgggtcaccaggcacaggccataaagggatgagggggccctctccagggacccgcaagatcttcctgggtatgtctgcatgaagccccacgtgtgcacacccatcttcatgtgtgtgtgtgccagcctcctgctctctgcagaacaaaaccagaaggaatggctctgggagttggagatctcagctcacaggccaagctttgcaagactctccaaagactgcccacagactgtgctgcttcctgggtctggcctgagactatcccagaagagagggttaaattctggaggtgaggttttgagcaagtgttcatccccccacactatgctccttcctgtctccatggccacatccttcaaggctctgtgctgttctctttttttctggatttctccacctccaccaagttcccctttctcacagctagtggaggcatgagtaggcaggtcccaggggctgggaactgggtagcattgccatgtgcagggactgtgttgggagctgcaggtacagagctcctctgtgctcaagagcttgccggtgagcctggacggaggcataggtgcagctaattaggataagacaggggccgcgctgtggtcagccgtgggaagccggcgaggggactggagttggggctacacttgcctccctcctatgctgcttcctgagccacgaagtggtcattgccagcatcccaggcaacaaacagcaagactcagacatctccaaggaaaccctttgagtggatctgtaccgttgttctcgtcttgctctcttgctgccctgccaccttcacagctgctttctgtttcctggttccaggaagacagcggggcacagggtccctgctttgtgaggagcagctggcttctccctttgcccccaggttttgccctcccacatgtctcccttctggtgacccggaccccagacaaactatgcctgcctccctgaagccaggcatcctgaggaacttgatagacaaacaatgacagtgttttccagaactgtgggtacgtgtctaatctcagatggtactatgaattcctggagatcaaagtttggatctaattcaacccctgatcctcgaaacggctttcttgcaaagtgtatatattggtttctttgctgaatgaatgaataaaacatggaaaatgtggtaattca

SEQ ID NO: 14 - DNA, 4446 bp, Homo sapiens
  source 1..4446: mol_type = unassigned DNA
ttcttaaccctttccagctttcccaccctctttggctttagccatggccttctgatctgtgtttctcaggggacctgcaggccccagatatagccccatgctgtcctcctaccccagagcacactgttcaggctacttccactggtactgaaatccagtatttcacttactctttttctttccaatatcctcatgacattcaatatttcacttactctaggtcctccctgcctaaggcccaagtcaactttctgtccagtgggatttgtaatccaatacctcctagccctagcagaatcccatgtggataatcagaaatgtgactggaaaaaggacagagctctatggctgtgggtcccagtccccactgctggcagtaagtccccagcagtgagctgtgtaagcaccttacattctgcgcttggttgaaaacagcaaggcaagcatccacttgagaaatgtcaacccctaggaaatcccagcctcaagtctttctcatcccttgggaagtgcaaattggatagagaagaaaccaattaaaaacaaaacaaacaaatcatacttagatattctggcttttctcaccagggctggattaaagcatgtacttcaaaataataacaacttaagtcaataaataaatgtaaggaagtccaaatgttcacctgaagacaactgtggtcattttttggcaatcccaggttctcttttctacctgtttgctcaatcgtggtctccctctccctctcttgttggggcccatgcccctgctttactgttgccagaggcttgtacttgtttgccttttaggtaggagcagttacttccactcccctcacctgccataaagcatctttataaacaaagcaagtagaagaaacacatcctggtatccaccacattcggcttttgttgattctgttcacttgggagcacctgctgctagggaataagaaggttgaggctgaagagtgaggactcttcagctcccctctggcaggacccgggagaggaaagagccctcagctggtccatcctccccactcctggtcagccttctgttctgagatcaaagtggtggggtcacattctcgagaactgtgctcagccccctcatctcacaccctttccctctccctgtgtgcctgcccccctcttacataaccatgctggtgattggcaccgtcataaatcaatactttgctcactttcacatcaagtaacactatccagggaggtggtttcaacaaaggaggaagtataaggagatctaggttcaaattaatgttgcccctagtggtaaaggacagagaccctcagactgatgaaatgcactcagaattacttagacaaagcggatatttgccactctcttccccttttcctgtgtttttgtagtgaagagacctgaaagaaaaaagtagggagaacataatgagaacaaatacggtaatctcttcatttgctagttcaagtgctggacttgggacttaggaggggcaatggagccgcttagtgcctacatctgacttggactgaaatataggtgagagacaagattgtctcatatccggggaaatcataacctatgactaggacgggaagaggaagcactgcctttacttcagtgggaatctcggcctcagcctgcaagccaagtgttcacagtgagaaaagcaagagaataagctaatactcctgtcctgaacaaggcagcggctccttggtaaagctactccttgatcgatcctttgcaccggattgttcaaagtggaccccaggggagaagtcggagcaaagaacttaccaccaagcagtccaagaggcccagaagcaaacctggaggtgagacccaaagaaagctggaaccatgctgactttgtacactgtgaggacacagagtctgttcctggaaagcccagtgtcaacgcagatgaggaagtcggaggtccccaaatctgccgtgtatgtggggacaaggccactggctatcacttcaatgtcatgacatgtgaaggatgcaagggctttttcaggagggccatgaaacgcaacgcccggctgaggtgccccttccggaagggcgcctgcgagatcacccggaagacccggcgacagtgccaggcctgccgcctgcgcaagtgcctggagagcggcatgaagaaggagatgatcatgtccgacgaggccgtggaggagaggcgggccttgatcaagcggaagaaaagtgaacggacagggactcagccactgggagtgcaggggctgacagaggagcagcggatgatgatcagggagctgatggacgctcagatgaaaacctttgacactaccttctcccatttcaagaatttccggctgccaggggtgcttagcagtggctgcgagttgccagagtctctgcaggccccatcgagggaagaagctgccaagtggagccaggtccggaaagatctgtgctctttgaaggtctctctgcagctgcggggggaggatggcagtgtctggaactacaaacccccagccgacagtggcgggaaagagatcttctccctgctgccccacatggctgacatgtcaacctacatgttcaaaggcatcatcagctttgccaaagtcatctcctacttcagggacttgcccatcgaggaccagatctccctgctgaagggggccgctttcgagctgtgtcaactgagattcaacacagtgttcaacgcggagactggaacctgggagtgtggccggctgtcctactgcttggaagacactgcaggtggcttccagcaacttctactggagcccatgctgaaattccactacatgctgaagaagctgcagctgcatgaggaggagtatgtgctgatgcaggccatctccctcttctccccagaccgcccaggtgtgctgcagcaccgcgtggtggaccagctgcaggagcaattcgccattactctgaagtcctacattgaatgcaatcggccccagcctgctcataggttcttgttcctgaagatcatggctatgctcaccgagctccgcagcatcaatgctcagcacacccagcggctgctgcgcatccaggacatacacccctttgctacgcccctcatgcaggagttgttcggcatcacaggtagctgagcggctgcccttgggtgacacctccgagaggcagccagacccagagccctctgagccgccactcccgggccaagacagatggacactgccaagagccgacaatgccctgctggcctgtctccctagggaattcctgctatgacagctggctagcattcctcaggaaggacatgggtgccccccacccccagttcagtctgtagggagtgaagccacagactcttacgtggagagtgcactgacctgtaggtcaggaccatcagagaggcaaggttgccctttccttttaaaaggccctgtggtctggggagaaatccctcagatcccactaaagtgtcaaggtgtggaagggaccaagcgaccaaggatgggccatctggggtctatgcccacatacccacgtttgttcgcttcctgagtcttttcattgctacctctaatagtcctgtctcccacttcccactcgttcccctcctcttccgagctgctttgtgggctccaggcctgtactcatcggcaggcgcatgagtatctgtgggagtcctctagagagatgagaagccaggaggcctgcaccaaatgtcagaagcttggcatgacctcattccggccacatcattctgtgtctctgcatccatttgaacacattattaagcaccgataataggtagcctgctgtggggtatacagcattgactcagatatagatcctgagctcacagagtttatagttaaaaaaacaaacagaaacacaaacaatttggatcaaaaggagaaatgataagtgacaaaagcagcacaaggaatttccctgtgtggatgctgagctgtgatggcgggcactgggtacccaagtgaaggttcccgaggacatgagtctgtaggagcaagggcacaaactgcagctgtgagtgcgtgtgtgtgatttggtgtaggtaggtctgtttgccacttgatggggcctgggtttgttcctggggctggaatgctgggtatgctctgtgacaaggctacgctgacaatcagttaaacacaccggagaagaaccatttacatgcaccttatatttctgtgtacacatctattctcaaagctaaagggtatgaaagtgcctgccttgtttatagccacttgtgagtaaaaatttttttgcattttcacaaattatactttatataaggcattccacacctaagaactagttttgggaaatgtagccctgggtttaatgtcaaatcaaggcaaaaggaattaaataatgtacttttggctaaaaaaaaaaaaaaaaaaaaaaaaaaaaaa

SEQ ID NO: 15 - DNA, 6855 bp, Homo sapiens
  source 1..6855: mol_type = unassigned DNA
ggccggagggcgcccgaggggccccgggccgcggcgctcagggcccgggcggccggcggcggccccggggctggggggagtccagcccggatattgagtgcagccattgagaaaagccaaactcttgtgtgtgcgcgtctcgatagcccccaagatggccgccaatgtgggatcgatgtttcaatattggaagcgatttgatctacggcgactccagaaggagcttaattccgtcgcttctgagctgtctgcacggcaggaggagagtgaacattctcataaacatttaattgaactccgccgggaatttaagaaaaatgtacctgaggaaatcagagagatggtggctcctgtattaaaaagcttccaagccgaggtggtggcccttagtaagagaagtcaggaggcggaggctgcttttctgagtgtttacaagcaattaattgaagcaccagaccccgtgcctgtgtttgaggcggcacgcagcctagacgacagactgcagccccccagctttgaccccagtgggcagccccggcgagacctccacacttcgtggaagaggaaccccgagctcctcagccccaaagagcagagagaggggacgtcgcctgccgggcccacgctgaccgagggaagccgcctcccaggcattcccgggaaagccctcctgacagaaaccttgctgcagagaaatgaggcggaaaaacaaaagggccttcaagaagtacagatcactttggcggccagactgggggaggcagaggagaaaatcaaagtcctacattcagcgctaaaggctacgcaggcagagctgctagagctgcggcggaagtacgacgaggaggcagcatccaaggcagatgaagtcggcctgatcatgaccaacctggagaaagctaatcagcgagctgaggctgcccagcgggaggtggaaagtctccgggaacagctggcctctgtcaacagctccatccgcctggcttgctgctctccccaggggcccagtggggataaggtgaacttcactctgtgctcgggccctcggctggaggccgcgctggcctccaaggacagggagatcctgcggctgctgaaggacgtgcagcacctccagagctcactgcaggagctggaggaggcatccgccaaccagatcgccgacctggagcggcagctcacggccaagtccgaggccatagaaaagctggaagagaagctccaggcccagtctgactatgaggaaattaaaacggagctgagcatcctgaaagccatgaagctggcctccagcacctgcagcctcccccagggcatggccaagcctgaagactcactgcttattgcaaaggaggccttcttccccacgcagaaattccttctggagaagcccagcctcctggccagccctgaggaagacccatcagaggacgattccatcaaggattcactgggcacggagcagtcctacccctcccctcagcagctcccacctccaccagggccagaagaccccctgtctcccagccccgggcagcccctgctgggccccagcttggggcctgacggcactcggactttctcgctgtcccccttccccagcctggcatcaggggagagactgatgatgcccccagccgccttcaagggagaggcgggcggcctgctggtgttccccccagccttctatggcgccaagccccccacagcccctgccaccccggcccctggccctgagccactgggcggtcctgagcccgcggatggtggtgggggcggagcggcggggcccggggcagaggaggagcagctggacacggcagagatcgccttccaggtgaaggagcagctgctgaaacacaacatcgggcagcgggtgtttgggcattacgtgctggggctgtcgcagggctcggtcagcgagatcctagcccggcccaagccctggcgcaagctcacggtgaagggcaaggagcccttcatcaagatgaagcagttcctgtcggatgagcagaatgtactggcgctcaggaccatccaagtgcggcagcgaggcagcatcaccccgagaatccgcacgcctgagacaggctcagacgacgccatcaagagcattctagagcaggccaagaaggagatcgagtcgcagaagggcggcgagcccaagacctcggtggccccgctgagcatcgccaacggcacgacccccgccagcacctcggaggacgccatcaagagcatcctggagcaggcacgccgtgagatgcaggcgcaacagcaggcgctgctggagatggaggtggcgcccaggggccgctcggtgcccccctcgcccccggagcggccatcactggccaccgcgagccagaacggggccccggccttggtgaagcaggaggagggcagcgggggccccgcgcaggcgccgctcccggtcctgtcccccgccgccttcgtgcagagcatcatccgcaaggtcaagtccgagatcggcgacgccggctacttcgaccaccactgggcctccgaccgcggcctgctcagccgcccctacgcctccgtgtcgccctcgctgtcctcctcctcctcctctggctactctggccagcccaacggccgcgcctggccccgcggggacgaggcccctgtgccccccgaggacgaggcggcggcaggggcggaggacgaaccccccaggacgggcgagctcaaggctgagggcgcgacggccgaggcgggcgcgcggctgccctactacccggcctacgtgccgcgcaccctgaagcccaccgtgccgccgctgacccccgagcagtacgagctgtacatgtaccgtgaggtagacacgctggagctcacccgccaggtcaaggagaagctggccaagaacggcatctgccagaggatcttcggggagaaggtgctgggcctgtcacagggcagcgtgagcgacatgctgtcccggccgaagccatggagcaagctgacgcagaaggggcgggagcccttcatccgcatgcagctgtggctctctgaccagctcggccaggcagtgggccagcagcctggtgcctcccaggccagtcccacagaaccaaggtcctcaccatccccaccccccagccccacagagcctgagaagagctcccaggagccgttgagcctgtccctggagagcagcaaggagaaccagcagccagagggccgctccagctcctcgttgagcgggaagatgtactcaggcagccaggccccagggggcatccaggagatcgtggccatgtcccccgagctggacacgtactccatcaccaagagggtgaaggaggtcctcacagacaacaatctagggcagcggctgtttggggaaagcatcctgggtctgacacagggctccgtgtctgacctgctgtcccggcccaaaccctggcacaagctgagcctgaaggggcgggagccttttgtccgcatgcagctgtggctcaatgacccccataacgtggagaagctgagggatatgaagaagctggagaagaaagcctacctgaaacgtcgctatggcctcatcagcaccggctcagacagtgagtccccggccacccgctcagagtgccccagcccctgcctgcagccccaggacctgagcctcctgcagatcaagaagccccgggtggtgctggcacccgaggagaaggaggcactgcggaaggcctatcagctggaaccctacccctcgcagcagaccatcgagctcctctccttccagctcaacctcaagaccaacaccgtcatcaactggttccacaactacaggtcccggatgcgccgggagatgttggtggaggggacccaggatgagccagaccttgatccaagcgggggtcctggaatcctaccgccaggccactcccacccagaccccaccccgcagagccctgactctgagactgaggaccagaagccaaccgtgaaggaactggagcttcaggagggccctgaggagaacagcacacccctgaccacccaggacaaggcccaagtgaggatcaagcaggaacagatggaggaggatgctgaggaagaggcaggcagccagccccaggactcaggggagctggacaaaggccaaggtccccccaaagaggagcatcccgaccctccgggtaatgatggactcccaaaagtggctcccgggcccctccttccaggtggatccaccccagactgtccctcacttcatccccaacaggagagtgaggccggggagcgacttcacccggaccctttaagttttaagtcagcctcagagtcctcacgctgcagcctggaggtgtcactgaactcgccctcggccgcctcctcaccaggcctcatgatgtctgtgtcacctgtcccctcctcctcagctcccatctccccatccccacctggcgccccccctgccaaagtgccgagtgccagccccactgctgacatggctggagccttgcaccccagtgccaaggtgaaccccaacttgcagcggcggcatgagaagatggccaatctgaacaacatcatttaccgagtagagcgggctgccaatcgggaggaggccctggagtgggagttctgaaggcagggtgagggggcaagggacataccctggtaactaccttccttctcgcacttactctcctcaacaggatggggtaagggagggaggaactcaaccatcaaaatgtggacagcaatgttatgccgtttacgttttttgttgtaatcctagttctatgaagctgtgtgagcaggtgggtcaaatgccattgcctccacttttctgcacccccctgctcctcttcaccctgacccctctgcaggaggcagaagcaaaatggcaccacatattcacctgaaaactccaaactcttttagaaaaataaataaatatttatagacctcttttagatattttaataaaggatcctttggaatttatcccagctgatgctgttttgatattacagagagttataaaatcaggatgctgtcacaactgttgcgaagtatacactgaagttgtgtcgtttttgccactagatgagattaaaagaagacaattattcaaagccatcacaaaacactataagactgaccaaaatttagataacctttgaaccacgatttttttccacatctgtctgtgagacacagcgcaatgctactgcccttccagaaactgtgctaaaaagagaaagtccaaaagactctaaacaaaaacctcgacgccgttgaggatgtgtttcattctggtggtctgttttgcaagcttgataacagaatgtccgtgccattgtaaatgttgtagagatgtgggccgtggcccaaccgtcctatatgagatgtagcatggtacagaacaaactgcttacacaggtctcactagttagaaacctgtgggccatggaggtcagacatccatcttgtccatctataggcaagaagtgtttccagatcctttggaaaggtgggcatggggcaggtgcttggagagtggcgtttgagccagagcgaccccatttcccgtgtgaaccataggcacaacccaggaagtttccccacttgtaggagtgtgggtattccagagcaagactgtggccaccatcttcccctcttggtgttttccgaaagtgacagtgttggtcatcccatgaccactgaagcttagtaaccagcgccaaaaagtagattcatcaaactagagaccccagctccccttctcgccatcttctttctcaagttgaccgtggtgctgtttctggaaggcatctgcaactccaagtccatgcagaactctggaaggccaagttcatcgcagcatgttcaccatatcccagcctccaaatctatcctcctaccttccaacgcatgacctgttggggagcagagacttaacccccaactcagaggaacccttcctccagcgtctttggcatggtttctagggtgagagttcccaatttggatagaacggccaccatattggttactgaatctctctcccttgtttttattacgtttcctttttcaaactgtccatgggaaggctgaattgagtgactccccagaatgaagatgagaaggtgaatataatcaatgccaatgtaatgccagcgggtgagatggccgatggaggtttcaaagatgtagctagcattttgaaaccatatgggcaaaacccggcaaccagaaggggacagataaggaccgttccagaaatcccaactctcacacccagcccaggctgcagtctccacaccaaacagtcaacaaaacacaaaccctgaaggaaaaccttttccatacacccaggctatgcattgaagagttttccactgtatacatttttatccagatgaaggtatttttatattttgacaataggaaacagtgaccattttcagagtaatcaaatctggaacaaatgaaacatcttttagccaccaccaccctgttgcaattaagacaaccgtgggggaacacaccactttttactgttgaaaccaacacaacgttgaaatccaggcttatacgcagactccgattcctagagaactaaatttggctttagtgtgacgggatttgattaagcacttagtatagtcttttgaacacggaaatcctgttgtacttaaagctagcggacccgtgaacaactttgtcaggttcacgtcctataacggttaaaaaacacacacacacatacacaaaccgtttctatgagagattgatgaactttgtttaaaattttaaaaaaaggaacacgttctgtaaacgagtcgctaaatacagaattgtataataaaaaaaaaaaaa

SEQ ID NO: 16 - DNA, 5524 bp, Homo sapiens
  source 1..5524: mol_type = unassigned DNA
cgggtgtggagcacggggagtcgggcgtggggcgggcagggagtggagtcggggtcttactccggtggctgcagggcgcagggtagccgtgtcaggcctgcccaggtgcagagcgctcttccgcgaccccaacagcctctggtccggtctggcgcgccctcgctttcccagagggcgacctgggctatggcggccgtggcgctggcgagcgggacacgcctcggccttgtcctcgagctgctcccgggacagcccgcgctgccccgggcgcgccgggagtcagtgaccttcgaggatgtggccgtctacttctctgagaacgaatggatcggcctgggccctgctcagagagccctgtacagggatgtgatgctggagaattatggggctgtggcttccctggcatttccatttcccaaaccggctctgatttcccagctggagcgaggggaaacaccctggtgctcggttcctcggggagctctggatggagaggccccaaggggcatctcctcagagggtgtgttgaagaggaagaaagaagattttattctgaaggaggaaattattgaggaagcacaggacctcatggtcctatcaagtggaccccagtggtgtggatcccaggaattatggtttgggaaaacctgtgaagagaaaagcaggttagggagatggcctggttacctcaatgggggacgtatggaaagttctacaaatgatattatagaagtgattgtcaaggatgagatgatctcagtagaagagagttcagggaatactgatgtcaataacctccttggtatacatcacaaaattctaaatgagcaaatattctatatatgtgaggaatgcggcaagtgttttgatcaaaatgaggactttgatcaacaccagaaaactcataatggagagaaggtctatggatgtaaggaatgtgggaaggctttcagttttcgatcacattgcattgcacatcagagaattcacagtggggtgaaaccctatgaatgtcaagaatgtgctaaggcctttgtttggaagtcaaacctgattcgtcaccagagaatacatactggagagaaaccctttgaatgtaaggaatgtgggaagggctttagtcagaacacaagccttacgcaacatcaacggatccacactggtgagaaaccatacacatgtaaggaatgtgggaaaagctttactcgaaacccagcccttcttcgacatcagagaatgcacactggggagaagccttacgaatgtaaggactgtgggaagggcttcatgtggaactcagatctttctcagcaccagagggtccacactggggacaagcctcatgaatgtactgactgtgggaaaagcttcttttgcaaggcacatcttattcgacatcaaagaatccatactggggaaagaccctataaatgtaatgactgtgggaaggccttcagtcagaattctgtcttaattaagcaccagaggcgccatgctagagacaaaccctataactgtcagatctctcaccttcttgaacattagagagtgcataatggtgatacttgtttataattcttatgctgcaggaaccctagagacaaaatgagatgaccattcacaatttgctgtaacccttaacttaaatagccagtattatcttgcccttttgaacatttaccatgtactctagcaagactggtccctctgttctatgatgttttaacaaggcatcatttagttgggcagctactctgtatcaggtgctaaccactttacatacattaatttgcataacaatcctattaaggtaggtgctcttctccccattttacaaatgagaaatctgagttgaaagaggttataaaactcattcagggttgctcagttagtaagttatagagttgaaattggagccaggcctatctgactgcagagtttactgttctttacttaattgtacatatttatgtctctgcccatttttatttgcttattttcctgtgcttttagtttcccttcatcactcagatctagctccttcaactaagaagatctctcttcctcttctacttgtaatcagtaccacccaagttagtatttaattatgtgccatcttatatttttctaatagtctcatgtcttttaatcttaaccccagctaaatgactctgaggaccaacagtacatttcttttatgtttttcaaatcctgaaacattaatctttgactagatataacatgctcatgataaaaaagaattgaaatagttgaaaagggtgttcagtgaaaagtaaatttccttgtcattcctatctcttgagttctccccagaggcaatcactgctactggttgtgtatctctgtagatactctttgtatacaagtgtttattagtattgcttttcataattctgtctcactgaaaaccttatttgatggaagcaacattgcagttaaattgtgaactctaagaccttttcttcagaagttgctttccttttgaggccaccaaagtaatttagggaaacagcagagggtaatccaggtctttttttttttttttttttttttagacagagtctcactctgttgctctggctggagtgcagtggtgctatctcagctcactgcaagctccacctcctgggttcatgccattcttctgcctcagcctcccaagtagctgggactacaggtgcccgccaccatgcctggctaatattttttatttttagtagagacggggtttcaccatgttagccaggctggtctcgatctcctgaccttgtgatccacctgtctcggcctcccaaagtgctgggattacaggcctcagctaccacgcctggccaatccaggtcttaagagacctcattgcctttgttttatgagatatcattctgggattgggaatatgtaaactcaactggagattttttttcataaaaatttatatagttccagccctctcattgcttcctatcctaaatcctcttccagtctgtccatccctcactaccatgatagtctacattctgataagctgtgaggccactgccaagggagggagaaatggtcactttctggtggtggttaatgctttgttagatagcttcatccagtcaatagttgaaaagttttcacataatccagtattggcatcagagccagaaatgccctccctaggtccaggaccaaagataaaacaaacacgaggaacatgtagcgtctacacaggaaagtaaagaattatagaattaactaattctacttgaaatcaggagttttataaaacaacatttttagacgtggtcatcttttattggtttccatcatctcttccccttctctctgggaacagttacccgggtattctttgggaagctatcctttctcagctatgtggtttggcaccaccaccatcttcatgagtggaccctgtttggcttgtgtcaatcagtttatcccatccccttggccacagagccattgtgatatgaggagatactggctcttctggaaaagagaggcttttcttcatcgagagctaccagaggagatattatctgtcctctgtgtggcacataggaaaatgtgagacctagaattatagcaactttttttttctgttaaaaggggagattctcaagcttccaggtgctaccatatggagcctaaggataaagccaataccaaagaaaacagtgactaaacagagagaaactaggtccttggtgacatcttttgagccactagaccaagctttacctgaagcagagctacctcagaacttttcagctatgtgagccaataaacatctgtcaaacgagttagagttgagttttctgttatttgcaacttagccacactaatactgttttgtgtttgaaatcactgttttctcatacagctcctcagtgtcaccttttcctcttgctcagtagtctcataagcttctcagttttatctcatctcagttgcttggaagttgagcatctaaataggtggcttttgctgggtgcagtggcttacgcctgtaatcccatcactttgggaggccaaggtgggcagatcacctgagattgggagttcaagaccagcctgaccaacatagagagaccccgtctctactaaaaatacaaaattagccaggtgtggtggcacatgccagtaatctcagctactcaggaggctgaggcaggagaattgcttgaatctgggaggtggaggttgcagtgagccgagattgtgccattgcactccagcctgggcaacaagagtgaaactccatctcaaaaaaatgaaaataataaataggtggctctcatgacctaaggttaatttcatgcatactactaagtgatgctttaagtcataccattagtgcaggaatttttgctccttagttcagctaaaatctgggttcttgtctcatgaccaggaaaaattagtcacagggacacattgaaaagtgaggagggcagaatttattaagtgaaaaggaaaactctcaacaaaaagaggggtcctgcatgcaggttttccatctcactaaactgaataccaggccaccacacatgagctgaagagcctagtcttctccccctgcatgaattcctggtggctacaccccgttctcccagtgtgcaggcaggcccttagtctgagccactccacattatttcccttactgtgtatgtgttaaggaacggaatttttcatcatgggcatgtttaggcaatccccctgtgcacaatgacctgggcagcatttggctgtctcctgattctatcattcccccctctaaagaagtacatctaacttagaataaggataaggataagggtagtgatcgatcttaactggttcctgctgatgggggcactgttttgggaaaatagcagtgagatctccctcagaggcctatctaagggtccctggtaaaaggtggccatcatttgaggttccaattgcatgaacattcagagttcaatggcctgaaggtgagaagagacaaaccaggttattagaagacaatcaaaatgaaacaaagcggggatggtaaggacagctaaaaaaaatcctaaggctgctgacacacccagataactggtagctatagttatgcctgctaagattggggtgtttggggcttggctttcgttagctcccttggtcttattttcccaaaaaagaaacctccaggttatgggcaccttatttagtctaatcatctggcaggatttgcagggtaattgcccagaactagaatattgatccagatttttacattactcatcccttttgctgcttctgagctgcagccagagattgctggttggttcacaggaataagcagtgttagtttaaaatgtgggcaaaaacttaaaaacaacgaatgagtctaaaatctaatgacaaatatataagtcttgaaacataatttctctccagttctcatttttgttaaaaataaatcatgataggactgagttgtttgcaaaataaactttagtcttgt

SEQ ID NO: 17 - DNA, 12204 bp, Homo sapiens
  source 1..12204: mol_type = unassigned DNA
gtgtgtgtgtgcgagggtgtgtgtgtgtgtttgtgtgtgtgtgcatatgtggggggtgtgagtgtgtgtgtgcgaggaagcgggggtgcgtgcgcgtgtgagtgcgtgtgtgagtgtctgtgtgtgtgtctgtgtgtgtgtgtgagtgagtgaattccagattttctgtctttccaaaacccgctcctgtcctctcgcatatcactcacagacggggatctgacagcagccacaaacctacagtgagtgatcgctctccccccggcacgaatccgccatagagatcggcgaggaggaggaggaggaggaggaagaaaagaaggaggaggtggaggaggaggtggaggaggaggaggaggagggaaagaggagaaggaagaagaagaaaaagaagaaacccactaccttcccaggattgccttttttttttccttatctttacgcgcgagtgtgcctgtggcgcgtgtgcgcccctcgtcccttccatccgaacccgggcttggatgtttaataaagaaatcaagtgtctcaacagtcaccaaaaaaaaaaaaaaccgcaaaaacaaaaccaaaaaaattccaaaagcaaaaacaaaaaagagagaggaaaaaaaattcaaaataaacaaacaaacaaacaaggcagaaccaacctctacttcaaagcagccggcacaagccacccgtgtctgccacccagagaggggggtctctggcccgtggtggaggagttgcaggggggatcgtcagggggacagaggccgagtgacgtcctaggagccaccgggcaagaggcggaggagacccagagaggccagagagacagcgggccccagcgcgcggctcggggctggggcgccagaagtgggactggagcgaagtagaggatgccgaggagaaaacagcaggcacccaagcgggcggcaggctacgcccaggaggaacagctgaaagaagaggaggaaataaaagaagaggaggaggaggaggacagcggttcagtagctcaactgcagggtggcaatgacacagggacggacgaggagctagaaacgggcccagagcaaaaaggctgcttcagctaccagaactctccaggaagtcatttgtccaatcaggatgccgagaacgagtctctgctgagtgacgccagtgatcaggtgtcggacatcaagagtgtctgcggcagagatgcctcagacaagaaagcacacactcacgtcaggcttccaaacgaagcacacaattgcatggataaaatgaccgctgtctacgccaacatcctgtcggattcctactggtcaggcctgggccttggcttcaagctgtccaatagtgagaggaggaactgtgacacccgaaacggcagcaacaagagtgattttgattggcaccaagacgctctgtccaaaagcctgcagcagaacttgccttctcggtccgtctcgaaacccagcctgttcagctcggtgcagttgtaccgacagagcagcaagatgtgcgggactgtgttcacaggggccagcagattccgatgccgacagtgcagcgcggcctatgacaccctagtcgagctgactgtgcacatgaatgaaacgggccactatcaagatgacaaccgcaaaaaggacaagctcagacccacgagctattcaaagcccaggaaaagggctttccaggatatggacaaagaggatgctcaaaaggttctgaaatgtatgttttgtggcgactcctttgattccctccaagatttgagcgtccacatgattaaaacaaaacattaccaaaaagtgcctttgaaggagccagtcccaaccatttcctcgaaaatggtcaccccggctaagaaacgcgtttttgatgtcaatcggccgtgttcccccgattcaaccacaggatcttttgcagattctttttcttctcagaagaacgccaacttgcagttgtcctccaacaaccgctatggctaccaaaatggagccagctacacctggcagtttgaggcctgcaagtcccagatcttaaagtgcatggagtgtgggagctcccatgacaccttgcagcagctcaccacccacatgatggtcacaggtcactttctcaaggtcaccagctctgcctccaagaaagggaagcagctggtattagacccgttagcagtggagaaaatgcagtcgttgtctgaggccccaaacagtgattctctggctcccaagccatccagtaactcagcatcagattgtacagcctctacaactgagttaaagaaagagagtaaaaaagaaaggccagaggaaaccagcaaggatgagaaagtcgtgaaaagcgaggactatgaagatcctctacaaaaacctttagaccctacaatcaaatatcaatacctaagggaggaagacttggaagatggctcaaagggtggaggggacattttgaaatctttggaaaatactgtcaccacagccatcaacaaagcccaaaacggggcccccagctggagtgcctaccccagcatccacgcagcctaccagctgtctgagggcaccaagccgcctttgcctatgggatcccaggtactgcagatccggcctaatctcaccaacaagctgaggcccattgcaccaaagtggaaagtgatgccactggtttctatgcccacacacctggccccttacactcaagtcaagaaagagtcagaagacaaagatgaagcggtgaaggagtgtgggaaagaaagtccccacgaagaggcctcatctttcagccacagtgagggcgattctttccgcaaaagtgaaacacctccagaagccaaaaagaccgagctgggtcccctgaaggaggaggagaagctgatgaaagagggcagcgagaaggagaaaccccagcccctggagcccacatctgctctgagcaatgggtgcgccctcgccaaccacgccccggccctgccatgcatcaacccactcagcgccctgcagtccgtcctgaacaatcacttgggcaaagccacggagcccttgcgctcaccttcctgctccagcccaagttcaagcacaatttccatgttccacaagtcgaatctcaatgtcatggacaagccggtcttgagtcctgcctccacaaggtcagccagcgtgtccaggcgctacctgtttgagaacagcgatcagcccattgacctgaccaagtccaaaagcaagaaagccgagtcctcgcaagcacaatcttgtatgtccccacctcagaagcacgctctgtctgacatcgccgacatggtcaaagtcctccccaaagccaccaccccaaagccagcctcctcctccagggtcccccccatgaagctggaaatggatgtcaggcgctttgaggatgtctccagtgaagtctcaactttgcataaaagaaaaggccggcagtccaactggaatcctcagcatcttctgattctacaagcccagtttgcctcgagcctcttccagacatcagagggcaaatacctgctgtctgatctgggcccacaagagcgtatgcaaatctctaagtttacgggactctcaatgaccactatcagtcactggctggccaacgtcaagtaccagcttaggaaaacgggcgggacaaaatttctgaaaaacatggacaaaggccaccccatcttttattgcagtgactgtgcctcccagttcagaaccccttctacctacatcagtcacttagaatctcacctgggtttccaaatgaaggacatgacccgcttgtcagtggaccagcaaagcaaggtggagcaagagatctcccgggtatcgtcggctcagaggtctccagaaacaatagctgccgaagaggacacagactctaaattcaagtgtaagttgtgctgtcggacatttgtgagcaaacatgcggtaaaactccacctaagcaaaacgcacagcaagtcacccgaacaccattcacagtttgtaacagacgtggatgaagaatagctctgcaggacgaatgccttagtttccactttccagcctggatcccctcacactgaacccttcttcgttgcaccatcctgcttctgacattgaactcattgaactcctcctgacaccctggctctgagaagactgccaaaaaaaaaaaaaaaaaaaaatcaccccagccatttctcttcatcctcactaacaatttggtaatgaagtattgatttccacttctctgcttatgggcggtattagattttcattgataaattgcaatggggctgtctcgtctccacagtacccttttcactgtcacaagaaaacaaagtgccaccgaagaaaagtaatgactgagagcattgatgtacttattttgtcagtttgtaacaggaaagtgggggggagtctaagtcttcatagtctaatgtccaagtgggttgcactagatgtagacacttggaggcttacttttcatggtaatgtccatttcctatttataacccctctgggaacgtttgtctaaaggaaatgtttctgttcagtgtaacaattacagttgcacctggattgcccagtcctgcccctgcactaggggaccattaatcactgcaaagtagaagaattattaagttaaaccagagtttgagccaagaaaacccctgaacaatgttcatcttctgtgaaacttgctcaaatagttaagcttaaccatgttgctgccaaagacttttcctatgcagtggtggggcaccttgatcatcatcattatcttgattggctgaaaaaaaaatagttttaagcacacaccactgtctatgagaactgcaaattgggagaataggtgaaatgcagaatctgagagaacgcgagaagatgagatcattacagggtggaaagttctgcagcagccttttctggtaatccctttctgcagaacctgatgtttatgggctctaaaacgcagcttagctttagaagcaacagaaagcatgaaatagggtgtccattttaaatgtgttcctgcaacttttttcattaaaactttgagggcccaattttaatttgtggaatattcccgttaataatgagatctaattaagacatccattaaaagcccgttaaagttaatttaacgtaaaaattccaatagaactgtattagattttctccattaaattaacgttatggatttttaacggatgtcttaattatacgttattattaacgggaatactgtattacacagattaaaatcaggtcctaagtcaacttggaagagctaagagcatgttttaatattaaaagtcttgcatacctagtgcacagtttggagacgcaaggatagatctgtttactctagttgaacattttctatacaattgaaagcaacctataatagataaatccatcattgcatttaaacaatgaatttccttattctcaaaggacaaatacgtctggattatgtggtaaattgctactcagctatggtgaaatatttatactattctaggcacaacactaggaactaggtgattctgaaacaaaaggaatattttctgttgttgctttaattaccaaggttatttttttttaatctcaacactgacaaaatgaaaccaaatatctcttcctcaccatttctcaaggaggctgcctgttggaattgttttggaaattttgacatgatccctaaattcaacattgggattaaaaaaaaaaaaaaacttcttatttacctcctagggaaagtgttgcccttatgccacatataatagcaaattgctttttttatggcatgcataacctagatgggaaaaaatatggcgcttcggggaaggagggaaaaagtaaatgaagttccaggaatgtcattctgaagtaatgaggcatggacagaaaatatacccctcacatcatcggattgagatggcagtcgaaatagcttcattgaagtgtcagcactcatccatcaatcaatcacccacaaggaaaaatagcaacagtacaacggggtggcttttatgggatttactcatgggcatagggaatagcggctcaaatgtagttctgacatgaaaagcaaggtgctgatattattttttatgatgggaggatcataaagtgaattgagaacagtgaggtctgtctttgcttaacctattcaaccagaaatgaatggagctcgactggaaaggaacagtcttcagatgggttaagattgaagggtggactggactctactgagcaccgtccttcaacaaggaaattctattaaaggaaaatcaatgcattagtattggggttctcgtagctgttaaaaattgtctgctccaatccagggttattaggccaaagttacataattcagatctcactgcaaccatccaaaagtggattctcgagcccttgctccaatggggggaggagatcaatacaattcccaattccatggaaattgtttcccttctaaggaagaaaaaataaatcatctgcttcaacatataatcgatatggttttgttagcgtaatttctatggtgggtggggtgggaggtgagagaaaaaaatattgataaatttggtaagacaggtgaattgccgcctggcaaccgtgcatgtcactgccgagggatggctgctaaggttcaccttagaaaacaagatctgggctggcactggggcatacatcaccactcagcatattcctagaggccaggcctgtcttcactcagccagccctctgaggcttctagaaacttctttctggaggaaaaaaactaaataacataaactcaggagaatgtctttacccaccttcataccactgctttctttttgctgaataaaacacagttctgataagtaagaactttagaattggaaaggaggctgacatgcaaatataatgcaaattaccctcaagtatcgccattcttccaccacctcttggtaccagtgagagcgagagattgccttttcttccccatccctccttccagctaagaccaccaaccagctgcaaattgagatgtccatttaaaaatttatatgtcaatatttaaatgttacatatttggccctattttgtagttcagcaaatcctccaaatacacagcatgttacaaggcactggtggcacagggcacaacaggaaatgatatttatttagcaaattcatttaacaaatattattgggcacctgttatgtgagacactgtcctaggcactgtgggataacaacagcaaacacttcacacaacagcctggccttcctgtgttttacaacagctcctaaagatagctgatatcaagacatttgagggacacagttcatgtagaatcaaaatattagtatttcagaataaggattttttttctgaaaagcatacagagaggaaacagcttaaaaataggtcaagacctaaaaacagaatataatcacggaataaactggataacccagacagtccccacagaatttctttcaggtcacagatttcttaaaactcacccccaaaatgtgcctgcttggttgtttgaatcttgcataattaatgtcacaggcgcaagccgctgaacttagttgagatgcagaaaacaaacaaatgcaatgacatatctgagaagcatttatgtaactccggttaagtggtgaggaggggtgtgtgaagacagtgtgcatgcatgagtgtgtattcatatatatgtgtatacatatgaatttcactgttattttccagggtctatggacaatgtggcagtaagagtctatgatgttctgaaacttttcacagtaaatccaaagattacagaccttacaaggtgcttgcattctgttgcttttccatctgtcacttctcaggttatttgactgtgttcaaaccttcttttctttttcattgagtttcattttttaagcttgttaaatgcttttgtttaaaaaaaaaaaaaaaaccccaaatgtcatttttcacattatcctctcttctctgcaacaaggatagtaagatgtagatgaatgcaaaaataataacaacaataaggaaatatattaaagctttaaaatatgcacatatgtagttctaaagagcaataacggtagtatctatttcgaacatgcattaggcaaaaaagaaatcaaaactgaaattttcgtgtatttttccccttgtaagatgttcaaatgctaacttcattttctcctttcctctatgtggcactttctcaaaatatctatgaaatacttttagacaaagattgagctggagaaagagatacaaatttccatccccccagacagagagacatatttccattgtaggaaggcattaaacattttgaaacttgtgaatcatctttagaatttctactggggaattttacttcttcatccaaagtaaaagccacttatctcctttggttcccagtgacagattcagaggcatacgcagatatacaattttcaggctctagttaatcttcttccaatagttacgaacaatgggctaacaggcgtgggtgtttctccaaaaattattcatgcacaaggcagcccaaagcttcagggaaaactagaaatgtgttatggattagaataggactgttttaaaatgctagtaccaggtggaacgctatttctgcaacaggactctgtccatttcctttggaacaatatattccaagtaaaatggctcttccaaggaatgacacctttacttgacacccttcggcatacaaatgattttaccaatagccatgattattattaaggccttttaaaatacaggctgtttgaaaaaagacagattaaatattcacagcctttgtatcatggttatttgcttaaaacagcttttagaagtacaagtaataactttttgataagaaaccccaggagaaactttttggtaagaaacctcaaaaaatttgaacaaaggcattacaaaaaaaaaaaaaaaactaaccactccattcaactctctcagaaaataaatttcaatgtgttcaatgaattgtcttgaacctgaaacctgcatttagatatcagtcccctgccaatagctaatattaacagaatttgaacaatcatacaattatgtctcaaatgtgaagactttgtacagtaatattttcactttctaaatgacccatataacattcaggaattatagatgtgtatgtatattttttaagtacagaaagttcagccagtcttcagagaagtaaaagtgatgtctattgtgcattgaagtaaatattacaaacattccagtttcgcaatacaatacttgagctttcgaacacctcagacactagaatgtgtaatgcgagtcaaaaaagctgacatacaaaacaattcccatttggctcagggttcctaaatgtcacaatatcttgggtaaaatatactttttgatttcctgatgatgtccttctaatcccttctgactttgattcctaacagccaggcactgttgacatgaatcattaacttccaaacccctttaaaatcaagaagctaggtgatcatacagtcatttcaatggccaaccagttcttgctctacagagcttttacacctttttgggaaacctgatatcaaacacatttatgttatatatttgctcccttgcattaattctagatttttttttaatttcttttagaaagggcaggggggaagtgggtcagagcaaggttcaagaatcacattcatccttgctctaaagtgtttacttgccagcaaagaaaggcaaacacatttttatattcagaaagcagaccggtcattttcaaagaaaaatgactgcaaccatgcctgtagaatgtttctgtgcaagcgcactaattttctatcacctgcatgctgtatataatacatttgcctgtatactaggaagaaaaaccaggctgttttccctgagtacaatgcagcttggatggctgggagcgtaagccttccgtgcatttttatagtgtacatatttgtatatactaactatatcgccatgtatgaacacagattttgttatatttgcttgtttctgtttcctaccaaactggcccacaatggggattcttttgtatagaaaaaatatgcttgtaattttttcctggtcattctctttcaatagcttatgaaagaattagatctgagtttacaaagaaactataagaaccaagtttgtctgtctgcatgagtcccgtccaattgctggatctagggaggaaccaacttcctaattcagagttttccttttaaaggcatgctttacccccatgggaaaactgcacactcatccatgtagaattattctctttgtattttatctaatagtgcctgaaaatttttttaatgtcttcttagaagaagaattcataattgtcaaaatttgaaacattagcttaattttgtttttatgacctcaagattcttctccttatttattcggttgctgttgtaatggggccccaggccattcctgacatcggcgtgttcttcttctgcattaaggatgtttttgaaattacagagattattgagccaacaggctgttttaatcaaaaccatgtttcacttctttttgatgattataaattgtccttgcaatgaaaaaaaaaaaagaacttttctgctaggaagattataccaccctgtggccaaacagattcatcacagataggcatctatgcccatttctctgggatctggaaaattcttcccttggctgaccccaatttcttttactccccattatcctgaatattagctttcaatgcagtcactatttgacatttccaaaggctttgccgcattgtcactgcccaaagacaaacaaccactggaaatgatggctttcctgcttgaaacgaagggggccaggtgcagtggctcaagcctgtaacccctgcactttggaaggctgaggcaggcggatcacttgaggtcaggagtttcagaccaacctggccaacatggcaaaacctcgtctctactaaaaatacaaaaaacattagcagggcatggtggtgcgtgcctgtagtcccagctacttgggaggctgaggcaagagaattgcttgagcccgggaggcgaaggttgcagtgagctgagatggtgccactgcactccagcctgggcaacagagcaagactgtgtctcaaaaaaaaaaaaaagaatggattttcagaaaaagtgctccctttcctgtcctgtggtgccaccatcctgtcctccttcgtaatcatgaacaatctgatcttgaactcccacataacttaaatcaggcaaaaagaaacattcacagcgtccccttgctgaataaaaatgactttgtttggaggcacttaagatgtatgcctgtgtgtggtgccgcagcattgaaattatctgtagaaggggaattttttttaaaaatacaattttatcactagaaataaattccgatggtggaaacgaagaaaacccttaaattatatcacaaaagccattattttttgcatccaaagagtttttttttttaaggaaaatcattctactttgagaactgtaattaaagccctaaataacagacactactttgttgagctattgtgaaaaaaaaacaacacattcgccaaggttatatggagcccctgatttccatcaaaaaggtttctataagtatattatttacatttttatacatgataactcttgcctttgtgttgaaaaaaaaaaagtctcttttttttcccccactcagcagttattggaaatagactgttcccatctgaaaccgtatcgtaatttgcatcaggaaacccaactgctgacattgaggacctgggtgtgttcaattatgattttgctggaggctgtccctcattttaatgctgcagctattgaaccaccttcctgaaacctagctgatacggaatagcagagacatgcctctcaacaccattagctttgcaaatggcttcatttcagtcaacgtcgacttctgctttggccaattgaaaaatgaaaattaaaggagagaagaaaaaaaacacagatgcacttaaaacatgaaaagaattatttatatgataaaaatatatttagcttttcaaagcacaagactgaatagaagtgctctttttatgctttctggagatgttactgttaaatgtctttctacatcaggcttaataaatctgtaatgacatttgatggattgaaaaaaaaaaaaaaaaaa

SEQ ID NO: 18 - DNA, 2088 bp, Homo sapiens
  source 1..2088: mol_type = unassigned DNA
atccgggagggccgtgctccgccacccagtatatatctgtccccagtccccggggccgcctcattccctgtcctcggatcacagtctcttctcactacagtgtcgccgcctctgcctgcgtagccccggccatggctctgtagcctcgacccctttgtgcccccggcccgtctccgcgctcaccacgcctgcgctctccgctcccaccttctttcttcagccgaggccgccgccgcctctccttgctgcagccatggagtcttccactttcgccttggtgcctgtcttcgcccacctgagcatcctccagagcctcgtgccagctgctggtgcagcctctcctgttgccatcagtgcccagcacctgtgctacagccatgtcactcctggcgaccctggggctggagctggacagggccctgctcccagctagtgggctgggatggctcgtagactatgggaaactccccccggcccctgcccccctggctccctatgaggtccttgggggagccctggagggcgggcttccagtggggggagagcccctggcaggtgatggcttctctgactggatgactgagcgagttgatttcacagctctcctccctctggagcctcccttaccccccggcaccctcccccaaccttccccaaccccacctgacctggaagctatggcctccctcctcaagaaggagctggaacagatggaagacttcttcctagatgccccgcccctcccaccaccctccccgccgccactaccaccaccaccactaccaccagccccctccctccccctgtccctcccctcctttgacctcccccagccccctgtcttggatactctggacttgctggccatctactgccgcaacgaggccgggcaggaggaagtggggatgccgcctctgcccccgccacagcagccccctcctccttctccacctcaaccttctcgcctggccccctacccacatcctgccaccacccgaggggaccgcaagcaaaagaagagagaccagaacaagtcggcggctctgaggtaccgccagcggaagcgggcagagggtgaggccctggagggcgagtgccaggggctggaggcacggaatcgcgagctgaaggaacgggcagagtccgtggagcgcgagatccagtacgtcaaggacctgctcatcgaggtttacaaggcccggagccagaggacccgtagctgctagaagggcaggggtgtggcttctgggggctggtcttcagctctggcgccttcatccccctgcctctaccttcattccaaacccctctcggccgggtgcagtggcttatgcttgtaatcccagcactttgggaggccaaggcaggaggatcgtttgaggccaggaggtcaataccagcctgggcaacatagtaagaccctgtctctattaaaaaaaaaaaatcaacccttcttccccaccaaaccacccaactcctctctactcttatccttttatcctctgtctctgcttatcacctctcttgcgtatttctggatctccttccctcctttctcgtccaaatcatgaaatgtttggccttagtcaatgtctatgcccgtcacataacagccgaggcaccgaggcccacagggaagcagctgggagcttggaaacctggtctcttgaatttcaaacctggtttcttacaggtggttgtctggggtgggtggagtggcgacaggatagagctgaaggactatgcaaatgaggaagtaagtcagggcgggctttgagaaggggacccatatcctacaggcaaaaagcaggctaggtgaccttgggacactacgctaagggagggaggctaaaggcggccaggtttgcagtgcgggaagatgagcaggccagtgggaggaggggcagggcagggctgtagttggtgactgggtgttcattttagctctaagaaaaaaaatcagtgtttcgtgaaggtgttggagaggggctgtgtctgggtgagggatggcggggtactgatttttttgggaggttatgagcaaaaataaaacgaaacatttcctctggcaaaaaaaaaaaaaaaaaa

SEQ ID NO: 19 - DNA, 9495 bp, Homo sapiens
  source 1..9495: mol_type = unassigned DNA
ggccgcggaggctcgggacccggctggccgcgcggcgccgcagccgccccctcccccacaccccctcccccccgcggcggcggcgcgagcgggcggcggctgtgcggtgcggtgcagagcggaggcggaggcgggcgcgcgggcagctcgcgggcacccggccgggccggcgcgggagcgggaaagggtgcgctatgcctttaacacccgcgtacagtaggcatgtatagtggagtgtagggaaactctaggcggggttaaagttcagctcatggagcggcaatagcgctggctggctggctgcagttgagccgacttggaaatgtgaacgcaagaagcaggcttgatttttttttctccccccttctctctctctctctctctctctcttcctctctccctctttctcctctctcacccacactcacgcacacctccaaaccgcacacccagacgcacacgcataccccagcgcccggcagttatgtattctccgctctgtctcacccaggatgaatttcatcctttcatcgaagcacttctgccccacgtccgagcctttgcctacacatggttcaacctgcaggcccgaaaacgaaaatacttcaaaaaacatgaaaagcgtatgtcaaaagaagaagagagagccgtgaaggatgaattgctaagtgaaaaaccagaggtcaagcagaagtgggcatctcgacttctggcaaagttgcggaaagatatccgacccgaatatcgagaggattttgttcttacagttacagggaaaaaacctccatgttgtgttctttccaacccagaccagaaaggcaagatgcgaagaattgactgcctccgccaggcagataaagtctggaggttggaccttgttatggtgattttgtttaaaggtattccgctggaaagtactgatggcgagcgccttgtaaagtccccacaatgctctaatccagggctctgtgtccaaccccatcacataggggtttctgttaaggaactcgatttatatttggcatactttgtgcatgcagcagattcaagtcaatctgaaagtcccagccagccaagtgacgctgacattaaggaccagccagaaaatggacatttgggcttccaggacagttttgtcacatcaggtgtttttagtgtcactgagctagtaagagtgtcacagacaccaatagctgcaggaactggcccaaatttttctctctcagatttggaaagttcttcatactacagcatgagtccaggagcaatgaggaggtctttacccagcacatcctctacgagctccacaaagcgcctcaagtctgtggaggatgaaatggacagtcctggtgaggagccattttatacaggccaagggcgctccccaggaagtggcagtcagtcaagtggatggcatgaagtggagccaggaatgccatctccaaccacactgaagaagtcggagaagtctggtttcagcagcccctccccttcacagacctcctccctgggaacggcgttcacacagcatcaccgacctgtcattacaggacccagagcaagtccgcatgcaacaccatcgactcttcatttcccgacatcacccattatccagcagcctgggccttacttctcacacccagccatccgctatcaccctcaggagacgctgaaagaatttgtccaacttgtctgccctgatgctggtcagcaggctggacaggtggggttcctcaatcccaatgggagcagccaaggcaaggtgcacaacccattccttcccaccccaatgttgccaccgccaccgccaccaccgatggccaggcctgtgcctctgccggtgccagacacaaagcctccaaccacgtcaacagaaggaggtgcagcctcccccacgtcaccaacctactcgacacccagcacctcccccgcaaaccgattcgtcagtgttggaccacgggatccaagctttgtaaatatccctcaacagacacagtcctggtacctgggataaaagttgcagcgtcccaccatccaccagacagaccacctgaccccttctcaactctgtaacatggacgcaacctcaacccagcgcagttacaacttcactatcagcggaaggggagaaaaaccgattcaaatcaacttgtacatggaaacagcaagcattatggtcaaacagcaaaggccataaccttttgggatttttttttttttaaaatactttagggactgttgtaatttctcatatggtgctggaaatggttgggctttgtaacatttgaagtgtttccatggtagcgtgagcattaggtgacgtggctagcggaggactacccttgctcactgacttcctgttgtaacacactttccttacggagcctggctgtttcacagtatttcatgaatttacccacacaggtgtgatcctccttgagcattgaggaggcacatggagaactaaatcttttgtagtagctgagatctgcaatatataacgggacagtcaaagggcaatgtttttctgtaacatattggaaaaagaaaatgcagttatattccttttttatttgttcctttagtttgttttggttcagcagtcagcagttaagtatataacatggcccgcaaggacaatgaatccactcacattgcagaacaattccgaaaatggcaaactactactactactgttcagttttttaaaagttttgaaatgctgcacttacatttaaaaaaacaacaacaacattttttcaacaatttcaacaatgacacaaaaattcacatggaaatggggaagatggtctgttttgacagaaactgacaggaatcaatcaaaacaatcgaattttgaattgagtaaagtgcaatttcattggatagctaaatatctttgtaagatagagattgttgaaaattctatttttgtttttctagtcctttcaccccaggactctaaattattggggtaaaaaacagccttgcaagaaaaaggggagctatttttgctttttatgttttttattgttaaacttgtatccctttaaaaactgaaggaaattaaaaaaaaaaaacaaaaaaacaaatctaatggtgcttttaccacaatatgttaactacattaaatgctaattaattattttctgttatcaaagcacatgactaaaatgaaatcatggtatctgttaattttataagctagaagtcactataatggattacgccaattctaaaaaattttacacctatctggcatcataggatttatcagttatcagacacctcattgtaccagagattgtccagaagttttaaagacctttgcatccctgaactgggctatgggaaataataatagtaataataataataataataatgatgaaaccaatactgacacaaatgctggtgcccattcagatcaagggtacttgttagggaaaaaaaaaaaagtttgcacccccaaacgtcctgtatcttatgaaaaaaaaaacaaaaaacaaaaacaaaaaaaaaacacaaaaaaccacagaaacaaaaacaaaaaaaagtgcaagtgatttttctaccagacagcgaagcacccctttgcttcccatgcgacttcaagaaggtttcctatactatacatatatatacgttctggttggcaagccctgctgatcagagaaagtctctgcatgttctagtgttagtaactaatttttatatagttaatgtaggataaagtagagtgcattaagacacaatattgtaatccctactctaggcacttgcctttaaactatgtttttcagcccttcagaagggttctactactgtcctatacaatcaagtaactgaaattcttgggaagacactttgctcctcatctttctccccgaaacaatgttgttttgttttgttttttttccttaatttgcacgaaaacaaaaattccatatcaatgtgccttgccctggatagcgattatttgtggaattgttgcacatgctcctctattgaaaggggtttttccctagtcaagcatttggagacactttttgtaaatgtgacttttatgtcagccatcgtcagtttcaacatctagaactaaatagaaagctagttgttccgcagataggagtagtctttattgtcctgtacggtcggtggcagtgctattctgagatctgtagatgcttagaatatcagtattttggatgttgctgcattttacaatttatttggagtcttcctttattttcccccagatatatgaaaatatgcaatacctgcttatatcatgtagaaaagcttagcaattattaatttttcttttatttttttttatttgaccaaagtcggtgctgcacttgacgcagtgtgttttaggtgtttgtctttgtacttttttgtgatttttgaatgcacgtgcgcaggaagggctcctcttagagaagcagtcaaactgtgaagcactaagctgaccctgcttcaagcaattttgtttttacaactgttcctttcacaagcaagccttaaaaaaaaaaaagacaacttcctttttcttcagctcccacaccccatttttcttagcagactgcagtcaatccacattcaataaaaagtatataatgcccatttttatatgcacgtttttaaacttccaagttctgaaaattgtttactggttatctctatttaaggaaaaaaaaataaaataaaacattttggattttcatatgtgtctgataagtggttgaatagtcgtttggcgctgttgtatggtgtgattgtcagtgtatggtgtcacttcctatagccagccagcatactttgccttcccctatagcacttagctgggcattactttattatgacatatgtgcactaaaaaatgaaaaaaaggaaaaaaagaaaaaaaaaaagaaaaaatagcagctttcagtgcttcacagtgaagggaaaaaagcctagacaaacattttgtcagaaccttgcaataagccaaggtattaccagtaaattggttgtatatacaataaaattgcacccttttttaaacaaaacaaactaagcaatagtttgggcagttttagttgtttttagtgagcatgttgtagtcatgactgcaaagagagagaataaactgcccgctcagaagatatgtaatttgtattgttgtatagttttattgattacactgatttattctaccctattttataatgcaggacttttgtaatgttgtttaaatgaggaaaaatttctgtcaaattagcctagtaaaatttctgatcgttcattataaaggcagcgttcatagaattgcttttctttctttttaccccccctttgggaactggatttaagtttaaaactttcctgtttcctttttttttttttttttgtaagtatttaaatacaattatttttttctctcaatggtatagcatattcctatgcttgagaagtataggtctactgaaaaaccattgtaaatggacgttacaggtatgctgtatttttgaaggtattttgttgtattaagtttgatgaagctaaaattagggaactctgaacagatttgcaggaaaaaatgttttaaaggctttaaaacattagggaggcagtctagggtgataacgaacaggggttaagtattaaatacacgaagttacatttttgttcatgtttcattgtccagaaagcagcaggaaactattcagttgtgatcaagcaggaaaaaagaaacaccaacagttgccagtgtttttgctttttagcttaaaagcatagtgaagatgcttgaggaagactttgctacctggggtgtgtagacagacagactgagagctatcagcatttgaaggcccagcccttgactctgagacacatttgaattttttctttcccatcaaatggcattaacaagattgggcaaagatgagtccctcaaatttctgtgttttttgtttgtttgtttgtttgttttttctttgggaactgaagtcagaggcacgaacactaactcttagcatttttctgtagactttttcttctggcccttgtccctgccagcaaaacgccccttttctgatcattcgtgcgcagagggcctcccagtaatgccacgctctccatgctagagagccttctctttcctctgaggtttgaactgatgttctgtgtcttcacaccctggcatgacagttacgtgtggtcagcccgctccccaggcccgtccctgccgccgccaggtgtgggctctaggcaggccgacaaggttacacctcccagagcttgtgatcttcattttctgacagtcaaagtgtgaaggaacccagacttccccgagccacggtgttcagtcagcccacaggaatatgcaagacccatctccaaaagtttgtctttgattttttccaagcccttagccccataagctttgaatcctgtagttacagtggcataaaggactgacaaaacctggataaggaaaaaccttttttttctatgaattttttttgttttttaggggaaagggattctaagaatgtcatttaatgtactttgcatcatgtctctagaaatatctttgtccatagtggtggtggagtctctctctctctctctctttttgtttgcttctgttttctttcttgtcttcattctttcttttcttttttatttctggtagcaggcctccatagaacaaatctaaaacacaaccaccatagtaatgtaaggagagcttcagtggcacctcaaaacccacccttcgagatctgtccaaagacagtctcagaaagctgcactgcccaccggctcagctttcattcaaaaaggcttccaaggccaattctgtcttgaagtcaatgcatgtatttactgtttgacagtaaacccgctctgccttctccacgtccaaggctgtgcattcgtctaattagcgtcgtgtatgttttccttttattttttccaataaaaaagcagtgggatgaaaattgctttgatatatagcaggtaacattgaagctattccatagcacttaactgtagtgaatactgtgtcaccaattttgaaatcaatttaatgtttaatgcaaatccattacatggtgctattataggctgacaaaatgatttacacaaatgtgacaacttgggctcaattcactctgctttccaacagtgtaaatgcatagcagtgtttatctgcatgagaactatgcactaatctatctgaagaaaaaaactatatcaactttggtatctactttccgtttacttcaatccttgcctttttggtcattgttataatgccagctttaggacagaaagaattataagaaaaccagcataatacctgatatattaaaatgtagtgcctgtgaaatctgtattatattgctcttctgaagtaagatttttctacaccggtagccttcgctgtctgtcagtcaggaccttctggtataggtgatgtaaaataaccgtacaatattaatgcatgcgattccataatgcttagtgaactgtatgaatattactcaaagttatgttagtctttttttccgacttggttcttgtcagctaggtttaaaggtatttcactgagaacgcaaattctgtcttttcttgatttcggctgttttcagtattttggaggtatacatttacttaaattcagtattactcgtgttttgtttttgtttttgttttttgttttctttttcctaggggacaagcatgggtgtttgatttcagaaatcagtacctggcgagatttttgtctcaaaacgactatttgaatttcaagaactgtgctgcgaagacactctgagaacatttgcaagtcaggggcattttccttgacccttgactgatgctatgcggagactgatacattttcttaatggacaatgttcaagccaggtacccatgcttgatctgtcttcacaccagacctcctcatattaaaaggaaaaataagaaaaaaaatgtaagaaatcacatggctatttagtttcatgcacagttgcaatattttcttcaaaaataaaactctgtacaaactttgggcccgattcataagaaaaagaagtttgctattaacacgggatttttttaatatactttttttggtctaaatttgaaattacttgcttcccaaattaaataaatttcatctcatttttttccctaaaccagcacccatctgccttttattccccaaagagttacctttcccagattagggggatggtatgtggggagcagatagcggaaatgcttagaaagataagggggaccacccacagctggtcgtgagaacagggagacagtgtgtgggggtgggacctcatctgtgtgcctggtatcctgagttttacatgtagatgcattcgcctatttgattcagaaaaataaactttcccaaaatgtgtctgaaccacaagagcatacagtggaagtgctacctctaatctaaccagagcaccttcatggtggaagacacccaccaggtcatacaatgtgaacttttgtatctctgcagtggtttcaaggacaaatagtgtccaatgtattgggccatttttcctgctgtttttatactcaacttctcaaaatgaaaaaagcttttatttttcctttgacttatttgtgttgttcttattttttaaatttttattttttgataatagtctgtaagttagcctttttgggttttttttttttttttttggcttttttttttgtttgtttttttttcttttgacattgcaaccgaaggtcataaggccgctagctccgctgggacagaggcttgagagaactaacggctcggtgccttctccctggtctcagaccatcgtctctgcactgcgaaggcatttggtagcctcgccactgagatactaactagacctagactaggagctttatcaggttctaggaggtcctttaggaagactctcaaaggcaaatccctgatcccccgccccacccttagccctgccctctcaccagagcaaaattcactggggacttttcccaccacacatggaaatctgtccactcggaatacctctgttttccatttcaaattgtagggggaggggatggaacacttccagtgatggtaagagatctgttatgaaacgaaacaccccccgtgttaataacttggtctgaaatctgtttttatgagccgggccccctgtgcctctagtatacttgtattgactctcatagttacccttttagttttactgtgttctgtgaaaatttgtaattggttgagaatcactgtgggcgtccattcttattcaactaaatctccacaggttttttgagctggtgtggattagtttaactcttgtattcaaccattagtgctaccaccttctcacattacaatacaattactggaagcaagtactgcatttcctatgcaacaaaaaaggaaaaataaaaaattgctaatgctaaaaaaaaaaaaaa

SEQ ID NO: 20 - DNA, 8285 bp, Homo sapiens
  source 1..8285: mol_type = unassigned DNA
gggctgtaaccttgaactttcccagcgcggtgacacattctccccgctctccctcccgcccgcccgctcgccctcctgcgccctcccgcgcccccctccccgccttttttgaaaaagcattttaccaccaaccaccaccccaatccaacccacaccgaaccttcgcgcaccccctacaccccaacaacaacaacaactgcaaaatagaaaacaaatccccaaacccaggcgaaaagcagccaacaccggcggcggcggcggcctcggcaagcacggccagcgcgctcggactgcaagagggttaaaagtgtagattggatttcacccctggaaatctagcacgccgagtgaacttgaatctttggctatttaaggaggactgggtttgttgtgaagttgcggtgatccagcgcagagccccgtcctgattgatcgcatcgcggggctcagatgactgtaaaatgaatagatgaaattcttgcttctcgaagattttcttgggcatctcccggaaagtgcgttttaaggcgaagtcatgatgtattctcccatctgtctcactcaggatgaatttcacccattcatcgaggcacttcttccacatgtccgtgcaattgcctatacttggttcaacctgcaggctcgaaaacgcaagtactttaaaaagcatgagaagcgaatgtcaaaggatgaagaaagagcagtcaaagatgagcttctcagtgaaaagcctgaaatcaaacagaagtgggcatccaggctccttgccaaactgcgcaaagatattcgccaggagtatcgagaggactttgtgctcaccgtgactggcaagaagcacccgtgctgtgtcttatccaatcccgaccagaagggtaagattaggagaatcgactgcctgcgacaggcagacaaagtctggcgtctggatctagtcatggtgatcctgttcaaaggcatccccttggaaagtaccgatggagagcggctcatgaaatccccacattgcacaaacccagcactttgtgtccagccacatcatatcacagtatcagttaaggagcttgatttgtttttggcatactacgtgcaggagcaagattctggacaatcaggaagtccaagccacaatgatcctgccaagaatcctccaggttaccttgaggatagttttgtaaaatctggagtcttcaatgtatcagaacttgtaagagtatccagaacgcccataacccagggaactggagtcaacttcccaattggagaaatcccaagccaaccatactatcatgacatgaactcgggggtcaatcttcagaggtctctgtcttctccaccaagcagcaaaagacccaaaactatatccatagatgaaaatatggaaccaagtcctacaggagacttttacccctctccaagttcaccagctgctggaagtcgaacatggcacgaaagagatcaagatatgtcttctccgactactatgaagaagcctgaaaagccattgttcagctctgcatctccacaggattcttccccaagactgagcactttcccccagcaccaccatcccggaatacctggagttgcacacagtgtcatctcaactcgaactccacctccaccttcaccgttgccatttccaacacaagctatccttcctccagccccatcgagctacttttctcatccaacaatcagatatcctccccacctgaatcctcaggatactctgaagaactatgtaccttcttatgacccatccagtccacaaaccagccagtcctggtacctgggctagcttggttcctttccaagtgtcaaataggacacccatcttaccggccaatgtccaaaattacggtttgaacataattggagaacctttccttcaagcagaaacaagcaactgagggaaaaagaaacacaacaatagtttaagaaattttttttttaaataaaaaaaaaggaaaagaggaagactggacaaaacaacacaaaggcagaaaggaaagaaactgaagaaagaagataatagaccagcaattgcagcacttacaatcactaattcccttaaggttgaaactgtaatgacataaaaagggtcgatgatatttcactgatggtagatcgcagcccctgcaacgtagcctttgttacatgaagtccgctgggaaatagatgttctgtctctatgacaatatattttaactgactttctagatgccttaatatttgcatgataagctagttttattggtttagtattcttgttgtttacgcatggaatcactattcctggttatctcaccaacgaaggctaggaggcggcgtcagaggtgctgggtgacagagccatgagccagccattttataagcactctgatttctaaaagttaaaaaaaatatatgaaatctctgtagcctttagttatcagtacagatttattaaatttcggcccttaacccagccttttccagtgtgtaacccagtttgaaatcttaaaaaaagaaaaaatgaaaaaaaaaggaaaaaaagaaaaaaggaaaaaaacagtttgaacacaaaggctctatggaagaaatgcctctatgtaggtgaagtgttctctctgcatgcaacagtaaaaattaatataatattttccccacaaaagaaacacttaacagaggcaagtgcaatttataaatttatatctaaaggggaatcatgattataagtccttcagcccttggactctaaattgaggggattaaaaagaatttaaaataattttgaacgaatttattttcccctcagtttttgagggcattaaaaaggcattaaatcaagacaaatcatgtgcttgagaaaaataaaattaatgaaaacacagcacttatgttggtttagctgcagcctccttggaggtagaatttatttatttaaaattactggttgcatcaagaacccatagggtgtacaaaaggttctataaaatctgcattatagagacaaagaggcaggcaaatccatgtcacaagggtaaagcttacagtttacaaactgggaacgccagggtgtaggatataaaaacgcactcttgagaaaacaaatgtaatcagggtgctgaaaacttgcatggtgctttcagacattagccttgttcaacaaatttcttgtattgacagatccatagtgtgcatgggcagacacattttgcctctatgtctcttaaaattttaattaaaaatactctttccagtaatcctaatttgcacgaagatataatgtccacattacgtgccttgccttgaaatctaaaaaacaaaaaacaaaaaaaaaaaaacaaaaaaatacaacaaagtgacatcactacacttgttttgctgcatttattatcattttaaatctttaccatttttatgacaaaatattttgtactccagacgaagaaaaatgtgtgacatcatggattttttagacagttatacctttatctcacatttataaagcatatcatggctgtgtatagttgccgcttaaaaattgtaatcgaccagcaatattttcagtattttggtgtttttttctattaacctttcatgtttttcatcttccaattaatatttgggggggaggggtttcaaatttatacgaattatgcaataccaagttttgcctatgtaggtagtgcttttagctgtattggttattataggtaagtacacagatttaaaaaaaaaataatgtatgcttttttgtttgtttgtttgttttaattgaccaaagtgggtactgctatttttgcagtgtgatgaggtccttttgtgtactgagagatggacaggggattttttttaatatacatatatatatattctggggtgggtgggaggatttttaacactttgcagtgtagctgtgaagcagtgcaccctgagatgggcctgggctgcaaagcgactgttctgcctactgtgacaaacttcaacttacacaggttcccctctctaacttcccacctgggttgcaagctgaactcattactggttttcataacaacacaatagtaagaacaagcaaacacaacaaattctcctggaggcagacttggcttaaaaaggcagacttggcttggtgatagtttttcttgaaagttccagatccacagtggagagtgagcctgtctcatatttggcaaaaatatttgttgaaatgtccacataggggatgttggatgtttaacacttttgagagtttaacacatgaatattctttctcctagaaaacacattagacctgttggagggagtctcccgtattccttttctgccacttttcgtccccatttcatttcattaatgataggatatgatttacctgtgacttactacttcaaatggatggcagtgcacttggatttttttttaatatccagaagattgaacagagggttgctattgttgaatgtatttggactgatagattaaaatcaaagttcaatttttaaggaacaaaaaagtaaatcctgttttcattttatctccccttttaaaactgagaaccagagcagaagggaaatatagaattttaagcaattaatcttcctgtggatgaattaaacccattagatgctgatgggatttttttaaggaatggtaccttaactatatatttgatttcgtttcccctgagggctagaggctgaatggaggctggttttattttgcctttccctcaccgcccagtcccattgagtgtattcattactagaaggaaaatctttcagaattggtgacacatggtaggctgtcttaaggagtcccctggcccccttcccctaggccatggcctaataaaataaactgtcaattgttctcacagcatatcatttaataatgaatactttagaacaatgcttatgggctggagaattgtatttgattagcccattcagtttgatagcccaaatgctgaacagcacagcgggatcctagcagtgcaagttcaaaagtaagtccaatcatttctgtgatactcgccctggtagcaaacagatcatctcagccaagctcttcatgtatctttgacctattaggtgaacaaatgaacctcacaggacacacagtattttttaaaggcagactcgctctcttttttgccagtgagcagttctagctaaccaagttacacactgtgggtattcctgcctgcctcttgaatacaaaggcctagttcaagtgttgctttttttatttcaaatcaattttttcttctttcctttttgagataaaactattaaaagtactactatatatataaaatctcaaatcaacttttcggcctcctcctcgtgtaccaggaagtatattctgacgaagggccccacttttgcaggtcttgcacgcccctcccttacccagaactgcagagcttcaggatggcgaaggtcacccaagggcatgagtagggagtggtgtctccaaccatcagttccgtggcactgttcagcctttgtgtgctgccctgccacccaccactcacagtgcctctgaagcgtgttacccctggagtgacgtgagcatttgaggcttgtctaaggaaaaaaataaaaggcagtgaaggagactgtacataaagacatggcaaaaatcttaattatagcaatatagttatcgggtaatgttcgggtgggcagctccattaaaaaatatgtgaatgaatctgtgaagctgcaagtagcgagaagagcgaaaggtcttcttaatgaaccgcctaccttgtagacagtaatttgtacactgtatagttttgttaagaattttttttaaattaaaattcccatgtttgtaaagctaactttttaacaattataatggaactatatgttgtttccatttttaaagtaaacaagaatattccttgtttagagactggacttgagttaaaactctccagtctcttaagttatgtattaaaaagaaaatctgtccatgttaggagttatttcacagattcctgtgcttgaaaagcataggatactaatcctttaaaaaagtgtaaatggagaaaagttatattttatgaaggttattttgttgtatttagtattggaaaagttggtttccagagcatttcagaatgtcgaagcaccactgtctttttattagtatatacggcctttagcaaaagtttttgtgattgttacgtgatggtatttaaggttaagtttcacagagcattcaggataggcagaaaactaaaacagtgctatgtctcacataacgtgtcctcagggagcagaatcttggatttgtgacttgtagcttcataaggactcaacgaaagagattgcacagggacatcttcagcggtgtgacagcaggacatgttctttacctagattcaaattctatgtactgtgtgaaatgatgaaggctgcagaaagttatcccatattcagtgtacagtattcatttttaatgaaacaactctacaatattgctggcagataggccccaagcatgacattcaatatagtttacatgttcctgtcaaggtcttttgttaacattaaccagctgcatgctttctggactttaagaaattgggtttctatagaaaactttttttttttttttttttttaatgtgcaggctattcaagttcaatagtaaaagctcaaaaatgaatgttctactccatgctgaaggagctgaaagctgccttcttcatattttgcactttctggtagttcccctgttttttctaattccctaaaattgtgtgggtggagtggagccctgcagttggggggtaacatggaccactgattttgccctttgaccctgcacaatgacctttgcatcagccaaactcattgccatgacaactctttgtactgtgtccgtgccacagatctgttggtcacattgttaatagtaaaggggacaagttggagacggtcaatttttacattttttgttgcaattttttcttcaatggttgtaagtagtttttttttttttttaataataaaagggttcactagttaatactctagaaatatctgtgtgttgcaattcaaatgtatgttgagattgtgaaaagcgcttcagtgccactagcttaccggtacactagactaagcccttgatgacttattgcatgatacagtaccaggaacaacaggtggcctaaatacatgaaaagcagtgtaagctagtgacactaaagccagtcttgtattactgtatttttgacagaatggttttgaaaactgtgctacagggactgatgtggcaaatatatctctttatgcagaaggaagtctttttttttcttttttttttttttaagaagtatggctttttatgcatccttcatcgagggcattgaagttgcatggactgataaaagttgatgcaaaacaagaaagaaacaaacaaaaaaaaaaaaccagcaaaatgtttaccaaaaaactcaaacaaatgagcagtgcctgttcaatttcacagtctctgttgagttcagttgtaaatatgtttcaaatgacattttcttgggaaaaaaaatctctacaacattgtagaatgtgaggggtaactacatcccaggcataggtttctcaaagctgcagtagattatgtcttcatcaagctgttaatttgtgcttatatcatatagaacttttagcatcctgggaagagctgcccccacctcaatgatatttctctgagaacaacttttgtaggactgtgtgtttctttagatacatttagtacaactgtaggtgacgagtagtcagttattgcttgctagctacacaccagggttgatccattttaaaacttttggcattttgtcctcatgggccataaatacagaaccttgtattttaattaaatttttttacaaaaggaggcacatgcacaatctccatgtaacaaacctttagcagtaggatgtattatacgacagttacttaatttctagagttcaggcctctgggatcaaccccagactgggccagaatgttagtgaaggttttattgtgcccggttggaggataacgttctttgggtactttttgtgggttgcaaatgaactcaattgccacaagttttaaactggtgtaaatcaagcttgacttaatgtgattgttactgttatatccagcctatactgctagcagctgctcatactgcagtcaattactggaagcggatatatttcctatgcaaaaactgtttaaacaataaaatgagctatgctacagactctgaaaaaaaaaaaaaaaaaaaaa

SEQ ID NO: 21 - DNA, 3919 bp, Homo sapiens
  source 1..3919: mol_type = unassigned DNA
ggatgtatgcgtatggttttgttgggagatgtgcccctttcccagccgaggagggacgcacctttgacctttctgaagagctgggcaggtcggtaaccagggaagggacaggcaccacccggctaaattcagaaccagtcccgctcctctgcttgccactccttaattgctcaaggaaaaactgcatagaaaatctaatggatgaagatgagaaagacagagccaagagagcttctcgaaacaagtctgagaagaagcgtcgggaccagttcaatgttctcatcaaagagctcagttccatgctccctggcaacacgcggaaaatggacaaaaccaccgtgttggaaaaggtcatcggatttttgcagaaacacaatgaagtctcagcgcaaacggaaatctgtgacattcagcaagactggaagccttcattcctcagtaatgaagaattcacccagctgatgttggaggcattagatggcttcattatcgcagtgacaacagacggcagcatcatctatgtctctgacagtatcacgcctctccttgggcatttaccgtcggatgtcatggatcagaatttgttaaatttcctcccagaacaagaacattcagaagtttataaaatcctttcttcccatatgcttgtgacggattccccctccccagaatacttaaaatctgacagcgatttagagttttattgccatcttctcagaggcagcttgaacccaaaggaatttccaacttatgaatacataaaatttgtaggaaattttcgctcttacaacaatgtgcctagcccctcctgtaatggttttgacaacaccctttcaagaccttgccgggtgccactaggaaaggaggtttgcttcattgccaccgttcgtctggcaacaccacaattcttaaaggaaatgtgcatagttgacgaacctttagaggaattcacttcaaggcatagcttggaatggaaatttttatttctggatcacagagcacctccaatcataggatacctgccttttgaagtgctgggaacctcaggctatgactactaccacattgatgacctggagctcctggccaggtgtcaccagcacctgatgcagtttggcaaagggaagtcgtgttgctaccggtttctgaccaaaggtcagcagtggatctggctgcagactcactactacatcacctaccatcagtggaactccaagcccgagttcatcgtgtgcacacactcggtggtcagttacgcagatgtccgggtggaaaggaggcaggagctggctctggaagacccgccatccgaggccctccactcctcagcactaaaggacaagggctcaagcctggaacctcggcagcactttaacacactcgacgtgggtgcctcgggccttaataccagtcattcgccatcggcgtcctcaagaagttcccacaaatcctcgcacacagccatgtcagaacccacctccactcccaccaagctgatggcagaggccagcaccccggctttgccaagatcagccaccctgccccaagagttacctgtccccgggctcagccaggcagccaccatgccggcccctctgccttccccatcgtcctgcgacctcacacagcagctcctgcctcagaccgttctgcagagcacgcccgctcccatggcacagttttcggcacagttcagcatgttccagaccatcaaagaccagctagagcagcggacgcggatcctgcaggccaatatccggtggcaacaggaagagctccacaagatccaggagcagctctgcctggtccaggactccaacgtccagatgttcctgcagcagccagctgtatccctgagcttcagcagcacccagcgacctgaggctcagcagcagctacagcaaaggtcagctgcagtgactcagccccagctcggggcgggcccccaacttccagggcagatctcctctgcccaggtcacaagccagcacctgctcagagaatcaagtgtgatatcaacccagggtccaaagccaatgagaagctcacagctaatgcagagcagcggccgctctggaagcagcctagtgtccccgttcagcagcgccacagctgcgctcccgccaagtctgaatctgaccacacctgcttccacctcccaggatgccagccagtgccagcccagcccagacttcagccatgatcggcagctcaggctgttgctgagccagcccatccagcccatgatgcccgggtcctgtgacgcaaggcagccctcggaagtcagcaggacgggacggcaagtcaagtacgcccagagccagaccgtgtttcaaaatccagacgcacaccccgccaacagcagcagcgccccgatgcccgtcctgctgatggggcaggcggtgctccaccccagcttccctgcctcccaaccatcgcccctgcagcctgcacaggcccggcagcagccaccgcagcactacctgcaggtacaggcaccaacctctttgcacagtgagcagcaggactcgctacttctctccacctactcacaacagccagggaccctgggctacccccaaccacccccagcacagccccagcccctacgtcctccccgaagggtcagcagtctgtctgagtcgtcaggcctccagcagccgccccgataatgccccggcactgaagtcgggacacaatcagctttaaccaatggatgaggggggtggccacaggagatggggagaggagtctgaactaaacccctggcttttgtgcacactgcatacgtttcagaactcctggatggtaaccatctctggagtgcagcgcttgctgcagtggaaatgatcaggaatactgaccgtgtttctcttgcctccgaggttcttgggcacactctatagccatactggacaggaaccaggtgccccgtgtaggcatcgtcggtcggtttgccgtcagagatggcgcatctcgctgcatcccccgagagtacaccggttgctctagccacctgcggcccgcccatctgcgctagctggccttcacgctcttgatcgtctttcctttgtattggagaaggactgggtcagagatctgttggagagagagaataaagagattatttttcattatttttaaatggttgtttttgttttaatttgcacagctacacagaggaaataacttaggcactttctgttttttttaaaaaaataataaggtctcatggcttcatttagagaccacagtaacaacagcagcccaccaatcagagaagctggttgttattaaccaagctacagattcacactttctggcctaaaccctaatgggatgaggcttttcaccccaggccatgctggtggtgattttttagcccctaaataaaacactggactatttcctgtttacttcattgattgcaactacaaaggtggactcaaagcaaagcacaatcatgccagccaacattccagaattctgctgagaactccaagtctgtgaggggagaggttttacaagccagacaggcctgggggactgcagtccccaaggagaccctgccacatgctggccctttgagtgagaatgctgcatctttctacatatcttcatgagaatactgagaattggattttccttttcaaaatgcactttgctttttttgtatgttttgttatgttgagatgtttctaaagaaaagattttatgtaattataagatgaagcgtagtgaattgtacagctgttgtaataatgacctatttctatataaaataaaattgtatggcttatgtgtaaattattttgtatctgagataccagttccttttcccaaatataaaagtataaaagttttcttgtgtttttctgtgagtgaaaattttgtaataaattaacaaatttgtacaatt

SEQ ID NO: 22 - DNA, 2158 bp, Homo sapiens
  source 1..2158: mol_type = unassigned DNA
attcataaaacgcttgttataaaagcagtggctgcggcgcctcgtactccaaccgcatctgcagcgagcatctgagaagccaagactgagccggcggccgcggcgcagcgaacgagcagtgaccgtgctcctacccagctctgctccacagcgcccacctgtctccgcccctcggcccctcgcccggctttgcctaaccgccacgatgatgttctcgggcttcaacgcagactacgaggcgtcatcctcccgctgcagcagcgcgtccccggccggggatagcctctcttactaccactcacccgcagactccttctccagcatgggctcgcctgtcaacgcgcaggacttctgcacggacctggccgtctccagtgccaacttcattcccacggtcactgccatctcgaccagtccggacctgcagtggctggtgcagcccgccctcgtctcctccgtggccccatcgcagaccagagcccctcaccctttcggagtccccgccccctccgctggggcttactccagggctggcgttgtgaagaccatgacaggaggccgagcgcagagcattggcaggaggggcaaggtggaacagttatctccagaagaagaagagaaaaggagaatccgaagggaaaggaataagatggctgcagccaaatgccgcaaccggaggagggagctgactgatacactccaagcggagacagaccaactagaagatgagaagtctgctttgcagaccgagattgccaacctgctgaaggagaaggaaaaactagagttcatcctggcagctcaccgacctgcctgcaagatccctgatgacctgggcttcccagaagagatgtctgtggcttcccttgatctgactgggggcctgccagaggttgccaccccggagtctgaggaggccttcaccctgcctctcctcaatgaccctgagcccaagccctcagtggaacctgtcaagagcatcagcagcatggagctgaagaccgagccctttgatgacttcctgttcccagcatcatccaggcccagtggctctgagacagcccgctccgtgccagacatggacctatctgggtccttctatgcagcagactgggagcctctgcacagtggctccctggggatggggcccatggccacagagctggagcccctgtgcactccggtggtcacctgtactcccagctgcactgcttacacgtcttccttcgtcttcacctaccccgaggctgactccttccccagctgtgcagctgcccaccgcaagggcagcagcagcaatgagccttcctctgactcgctcagctcacccacgctgctggccctgtgagggggcagggaaggggaggcagccggcacccacaagtgccactgcccgagctggtgcattacagagaggagaaacacatcttccctagagggttcctgtagacctagggaggaccttatctgtgcgtgaaacacaccaggctgtgggcctcaaggacttgaaagcatccatgtgtggactcaagtccttacctcttccggagatgtagcaaaacgcatggagtgtgtattgttcccagtgacacttcagagagctggtagttagtagcatgttgagccaggcctgggtctgtgtctcttttctctttctccttagtcttctcatagcattaactaatctattgggttcattattggaattaacctggtgctggatattttcaaattgtatctagtgcagctgattttaacaataactactgtgttcctggcaatagtgtgttctgattagaaatgaccaatattatactaagaaaagatacgactttattttctggtagatagaaataaatagctatatccatgtactgtagtttttcttcaacatcaatgttcattgtaatgttactgatcatgcattgttgaggtggtctgaatgttctgacattaacagttttccatgaaaacgttttattgtgtttttaatttatttattaagatggattctcagatatttatatttttattttatttttttctaccttgaggtcttttgacatgtggaaagtgaatttgaatgaaaaatttaagcattgtttgcttattgttccaagacattgtcaataaaagcatttaagttgaatgcgaccaa

SEQ ID NO: 23 - DNA, 16142 bp, Homo sapiens
  source 1..16142: mol_type = unassigned DNA
gcccccgccgcccccgggccctgatggactgaatgaaggctgcctacaccgcctatcgatgcctcaccaaagacctagaaggctgcgccatgaacccggagctgacaatggaaagtctgggcactttgcacgggccggccggcggcggcagtggcgggggcggcggcgggggcggcgggggcggcggcgggggcccgggccatgagcaggagctgctggccagccccagcccccaccacgcgggccgcggcgccgctggctcgctgcggggccctccgccgcctccaaccgcgcaccaggagctgggcacggcggcagcggcggcagcggcggcgtcgcgctcggccatggtcaccagcatggcctcgatcctggacggcggcgactaccggcccgagctctccatcccgctgcaccacgccatgagcatgtcctgcgactcgtctccgcctggcatgggcatgagcaacacctacaccacgctgacaccgctccagccgctgccacccatctccaccgtgtctgacaagttccaccaccctcacccgcaccaccatccgcaccaccaccaccaccaccaccaccagcgcctgtccggcaacgtcagcggcagcttcaccctcatgcgcgacgagcgcgggctcccggccatgaacaacctctacagtccctacaaggagatgcccggcatgagccagagcctgtccccgctggccgccacgccgctgggcaacgggctaggcggcctccacaacgcgcagcagagtctgcccaactacggtccgccgggccacgacaaaatgctcagccccaacttcgacgcgcaccacactgccatgctgacccgcggtgagcaacacctgtcccgcggcctgggcaccccacctgcggccatgatgtcgcacctgaacggcctgcaccacccgggccacactcagtctcacgggccggtgctggcacccagtcgcgagcggccaccctcgtcctcatcgggctcgcaggtggccacgtcgggccagctggaagaaatcaacaccaaagaggtggcccagcgcatcacagcggagctgaagcgctacagtatcccccaggcgatctttgcgcagagggtgctgtgccggtctcaggggactctctccgacctgctccggaatccaaaaccgtggagtaaactcaaatctggcagggagaccttccgcaggatgtggaagtggcttcaggagcccgagttccagcgcatgtccgccttacgcctggcagcgtgcaaacgcaaagagcaagaaccaaacaaagacaggaacaattcccagaagaagtcccgcctggtgttcactgacctccaacgccgaacactcttcgccatcttcaaggagaacaaacgcccgtcaaaggagatgcagatcaccatttcccagcagctgggcctggagctcacaaccgtcagcaacttcttcatgaacgcccggcgccgcagcctggagaagtggcaagacgatctgagcacagggggctcctcgtccacctccagcacgtgtaccaaagcatgatggaaggactctcacttgggcacaagtcacctccaaatgaggacaacagataccaaaagaaaacaaaggaaaaagacaccggattcctagctggggcccttcactggtgatttgaaagcacaattctcttgcaaagaaacttatattctagctgtaatcataggccaggtgttcttcttttgtttttaatggctatggagtccaagtgcaagctgaaaaattaatctcttagaaccagacactgttctctgagcatgctaagcatcccagaaacccaaatggggccttcctggagcgagttaattccagtatggtgtcaaccaagctcgggattgcttaaaatatcatccatcccacttcaggtcctgtcagcttcttgcagtcagagttcctatgagtaacaataggagtttggcctatgtaaggactctgagtttaggcttccaagatacaacaataagagaagaatctagcaacgagaatgacctcatttgctttccacatgcttagcctcattataccatgttatgtccaagttcacagccacaacatcagaatggtaattactgagcacaagttttaaatatggacgttaaaaaaaaaaatccaaggacctgtttttccaacccagacatcttttcattgaatgatttagaaagctttaagttgatccagcttacaattttttttttctttacctcctggaaatctcatatggtcttggatccgtcaaaaaaaccagtcagttcacttgcgctcaaagtatcaagcacaacaaagataaacagaagtgaggaaggttctgggttcactacatctggattttcaagacacctattgtgaagtcattagggaattgatgagaatatggcttcaagcacattttgcagtttgctacaaattctgttgtacataatgcagacgcacactcaggaggccaatttaactgttaacagtgcatggagcgaatgcagcattttaaaagatctaggtttttttaggtcattaatgtgtccttggttgatcagtcatctggtccctcctactgtgtgttatgaccaccacgtaatccattctcgctctttctgatttggggtttttcctcatccatcccattagtagggatgttttctgtgttttctagcaagaaaaaaaaatcaatcaatcaaacctgcatacatgttactcatgactgtcatctagtcctaaatctcttctgttgttgaatcatccttgcaaaacagctgaatacatctggagaaaacacagcacaccaaagaagcagaatactgcaaaccaaagacatttatgacttgtcattttctagcctaaaaatactgtgattacttttagaaatcagaaaacctctgcaactccgaatggcattcagctcttgcatttggcgcatcatcgggctgagcggaccagctacaccaaggacattagccaagccacccagaggggtggctttgccacaccagttgtcaccttcccatagcaagtggaagagcgcccacagaactctgggagattgcaaaggtcacaatgtgcatatttaccagtgaatggccccgggtggggccacgtgggggtgttcaaagcaagccaaacgctgcaatcattctttacagacacttgagactgacttttttatgaattacttagtcgaaaccaaagaaactttttctgcacctacttctgcaacaaacaaaactgtcccattaaaatgaataaataaatccgtaaatcaatggaaatcaccaccaataagaaggaagcacgccagaaaataaacgaaaacaaaaacagggagacacactgtgttcaaacagacctcttgggacattttttggaagcagattttaaagaaagggttgagacaaagatagaaataaggaagagcctcagtggctgctgcttcatttgacaactcacacggtaatcttaaagctgaagattgtctttaatttgtgcctatgcagtttttcaaaagaacacggaacagagcaacagaaacctcaacagctacaataccaaagatgaggatttctcacaccttttgtttcagttcattatctcctcttgcctggctaaaatactaatagcgccattgaactgtataaaggtaatcaattatgtttctctgagcaacaaaaggaaagggccatttatttgattttattgtttcatttcaattttgtcttatggttttttgccccaacatggaatctctcaaaagtttccatggactccaagtttaagatgttgggatattgaacagttctctctgctcagcagagggtagggaataacattatcacttgaatgttctttgcttaacccttagacttggttccttctatgttcagagtctcatcatcaggggaaggaaagggagtgagggtcagggataggggtcttggtgatgcatcctctcccgagccacagaaccaaagagtttatagaggaatttacagcctcgttttcatgtgattgctacatcctaacagggcttcatttgggggtggggggaaacatgtaaaaataattgccagtttctacttttctattagctttttaaaaatcagctgtaaagttgcatttctaaagaaagatatatataatatataaaatacatatatagatcaacttgacattggtgataaccaaaattattgctgtccaaattcatgtcttgttttggtccagtgcttcatttgctaagtattcggttcagaatttttctcatttctcatgccattccagagttaatttgccactgtggatgatttgaagtattcagatctctatggaagtttctgggacaggtttaaagtcaagatcaagcattttagcatttaacctgttgataaatggatccatggtgtacatgagttttatttgtattcggagtcatctctattctatccctcagcctcgattaaggtggtgagtgaagtgcatccaacagactcggcccagaactgggtcctgacagtggggtgctcatcttctgtaactgttgggaaggctcggtggtccattttcaccagttaaagaatatgaggccagcccagaaatctgttctccaggagctgccctgtcccatctgggtgtgccagaccccctcagtgagcaggtccaccaaagggacttctcacaggggaagcccaactcctgttgcaatgggttgatagatttcctcagggtggtaattaccaattcgtattttgacaagcctatgtgcaaccacagctggcactggggtgggcagtggtgttgggtgggatgggggagagtgtctcaatcctgaagagaaaatataaagcaggttttggggagacttctggagtcctgcccctagagagccccattgttgttctttgtgccccctcctcattccccctatgtgggtctccctatgcaggagctgtgagagaatgtgactctccacaatttttataattcatccttcctaggagattgttcattggctcttcccttgtgtccctttgtcccttgctcatactccatgtttcctttgtcaaaggactaagaaaagagcatatttcagcagaggagtgttcccatgtgggttgatttcaacttgggtatttctaaaagagtccttgtgacatgtgtccagtggaaatggttgctcttttccagactggattgaggaatggagcctgtttgatttggttagtgattctttgacatactaatctcagcgtttgggtctccagcatcctctgaagatgtctagactagtagaggctgcctttgtgacctgacattacaacattggtcaaaccagtcctctgataatcagaagaacatgtcataattgtttaaaaaaaaaaaaaaggcaagaatttctctccaaggagctttaataaatgtctcattccagataatgtcataccagagaaaagtgcttgcttttagaaaattatttacatacatatataaatatatatgtgtatctatacagttatgtatcaaaattttaagccctgcagaatttcaatttgttagaaatctaacagaaaaaaatttctatattgaaaggtaatagaatttaacccagtgagtttactcaaggatttttaaatttaagttaataatttcagagaaaataaccatttgggtgtggttatagtttagtatccattacctcaatccaaggaaaattccaggcattcctcaaccatcaggaaaaggtacagtgtgaaggaacagttctcagccaaatttcacattcttgaggcaacagaaatcaaaacactcagagccattgagtggaaaaacaatttactttattcctttacacaaataggcttgcattgtttttgttttaatgtgattttggtactagggatataattatttcattccaggaaataataaaaaaaaacagacagagccaatacatttctttttttaaaggaaacagcaacaacaataaaaactcagcaccaatatttaaaagcttttccaaaatgtaaaagaagtgtttagcttgcaccatgcataaaggtgcaggctagttgaaccaggaagcatggcacttcctctggagaaatccagaaagagttgcttctaagctcccttttccccctgcaggctcttggcaattgtaggctttagcaaatccagaataattttcaattcaagctaaaataaaatcaacatttggaatgtaaatctgatacacacacacttttctaagtcaaacaacatatttcaaaaccaaaaataaataccttttagataatcagttattttctttgtctatactgggcacccacctactagtgccagtaaattcaagttgaacagatttttaaaatcactattatctgggtatgggggaaacttccccacttttgaaaatgttggtagaattataggaatgtctgtttgattatcattaccaaagtgtcatgacagtatgcctttgtagtgaactcggattttcaggagtttgaatagttggatattttaaaatctaagaagaaaaggcctgtttccaatgttgttgaagaataatgaactctattaaaaagtggagaaaaagataatacatgtggtcaaggttgaccacaaggcccaggcacaactaccttggcgataatcttctagattcgtaacaggttagagctgactttttgtttttgttgttgctgatgctgtgtgattcagacttctcagcctaaccaggaagagtaagtggaaatggtagatgaagaaggggtagagctggtgtatctataactttctgatatttgtctgccaaacttgatatattagtaatttttttatctttagctaagatcaagtcacccctgaaacaacaggagattctagttttaaaataaggccacaaaaatccttacggaatgaagaatggcaccccagttggttgtataagtctcataagataatgatgttgattttaaatatggatgtctcaatgcctgttttctatcaatgatttgtttgtttccaaggtcggggagggaaagaggggagggtttatctgttttagaaagtctcagaatacttataaaatacagaagtagttattaaaatatataggacctcacataggtagatacagaacttaccattgaggctgatgggctgttgtgtgaatcacacaggaccttaaatgaggctcattattctcacacaccaaaatgactctgacagcctgaagcagttattgctagagcccaagctttccttggaggttttggagttaggttgattggaagtaaccagctaataccttttctagtggagaaaaagacattgctaccagcttgttcatcccatagaagtcttccactctgctccatttttagcagcaagcatttcatgtagcataaaccttggcagataagtgtgcctaaggtttatacagtctgtccgcttggatgtatacaaatttagatacatattttaacatgtgttctcatagatgactttataacaacacacattacctataggtgtctagactgtgtacatacaagtgtgtacagacaagcttcatacgtatatactgtaatccgttacaacaaataaattttaaatcatcgtttaacatgtatgtggtacttctacagtgtacattgttttcattatttattgtaacattgaaaaccacagtgcagggaaaacaaaagtatcccagcatcttcatcctgtacacttggaattaatttcatttgggcatatccaagataaactcaactttcaagaaatcttgtatattatttaatcatctgtgttaggatgacacctatgattgatgacttcggttgaatagctttattctggatttttcataactaaagctaaatccaaagacctgaaaaaggacaaaaagaaaaaaaaaaaaagaaaaaacaaagaaaaagaagaaaaaataataaagtcaagcgcaaactgatggggagacagtgggctctggtttccaggattgagacaatggtactgcggtcttggggagactgcgttagctagtggggagtggtgatttttttcatgcttgtcacatctaaatggtctttaacatgagaaagttttagaggttataatttcctgctttgtttttatttagactatcaaatgaagttatacatgttgtcagtcaaaaaatgaagacaccctctgccccaccccacagaatgctttttatcttgtctctttgggttatgacccaacaagctaagtaccattaatgtaattaacttatttaaattagttcctagtacataaatgtataggatttgggtaattatttaatcatccttccttagtttgattctactccttgtacttatttatcaaaacctagaccaatggtgcatcagagatgcaaaattctacttggaatactcttgaagtttagtttgctttataaagcagtgaaattctgttacagacagggaagaaatacaggttacaaaaagagaatttgggatattcttccctcttaaattaacttttaaaatagtctaagtaacaatttttaaattatttaacttaagttcgcagccccacctggtaccaggcgaacttcacctcttaattattgtggccctcggagccttcatattgtaacttatttatttaacttattcagcatctgtgaaaggtgcactgtatagtttatatttttaatttaaaacaacagagagcactgcagtttgtttgctgtcagaacaacagagcaaattttgtggacaagcaatgactattcagcctgaacctgtgcattcagaaaacataagctgagaccctgcttcaccagcctggatttcggggcttctatacagaaactggaaaaataaattttaaaaaaatcgtaaacaaaaagagagaaacccttacactagctgcttccaagaatgaactctgtgtgtatgtaaagcaacaaaacaaaaaaggaaaaaaacaaaaagcagaaaaaagaaaaaaaaaatgaaaaactttctatttctagtgagaaccaaagaaggctacctcactgactttttccatttgtaattttaatcgtgttgatgacaccaaagataccaaagatttctttctctgtgcggtctgcattttgcttgtgctcttttataatttgaacgattttctctgacatatggtatgtacagccacagctcagataccccaaagaaataattatctatgcgacggcggctgctaatttggaaagggatattttctgtgtttctcttatatgtttgctgtctgctcgacatgttcaagatgcgagttcagatgctgctgtaattggattccttaaattctgattacaaattgaggaaggaaactggttggaaatggccttcagtcctagccatggcctctatccccgctgggacctgtcacagtaaagactgccaattactgaaccacagaagctctgaccattgagtagttgagctggaagagaccttaggaatcatttagtccaagccccggtggcccagaggaatgaaatagttatccaaatcaaataactcttgagagtgaaagcccacacatgcctcctggttcctgccccagtgctccgcttattgtacagtgctacctctgcatgagagcggtcccacattgacaaataggatggtggcaatcctttagcaatgagcagggactggggtttatctcttaacattttcagctgtaaaattagtcacaagcattttcagtgtcccattagtacatagtcacatatggtcggttgcttcgtgaaggtggcctgtcttgaaatactagggctcatacgggatttttgccctaggaaaaacatgttgatcccaatgatgtgatcacttttgaacctttccattacaaagcattgtatagataactttttaattcagtaggaggagaaagttcattcttggcctgttggctttgattattatgggtactttaaagtcagtatttatcaagaaagggaacttgaccaccattggcacatgtgacatttaagctcttcagccttttcctttttagttgtaggtgtttacatttcatttctaagccaactctgtatttatgagagaagtttaagccttacatcatttgatactaaagggttatttgtggtaaatgaaaaatgaccccaaaattacagaggaatatgccagtttaagaaatggctacttaaagttgcttctctctttccttcttactcatgaaattaattggtcttcttcaagtttctttagattccattaaatgattaaatcactattaagagccattcatcaacgtgatttgtgtgttagccaatgaatctgtctcagcttttgaccaaatgggttttagacaaatgcaaagatctgcctctagtccatatggctctttttgagtgctagtattttgcatttcacataatgtagttattttgagcttttaaagagagcatttagacaaagaagcaaagagaggaagggaccaatcaactcatcagttccatgcatcaacaaagcatagctagtagaggaatataaatgacagattgacaaactgtaggaaacactgttactctctttctgaagttttcaagcaccatcctatgtgaaagttccctcctgtccaaacaagctcaaggcccatcttctccctatacaaggcaaacctgtaaggccttccttccaaagagtacattgctttggttttcttcctaaattcctattggaattagaactctcagaatccctgggagacagagcaaagatgacttaattcattgagcagcagagctccctataagtgaacatcaccttccccatctttcctactgccacacccatacgagagaggatctagaaagagcgatggcagcctgaacacagaaaacatccccacttggcagacctctcctcagcaatccccccagcctcatgcttcacttgcaaagtgtgacataaccacgggacgagtgccttgcttgaaccaaagcaacgatttagccagtctggacctctctgtgctttttttaattcttcctgtgaatacctcagcttcaactgggcctccatacagtcagttggtgggcttattgtactgtggtgctttgcaatgcaaccctgcaaagaacaagatttgtactaataccaaaggttctttctctatgtctcctcctctgcctccctcgttcttcccttttttctagttcttcacggttccaaagctttactatgaacctgggcatgttggcaatgcagaccgcgcaattccttaccgaattttctcagatatacctcatagacaatagtgtttagagtaatgttattatagcgtatgtaataaattattcactgtttcttttggtaactgtgatttaaaaaaagaaaaaagaaaaaaaagctttatacgttttaggttgtgcttttgtaatagatgaaaaaaggtgcgcttaaaaagaaaatgtatgtttttttccccctttggattttatttatgctggattggggaaagttgcagaatgagcccaaagtttacagtttcatattttgctgaagaaacaatctgtgttcatttgctctgttgaaaagaataattattttctacatttgtgccacttggtctgaacaattaattgttccgtgttaacagtgtagtattatgattagcaactgccaatcagtgctataattttatgcatgaggctaaaaatttagcagtgtgatgcattgtggtcttaatagcaacatttttcattttgaactagatcttcccctttggttcaatggactttatttatgcatgggcgcctattgtttgttagcagttgtggaacagttgtgtatacattaaactgtgaaaatgtacacagttcagcctcagacggtggtaatattggttttattgggagatgtgtcacctcgaaaataccctttacatctgttgggatctgaaaatgagtcacattgaattgggttccagctttataatgagaaacgttattcctaatttttgagttagccaatttgcattccacaaattgggatcctcataacccaaatatatcaccgtatgtgagagggatttgaaagcgagtattgaaaaactcacctttgcatatttaatttccaccaaaaggagttattttggctttatgctcatgaacttagacctaactggccatgtatatgtagatgcaaattcatctagctgtggccctctttgatctctgcttgggaatggctatttttgactatgcgtggtttcttctcgtattttgtgatcaggtcagctcccagtagaaactcaaatggcatcaatattactaactcttctctgcccacttctcttttgtccactctcctagacattcccaccaactgttccagtgatttgggcaaaaatacgcagccatttcccaaaacttcacatgtgcagctatcatggctgtccctccctagacttggaggtgactctcacttaatttttacctgcccaacaatgttccatctaccatctaaaaggtaatataagaagaagttttgaaacccactttaggaaaaccatcttctttaaatccttcaattatctgaggcctctatatgtcaaaactatttttcagttgcaggggattgggcaaacttgttctttcttatacttgggttcaaagacccattctccagtttcatatttcccaaaccaaaatgcttgacataaagccaaatcaactgccaagcacactttattttgcataggagtatgcagcctagggaaccttggttgaaaagcagcagtctgctatgcaaaatattggaaatcactgacagtgtagcattcatattatctgtcaatgagggtatattgggaacgtgctctcgtgaataataaaaagcaacatatttttatttggccttataaattaggttgtggtaatgtaaactttgatatatagtctttttatttttctcttattaatctgccaaagatgggaacagatacaagaatttttcaaattggcttttgtaagacaattgatgattgtaatagtgtttaatcttccagaaagctttatatgttgttccacaataaaattgatatttgtttcagcaaagttttcctgacactcacaaacccacaaactgttcctcttaatgcagatattgtagaatctacaaagttcaaatccatttttgatccaaagaaagtagaggagtatttgagacatgagtgtacccagccctttttttaatcacaggcaatgcatgggtctggctggttacactttgccaagaagacttgtcttatgaaacccaaggtatattttgttatgccattttatgtccttttcttttaacattgtggaaagtggtatgttgaatcaagtgtaagctgagttttccagacaactgaagtagctacatcatgaatgttattttgttattaaagggtttttactcagtgctttgtgccaatggatgtccttttccttggagacacataactacaaaattacctcagcttggcctggttttctctcctgccctcttggggaaacatgggcctggcctgggaaaaggcaggtcatgggctggaaggtaggttttggtactaggaagaaatctctgtatctgtcagctttaaagagaactgggccaaaaatctctaacctcactctctctggactccaacacttccctgcaatcctttggtcttgagcatgtgccagcatgaaggcagactccagttcatacatgaaaggcaagaaaaagaaaatagtaaccttgaatcttctgtgggccaccaggcactcacctttccccaccttgcacactatccagtcaaggctattgcagcccatctggtggctttacatgggacattaccaaaggcttcttcctccatcctggggttgcaaaggatccaggtcccctccatccagtggggctcttccacatcagaagtccccctcccaccatcctctgcatcctgtttagctatcccatctataccttttggagatgattatttagaaaacaaagaaaggtatggaatggggtttcctattgtttgctaggttatattttagcaattctcaattctttgatctggaaaaatacaagagggaaaaggagaccccactatctccctgtgctttgctcccatctcagggggcaggggcagtgcacattgcctatgctgttgatctgtcttgggcgacaggctgaatcacagctattgccccagccaaaaacatggcccatcaatgcctactttatctctgcttgaaaatcctattcaaaaagttgtagagtttgaggtttttatccccccatatcctttgctttggtccagtttggcctttagcataagagtcagctttatctctaggaaagttttttcagattatgacaaggaacctgccacctgggaagaaaagagtccgaagactagcaatcggataggtagtcataccattaacagatacttccttgaaggtagaatattatttcctttctttacagttttgtgttacacaagtccaagtggtgccagcaaacttcttaccgtgaaatgttgtaaaacacctggcatactgaaatttctgaaacaaaaacacaagctccacattgataacttgataaataaccactaaagtttagatgcagggactgagatgatacaggcaaaatcttggtgttggtttctcttttaattcgtatcttcgatcacctaacctttctcaatccaagagcagttcagtcttttctccccaagtctaggatgccaaagagcatcataggaaaagataattagggattgaccagcatttcaattagttctcttcttcatctttgcatttctcaaaagtgttctcctggaccagagggaaagagctggtccattttttttcattctttctattcaaatttttccacccagacaatactttattaacacagatactgtagatccttccttggtcagtgaattattacaagaggagctatccttccaccaaagtgagtgaaaacaagttccagtatcttttcttccatccagttttgttctcagaatccaagtcagtcctgggtcttttctcactttagaccctggcctcagatgtgtttattcttgctatttaaaaatacctttaaatttcacatgctggcctgcagaacttgcatcctttgttctatactgttgactgcttgatggtattgaaaggtgactataatgagggaagaaaggaggaggtaaagagagaagaatttgtcccagatctgtttaaagtttcaaaatttaaaaagggacccattaaattatgggaaaatggctatagagtgtgagcctccgttgaccatatgctcaaagaccgtactctgccacctgccttccaggtagctattctagaaactcagtcctttgtggaaacccaactaccttttaaaagtctctttccagattccaaaaggacaagagatcagagagtcacatatacgcctcttgttttattttcttgctttcacgggtattattgccaagaaaatcgtagggaaaaactttaaacttttcttttcagttgatccctttgacatcacctctcatgtttaaaatcaggaaaacacacccctaaaatttgcactctcttccgttttgaaaaagaaaacccacacacaaatgcacactattaccgtctttcaccctgcgctatatttccaaagtgtattataatccagatattgccccatctcaaacatgttaagtcagactgtgctgaaagactttccagggacggtcaacagggtatatgttcagtggctgccctgaaatcctggtggggatgaggatcacgcttcatcatcaaggggatgcccatcccctgataagctcccagtccttttggaagatttctttgaatgttaattgcattttcagttttgctcatttcccaccccaatgttttgtctgcaacatcgcttacactggattctttctatttttattcctatcattaaatggtagtgctgtaaattctgcaattaatgttaaataaactgctttaattcattgaaaaaaaaaaaaaaaaaaaaa

SEQ ID NO: 24 - DNA, 8468 bp, Homo sapiens
  source 1..8468: mol_type = unassigned DNA
agctgagggagcgctctgaaataatacaccattgcagccggggaaagcagagcggcgcaaaagagctctcgccgggtccgcctgctccctctccgcttcgctcctcttctcttctttacccttctcctctctcctcctctgctgctctctcctctcctcccgctcttctctctcctcctctcctgctctctcctcttcccttagctcctcttcttttcttctcctcttcttccctctcctcgcctctcccctgctcctcttctctcgtctcccctcccctcccgcctctctctcccctctccctctcccactcgccccgctcgctcgctcgctgtcgcacagactcaccgtcccttgtccaattatcatattcatcacccgcaagatatcaccgtgtgtgcactcgcgtgttttcctctctctgccgggggaaaaaaaagagagagagagagatagagagagagagagagagagagagagagaggctcggtcccactgctccctgcaccgcggtcccgggattcttgagctgtgcccagctgacgagcttttgaagatggcacaataaccgtccagtgatgcctgaccatgacagcacagccctcttaagccggcaaaccaagaggagaagagttgacattggagtgaaaaggacggtagggacagcatctgcattttttgctaaggcaagagcaacgttttttagtgccatgaatccccaaggttctgagcaggatgttgagtattcagtggtgcagcatgcagatggggaaaagtcaaatgtactccgcaagctgctgaagagggcgaactcgtatgaagatgccatgatgccttttccaggagcaaccataatttcccagctgttgaaaaataacatgaacaaaaatggtggcacggagcccagtttccaagccagcggtctctctagtacaggctccgaagtacatcaggaggatatatgcagcaactcttcaagagacagccccccagagtgtctttccccttttggcaggcctactatgagccagtttgatatggatcgcttatgtgatgagcacctgagagcaaagcgcgcccgggttgagaatataattcggggtatgagccattcccccagtgtggcattaaggggcaatgaaaatgaaagagagatggccccgcagtctgtgagtccccgagaaagttacagagaaaacaaacgcaagcaaaagcttccccagcagcagcaacagagtttccagcagctggtttcagcccgaaaagaacagaagcgagaggagcgccgacagctgaaacagcagctggaggacatgcagaaacagctgcgccagctgcaggaaaagttctaccaaatctatgacagcactgattcggaaaatgatgaagatggtaacctgtctgaagacagcatgcgctcggagatcctggatgccagggcccaggactctgtcggaaggtcagataatgagatgtgcgagctagacccaggacagtttattgaccgagctcgagccctgatcagagagcaggaaatggctgaaaacaagccgaagcgagaaggcaacaacaaagaaagagaccatgggccaaactccttacaaccggaaggcaaacatttggctgagaccttgaaacaggaactgaacactgccatgtcgcaagttgtggacactgtggtcaaagtcttttcggccaagccctcccgccaggttcctcaggtcttcccacctctccagatcccccaggccagatttgcagtcaatggggaaaaccacaatttccacaccgccaaccagcgcctgcagtgctttggcgacgtcatcattccgaaccccctggacacctttggcaatgtgcagatggccagttccactgaccagacagaagcactgcccctggttgtccgcaaaaactcctctgaccagtctgcctccggccctgccgctggcggccaccaccagcccctgcaccagtcgcctctctctgccaccacgggcttcaccacgtccaccttccgccaccccttcccccttcccttgatggcctatccatttcagagcccattaggtgctccctccggctccttctctggaaaagacagagcctctcctgaatccttagacttaactagggataccacgagtctgaggaccaagatgtcatctcaccacctgagccaccacccttgttcaccagcacacccgcccagcaccgccgaagggctctccttgtcgctcataaagtccgagtgcggcgatcttcaagatatgtctgaaatatcaccttattcgggaagtgcaatgcaggaaggattgtcacccaatcacttgaaaaaagcaaagctcatgtttttttatacccgttatcccagctccaatatgctgaagacctacttctccgacgtaaagttcaacagatgcattacctctcagctcatcaagtggtttagcaatttccgtgagttttactacattcagatggagaagtacgcacgtcaagccatcaacgatggggtcaccagtactgaagagctgtctataaccagagactgtgagctgtacagggctctgaacatgcactacaataaagcaaatgactttgaggttccagagagattcctggaagttgctcagatcacattacgggagtttttcaatgccattatcgcaggcaaagatgttgatccttcctggaagaaggccatatacaaggtcatctgcaagctggatagtgaagtccctgagattttcaaatccccgaactgcctacaagagctgcttcatgagtagaaatttcaacaactctttttgaatgtatgaagagtagcagtcccctttggatgtccaagttatatgtgtctagattttgatttcatatatatgtgtatgggaggcatggatatgttatgaaatcagctggtaattcctcctcatcacgtttctctcattttcttttgttttccattgcaaggggatggttgttttctttctgcctttagtttgcttttgcccaaggcccttaacatttggacacttaaaatagggttaattttcagggaaaaagaatgttggcgtgtgtaaagtctctattagcaatgaagggaatttgttaacgatgcatccacttgattgatgacttattgcaaatggcggttggctgaggaaaacccatgacacagcacaactctacagacagtgatgtgtctcttgtttctactgctaagaaggtctgaaaatttaatgaaaccacttcatacatttaagtattttgtttggtttgaactcaatcagtagcttttccttacatgtttaaaaataattccaatgacagatgagcagctcacttttccaaagtaccccaaaaggccaaattaaaaaagaaaaataatcactctcaagccttgtctaagaaaagaggcaaactctgaaagtcgtaccagtttcttctggaggcaaagcaattttgcacaaaaccagctctctcaagatgagactagaaattcatacctggtcttgtagccacctctctaaacttgaaaataggttcttcttcataagtgagcttacatcattcttcataaagaaaaatcctataacttgttatcatttttgcttcagatactaaaaggcactaagtttccaatttacgctgctcaactttgtttatatgcttaaaaggattctgtttacttaacaattttttcccctaaaatactattttctgaatacttccttccagtaaggaataaaggaaagcccaacttggccataaaattcttgcctacactagaagtttgttgacagccattagctgacttgatcgtcatctcctaagaggaacacatatattttcacaagcaattccacactatcctgatgggtatgcaaagtggtgacagtctaactcagtgtttcttcattttaggtataacattttaaagcaattgataatgcctcttccaattcagaagctagtattgaccaaaatgtgagaagagtgtatagcataggaaaatttggggttaacccaaaagacacaattccagcacacataagaaagctagctgctattttatgctttcttccatggttctcctcttttttcccttttatttttccctgtttttcaatgatgtacagtgttccctacttgcattgaaaaaactcgtatggcattcacactttttttcttaggtgggtttttgtgtccagatgcagtaagaattcattgttcatcctaaaactgttttccagacccttccttccccttaggtaatttgatatacacctcctaaaatgacacagtaacaaatctggtatttagaacatatagaacataaatgccattttttaattcaactttaataagaattacatttgactttggagaatacaggtcttgacccatgtgactgactagctgacccgatcgctgtaatttaacgtcatttataaattctgctgatggacaggaatgtatgaactcaattattgtcagcacaaagccttaaaacctgctgactttaaattaaatggtgcagtcctatgatgccctgcaccatccaggggactaacagggcctcgcagtgtagacagagggtgcagccacacgggcgggggcaccagccacctcactctgcacccgcggcctcacacatctcccagctcacactctactaatgcacagagtcattagatccaatttgttatttttctcacttgctttaaaaaaaagcagtttggataatcatgacattggaataaagtgggaaggaaaaattccatcagcacaaaatagggaagtaatcccaacttgtagtcacagttttctgactggctttgttttaaaagaggatggcagtccttgttcgtgtcagtgtgccactgggtttttgctgttccgtgtaattcatatcaactttgtgttgccatttgcaaggtaaaaggcaaagctgtagtgtattcacctatgtagacagattgctagatatctttttgatctggggcgagttcaatattgattccagacttatttggatttttttagtattattttcccctccctttctaatttaaatagacaaattaagcaaaagtgtgtgttcacaaccaaatgttgatgcccttatctactgataatatcctctcaatgttcactgaggcatagaaattatttcagagtagaaattgcagcatgaggataaactcacctctttgttctgaaaatagaactttatcactatgctttccggtggttttcccttttacaatcgaaatcttgtgcctcccaagtgcattggaaaatgacaaaagcctgtctctccaaattcctatttaacagtttgattttttttttttaatcaccatctttcaaatcttagctcaactctcaccaagtgaaaattggctacttgggagaaagttaactttctatggtgggatggtgaaggatgagggacagtttacataggaaaagaaaaaaaaaagtctaaagtccatgttgaaaaaccacactaccacttattttctgctaaccctaaattatttttgcgtatacgcttgaggttatagtctgtgcctagacctaaaatgcaccagcgggggggattttaaaaaatccttcaaaataccagttttttcccaacaagtacaattgttcttgtgccttctgtggctttcgatttcatctttttgactttatttccaattactacagctgcaataaacactagattttttttctggctgtttgacataacgttgatagctatgcatattttgtgtctttttaaaacaaagcgggagaatacgtttttgaagaagagaatttttagaacagtttgataccgcaaattattttttcctcaattgtttgagcagcattcgagttttgaaaattcttgtagaagccaattttttgtaactgtggtgcaaatcttgtgttttcttagcctaatgaaaagtagtatagaagcaatatttcataccatgtgctatatatgtgtgcgcagatgtgtgaacataaaatcacatacacacatatacacacatgtaaaaatatacatatatatatatgcgtgtgaagtggaaagcttaccttttcctatctagatttaagaacctattttagacatttgttatgttttgtgaaaagaatgttctatttgcaacaaaacatttaattcttactgtatctctggctgtttaatgaggacgtttcacattaaatggtaaaacacatggaagatgttagaatgtagtaattatttaagtaaacgttcacccacatattcctgaagtttgctttgtgcctccgagtattatttaattaaagaagtgttttatgtttgcagaatctttgtcactgtactagggatgtgggtgaatatcatttaaaaaaatttaaaacaacaaaaaaaaagcaaaacagaaacactaaagcaagaggggaacttttataaagcaatgtaaatatttaacctcatggctgtcattatgtaagacatgagattttaataaataactacattctcacgacatctgttgaatttactaggaacactacagtgactgtatagacagttgaaagcattcttgaaaatcctgctctctccttttaaaagttaacaatctcttttatcagatgtcaagggcaagggtaatgcagtttctgtaaatttatgaaatttctttttctatgtacatgaagacatttagtaagtaacacccccccttcccatgcgcacatgtgcgcatacacacacacacacacacacacacacacacaaacacacacactgtcataaagctaatgatttggggactttaaaaaataggatgtcctccaggaacaatcataaatttatgaaagaaagagtagtttacagactcccctgaaagaagcagtgtatatgtgaagacagtgcaaaaatctctttgccatgtatattatagcgtattcattggtgtgaatagtacaaatgtttccttctggtacaaactctgtgtttgcaaatttacaagaagcattgttttcaaaaagctccccttaaaaaatgtaactggtttatatgagtaagcagttaccgtattgcacttaaatgttatgttgaaggaaatgcagttttgttttctgtagatctgttggttgtaaaccatctataaaactaaagctaaaatgctcatattcagagctgggatcaaaactggtatttaacctttgcatcttcttataattatccttctaagaatataacagaatgtggaagtgtctggactttgagtcttttcaactgagccttctctcaaatctgacaccccctcagaatgcacaaacataagcagaaaaggcaaacaagcttaccttcttttgtgaaaacgtattcattctgtatttttttaaatattcaattcccctaaaaatggggagaaaatattttaaaattgtatattacgacttcaaatttagaactaagaaaaaaatgtatttgggattggtctcagcgctacctagaagaatcaaaggtcatggcttccctcaatattgtcccagccatttctcatatgtatatagtataaaccgtgacaaaacactgcctttatattatttagcaatatgttgtaaatagcattattaagctcttttttgtaataaagaccctttgatttgaatatagtacaataactgaactgataaagtcaatttttgatttttgtttgttttttttagctagaggcaatttcaattgtgaatttttgttgttgtctattgttctgaagactttgcataatttattggtttaatttatcctaatttatttgatgaaggtgtacaattttgtattaccaaggatgtactgtaatattaattgatatgataaacacaatgagactccctgtccatattaaaaagaaaataaaaaggtgcagtagacaattgattttaaaggaaaagttaaaaaaattagtttggcagctactaaattttaaaacaggaaaaaaaaaagttgttgtggggagggtgggaaaggggttttactttgtgtgttttaagcttttgtatactctccaaacttttaccttttgctttgtaccacttaaaggatacagtagtccaattgccttgtgtgccttccatctcctcttaaactgaatgtatgtgcagtatatatgcaagcttgtgcaaaataaaatatacattacaagctcagtgccgtttgattttcttaaagaaagagtgacttttaatttttggacctgtatccaattgtaggacagtaggctagttgtgccagtaatgtcaagtatggagattttctttcactacaattcttcattctgttagcctaacgtgcagctcctagaaacaacctcttttactttagatgcttggaataattgcttggatttctctctctgaaacatctttcaggcttaactttatttagccctgaaacttaaaaaaaa

SEQ ID NO: 25 - DNA, 2325 bp, Homo sapiens
  source 1..2325: mol_type = unassigned DNA
tctatgtttatatcatttagcagggaaggattgttaatgactaatctgtgtccatgaggcacagagccaaggaagagatgctgctgctagcccagaaggccgcctgtgatcatgcacagtacactggaactctctcctcctcctcacctcattgtctccccgacttatcctaatgcgaaattggattctgagcatttgtagcaaaatcgctgggatctggagaggaagactcagtccagaatcctcccagggccttgaaagtccatctctgacccaaaacaatccaaggaggtagaagacatcgtagaaggagtgaaagaagaaaagaagacttagaaacatagctcaaagtgaacactgcttctcttagtttcctggatttcttctggacatttcctcaagatgaaacttcagacactttggagttttttttgaagaccaccataaagaaagtgcatttcaattgaaaaatttggatgggatcaaaaatgaatctcattgaacattcccatttacctaccacagatgaattttctttttctgaaaatttatttggtgttttaacagaacaagtggcaggtcctctgggacagaacctggaagtggaaccatactcgcaatacagcaatgttcagtttccccaagttcaaccacagatttcctcgtcatcctattattccaacctgggtttctacccccagcagcctgaagagtggtactctcctggaatatatgaactcaggcgtatgccagctgagactctctaccagggagaaactgaggtagcagagatgcctgtaacaaagaagccccgcatgggcgcgtcagcagggaggatcaaaggggatgagctgtgtgttgtttgtggagacagagcctctggataccactataatgcactgacctgtgaggggtgtaaaggtttcttcaggagaagcattaccaaaaacgctgtgtacaagtgtaaaaacgggggcaactgtgtgatggatatgtacatgcgaagaaagtgtcaagagtgtcgactaaggaaatgcaaagagatgggaatgttggctgaatgtatgtatacaggcttgttaactgaaattcagtgtaaatctaagcgactgagaaaaaatgtgaagcagcatgcagatcagaccgtgaatgaagacagtgaaggtcgtgacttgcgacaagtgacctcgacaacaaagtcatgcagggagaaaactgaactcaccccagatcaacagactcttctacattttattatggattcatataacaaacagaggatgcctcaggaaataacaaataaaattttaaaagaagaattcagtgcagaagaaaattttctcattttgacggaaatggcaaccaatcatgtacaggttcttgtagaattcacaaaaaagctaccaggatttcagactttggaccatgaagaccagattgctttgctgaaagggtctgcggttgaagctatgttccttcgttcagctgagattttcaataagaaacttccgtctgggcattctgacctattggaagaaagaattcgaaatagtggtatctctgatgaatatataacacctatgtttagtttttataaaagtattggggaactgaaaatgactcaagaggagtatgctctgcttacagcaattgttatcctgtctccagatagacaatacataaaggatagagaggcagtagagaagcttcaggagccacttcttgatgtgctacaaaagttgtgtaagattcaccagcctgaaaatcctcaacactttgcctgtctcctgggtcgcctgactgaattacggacattcaatcatcaccacgctgagatgctgatgtcatggagagtaaacgaccacaagtttaccccacttctctgtgaaatctgggacgtgcagtgatggggattacaggggaggggtctagctcctttttctctctcatattaatctgatgtataactttcctttatttcacttgtacccagtttcactcaagaaatcttgatgaatatttatgttgtaattacatgtgtaacttccacaactgtaaatattgggctagatagaacaactttctctacattgtgttttaaaaggctccagggaatcctgcattctaattggcaagccctgtttgcctaattaaattgattgttacttcaattctatctgttgaactagggaaaatctcattttgctcatcttaccatattgcatatattttattaaagagttgtattcaatcttggcaataaagcaaacataatggcaacaggaaaaaaaaaaaaaaaaaaaaaaaaaaaaaaaaaaaaaaaaa

SEQ ID NO: 26 - DNA, 3288 bp, Homo sapiens
  source 1..3288: mol_type = unassigned DNA
ccccgcgctgcgcggagcagggaccaggcggttgcggcggcgacagccatggccggcgcgctggcaggtctggccgcgggcttgcaggtcccgcgggtcgcgcccagcccagactcggactcggacacagactcggaggacccgagtctccggcgcagcgcgggcggcttgctccgctcgcaggtcatccacagcggtcacttcatggtgtcgtcgccgcacagcgactcgctgccccggcggcgcgaccaggaggggtccgtggggccctccgacttcgggccgcgcagtatcgaccccacactcacacgcctcttcgagtgcttgagcctggcctacagtggcaagctggtgtctcccaagtggaagaatttcaaaggcctcaagctgctctgcagagacaagatccgcctgaacaacgccatctggagggcctggtatatccagtatgtgaagcggaggaagagccccgtgtgtggcttcgtgacccccctgcaggggcctgaggctgatgcgcaccggaagccggaggccgtggtcctggaggggaactactggaagcggcgcatcgaggtggtgatgcgggaataccacaagtggcgcatctactacaagaagcggctccgtaagcccagcagggaagatgacctcctggcccctaagcaggcggaaggcaggtggccgccgccggagcaatggtgcaaacagctcttctccagtgtggtccccgtgctgctgggggacccagaggaggagccgggtgggcggcagctcctggacctcaattgctttttgtccgacatctcagacactctcttcaccatgactcagtccggcccttcgcccctgcagctgccgcctgaggatgcctacgtcggcaatgctgacatgatccagccggacctgacgccactgcagccaagcctggatgacttcatggacatctcagatttctttaccaactcccgcctcccacagccgcccatgccttcaaacttcccagagccccccagcttcagccccgtggttgactccctcttcagcagtgggaccctgggcccagaggtgcccccggcttcctcggccatgacccacctctctggacacagccgtctgcaggctcggaacagctgccctggccccttggactccagcgccttcctgagttctgatttcctccttcctgaagaccccaagccccggctcccaccccctcctgtacccccacctctgctgcattaccctccccctgccaaggtgccaggcctggagccctgccccccacctcccttccctcccatggcaccacccactgctttgctgcaggaagagcctctcttctctcccaggtttcccttccccaccgtccctcctgccccaggagtgtctccgctgcctgctcctgcagccttcccacccaccccacagtctgtccccagcccagcccccacccccttccccatagagcttctacccttggggtattcggagcctgcctttgggccttgcttctccatgcccagaggcaagccccccgccccatcccctaggggacagaaagccagcccccctaccttagcccctgccactgccagtccccccaccactgcggggagcaacaacccctgcctcacacagctgctcacagcagctaagccggagcaagccctggagccaccacttgtatccagcaccctcctccggtccccagggtccccgcaggagacagtccctgaattcccctgcacattccttcccccgaccccggcccctacaccgccccggccacctccaggcccggccacattggccccttccaggcccctgcttgtccccaaagcggagcggctctcacccccagcgcccagcggcagtgaacggcggctgtcaggggacctcagctccatgccaggccctgggactctgagcgtccgtgtctctcccccgcaacccatcctcagccggggccgtccagacagcaacaagaccgagaaccggcgtatcacacacatctccgcggagcagaagcggcgcttcaacatcaagctggggtttgacacccttcatgggctcgtgagcacactcagtgcccagcccagcctcaaggtgagcaaagctaccacgctgcagaagacagctgagtacatccttatgctacagcaggagcgtgcgggcttgcaggaggaggcccagcagctgcgggatgagattgaggagctcaatgccgccattaacctgtgccagcagcagctgcccgccacaggggtacccatcacacaccagcgttttgaccagatgcgagacatgtttgatgactacgtccgaacccgtacgctgcacaactggaagttctgggtgttcagcatcctcatccggcctctgtttgagtccttcaacgggatggtgtccacggcaagtgtgcacaccctccgccagacctcactggcctggctggaccagtactgctctctgcccgctctccggccaactgtcctgaactccctacgccagctgggcacatctaccagtatcctgaccgacccgggccgcatccctgagcaagccacacgggcagtcacagagggcacccttggcaaacctttatagtcctggccagaccctgctgctcactcagctgccctgggggctgctttccctgggcacgggctccagggatcatctctgggcactcccttcctgccccaggccctggctctgcccttccctggggggtggagcagggtccaggtttcacacttgccacctcctggaggtcaagaagagcagagtccccgtccctgctctgccactgtgctccagcaccgtgaccttgggtgactcgtccgctgtctttggaccgctgtgtttcaatctgcaaaatggggatggggaaggttcaatcagcagatgacccccaggccttggcagctgtgacattgggggcctaggctggcaactccgggggctcaacggtggaaagaggaggatgctgtttctctgtcacctccacttgctccccgacaggtggggcacagacctctgttcctgagcagagaagcagaaaaggaggttccctctctctgctccttcactgctgacccagaggggctgcaggatggtttcccctgggagaggccaggagggcctgatcccaggagacaccagggccagagtgaccacagcagggcaggcatcatgtgtgtgtgtgtgtgtggatgtgtgtgtgtgggttttgtaaagaattcttgaccaataaaagcaaaaactgtctgctggttaaaaaaaaaa

SEQ ID NO: 27 - DNA, 6193 bp, Homo sapiens
  source 1..6193: mol_type = unassigned DNA
agaggcgctttcggcttccaagggggaagtgctgggctataattaatgtttttattaaatttggagggaagtttttgcagcctttcgcctagcgtggccttcaggttgatagaagtccagatcctgaggaaatctccagctaaatgctcaaaatataaaatactgagctgagatttgcgaagagcagcagcatggatggattttatgaccagcaagtgccttacatggtcaccaatagtcagcgtgggagaaattgtaacgagaaaccaacaaatgtcaggaaaagaaaattcattaacagagatctggctcatgattcagaagaactctttcaagatctaagtcaattacaggaaacatggcttgcagaagctcaggtacctgacaatgatgagcagtttgtaccagactatcaggctgaaagtttggcttttcatggcctgccactgaaaatcaagaaagaaccccacagtccatgttcagaaatcagctctgcctgcagtcaagaacagccctttaaattcagctatggagaaaagtgcctgtacaatgtcagtgcctatgatcagaagccacaagtgggaatgaggccctccaacccccccacaccatccagcacgccagtgtccccactgcatcatgcatctccaaactcaactcatacaccgaaacctgaccgggccttcccagctcacctccctccatcgcagtccataccagatagcagctaccccatggaccacagatttcgccgccagctttctgaaccctgtaactcctttcctcctttgccgacgatgccaagggaaggacgtcctatgtaccaacgccagatgtctgagccaaacatccccttcccaccacaaggctttaagcaggagtaccacgacccagtgtatgaacacaacaccatggttggcagtgcggccagccaaagctttccccctcctctgatgattaaacaggaacccagagattttgcatatgactcaggctgtatgtttgaaaagggccccaggcagttttatgatgacacctgtgttgtcccagaaaaattcgatggagacatcaaacaagagccaggaatgtatcgggaaggacccacataccaacggcgaggatcacttcagctctggcagtttttggtagctcttctggatgacccttcaaattctcattttattgcctggactggtcgaggcatggaatttaaactgattgagcctgaagaggtggcccgacgttggggcattcagaaaaacaggccagctatgaactatgataaacttagccgttcactccgctattactatgagaaaggaattatgcaaaaggtggctggagagagatatgtctacaagtttgtgtgtgatccagaagcccttttctccatggcctttccagataatcagcgtccactgctgaagacagacatggaacgtcacatcaacgaggaggacacagtgcctctttctcactttgatgagagcatggcctacatgccggaagggggctgctgcaacccccacccctacaacgaaggctacgtgtattaacacaagtgacagtcaagcagggcgtttttgcgcttttccttttttctgcaagatacagagaattgctgaatctttgttttatttctgttgtttgtattttatttttaaataataatacacaaaaaggggcttttcctgttgcattattctatggtctgccatggactgtgcactttatttgagggtgggtgggagtaatctaaacatttattctgtgtaacaggaagctaatgggtgaatgggcagagggatttggggattactttttacttaggcttgggatggggtcctacaagttttgagtatgatgaaactatatcatgtctgtttgatttcataacaacataagataatgtttattttatcggggtatctatggtacagttaatttcacgttgtgtaaatatccacttggagactatttgccttgggcattttcccctgtcatttatgagtctctgcaggtgtacaaaaaaaccccaatctactgtaaatggcagtttaattgttagaaatgactgtttttgcaccacttgtaaaaaggtatttagcgattgcatttgctgtttgttgttttattttgctttatatatgacttgcagaggataaccataaaatgggtaattctctctgaagttgaataatcaccatgactgtaaatgaggggcacaattttggactctggcgccaaactgagtcataggccagtagcattacgtgtatctggtgccaccttgctgtttagatacaaatcataccgtcttttaaatattttgaagcccatttcagttaaataatgacatgtcatggtcctttggaatcttcatttaaatgttaaatctggaatcaaaatgaagcaaaaaatatctgtctccttttcactttcttcagtacataaatacattatttaatcaataagaattaactgtactaaatcatgtattatgctgttctagttacagcaaacactctttaagaaaaatatccaatacactaaataggtactatagtaatttttagacatggtacccattgatatgcatttaaaccttttactgctgtgttatgttgataacatatataaatattagataatgctaatgcttctgctgctgtcttttctgtaatattctctttcatgctgaatttactatgaccatttataagcagtgcagttaactacagatagcatttcaggacaaaatagatgactcaaaccatttattgcttaaaaaatagcttacgccatgctatgctataagcagcttttatgcacattgacaaatgaagagtaagcttcagcttgctaaaggaaactgtggaaccttttgtaacttttggtgatatggaaaattatttacaaaccgtcaaagaatatgaggaagttgctgtatgacatagtgctggcactgatattatccatcatctctttttggacacttctgtaaatgtgattggattgtttgaaagaagatttaaagtttcaaagttttttgttctgtttttgctttgcatttggagaaaatattgaaagcagggtatgttgtttcattcaccttgaaaaaaccatgagtaaatggggatatagaatctctgaatagctcgctaaaagattcaagcaagggacatgaattttgttccatctatcaataatatccagaagaacaacttttttaaagagtctatagcaaaaagcaaaaaaaaaaaaaaattctaaacacaaagtcaaaataaacctattgtaaaagcatttcgtgatgagcatgaaaaagattgtttaaagatgatccccccagctacccattttccaaaactacacagatcacagctcatttctctaagtggagcagttatcaagaaacccaaacaccaaaattgctactcttcacatttaatcctacaaaaagtactccaatttcaaaatatgtatgtaacctgcgatttcaatgattgttgttcatatacatcatgtattattttggcccattttgggcctaaaaaagaaaactatgccttaaaaatcagaaccttttctccccactatgcttatgtggccatctacagcacttagaataaaaacagatgttaaaatattcagtgaaagttttattggaaaaaggaattgagatatataattgagatttggtgaaattgaaggagaaaatttaagtgagtctttaaaatatattctgaatgaaaactgtattgaggattcatttttgttccttttttttctttttctcttttctcctttttcttctttttaatagtctagttttagtcagtcagtgaggaagaattgggccatgctaacgttatcacaagagaacaatggcagaaatggtattagttatataatatttaaggacaaactatatgttttgctgttttaacgtagtgactcactgaactaaatacataattgaccaacattaagtgtatttccaatacagaagggttgaaaatattacattataaactcttttgaaaaatgtatctaaaattttttaagttctgttttgattccactttttggttgagtttttatgtttttgttttcaggtagattaataaatctggcagctgatttctgcaagattcttgtgttttgaatttctcattgaattggctactcaaacatagaaatcatttgttaatgatgtaatgtcttctctcagcttttatcttcactgctgtttgctgtctcttgatgatgacatgttaatacccaatagattaattgcaacaaacacttatactcaaataactaagtaaaaataatttttcttgttatgtccatgaaaagtgcttcagaataaaaatccacaagactgacagtgcagaacatttttctcaaatcatgggcggatcttggaggtctagtttcccgtagatgctgtaaccaattaccacaacttcagtaatttacacaaatttatcttatagttctggaggcagaagttcaaaagaagccttaagagactaaaaccaagatgtccttaggtctggttccttctggaggctccaggggagattcttccagctttcacttctagagtctgctgacattccttggctcctggctacatcacttcaatctctgcttccatggtcacatactcttctactatagtcaaatttccttcctgcctcttataaggatgcttgtgattacatttaggggatgctcagataatccaggacaatctctccatctcaagatccttaacttaatgacgtgtgccaagtccctttggctagataattattcataggtcccagggattaggacatggatgtaaggggtgagggcagggctgttattcagaacaccgcacggaggaggaagactgtgtagcaaagactctaattgatttactcaggaacagtggagttctgctgagggatctaggatttgaaagtactagagtttgcttttatttaccactgagatattttccccttattctgcataaataattttgaaaactttctatattaaatttcaactattccactaaaatgtctggtaatcacatcaagcctttagattattcaaatccttccccagcccccaggaaaacactaagtcatgaaacagaaaaacagaaggtatgataataatagtaataacagttaaatcagtggtctaatccagattttattttttaatacatttcttttggtgttaatatgggttactatgtgatcttatcatttgctagtgattattacttattaggtaagaacaatgtgtaaaatatgtctattactcaaaagaacaattgcaaaatgagtcaacttatctttatataaccaggaaagaaatatattgccagaagctacagaattttgccagatgatagggatttctaaaatgagccactttgtctatcatgcagccttttcagagcttgtaatgagaaaacattacagaggagaaggtcatttggatgtttgttacttggaatcctagaaaacaaaaactaaaatttaaaaataagaagtgagtaagctattttccatttgcgatttggtatggagaagagaggaaatagaattattaaaaaaatacaaattgggtaaaagtgatggtggaaaaaatataaagaaggcaaatgtacatattaagcaattctactaagaattggaaaaatcaagtttcaaaaagatggtaatagttgggcatgatactagaaaatttcacccagtttattcagagctcaactagtacttttaggacttctttttttatatacatgagactcactttgacatacttaaaaaaaaaacagtttatggaaagtacagtttaagaggagaatttgattagactaagtggatatctttatagaaatattaatgatttcagaattttcagttacaagtgtatataccgtggctattgtttatggattcatatgtaaggtagggtcttttttgcatatagactccagtattagttactttcattctaaaattatatttatgcttctatggggaagaaaatttttaattcacttggttgtattaaaattatacttacggtttgagaaaacatgctatgaaaatcatgattatagcaaattaaatatgctcaaaatttaaatctaaaataaaagcccagaaactgaaaa

SEQ ID NO: 28 - DNA, 10661 bp, Homo sapiens
  source 1..10661: mol_type = unassigned DNA
cgagatcccggggagccagcttgctgggagagcgggacggtccggagcaagcccagaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaacaagcaaacaaaaaaaaaaagcaaaaacaaaacaaaaaataagccaaaaaaccttgctagtgttttttcctcaaaaataaataaataaataaataaatacgtacatacatacacacatacatacaaacatatagaaatccccaaagaggccaatagtgacgagaaggtgaaaattgcaggcccatggggagttactgattttttcatctcctccctccacgggagactttattttctgccaatggctattgccattagagggcagagtgaccccagagctgagttgggcaggggggtggacagagaggagaggacaaggagggcaatggagcatcagtacctgcccacagccttggtccctgggggctagactgctcaactgtggagcaattcattatactgaaaatgtgcttgttgttgaaaatttgtctgcatgttaatgcctcacccccaaacccttttctctctcactctctgcctccaacttcagattgactttcaatagtttttctaagacctttgaactgaatgttctcttcagccaaaacttggcgacttccacagaaaagtctgaccactgagaagaaggagagcagagatttaaccctttgtaaggccccatttggatccaggtctgctttctcatgtgtgagtcagggaggagctggagccagaggagaagaaaatgatagcttggctgttctcctgcttaggacactgactgaatagttaaactctcactgccactaccttttccccacctttaaaagacctgaatgaagttttctgccaaactccgtgaagccacaagcaccttatgtcctcccttcagtgttttgtgggcctgaatttcatcacactgcatttcagccatggtcatcaagcctgtttgcttcttttgggcatgttcacagattctctgttaagagcccccaccaccaagaaggttagcaggccaacagctctgacatctatctgtagatgccagtagtcacaaagatttcttaccaactctcagatcgctggagcccttagacaaactggaaagaaggcatcaaagggatcaggcaagctgggcgtcttgcccttgtcccccagagatgataccctcccagcaagtggagaagttctcacttccttctttagagcagctaaaggggctacccagatcagggttgaagagaaaactcaattaccagggtgggaagaatgaaggcactagaaccagaaaccctgcaaatgctcttcttgtcacccagcatatccacctgcagaagtcatgagaagagagaaggaacaaagaggagactctgactactgaattaaaatcttcagcggcaaagcctaaagccagatggacaccatctggtgagtttactcatcatcctcctctgctgctgattctgggctctgacattgcccatactcactcagattccccacctttgttgctgcctcttagtcagagggaggccaaaccattgagactttctacagaaccatggcttctttcggaaaggtctggttggtgtggctccaatactttgccacccatgaactcagggtgtgccctgggacactggttttatatagtcttttggcacacctgtgttctgttgacttcgttcttcaagcccaagtgcaagggaaaatgtccacctactttctcatcttggcctctgcctccttacttagctcttaatctcatctgttgaactcaagaaatcaagggccagtcatcaagctgcccattttaattgattcactctgtttgttgagaggatagtttctgagtgacatgatatgatccacaagggtttccttccctgatttctgcattgatattaatagccaaacgaacttcaaaacagctttaaataacaagggagaggggaacctaagatgagtaatatgccaatccaagactgctggagaaaactaaagctgacaggttccctttttggggtgggatagacatgttctggttttctttattattacacaatctggctcatgtacaggatcacttttagctgttttaaacagaaaaaaatatccaccactcttttcagttacactaggttacattttaataggtcctttacatctgttttggaatgattttcatcttttgtgatacacagattgaattatatcattttcatatctctccttgtaaatactagaagctctcctttacatttctctatcaaatttttcatctttatgggtttcccaattgtgactcttgtcttcatgaatatatgtttttcatttgcaaaagccaaaaatcagtgaaacagcagtgtaattaaaagcaacaactggattactccaaatttccaaatgacaaaactagggaaaaatagcctacacaagcctttaggcctactctttctgtgcttgggtttgagtgaacaaaggagattttagcttggctctgttctcccatggatgaaaggaggaggattttttttttcttttggccattgatgttctagccaatgtaattgacagaagtctcattttgcatgcgctctgctctacaaacagagttggtatggttggtatactgtactcacctgtgagggactggccactcagacccacttagctggtgagctagaagatgaggatcactcactggaaaagtcacaaggaccatctccaaacaagttggcagtgctcgatgtggacgaagagtgaggaagagaaaaagaaggagcaccagggagaaggctccgtctgtgctgggcagcagacagctgccaggatcacgaactctgtagtcaaagaaaagagtcgtgtggcagtttcagctctcgttcattgggcagctcgcctaggcccagcctctgagctgacatgggagttgttggattctttgtttcatagctttttctatgccataggcaatattgttgttcttggaaagtttattatttttttaactcccttactctgagaaagggatattttgaaggactgtcatatatctttgaaaaaagaaaatctgtaatacatatatttttatgtatgttcactggcactaaaaaatatagagagcttcattctgtcctttgggtagttgctgaggtaattgtccaggttgaaaaataatgtgctgatgctagagtccctctctgtccatactctacttctaaatacatataggcatacatagcaagttttatttgacttgtactttaagagaaaatatgtccaccatccacatgatgcacaaatgagctaacattgagcttcaagtagcttctaagtgtttgtttcattaggcacagcacagatgtggcctttccccccttctctcccttgatatctggcagggcataaaggcccaggccacttcctctgccccttcccagccctgcaccaaagctgcatttcaggagactctctccagacagcccagtaactacccgagcatggcccctgcatagccctggaaaaataagaggctgactgtctacgaattatcttgtgccagttgcccaggtgagagggcactgggccaagggagtggttttcatgtttgacccactacaaggggtcatgggaatcaggaatgccaaagcaccagatcaaatccaaaacttaaagtcaaaataagccattcagcatgttcagtttcttggaaaaggaagtttctacccctgatgcctttgtaggcagatctgttctcaccattaatctttttgaaaatcttttaaagcagtttttaaaaagagagatgaaagcatcacattatataaccaaagattacattgtacctgctaagataccaaaattcataagggcagggggggagcaagcattagtgcctctttgataagctgtccaaagacagactaaaggactctgctggtgactgacttataagagctttgtgggtttttttttccctaataatatacatgtttagaagaattgaaaataatttcgggaaaatgggattatgggtccttcactaagtgattttataagcagaactggctttccttttctctagtagttgctgagcaaattgttgaagctccatcattgcatggttggaaatggagctgttcttagccactgtgtttgctagtgcccatgttagcttatctgaagatgtgaaacccttgctgataagggagcatttaaagtactagattttgcactagagggacagcaggcagaaatccttatttctgcccactttggatggcacaaaaagttatctgcagttgaaggcagaaagttgaaatacattgtaaatgaatatttgtatccatgtttcaaaattgaaatatatatatatatatatatatatatatatatatatatatagtgtgtgtgtgtgttctgatagctttaactttctctgcatctttatatttggttccagatcacacctgatgccatgtacttgtgagagaggatgcagttttgttttggaagctctctcagaacaaacaagacacctggattgatcagttaactaaaagttttctcccctattgggtttgacccacaggtcctgtgaaggagcagagggataaaaagagtagaggacatgatacattgtactttactagttcaagacagatgaatgtggaaagcataaaaactcaatggaactgactgagatttaccacagggaaggcccaaacttggggccaaaagcctacccaagtgattgaccagtggccccctaatgggacctgagctgttggaagaagagaactgttccttggtcttcaccatccttgtgagagaagggcagtttcctgcattggaacctggagcaagcgctctatctttcacacaaattccctcacctgagattgaggtgctcttgttactgggtgtctgtgtgctgtaattctggttttggatatgttctgtaaagattttgacaaatgaaaatgtgtttttctctgttaaaacttgtcagagtactagaagttgtatctctgtaggtgcaggtccatttctgcccacaggtagggtgtttttctttgattaagagattgacacttctgttgcctaggacctcccaactcaaccatttctaggtgaaggcagaaaaatccacattagttactcctcttcagacatttcagctgagataacaaatcttttggaattttttcacccatagaaagagtggtagatatttgaatttagcaggtggagtttcatagtaaaaacagcttttgactcagctttgatttatcctcatttgatttggccagaaagtaggtaatatgcattgattggcttctgattccaattcagtatagcaaggtgctaggttttttcctttccccacctgtctcttagcctggggaattaaatgagaagccttagaatgggtggcccttgtgacctgaaacacttcccacataagctacttaacaagattgtcatggagctgcagattccattgcccaccaaagactagaacacacacatatccatacaccaaaggaaagacaattctgaaatgctgtttctctggtggttccctctctggctgctgcctcacagtatgggaacctgtactctgcagaggtgacaggccagatttgcattatctcacaaccttagcccttggtgctaactgtcctacagtgaagtgcctggggggttgtcctatcccataagccacttggatgctgacagcagccaccatcagaatgacccacgcaaaaaaaagaaaaaaaaaattaaaaagtcccctcacaacccagtgacacctttctgctttcctctagactggaacattgattagggagtgcctcagacatgacattcttgtgctgtccttggaattaatctggcagcaggagggagcagactatgtaaacagagataaaaattaattttcaatattgaaggaaaaaagaaataagaagagagagagaaagaaagcatcacacaaagattttcttaaaagaaacaattttgcttgaaatctctttagatggggctcatttctcacggtggcacttggcctccactgggcagcaggaccagctccaagcgctagtgttctgttctctttttgtaatcttggaatcttttgttgctctaaatacaattaaaaatggcagaaacttgtttgttggactacatgtgtgactttgggtctgtctctgcctctgctttcagaaatgtcatccattgtgtaaaatattggcttactggtctgccagctaaaacttggccacatcccctgttatggctgcaggatcgagttattgttaacaaagagacccaagaaaagctgctaatgtcctcttatcattgttgttaatttgttaaaacataaagaaatctaaaatttcaaaaaa

SEQ ID NO: 29 - DNA, 2113 bp, Homo sapiens
  source 1..2113: mol_type = unassigned DNA
tcccaatcccggggcggccgggcgggggtgggcagggggcgtgaggccgcccctgcgtcccgggggccccccgaaaacgcgctccgggtgcccggtccctccgctgcgccctgccgccgtcctcccgggggtctcgggcggccgcggccgtgtccttcgcgtcccggcggcgcggcgggaggggccggcgtgacgcagcggttgctacgggccgcccttataaataaccgggctcaggagaaactttagcgagtcagagccgcgcacgggactgggaaggggacccacccgagggtccagccaccagccccctcactaatagcggccaccccggcagcggcggcagcagcagcagcgacgcagcggcgacagctcagagcagggaggccgcgccacctgcgggccggccggagcgggcagccccaggccccctccccgggcacccgcgttcatgcaacgcctggtggcctgggacccagcatgtctccccctgccgccgccgccgcctgcctttaaatccatggaagtggccaacttctactacgaggcggactgcttggctgctgcgtacggcggcaaggcggcccccgcggcgccccccgcggccagacccgggccgcgcccccccgccggcgagctgggcagcatcggcgaccacgagcgcgccatcgacttcagcccgtacctggagccgctgggcgcgccgcaggccccggcgcccgccacggccacggacaccttcgaggcggctccgcccgcgcccgcccccgcgcccgcctcctccgggcagcaccacgacttcctctccgacctcttctccgacgactacgggggcaagaactgcaagaagccggccgagtacggctacgtgagcctggggcgcctgggggccgccaagggcgcgctgcaccccggctgcttcgcgcccctgcacccaccgcccccgccgccgccgccgcccgccgagctcaaggcggagccgggcttcgagcccgcggactgcaagcggaaggaggaggccggggcgccgggcggcggcgcaggcatggcggcgggcttcccgtacgcgctgcgcgcttacctcggctaccaggcggtgccgagcggcagcagcgggagcctctccacgtcctcctcgtccagcccgcccggcacgccgagccccgctgacgccaaggcgcccccgaccgcctgctacgcgggggccgcgccggcgccctcgcaggtcaagagcaaggccaagaagaccgtggacaagcacagcgacgagtacaagatccggcgcgagcgcaacaacatcgccgtgcgcaagagccgcgacaaggccaagatgcgcaacctggagacgcagcacaaggtcctggagctcacggccgagaacgagcggctgcagaagaaggtggagcagctgtcgcgcgagctcagcaccctgcggaacttgttcaagcagctgcccgagcccctgctcgcctcctccggccactgctagcgcggcccccgcgcgcgtccccctgccggccggggctgagactccggggagcgcccgcgcccgcgccctcgcccccgcccccggcggcgccggcaaaactttggcactggggcacttggcagcgcggggagcccgtcggtaattttaatattttattatatatatatatctatatttttgtccaaaccaaccgcacatgcagatggggctcccgcccgtggtgttatttaaagaagaaacgtctatgtgtacagatgaatgataaactctctgcttctccctctgcccctctccaggcgccggcgggcgggccggtttcgaagttgatgcaatcggtttaaacatggctgaacgcgtgtgtacacgggactgacgcaacccacgtgtaactgtcagccgggccctgagtaatcgcttaaagatgttcctacgggcttgttgctgttgatgttttgttttgttttgttttttggtctttttttgtattataaaaaataatctatttctatgagaaaagaggcgtctgtatattttgggaatcttttccgtttcaagcattaagaacacttttaataaacttttttttgagaatggttacaaagccttttgggggcagtaaaaaaa

SEQ ID NO: 30 - DNA, 2793 bp, Homo sapiens
  source 1..2793: mol_type = unassigned DNA
gggcacgaggcgctccctgggatcacatggtacctgctccagtgccgcgtgcggcccgggaaccctgggctgctggcgcctgcgcagagccctctgtcccagggaaaggctcgggcaaaaggcggctgagattggcagagtgaaatattactgccgagggaacgtagcagggcacacgtctcgcctctttgcgactcggtgccccgtttctccccatcacctacttacttcctggttgcaacctctcttcctctgggacttttgcaccgggagctccagattcgccaccccgcagcgctgcggagccggcaggcagaggcaccccgtacactgcagagacccgaccctccttgctaccttctagccagaactactgcaggctgattccccctacacactctctctgctcttcccatgcaaagcagaactccgttgcctcaacgtccaacccttctgcagggctgcagtccggccaccccaagaccttgctgcagggtgcttcggatcctgatcgtgagtcgcggggtccactccccgcccttagccagtgcccagggggcaacagcggcgatcgcaacctctagtttgagtcaaggtccagtttgaatgaccgctctcagctggtgaagacatgacgaccctggactccaacaacaacacaggtggcgtcatcacctacattggctccagtggctcctccccaagccgcaccagccctgaatccctctatagtgacaactccaatggcagcttccagtccctgacccaaggctgtcccacctacttcccaccatcccccactggctccctcacccaagacccggctcgctcctttgggagcattccacccagcctgagtgatgacggctccccttcttcctcatcttcctcgtcgtcatcctcctcctccttctataatgggagcccccctgggagtctacaagtggccatggaggacagcagccgagtgtcccccagcaagagcaccagcaacatcaccaagctgaatggcatggtgttactgtgtaaagtgtgtggggacgttgcctcgggcttccactacggtgtgcacgcctgcgagggctgcaagggctttttccgtcggagcatccagcagaacatccagtacaaaaggtgtctgaagaatgagaattgctccatcgtccgcatcaatcgcaaccgctgccagcaatgtcgcttcaagaagtgtctctctgtgggcatgtctcgagacgctgtgcgttttgggcgcatccccaaacgagagaagcagcggatgcttgctgagatgcagagtgccatgaacctggccaacaaccagttgagcagccagtgcccgctggagacttcacccacccagcaccccaccccaggccccatgggcccctcgccaccccctgctccggtcccctcacccctggtgggcttctcccagtttccacaacagctgacgcctcccagatccccaagccctgagcccacagtggaggatgtgatatcccaggtggcccgggcccatcgagagatcttcacctacgcccatgacaagctgggcagctcacctggcaacttcaatgccaaccatgcatcaggtagccctccagccaccaccccacatcgctgggaaaatcagggctgcccacctgcccccaatgacaacaacaccttggctgcccagcgtcataacgaggccctaaatggtctgcgccaggctccctcctcctaccctcccacctggcctcctggccctgcacaccacagctgccaccagtccaacagcaacgggcaccgtctatgccccacccacgtgtatgcagccccagaaggcaaggcacctgccaacagtccccggcagggcaactcaaagaatgttctgctggcatgtcctatgaacatgtacccgcatggacgcagtgggcgaacggtgcaggagatctgggaggatttctccatgagcttcacgcccgctgtgcgggaggtggtagagtttgccaaacacatcccgggcttccgtgacctttctcagcatgaccaagtcaccctgcttaaggctggcacctttgaggtgctgatggtgcgctttgcttcgttgttcaacgtgaaggaccagacagtgatgttcctaagccgcaccacctacagcctgcaggagcttggtgccatgggcatgggagacctgctcagtgccatgttcgacttcagcgagaagctcaactccctggcgcttaccgaggaggagctgggcctcttcaccgcggtggtgcttgtctctgcagaccgctcgggcatggagaattccgcttcggtggagcagctccaggagacgctgctgcgggctcttcgggctctggtgctgaagaaccggcccttggagacttcccgcttcaccaagctgctgctcaagctgccggacctgcggaccctgaacaacatgcattccgagaagctgctgtccttccgggtggacgcccagtgacccgcccggccggccttctgccgctgcccccttgtacagaatcgaactctgcacttctctctcctttacgagacgaaaaggaaaagcaaaccagaatcttatttatattgttataaaatattccaagatgagcctctggccccctgagccttcttgtaaatacctgcctccctcccccatcaccgaacttcccctcctcccctatttaaaccactctgtctcccccacaaccctcccctggccctctgatttgttctgttcctgtctcaaatccaatagttcacagctgagctggcttcaaaaaaaaaaaaaaaaaa

SEQ ID NO: 31 - DNA, 2672 bp, Homo sapiens
  source 1..2672: mol_type = unassigned DNA
gcgtggccggcgccggctcttgcggccgagcagagttgcggcgtgggaaagagccgctaggagcagaccgcgccgccgccggagccgcgcctgcccaggcccggggagggaggaggcgggcgtcagggtgctgcgccccgctcggcgtccgagcttccggccgggctgtgccccgcgcggtcttcgccgggatgaagcgcccctgcgaggagacgacctccgagagcgacatggacgagaccatcgacgtggggagcgagaacaattactcggggcaaagtactagctctgtgattagattgaattctccaacaacaacatctcagattatggcaagaaagaaaaggagagggattatagagaaaaggcgtcgggatcggataaataacagtttatctgagttgagaagacttgtgccaactgcttttgaaaaacaaggatctgcaaagttagaaaaagctgaaatattgcaaatgacagtggatcatttgaagatgcttcaggcaacagggggtaaaggctactttgacgcacacgctcttgccatggacttcatgagcataggattccgagagtgcctaacagaagttgcgcggtacctgagctccgtggaaggcctggactcctcggatccgctgcgggtgcggcttgtgtctcatctcagcacttgcgccacccagcgggaggcggcggccatgacatcctccatggcccaccaccatcatccgctccacccgcatcactgggccgccgccttccaccacctgcccgcagccctgctccagcccaacggcctccatgcctcagagtcaaccccttgtcgcctctccacaacttcagaagtgcctcctgcccacggctctgctctcctcacggccacgtttgcccatgcggattcagccctccgaatgccatccacgggcagcgtcgccccctgcgtgccacctctctccacctctctcttgtccctctctgccaccgtccacgccgcagccgcagcagccaccgcggctgcacacagcttccctctgtccttcgcgggggcattccccatgcttcccccaaacgcagcagcagcagtggccgcggccacagccatcagcccgcccttgtcagtatcagccacgtccagtcctcagcagaccagcagtggaacaaacaataaaccttaccgaccctgggggacagaagttggagctttttaaatttttcttgaacttcttgcaatagtaactgaatgtcctccatttcagagtcagcttaaaacctctgcaccctgaaggtagccatacagatgccgacagatccacaaaggaacaataaagctatttgagacacaaacctcacgagtggaaatgtggtattctcttttttttctctcccttttttgtttggttcaaggcagctcggtaactgacatcagcaacttttgaaaacttcacacttgttaccatttagaagtttcctggaaaatatatggaccgtaccatccagcagtgcatcagtatgtctgaattggggaagtaaaatgccctgactgaattctcttgagactagatgggacatacatatatagagagagagtgagagagtcgtgtttcgtaagtgcctgagcttaggaagttttcttctggatatataacattgcacaagggaagacgagtgtggaggataggttaagaaaggaaagggacagaagtcttgcaataggctgcagacattttaataccatgccagagaagagtattctgctgaaaccaacaggttttactggtcaaaatgactgctgaaaataattttcaagttgaaagatctagttttatcttagtttgccttctttgtacagacatgccaagaggtgacatttagcagtgcattggtataagcaattatttcatcagttctcagattaacaagcatttctgctctgcctgcaggcccccaggcacttttttttttggatggctcaaaatatggtgctgctttatataaaccttacatttatatagtgcacctatgagcagttgcctaccatgtgtccaccagaggctatttaattcatgccaacttgaaaactctccagtttgtaggagtttggtttaatttattcagtttcattaggactatttttatatatttatcctcttcattttctcctaatgatgcaacatctattcttgtcaccctttgggagaagttacatttctggaggtgatgaagcaaggagggagcactaggaagagaaaagctacaatttttaaagctctttgtcaagttagtgattgcatttgatcccaaaacaagatgaatgtatgcaatgggatgtacataagttatttttgcccatgcctaaactagtgctatgtaatggggttgtggttttgtttttttcgatttcgtttaatgacaaaataatctcttaatatgctgaaatcaagcacgtgagagtttttgtttaaaagataagagacacagcatgtattatgcacttcatttctctactgtgtggagaaagcaataaacattatgagaatgttaaacgttatgcaaaattatacttttaaatatttgttttgaaattactgtacctagtcttttttgcattactttgtaacctttttctatgcaagagtctttacataccactaattaaatgaagtcctttttgacta

SEQ ID NO: 32 - DNA, 1239 bp, Homo sapiens
  source 1..1239: mol_type = unassigned DNA
atggaggccctgcagaagcagcaggcagctcggctggcccagggggtggggccattggcccctgcatgcccgctgctgccaccgcagcctcccctgcctgaccaccggaccctacaggcccctgagggggccttggggaatgttggggctgaggaagaggaagatgctgaagaagatgaggagaagcgggaggaagccggggcagaggaggaggcagctgaggagagccgtccaggggcccagggccccagctcgccttctagccagccccctggactccatccccacgagtggacctacgaggaacaattcaagcagctgtatgagctcgatgcagaccccaagaggaaggaatttctggatgacctgtttagcttcatgcaaaagagggggacgccagtgaaccgcgtgcccatcatggcgaagcaggtgctcgacctgtacgctctgtttcgcctggtgaccgccaagggcggcctggtggaagtcatcaaccgcaaagtgtggcgggaagtcacgcgcggcctcagcctacccaccaccatcacctcggccgccttcactctacgcacccagtacatgaagtacctgtacccgtacgagtgcgagactcgagcgctcagctccccaggggagctccaggccgccatagacagcaatcggcgcgagggccgtcgccaggcttacaccgctactccgctcttcggcttggcagggccgccccctcggggcgctcaggacccagccttgggtcccggccccgcccctccggcgacccagtccagccctggcccagcccagggttccacctccggcctgccagcgcatgcatgcgctcagctgagtccaagccctattaagaaagaggagagtggaattccaaacccttgtctggcactgcctgtgggcctggcactgggacctacacgggagaaattggcaccagaggagcccccagagaagagagctgtgctgatggggcctatggacccacctcgaccttgcatgccccccagtttcctgccccgtggcaaggttcccctgagggaagagcggctggatgggcctcttaatctggcaggcagtggcatcagcagtatcaacatggccctagagatcaacggggtggtctacactggtgtcctctttgcccgccgccagcctgtgccagcttcccagggtccaaccaaccctgcacccccaccctccacagggcccccttccagcatcttgccctga

SEQ ID NO: 33 - DNA, 5208 bp, Homo sapiens
  source 1..5208: mol_type = unassigned DNA
cttactcatttgtgtttattcttggacttatcctgacataatggggtttttttaattatagattcacactgcatttattcatcacccctgtcctctcatccataactcaaatttactaccagcaacacaaaatacaaagatgtgtccagtttcactacagctcttcgcgtttacaagtgtcgagcgcttgctttcggaacgcccttgtgattggccgagccaatgccagtgacatcaaccaacttacttttgattggaaggctggttgctgggactgtagcgtttgcaggaagtcacttaactgtttgggagctggaaaaccgaagctgaagttctcttttgccataggaacgagcgcaactgactaggaaagatgtgtcccaaagctccgcaagctggaacgtgagccaggaggcccggaccggccacgggaccgcgaggcactccgaaagtgtgcggctgccccttccctgcctcccagctgttacccttttaaatgtcagtgttcgaggctgtaggggtagcacgaggcagcgaaacggaacagtcggattggccgcacgcctcagttctagacgcacctctccaccgaaggccgttctgactggcagggggagaaagtaaacagagttgaatcaccctccccactggccaattggagggggtttggtttgtgacgtgatgggattctgcgaaattgttactgagcaagagaatgccggaacggtgcggaccggccggagcaggggttcagaagccgtcagtggactcgggaaaaagtgtctcttagacctggcgctcggcgggaccctcgccacccgcgtcggggtgatcgggtgaatgtcctggggctttggctcgacggcgaggcggccgagggcgtgcacctctcttgcagtttcctctcccagcgcctcgggggcgttttcagtcgaataaacttgcgaccgccacgtgtggcatctttccaagggagccggctcagaggggccggcgcgcccgtcgggggatcgcggccggcgcggggcaggggcggcggctagaggcggcggcgcggcggagcccggggccgtggatgctgcgtgcggaggcgctgccggttacgtaaagatgaggggctgaggtcgcctcggcgctcctgcgagtcggaagcgccccgcgcccccgcccccttggccgccgcgccgtgccgcgccgcgccgcgctcgtcgtccgaggccagggcagggcgagccgaacctccgcagccaccgccaagtttgtccgcgccgcctgggctgccgtcgcccgcaccatgtccgcggccgcctacatggacttcgtggctgcccagtgtctggtttccatttcgaaccgcgctgcggtgccggagcatggggtcgctccggacgccgagcggctgcgactacctgagcgcgaggtgaccaaggagcacggtgacccgggggacacctggaaggattactgcacactggtcaccatcgccaagagcttgttggacctgaacaagtaccgacccatccagaccccctccgtgtgcagcgacagtctggaaagtccagatgaggatatgggatccgacagcgacgtgaccaccgaatctgggtcgagtccttcccacagcccggaggagagacaggatcctggcagcgcgcccagcccgctctccctcctccatcctggagtggctgcgaaggggaaacacgcctccgaaaagaggcacaagtgcccctacagtggctgtgggaaagtctatggaaaatcctcccatctcaaagcccattacagagtgcatacaggtgaacggccctttccctgcacgtggccagactgccttaaaaagttctcccgctcagacgagctgacccgccactaccggacccacactggggaaaagcagttccgctgtccgctgtgtgagaagcgcttcatgaggagtgaccacctcacaaagcacgcccggcggcacaccgagttccaccccagcatgatcaagcgatcgaaaaaggcgctggccaacgctttgtgaggtgctgcccgtggaagccagggagggatggaccccgaaaggacaaaagtactcccaggaaacagacgcgtgaaaactgagccccagaagaggcacacttgacggcacaggaagtcactgctctttggtcaatattctgattttcctctccctgcattgtttttaaaaagcacattgtagcctaagatcaaagtcaacaacactcggtccccttgaagaggcaactctctgaacccgtctctgactgttggagggaaggcaaatgcttttgggttttttggtttttgtttttgtttttttttctccttttatttttttgcgggggagggtagggagtgggtgggggggaggggggtaaggccaagactggggtagaattttaaagattcaacactggtgtacatatgtccgctgggtgagttgacctgtggcctcgcacagtgattctgggccctttatgcttgctgtctctcagaattgttttcttaccttttaatgtaatgacgagtgtgcttcagtttgtttagcaaaaccactctcttgaatcacgttaacttttgagattaaaaaaaaaaacgccatagcacagctgtctttatgcaagcaagagcacatctactccagcatgatctgtcatctaaagacttgaaaacaaaaaacagttacttatagtcaatgggtaagcagagtctgaatttatactaatcaagacaaacctttgaaaggttacactaagtacagaacttttaaaccttgctttgtatgagttgtactttttgaacataagctgcacttttattttctaatgcagaggatgaataagttaaatacatgctttgaggatagaagcagatgttctgtttggcaccacgttataatctgcttattttacaatatacacgtttccctaagaaatcatggcagagatgtgagggcagaatatacacaacagatgctgaaggagaaggagggtagtgttttgcaaaagaaaaagaaaagaaccaacagaattttaactctattaacttttccaaattttcctatgcttttagttaacatcattattgtatcctaatgccactaggggagagagcttttgactctgttgggttttatttgaatgtgtgcataacagtaatgagatctggaaacacctattttttggggaaaaaggtttgttggtctccttcctgtgttcctacaaaactcccactctcaggtgcaagagttatgtagaaggaaagggagctgaaataggaacagaaaaatcaacccctataactagtgaacaccaagggaaaataccacaatgatttcagaggagactctgcaaaatcgtcccttgtggagaatgcaggcaacatggaatactaggaatgaaatcacatcactgtatcttttacatcaatagcctcaccactaatatatcttgtatctaggtgtctataatggctgaaaccactacatccatctatgccatttacctgaaaacttaactgtggcctttatgaggccagaaaagtgaactgagttttcgtagttaagacctcaaatgaggggagtcagcagtgatcatgggggaaatgtttacattttttttttcttcagaagtaacgctttctgatgattttatctgatatttaaaacagggagctatggtgcactctagtttatacttgcgctctgaaatgtgtaaacatagggtgcctacctatttcacctgacccatactcgtttctgattcagaatcagtgtgggctcctgcagtgggcgcgggtcacggctgactccaacttccaatacaacagccatcactagcacagtgtttttttgtttaaccaacgtagttgtattagtagttctataaagagaactgcttttaacattagggactgggagcagtccatgggataaaaaggaaagtgttttctcacgagaaaacatgtcaggaaaaataaagaacactttctacctctgtttcagatttttgaaacacttattttaaaccaaattttaatttctgtgtccaaaataagttttaaggacatctgttcttccatacgaaataggttaggctgcctatttctcactgagctcatggaatggttctgcttatgatactctgcacgctgccttttagtgagtgaggagtttggggttgcctagcaacttgctaacttgtaaaaagtcatctttccctcacagaaagaaacgaaagaaagcaaagcaaagtcagtgaaagacaatctttatagtttcaggagtaaatctaaatgtggcttttgtcaagcacttagatggatataaatgcagcaacttgttttaaaaaaatgcacaatttacttcccaaaaaagttgttacttgccttttcaagttgttgacaaacacacatttgatattctcttatatgttatagtaatgtaacgtataaactcaagcctttttattctttgtgattaaatcctgttttaaaatgtcacaaaacaggaaccagcattctaattagatttactatatcaagatatggttcaaataggactactagagttcattgaacactaaaactatgaaacaattactttttatattaaaaagaccatggatttaacttatgaaaatccaaatgcaggatagtaatttttgtttacttttttaaccaaactgaatttttgaaagactattgcaggtgtttaaaaagaaagaaaagttgttttatctaatactgtaagtagttgtcatattctggaaaatttaatagttttagagttaagatatctcctctctttggttagggaagaagaaagcccttcaccattgtggaatgatgccctggctttaaggtttagctccacatcatgcttctcttgagaattctatttggtagttacaattacagaaactgattagtttgtcagtttgcagatagatttagcacagtactcatcactcggatagattgagatgttctttcacatcagatgatctgtaacactgtaagatactgatctttacaactgtttaatcagttttatttttgtacagtattagtgacctaagttattttgctgtcccgtttttgtaaatcaaatgaaattataaaagaggattctgacagtaggtattttgtacatatgtatatatgttgtccaaataaaaataataaatgataaagactgaa

SEQ ID NO: 34 - DNA, 2302 bp, Homo sapiens
  source 1..2302: mol_type = unassigned DNA
ctctgccaggctcacgggacagctgcacctctcagcgtctccagctccaggacgcggtcgtcccaactccttccgagtggaaagagtgtaaaacttttgtccgtgcgcgggtggagctcagtaggaccacggcgcgtcctgccccggcttccccagcctcccagcagggttagctgcggtcagcgcactttccacttgggactcccggccagaaatttctcgggaatggagcggtcacagtgtggcagcagagaccgaggcgttagcggccgacctcacttggcccctgggctagtggtggctgcccctccgcccccgtccccggcgttgccggtaccatcggggatgcaggttcccccagcgttcctgcggccgcccagcctctttctgcgagcagcggccgcggccgccgccgccgctgccgccacctcgggaagcggaggctgcccgccggctcccgggctggagagcggggtaggcgcggtgggctgcggctacccgcggacgcccaagtgcgcccgctgtcgtaaccatggtgtggtgtcagcgctcaagggccacaagcgcttctgccgctggcgggactgcgcgtgtgccaagtgcaccctgatcgccgagcgccagcgcgtcatggccgcccaggtggcgctgcgcaggcagcaggcgcaggaggagagcgaagcccgggggctacagaggctcctgtgctcggggctctcctggccccccggtggtcgggcatccgggggcggcggcagagccgagaatccacagtccacgggcggccctgcggcgggggctgcgctgggactgggtgccttgagacaggccagtggttccgcgacccccgctttcgaagttttccagcaagattatcctgaggaaaaacaagaacaaaaagagagtaaatgtgagtcatgccagaatggacaagaagaactgatctccaaatcccatcagctttacctaggatcatcttctaggtctaatggtgtcattgggaaacaaagtatcgggtcatctatttcagaatactccaacaagcctgatagtatcctgtctcctcatcctggagagcaatcaggaggtgaagagagtcccaggtccttatcatcctctgatctggaatcaggaaatgaaagtgaatgggtcaaagacttgactgcgaccaaggcaagccttccgacagtgtcctcaagaccaagagatcctcttgatatccttactaagattttcccaaattacaggcgcagccggctagaaggcattctacggttctgcaaaggggatgtggtccaagccattgaacaggttttaaatggcaaagaacacaagccagacaacaggaacctagcaaactcagaagaactggaaaacacagcctttcagagagcttcaagttttagtcttgctggaattggttttggaactctaggtaataaatcagctttctctcctcttcaaactacttctgcttcttatggaggtgattcaagtctctacggcgtaaatcctagagtaggtatcagtccattaaggctggcatattcttctgcaggaagagggttatctggttttatgtcaccctacctaacacctgggttagtaccaaccttaccttttcggccagctttggattatgccttttcagggatgattagagattcttcctacctttccagtaaagactcaataacttgtggcagactgtacttcagaccaaatcaggacaatccgtaatgtatatgcccattctctctttctggagtttttccagcatacaatacatgcacgtgcacacacatacacacacatccattaatatacttcagtaagtatgtgagtggattatgaggtcttaaaatgctgggttttttttttttcaagcaatataataggtcttagatctgaaaactcttcattaggatttatcaagtgaaagaagtaaatctgaacattatatgtgccttgaataaagctatttcaggaaatatttaatgaattttctccctaaattatcatttgtaaacatttttattttaaaactagtttttattttattgaaaagtggaatttttagtgataaaatacatttgtaagtgtaaagcaatacagcataatagaatagaatataaaccgaaaggaagaactgaacaattaaggcaattctaaataattaccatttcaaaactgtttcttctattcctggttcataggaaagaaaaaagttattcaaagtatttttaaagcatttgatttgcagatgggtgattcgtaataaataaaacatttgagcattttg

SEQ ID NO: 35 - protein, 21 aa, synthetic construct
  REGION 1..21: note = Description of Artificial Sequence: Synthetic peptide
GSGEGRGSLLTCGDVEENPGP

SEQ ID NO: 36 - protein, 22 aa, synthetic construct
  REGION 1..22: note = Description of Artificial Sequence: Synthetic peptide
GSGATNFSLLKQAGDVEENPGP

SEQ ID NO: 37 - protein, 23 aa, synthetic construct
  REGION 1..23: note = Description of Artificial Sequence: Synthetic peptide
GSGQCTNYALLKLAGDVESNPGP

SEQ ID NO: 38 - protein, 25 aa, synthetic construct
  REGION 1..25: note = Description of Artificial Sequence: Synthetic peptide
GSGVKQTLNFDLLKLAGDVESNPGP

SEQ ID NO: 39 - DNA, 8146 bp, Homo sapiens
  source 1..8146: mol_type = unassigned DNA
acttgcactgtcttgttcttgaatgagaaaggaagaaaagagcctcccattactcagacccgtgtaaacattattccccccaggagaaaatggtgttattcaaatgaatcataataaaatagcctctaaacagtttctaagcgggagcctccgtggaactcagcgctccgctcctcccagttcctaagaggtcccgggattcttgagctgtgcccagctgacgagcttttgaagatggcacaataaccgtccagtgatgcctgaccatgacagcacagccctcttaagccggcaaaccaagaggagaagagttgacattggagtgaaaaggacggtagggacagcatctgcattttttgctaaggcaagagcaacgttttttagtgccatgaatccccaaggttctgagcaggatgttgagtattcagtggtgcagcatgcagatggggaaaagtcaaatgtactccgcaagctgctgaagagggcgaactcgtatgaagatgccatgatgccttttccaggagcaaccataatttcccagctgttgaaaaataacatgaacaaaaatggtggcacggagcccagtttccaagccagcggtctctctagtacaggctccgaagtacatcaggaggatatatgcagcaactcttcaagagacagccccccagagtgtctttccccttttggcaggcctactatgagccagtttgatatggatcgcttatgtgatgagcacctgagagcaaagcgcgcccgggttgagaatataattcggggtatgagccattcccccagtgtggcattaaggggcaatgaaaatgaaagagagatggccccgcagtctgtgagtccccgagaaagttacagagaaaacaaacgcaagcaaaagcttccccagcagcagcaacagagtttccagcagctggtttcagcccgaaaagaacagaagcgagaggagcgccgacagctgaaacagcagctggaggacatgcagaaacagctgcgccagctgcaggaaaagttctaccaaatctatgacagcactgattcggaaaatgatgaagatggtaacctgtctgaagacagcatgcgctcggagatcctggatgccagggcccaggactctgtcggaaggtcagataatgagatgtgcgagctagacccaggacagtttattgaccgagctcgagccctgatcagagagcaggaaatggctgaaaacaagccgaagcgagaaggcaacaacaaagaaagagaccatgggccaaactccttacaaccggaaggcaaacatttggctgagaccttgaaacaggaactgaacactgccatgtcgcaagttgtggacactgtggtcaaagtcttttcggccaagccctcccgccaggttcctcaggtcttcccacctctccagatcccccaggccagatttgcagtcaatggggaaaaccacaatttccacaccgccaaccagcgcctgcagtgctttggcgacgtcatcattccgaaccccctggacacctttggcaatgtgcagatggccagttccactgaccagacagaagcactgcccctggttgtccgcaaaaactcctctgaccagtctgcctccggccctgccgctggcggccaccaccagcccctgcaccagtcgcctctctctgccaccacgggcttcaccacgtccaccttccgccaccccttcccccttcccttgatggcctatccatttcagagcccattaggtgctccctccggctccttctctggaaaagacagagcctctcctgaatccttagacttaactagggataccacgagtctgaggaccaagatgtcatctcaccacctgagccaccacccttgttcaccagcacacccgcccagcaccgccgaagggctctccttgtcgctcataaagtccgagtgcggcgatcttcaagatatgtctgaaatatcaccttattcgggaagtgcaatgcaggaaggattgtcacccaatcacttgaaaaaagcaaagctcatgtttttttatacccgttatcccagctccaatatgctgaagacctacttctccgacgtaaagttcaacagatgcattacctctcagctcatcaagtggtttagcaatttccgtgagttttactacattcagatggagaagtacgcacgtcaagccatcaacgatggggtcaccagtactgaagagctgtctataaccagagactgtgagctgtacagggctctgaacatgcactacaataaagcaaatgactttgaggttccagagagattcctggaagttgctcagatcacattacgggagtttttcaatgccattatcgcaggcaaagatgttgatccttcctggaagaaggccatatacaaggtcatctgcaagctggatagtgaagtccctgagattttcaaatccccgaactgcctacaagagctgcttcatgagtagaaatttcaacaactctttttgaatgtatgaagagtagcagtcccctttggatgtccaagttatatgtgtctagattttgatttcatatatatgtgtatgggaggcatggatatgttatgaaatcagctggtaattcctcctcatcacgtttctctcattttcttttgttttccattgcaaggggatggttgttttctttctgcctttagtttgcttttgcccaaggcccttaacatttggacacttaaaatagggttaattttcagggaaaaagaatgttggcgtgtgtaaagtctctattagcaatgaagggaatttgttaacgatgcatccacttgattgatgacttattgcaaatggcggttggctgaggaaaacccatgacacagcacaactctacagacagtgatgtgtctcttgtttctactgctaagaaggtctgaaaatttaatgaaaccacttcatacatttaagtattttgtttggtttgaactcaatcagtagcttttccttacatgtttaaaaataattccaatgacagatgagcagctcacttttccaaagtaccccaaaaggccaaattaaaaaagaaaaataatcactctcaagccttgtctaagaaaagaggcaaactctgaaagtcgtaccagtttcttctggaggcaaagcaattttgcacaaaaccagctctctcaagatgagactagaaattcatacctggtcttgtagccacctctctaaacttgaaaataggttcttcttcataagtgagcttacatcattcttcataaagaaaaatcctataacttgttatcatttttgcttcagatactaaaaggcactaagtttccaatttacgctgctcaactttgtttatatgcttaaaaggattctgtttacttaacaattttttcccctaaaatactattttctgaatacttccttccagtaaggaataaaggaaagcccaacttggccataaaattcttgcctacactagaagtttgttgacagccattagctgacttgatcgtcatctcctaagaggaacacatatattttcacaagcaattccacactatcctgatgggtatgcaaagtggtgacagtctaactcagtgtttcttcattttaggtataacattttaaagcaattgataatgcctcttccaattcagaagctagtattgaccaaaatgtgagaagagtgtatagcataggaaaatttggggttaacccaaaagacacaattccagcacacataagaaagctagctgctattttatgctttcttccatggttctcctcttttttcccttttatttttccctgtttttcaatgatgtacagtgttccctacttgcattgaaaaaactcgtatggcattcacactttttttcttaggtgggtttttgtgtccagatgcagtaagaattcattgttcatcctaaaactgttttccagacccttccttccccttaggtaatttgatatacacctcctaaaatgacacagtaacaaatctggtatttagaacatatagaacataaatgccattttttaattcaactttaataagaattacatttgactttggagaatacaggtcttgacccatgtgactgactagctgacccgatcgctgtaatttaacgtcatttataaattctgctgatggacaggaatgtatgaactcaattattgtcagcacaaagccttaaaacctgctgactttaaattaaatggtgcagtcctatgatgccctgcaccatccaggggactaacagggcctcgcagtgtagacagagggtgcagccacacgggcgggggcaccagccacctcactctgcacccgcggcctcacacatctcccagctcacactctactaatgcacagagtcattagatccaatttgttatttttctcacttgctttaaaaaaaagcagtttggataatcatgacattggaataaagtgggaaggaaaaattccatcagcacaaaatagggaagtaatcccaacttgtagtcacagttttctgactggctttgttttaaaagaggatggcagtccttgttcgtgtcagtgtgccactgggtttttgctgttccgtgtaattcatatcaactttgtgttgccatttgcaaggtaaaaggcaaagctgtagtgtattcacctatgtagacagattgctagatatctttttgatctggggcgagttcaatattgattccagacttatttggatttttttagtattattttcccctccctttctaatttaaatagacaaattaagcaaaagtgtgtgttcacaaccaaatgttgatgcccttatctactgataatatcctctcaatgttcactgaggcatagaaattatttcagagtagaaattgcagcatgaggataaactcacctctttgttctgaaaatagaactttatcactatgctttccggtggttttcccttttacaatcgaaatcttgtgcctcccaagtgcattggaaaatgacaaaagcctgtctctccaaattcctatttaacagtttgattttttttttttaatcaccatctttcaaatcttagctcaactctcaccaagtgaaaattggctacttgggagaaagttaactttctatggtgggatggtgaaggatgagggacagtttacataggaaaagaaaaaaaaaagtctaaagtccatgttgaaaaaccacactaccacttattttctgctaaccctaaattatttttgcgtatacgcttgaggttatagtctgtgcctagacctaaaatgcaccagcgggggggattttaaaaaatccttcaaaataccagttttttcccaacaagtacaattgttcttgtgccttctgtggctttcgatttcatctttttgactttatttccaattactacagctgcaataaacactagattttttttctggctgtttgacataacgttgatagctatgcatattttgtgtctttttaaaacaaagcgggagaatacgtttttgaagaagagaatttttagaacagtttgataccgcaaattattttttcctcaattgtttgagcagcattcgagttttgaaaattcttgtagaagccaattttttgtaactgtggtgcaaatcttgtgttttcttagcctaatgaaaagtagtatagaagcaatatttcataccatgtgctatatatgtgtgcgcagatgtgtgaacataaaatcacatacacacatatacacacatgtaaaaatatacatatatatatatgcgtgtgaagtggaaagcttaccttttcctatctagatttaagaacctattttagacatttgttatgttttgtgaaaagaatgttctatttgcaacaaaacatttaattcttactgtatctctggctgtttaatgaggacgtttcacattaaatggtaaaacacatggaagatgttagaatgtagtaattatttaagtaaacgttcacccacatattcctgaagtttgctttgtgcctccgagtattatttaattaaagaagtgttttatgtttgcagaatctttgtcactgtactagggatgtgggtgaatatcatttaaaaaaatttaaaacaacaaaaaaaaagcaaaacagaaacactaaagcaagaggggaacttttataaagcaatgtaaatatttaacctcatggctgtcattatgtaagacatgagattttaataaataactacattctcacgacatctgttgaatttactaggaacactacagtgactgtatagacagttgaaagcattcttgaaaatcctgctctctccttttaaaagttaacaatctcttttatcagatgtcaagggcaagggtaatgcagtttctgtaaatttatgaaatttctttttctatgtacatgaagacatttagtaagtaacacccccccttcccatgcgcacatgtgcgcatacacacacacacacacacacacacacacacaaacacacacactgtcataaagctaatgatttggggactttaaaaaataggatgtcctccaggaacaatcataaatttatgaaagaaagagtagtttacagactcccctgaaagaagcagtgtatatgtgaagacagtgcaaaaatctctttgccatgtatattatagcgtattcattggtgtgaatagtacaaatgtttccttctggtacaaactctgtgtttgcaaatttacaagaagcattgttttcaaaaagctccccttaaaaaatgtaactggtttatatgagtaagcagttaccgtattgcacttaaatgttatgttgaaggaaatgcagttttgttttctgtagatctgttggttgtaaaccatctataaaactaaagctaaaatgctcatattcagagctgggatcaaaactggtatttaacctttgcatcttcttataattatccttctaagaatataacagaatgtggaagtgtctggactttgagtcttttcaactgagccttctctcaaatctgacaccccctcagaatgcacaaacataagcagaaaaggcaaacaagcttaccttcttttgtgaaaacgtattcattctgtatttttttaaatattcaattcccctaaaaatggggagaaaatattttaaaattgtatattacgacttcaaatttagaactaagaaaaaaatgtatttgggattggtctcagcgctacctagaagaatcaaaggtcatggcttccctcaatattgtcccagccatttctcatatgtatatagtataaaccgtgacaaaacactgcctttatattatttagcaatatgttgtaaatagcattattaagctcttttttgtaataaagaccctttgatttgaatatagtacaataactgaactgataaagtcaatttttgatttttgtttgttttttttagctagaggcaatttcaattgtgaatttttgttgttgtctattgttctgaagactttgcataatttattggtttaatttatcctaatttatttgatgaaggtgtacaattttgtattaccaaggatgtactgtaatattaattgatatgataaacacaatgagactccctgtccatattaaaaagaaaataaaaaggtgcagtagacaattgattttaaaggaaaagttaaaaaaattagtttggcagctactaaattttaaaacaggaaaaaaaaaagttgttgtggggagggtgggaaaggggttttactttgtgtgttttaagcttttgtatactctccaaacttttaccttttgctttgtaccacttaaaggatacagtagtccaattgccttgtgtgccttccatctcctcttaaactgaatgtatgtgcagtatatatgcaagcttgtgcaaaataaaatatacattacaagctcagtgccgtttgattttcttaaagaaagagtgacttttaatttttggacctgtatccaattgtaggacagtaggctagttgtgccagtaatgtcaagtatggagattttctttcactacaattcttcattctgttagcctaacgtgcagctcctagaaacaacctcttttactttagatgcttggaataattgcttggatttctctctctgaaacatctttcaggcttaactttatttagccctgaaacttaaaaaaaa

SEQ ID NO: 40 - protein, 441 aa, Homo sapiens
MYSPYCLTQDEFHPFIEALLPHVRAFSYTWFNLQARKRKYFKKHEKRMSKDEERAVKDELLGEKPEIKQKWASRLLAKLRKDIRPEFREDFVLTITGKKPPCCVLSNPDQKGKIRRIDCLRQADKVWRLDLVMVILFKGIPLESTDGERLYKSPQCSNPGLCVQPHHIGVTIKELDLYLAYFVHTPESGQSDSSNQQGDADIKPLPNGHLSFQDCFVTSGVWNVTELVRVSQTPVATASGPNFSLADLESPSYYNINQVTLGRRSITSPPSTSTTKRPKSIDDSEMESPVDDVFYPGTGRSPAAGSSQSSGWPNDVDAGPASLKKSGKLDFCSALSSQGSSPRMAFTHHPLPVLAGVRPGSPRATASALHFPSTSIIQQSSPYFTHPTIRYHHHHGQDSLKEFVQFVCSDGSGQATGQHSQRQAPPLPTGLSASDPGTATF

SEQ ID NO: 41 - protein, 508 aa, Homo sapiens
MYSSPLCLTQDEFHPFIEALLPHVRAFAYTWFNLQARKRKYFKKHEKRMSKDEERAVKDELLGEKPEVKQKWASRLLAKLRKDIRPECREDFVLSITGKKAPGCVLSNPDQKGKMRRIDCLRQADKVWRLDLVMVILFKGIPLESTDGERLVKAAQCGHPVLCVQPHHIGVAVKELDLYLAYFVRERDAEQSGSPRTGMGSDQEDSKPITLDTTDFQESFVTSGVFSVTELIQVSRTPVVTGTGPNFSLGELQGHLAYDLNPASTGLRRTLPSTSSSGSKRHKSGSMEEDVDTSPGGDYYTSPSSPTSSSRNWTEDMEGGISSPVKKTEMDKSPFNSPSPQDSPRLSSFTQHHRPVIAVHSGIARSPHPSSALHFPTTSILPQTASTYFPHTAIRYPPHLNPQDPLKDLVSLACDPASQQPGPLNGSGQLKMPSHCLSAQMLAPPPPGLPRLALPPATKPATTSEGGATSPTSPSYSPPDTSPANRSFVGLGPRDPAGIYQAQSWYLG

SEQ ID NO: 42 - protein, 499 aa, Homo sapiens
MDEFHPFIEALLPHVRAFAYTWFNLQARKRKYFKKHEKRMSKDEERAVKDELLGEKPEVKQKWASRLLAKLRKDIRPECREDFVLSITGKKAPGCVLSNPDQKGKMRRIDCLRQADKVWRLDLVMVILFKGIPLESTDGERLVKAAQCGHPVLCVQPHHIGVAVKELDLYLAYFVRERDAEQSGSPRTGMGSDQEDSKPITLDTTDFQESFVTSGVFSVTELIQVSRTPVVTGTGPNFSLGELQGHLAYDLNPASTGLRRTLPSTSSSGSKRHKSGSMEEDVDTSPGGDYYTSPSSPTSSSRNWTEDMEGGISSPVKKTEMDKSPFNSPSPQDSPRLSSFTQHHRPVIAVHSGIARSPHPSSALHFPTTSILPQTASTYFPHTAIRYPPHLNPQDPLKDLVSLACDPASQQPGPLNGSGQLKMPSHCLSAQMLAPPPPGLPRLALPPATKPATTSEGGATSPTSPSYSPPDTSPANRSFVGLGPRDPAGIYQAQSWYLG

SEQ ID NO: 43 - protein, 439 aa, Homo sapiens
MYSSPLCLTQDEFHPFIEALLPHVRAFAYTWFNLQARKRKYFKKHEKRMSKDEERAVKDELLGEKPEVKQKWASRLLAKLRKDIRPECREDFVLSITGKKAPGCVLSNPDQKGKMRRIDCLRQADKVWRLDLVMVILFKGIPLESTDGERLVKAAQCGHPVLCVQPHHIGVAVKELDLYLAYFVRERDAEQSGSPRTGMGSDQEDSKPITLDTTDFQESFVTSGVFSVTELIQVSRTPVVTGTGPNFSLGELQGHLAYDLNPASTGLRRTLPSTSSSGSKRHKSGSMEEDVDTSPGGDYYTSPSSPTSSSRNWTEDMEGGISSPVKKTEMDKSPFNSPSPQDSPRLSSFTQHHRPVIAVHSGIARSPHPSSALHFPTTSILPQTASTYFPHTAIRYPPHLNPQDPLKDLVSLACDPASQQPGPPTLRPTRPLQTVPLWD

SEQ ID NO: 44 - protein, 430 aa, Homo sapiens
MDEFHPFIEALLPHVRAFAYTWFNLQARKRKYFKKHEKRMSKDEERAVKDELLGEKPEVKQKWASRLLAKLRKDIRPECREDFVLSITGKKAPGCVLSNPDQKGKMRRIDCLRQADKVWRLDLVMVILFKGIPLESTDGERLVKAAQCGHPVLCVQPHHIGVAVKELDLYLAYFVRERDAEQSGSPRTGMGSDQEDSKPITLDTTDFQESFVTSGVFSVTELIQVSRTPVVTGTGPNFSLGELQGHLAYDLNPASTGLRRTLPSTSSSGSKRHKSGSMEEDVDTSPGGDYYTSPSSPTSSSRNWTEDMEGGISSPVKKTEMDKSPFNSPSPQDSPRLSSFTQHHRPVIAVHSGIARSPHPSSALHFPTTSILPQTASTYFPHTAIRYPPHLNPQDPLKDLVSLACDPASQQPGPPTLRPTRPLQTVPLWD

SEQ ID NO: 45 - protein, 428 aa, Homo sapiens
MYSSPLCLTQDEFHPFIEALLPHVRAFAYTWFNLQARKRKYFKKHEKRMSKDEERAVKDELLGEKPEVKQKWASRLLAKLRKDIRPECREDFVLSITGKKAPGCVLSNPDQKGKMRRIDCLRQADKVWRLDLVMVILFKGIPLESTDGERLVKAAQCGHPVLCVQPHHIGVAVKELDLYLAYFVRERDAEQSGSPRTGMGSDQEDSKPITLDTTDFQESFVTSGVFSVTELIQVSRTPVVTGTGPNFSLGELQGHLAYDLNPASTGLRRTLPSTSSSGSKRHKSGSMEEDVDTSPGGDYYTSPSSPTSSSRNWTEDMEGGISSPVKKTEMDKSPFNSPSPQDSPRLSSFTQHHRPVIAVHSGIARSPHPSSALHFPTTSILPQTASTYFPHTAIRYPPHLNPQDPLKDLVSLACDPASQQPGPSWYLG

SEQ ID NO: 46 - DNA, 15 bp, synthetic construct
  misc_feature 1..15: note = Description of Artificial Sequence: Synthetic oligonucleotide
  misc_difference 6..10: note = modified_base - a, c, t, g, unknown or other
  source 1..15: mol_type = other DNA
ttggcnnnnngccaa

(19) United States
(12) Patent Application Publication
(10) Pub. No.: US 2024/0218332 A1
(43) Pub. Date: Jul. 4, 2024

(54) METHODS OF GENERATING MATURE HEPATOCYTES

(71) Applicant: Astellas Institute for Regenerative Medicine, Westborough, MA (US)

(72) Inventors: Ana D'alessio, Westborough, MA (US); Erin Kimbrel, Westborough, MA (US)

(21) Appl. No.: 18/386,860

(22) Filed: Nov. 3, 2023

Related U.S. Application Data
(63) Continuation of application No. PCT/US2022/027776, filed on May 5, 2022.
(60) Provisional application No. 63/185,735, filed on May 7, 2021.

(51) Int. Cl. C12N 5/071; A61K 35/407; C12N 15/86

(52) U.S. Cl. CPC C12N 5/067; A61K 35/407; C12N 15/86; C12N 2501/12; C12N 2501/237; C12N 2501/39; C12N 2501/727; C12N 2506/02; C12N 2506/45; C12N 2740/15043

(57) ABSTRACT

The present invention provides methods of generating mature hepatocytes by increasing expression of at least one transcription factor selected from the group consisting of Nuclear Factor I X (NFIX) and Nuclear Factor I C (NFIC) in immature hepatocytes, and compositions thereof.

### RELATED APPLICATIONS

This application is a 35 § U.S.C. 111(a) continuation application which claims the benefit of priority to International Application No. PCT/US2022/027776, filed on May 5, 2022, which in turn claims the benefit of priority under 35 U.S.C. § 119(e) to U.S. Provisional Application No. 63/185,735, filed on May 7, 2021. The entire contents of each of the foregoing applications are incorporated herein by reference.

### SEQUENCE LISTING

The instant application contains a Sequence Listing which has been submitted electronically in XML format and is hereby incorporated by reference in its entirety. Said XML copy, created on Nov. 16, 2023, is named 127206-08202_SL.xml and is 244,417 bytes in size.

### FIELD OF THE INVENTION

The present invention relates to methods of generating mature hepatocytes, and compositions thereof.

### BACKGROUND

Hepatocytes are responsible for drug metabolism and control of xenobiotic elimination from the body (Gebhardt et al., 2003, Drug Metab Rev 35, 145-213; and Hewitt et al., 2007, Drug Metab Rev 39, 159-234). Due to their critical function in the detoxification of drugs, xenobiotics as well as endogenous substrates, hepatocytes are used in drug toxicity screening and development programs. Human primary hepatocytes, however, quickly lose their functions when cultured in vitro. Moreover, the drug metabolic ability of human primary hepatocytes exhibits a significant difference between different individuals (Byers et al., 2007, Drug Metab Lett 1, 91-95).

In addition to providing new platforms for drug testing, hepatocytes offer potential new therapies for patients with liver disease. Although liver transplantation provides an effective treatment for end-stage liver disease, a shortage of viable donor organs limits the patient population that can be treated with hepatocytes (Kawasaki et al., 1998, Ann Surg 227, 269-274; and Miro et al., 2006, J Hepatol 44, 5140-145). Hepatocyte transplantation and bioartificial liver devices developed with hepatocytes represent alternative life-saving therapies for patients with specific types of liver disease. Given the important functional roles of hepatocytes, and the fact that individuals can differ in their ability to metabolize a particular drug, there is a need for access to mature and functional hepatocytes.

Reproducible and efficient generation of mature hepatocytes has been challenging to date, due to the fact that the regulatory pathways that control hepatocyte maturation are poorly understood. Almost all approaches have attempted to recapitulate the key stages of liver development in differentiation cultures, including the induction of definitive endoderm, the specification of the endoderm to a hepatic fate, and the generation of hepatic progenitors. While these early differentiation steps are reasonably well-established, conditions that promote the maturation of the hepatocytes are not well-understood. Further, the populations produced with the different protocols vary considerably in their maturation status and represent immature hepatocytes.

Accordingly, there is a need in the art for simple and effective methods for producing mature hepatocytes.

### SUMMARY

The present invention meets this need in the art by providing efficient and effective methods for producing mature hepatocytes by increasing expression of at least one transcription factor selected from the group consisting of Nuclear Factor I X (NFIX) and Nuclear Factor I C (NFIC) in immature hepatocytes. In one aspect, the invention provides novel and effective methods for generating mature hepatocytes by increasing expression of at least one transcription factor selected from the group consisting of Nuclear Factor I X (NFIX) and Nuclear Factor I C (NFIC) in immature hepatocytes.

The methods of the invention are both simple, efficient and effective, and result in the production of mature hepatocytes that can be used for a variety of applications disclosed herein, for example, treatment of liver diseases.

In one aspect, the invention provides a method of generating mature hepatocytes, the method comprising increasing expression of at least one transcription factor selected from the group consisting of Nuclear Factor I X (NFIX) and Nuclear Factor I C (NFIC), in immature hepatocytes, thereby generating mature hepatocytes.

In some embodiments, the transcription factor is NFIX.

In some embodiments, the transcription factor is NFIC.

In some embodiments, the transcription factor is NFIX and NFIC.

In some embodiments, the NFIC is at least one alternatively spliced NFIC variant selected from the group consisting of NFIC, transcript variant 1; NFIC, transcript variant 2; NFIC, transcript variant 3; NFIC, transcript variant 4; and NFIC, transcript variant 5. In some embodiments, the alternatively spliced NFIC variant is NFIC, transcript variant 1. In some embodiments, the alternatively spliced NFIC variant is NFIC, transcript variant 3. In some embodiments, the alternatively spliced NFIC variant is NFIC, transcript variant 1 and NFIC, transcript variant 3.

In some embodiments, the method further comprises increasing expression of one or more transcription factors selected from the group consisting of RORC, NR0B2, ESR1, THRSP, TBX15, HLF, ATOH8, NR112, CUX2, ZNF662, TSHZ2, ATF5, NFIA, NFIB, NPAS2, FOS, ONECUT2, PROX1, NR1H4, MLXIPL, ETV1, AR, CEBPB, NR1D1, HEY2, ARID3C, KLF9, and DMRTA1 in the immature hepatocytes.

In some embodiments, the method further comprises culturing the immature hepatocytes in a culture media comprising dexamethasone, 8-Bromoadenosine 3′, 5′-cyclic monophosphate (8-Br-cAMP), or a combination thereof. In some embodiments, the culturing is performed for at least 2, 3, 4, 5, 6, 7, 8 or 9 days. In some embodiments, the concentration of 8-Br-CAMP is at least 0.1 mM, 0.2 mM, 0.4 mM, 0.6 mM, 0.8 nM or 1 mM. In some embodiments, the concentration of dexamethasone is at least 5 nM, 10 nM, 20 nM, 40 nM, 60 nM, 80 nM or 100 nM.

In some embodiments, increasing the expression of the at least one transcription factor in the immature hepatocytes comprises contacting the immature hepatocytes with the at least one transcription factor.

In some embodiments, the immature hepatocytes comprise an expression vector comprising a nucleic acid encoding the at least one transcription factor. In some embodiments, the expression vector is a viral vector. In some embodiments, the expression vector is a non-viral vector. In some embodiments, the expression vector is an inducible expression vector. In some embodiments, the expression vector comprises a promoter operably linked to a nucleic acid encoding the at least one transcription factor. In some embodiments, the promoter is an endogenous promoter. In some embodiments, the promoter is an artificial promoter. In some embodiments, the promoter is an inducible promoter.

In some embodiments, increasing the expression of the at least one transcription factor in the immature hepatocytes comprises transduction of immature hepatocytes with a viral vector encoding the at least one transcription factor.

In some embodiments, increasing the expression of the at least one transcription factor in the immature hepatocytes comprises transfection of immature hepatocytes with an expression vector encoding the at least one transcription factor.

In some embodiments, the immature hepatocytes are cultured for at least 2, 3, 4 or 5 days before increasing the expression of the at least one transcription factor.

In some embodiments, the immature hepatocytes are cultured for at least 2, 3, 4, 5, 6, 7, 8 or 9 days after increasing the expression of the at least one transcription factor.

In some embodiments, increasing the expression of NFIX comprises an increase of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to endogenous expression levels of NFIX in the immature hepatocytes.

In some embodiments, increasing the expression of NFIC comprises an increase of at least 0.1-fold, 0.2-fold, 0.5-fold, 1-fold, 2-fold, 5-fold, 10-fold, 20-fold, 50-fold, 100-fold, 200-fold, 500-fold, 1000-fold, or 10,000-fold relative to endogenous expression levels of NFIC in the immature hepatocytes.

In some embodiments, the mature hepatocytes exhibit an increased expression of albumin (ALB), cytochrome P450 enzyme 1A2 (CYP1A2), cytochrome P450 enzyme 3A4 (CYP3A4), tyrosine aminotransferase (TAT), and/or UDP-glucuronosyltransferase 1A-1 (UGT1A1) relative to immature hepatocytes. In some embodiments, the increased expression of CYP1A2 comprises an increase of at least 2-fold, 5-fold, 10-fold, 50-fold, 100-fold, 200-fold, 500-fold, 1,000-fold, 2,000-fold, 5,000-fold, or 10,000-fold relative to immature hepatocytes. In some embodiments, the increased expression of CYP3A4 comprises an increase of at least 2-fold, 5-fold, 10-fold, 50-fold, 100-fold, 200-fold, 500-fold, 1,000-fold, 2,000-fold, 5,000-fold, or 10,000-fold relative to immature hepatocytes. In some embodiments, the increased expression of TAT comprises an increase of at least 2-fold, 5-fold, 10-fold, 50-fold, 100-fold, 200-fold, 500-fold, 1,000-fold, 2,000-fold, 5,000-fold, or 10,000-fold relative to immature hepatocytes. In some embodiments, the increased expression of UGT1A1 comprises an increase of at least 2-fold, 5-fold, 10-fold, 50-fold, 100-fold, 500-fold, 1,000-fold, 2,000-fold, 5,000-fold, or 10,000-fold relative to immature hepatocytes.

In some embodiments, the mature hepatocytes exhibit a decreased expression of alpha fetoprotein (AFP) relative to immature hepatocytes. In some embodiments, the decreased expression of AFP comprises a decrease of at least 0.1-fold, 0.2-fold, 0.5-fold, 1-fold, 2-fold, 3-fold, or 4-fold relative to immature hepatocytes.

In some embodiments, the mature hepatocytes exhibit an increased secretion of albumin (ALB), a decreased secretion of AFP, and/or an increased activity of CYP1A2, relative to immature hepatocytes. In some embodiments, the increased secretion of ALB comprises an increase of at least 5%, 10%, 15%, 20% or 25% relative to immature hepatocytes. In some embodiments, the decreased secretion of AFP comprises a decrease of at least 5%, 10%, 20%, 40%, or 60% relative to immature hepatocytes. In some embodiments, the increased activity of CYP1A2 comprises an increase of at least 2-fold, 5-fold, 10-fold, 50-fold, 100-fold, 200-fold, or 400-fold relative to immature hepatocytes.

In some embodiments, increasing the expression of the at least one transcription factor shifts the transcriptome of immature hepatocytes towards the transcriptome of mature hepatocytes by at least 1%, 5%, 10%, 20%, 30%, 40%, or 50%.

In some embodiments, the immature hepatocytes are derived from pluripotent stem cells. In some embodiments, the pluripotent stem cells are embryonic stem cells or induced pluripotent stem cells.

In some embodiments, increasing the expression of the at least one transcription factor in the immature hepatocytes comprises use of a gene switch construct encoding the at least one transcription factor. In some embodiments, the gene switch construct is a transcriptional gene switch construct or a post-transcriptional gene switch construct.

In some embodiments, the expression vector further comprises a self-cleaving sequence.

In some embodiments, NFIX comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence as set forth in SEQ ID NO: 1.

In some embodiments, NFIC comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by any one of the nucleotide sequences of SEQ ID NO: 2 to SEQ ID NO: 6.

In some embodiments, NFIX comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence set forth in SEQ ID NO: 40.

In some embodiments, NFIC comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to any one of the amino acid sequences set forth in SEQ ID NO: 41-SEQ ID NO: 45.

In another aspect, the invention provides a method of generating pluripotent stem cell-derived mature hepatocytes, the method comprising: (a) differentiating pluripotent stem cells to immature hepatocytes, wherein the pluripotent stem cells comprise an expression vector comprising a nucleic acid encoding the at least one transcription factor selected from the group consisting of Nuclear Factor I X (NFIX) and Nuclear Factor I C (NFIC), and (b) increasing expression of the at least one transcription factor from the expression vector in the immature hepatocytes, thereby generating mature hepatocytes.

In some embodiments, the pluripotent stem cells are embryonic stem cells.

In some embodiments, the pluripotent stem cells are induced pluripotent stem cells.

In some embodiments, the immature hepatocytes comprise hepatoblasts.

In some embodiments, the immature hepatocytes comprise hepatic stem cells.

In some embodiments, the transcription factor is NFIX.

In some embodiments, the transcription factor is NFIC.

In some embodiments, the transcription factor is NFIX and NFIC.

In some embodiments, the NFIC is at least one alternatively spliced NFIC variant selected from the group consisting of NFIC, transcript variant 1; NFIC, transcript variant 2; NFIC, transcript variant 3; NFIC, transcript variant 4; and NFIC, transcript variant 5. In some embodiments, the alternatively spliced NFIC variant is NFIC, transcript variant 1. In some embodiments, the alternatively spliced NFIC variant is NFIC, transcript variant 3. In some embodiments, the alternatively spliced NFIC variant is NFIC, transcript variant 1 and NFIC, transcript variant 3.

In some embodiments, the method further comprises increasing expression of one or more transcription factors selected from the group consisting of RORC, NR0B2, ESR1, THRSP, TBX15, HLF, ATOH8, NR1I2, CUX2, ZNF662, TSHZ2, ATF5, NFIA, NFIB, NPAS2, FOS, ONECUT2, PROX1, NR1H4, MLXIPL, ETV1, AR, CEBPB, NR1D1, HEY2, ARID3C, KLF9, and DMRTA1 in the immature hepatocytes.

In some embodiments, the method further comprises culturing the immature hepatocytes in a culture media comprising dexamethasone, 8-Bromoadenosine 3′, 5′-cyclic monophosphate (8-Br-cAMP), or a combination thereof. In some embodiments, the culturing is performed for at least 2, 3, 4, 5, 6, 7, 8 or 9 days. In some embodiments, the concentration of 8-Br-CAMP is at least 0.1 mM, 0.2 mM, 0.4 mM, 0.6 mM, 0.8 nM or 1 mM. In some embodiments, the concentration of dexamethasone is at least 5 nM, 10, nM, 20 nM, 40 nM, 60 nM, 80 nM or 100 nM.

In some embodiments, the immature hepatocytes comprise the expression vector comprising the nucleic acid encoding the at least one transcription factor.

In some embodiments, the expression vector is a viral vector.

In some embodiments, the expression vector is a non-viral vector.

In some embodiments, the expression vector is an inducible expression vector.

In some embodiments, the expression vector comprises a promoter operably linked to a nucleic acid encoding the at least one transcription factor. In some embodiments, the promoter is an endogenous promoter. In some embodiments, the promoter is an artificial promoter. In some embodiments, the promoter is an inducible promoter.

In some embodiments, increasing the expression of the at least one transcription factor in the immature hepatocytes comprises inducing expression of the at least one transcription factor in the immature hepatocytes. In some embodiments, inducing the expression of the at least one transcription factor in the immature hepatocytes comprises use of a gene switch construct encoding the at least one transcription factor. In some embodiments, the gene switch construct is a transcriptional gene switch construct or a post-transcriptional gene switch construct.

In some embodiments, the expression vector further comprises a self-cleaving sequence.

In some embodiments, the pluripotent stem cells are transduced with a viral vector encoding the at least one transcription factor.

In some embodiments, the pluripotent stem cells are transfected with an expression vector encoding the at least one transcription factor.

In some embodiments, step (a) of the method comprises culturing the pluripotent stem cells in a first differentiation media comprising Activin A, a second differentiation media comprising at least one of BMP4 and FGF2, and a third differentiation media comprising HGF, thereby generating the immature hepatocytes. In some embodiments, the first differentiation media, the second differentiation media and the third differentiation media are each cultured for at least 5 days.

In some embodiments, the immature hepatocytes are cultured for at least 2, 3, 4 or 5 days before increasing the expression of the at least one transcription factor. In some embodiments, the immature hepatocytes are cultured in a culture media comprising hepatocyte growth factor (HGF).

In some embodiments, the immature hepatocytes are cultured for at least 2, 3, 4, 5, 6, 7, 8 or 9 days after increasing the expression of the at least one transcription factor. In some embodiments, the immature hepatocytes are cultured in a culture media comprising oncostatin-M (OSM).

In some embodiments, increasing the expression of NFIX comprises an increase of at least 0.1-fold, 0.2-fold, 0.5-fold, 1-fold, 2-fold, 5-fold, 10-fold, 20-fold, 50-fold, 100-fold, 200-fold, 500-fold, 1000-fold, or 10,000-fold relative to endogenous expression levels of NFIX in the immature hepatocytes.

In some embodiments, increasing the expression of NFIC comprises an increase of at least 0.1-fold, 0.2-fold, 0.5-fold, 1-fold, 2-fold, 5-fold, 10-fold, 20-fold, 50-fold, 100-fold, 200-fold, 500-fold, 1000-fold, or 10,000-fold relative to endogenous expression levels of NFIC in the immature hepatocytes.

In some embodiments, the mature hepatocytes exhibit an increased expression of albumin (ALB), cytochrome P450 enzyme 1A2 (CYP1A2), cytochrome P450 enzyme 3A4 (CYP3A4), tyrosine aminotransferase (TAT), and/or UDP-glucuronosyltransferase 1A-1 (UGT1A1) relative to immature hepatocytes. In some embodiments, the increased expression of CYP1A2 comprises an increase of at least 2-fold, 5-fold, 10-fold, 50-fold, 100-fold, 200-fold, 500-fold, 1,000-fold, 2,000-fold, 5,000-fold or 10,000-fold relative to immature hepatocytes. In some embodiments, the increased expression of CYP3A4 comprises an increase of at least 2-fold, 5-fold, 10-fold, 50-fold, 100-fold, 200-fold, 500-fold, 1,000-fold, 2,000-fold, 5,000-fold or 10,000-fold relative to immature hepatocytes. In some embodiments, the increased expression of TAT comprises an increase of at least 2-fold, 5-fold, 10-fold, 50-fold, 100-fold, 200-fold, 500-fold, 1,000-fold, 2,000-fold, 5,000-fold or 10,000-fold relative to immature hepatocytes. In some embodiments, the increased expression of UGT1A1 comprises an increase of at least 2-fold, 5-fold, 10-fold, 50-fold, 100-fold, 200-fold, 500-fold, 1,000-fold, 2,000-fold, 5,000-fold or 10,000-fold relative to immature hepatocytes.

In some embodiments, the mature hepatocytes exhibit a decreased expression of alpha fetoprotein (AFP) relative to immature hepatocytes. In some embodiments, the decreased expression of AFP comprises a decrease of at least 0.1-fold, 0.2-fold, 0.5-fold, 1-fold, 2-fold, 3-fold, or 4-fold relative to immature hepatocytes.

In some embodiments, the mature hepatocytes exhibit an increased secretion of albumin (ALB), a decreased secretion of AFP, and/or an increased activity of CYP1A2, relative to immature hepatocytes. In some embodiments, the increased secretion of ALB comprises an increase of at least 5%, 10%, 15%, 20% or 25% relative to immature hepatocytes. In some embodiments, the decreased secretion of AFP comprises a decrease of at least 5%, 10%, 20%, 40%, or 60% relative to immature hepatocytes. In some embodiments, the increased activity of CYP1A2 comprises an increase of at least 2-fold, 5-fold, 10-fold, 50-fold, 100-fold, 200-fold, or 400-fold relative to immature hepatocytes.

In some embodiments, increasing the expression of the at least one transcription factor shifts the transcriptome of immature hepatocytes towards the transcriptome of mature hepatocytes by at least 1%, 5%, 10%, 20%, 30%, 40%, or 50%.

In another aspect, the invention provides a composition comprising a population of mature hepatocytes produced by any one or more of the methods disclosed herein.

In another aspect, the invention provides a pharmaceutical composition comprising a population of mature hepatocytes produced by any one or more of the methods disclosed herein, and a pharmaceutically acceptable carrier.

In some embodiments, NFIX comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence as set forth in SEQ ID NO: 1.

In some embodiments, NFIC comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by any one of the nucleotide sequences of SEQ ID NO: 2 to SEQ ID NO: 6.

In some embodiments, NFIX comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence set forth in SEQ ID NO: 40.

In some embodiments, NFIC comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to any one of the amino acid sequences set forth in SEQ ID NO: 41-SEQ ID NO: 45.

In another aspect, the invention provides a composition comprising a population of hepatocytes comprising increased expression levels of at least one transcription factor selected from the group consisting of Nuclear Factor I X (NFIX) and Nuclear Factor I C (NFIC), relative to endogenous expression levels of the transcription factor in the population of hepatocytes.

In some embodiments, the transcription factor is NFIX.

In some embodiments, the transcription factor is NFIC.

In some embodiments, the transcription factor is NFIX and NFIC.

In some embodiments, the NFIC is at least one alternatively spliced NFIC variant selected from the group consisting of NFIC, transcript variant 1; NFIC, transcript variant 2; NFIC, transcript variant 3; NFIC, transcript variant 4; and NFIC, transcript variant 5. In some embodiments, the alternatively spliced NFIC variant is NFIC, transcript variant 1. In some embodiments, the alternatively spliced NFIC variant is NFIC, transcript variant 3. In some embodiments, the alternatively spliced NFIC variant is NFIC, transcript variant 1 and NFIC, transcript variant 3.

In some embodiments, the hepatocytes further comprise increased expression levels of one or more transcription factors selected from the group consisting of RORC, NR0B2, ESR1, THRSP, TBX15, HLF, ATOH8, NR112, CUX2, ZNF662, TSHZ2, ATF5, NFIA, NFIB, NPAS2, FOS, ONECUT2, PROX1, NR1H4, MLXIPL, ETV1, AR, CEBPB, NR1D1, HEY2, ARID3C, KLF9, and DMRTA1 relative to endogenous expression levels of the one or more transcription factors in the population of hepatocytes.

In some embodiments, the increased expression comprises exogenous expression of the at least one transcription factor.

In some embodiments, the hepatocytes comprise an expression vector comprising a nucleic acid encoding the at least one transcription factor.

In some embodiments, the expression vector is a viral vector. In some embodiments, the viral vector is selected from the group consisting of an adeno-associated virus (AAV) vector, an adenovirus vector, a lentivirus vector, a herpes simplex virus vector, a sendai virus vector, and a retrovirus vector.

In some embodiments, the expression vector is a non-viral vector. In some embodiments, the non-viral vector is selected from the group consisting of a plasmid DNA, a linear double-stranded DNA (dsDNA), a linear single-stranded DNA (ssDNA), a nanoplasmid, a minicircle DNA, a single-stranded oligodeoxynucleotides (ssODN), a DDNA oligonucleotide, a single-stranded mRNA (ssRNA), and a double-stranded mRNA (dsRNA). In some embodiments, the non-viral vector comprises a naked nucleic acid, a liposome, a dendrimer, a nanoparticle, a lipid-polymer system, a solid lipid nanoparticle, and/or a liposome protamine/DNA lipoplex (LPD).

In some embodiments, the expression vector is an inducible expression vector.

In some embodiments, the expression vector comprises a promoter operably linked to a nucleic acid encoding the at least one transcription factor. In some embodiments, the promoter is an endogenous promoter. In some embodiments, the promoter is an artificial promoter. In some embodiments, the promoter is an inducible promoter.

In some embodiments, the expression vector comprises a gene switch construct encoding the at least one transcription factor. In some embodiments, the gene switch construct is a transcriptional gene switch construct or a post-transcriptional gene switch construct.

In some embodiments, the expression vector further comprises a self-cleaving sequence. In some embodiments, the self-cleaving sequence is selected from the group consisting of T2A, P2A, E2A and F2A.

In some embodiments, the increased expression of NFIX comprises an increase of at least 0.1-fold, 0.2-fold, 0.5-fold, 1-fold, 2-fold, 5-fold, 10-fold, 20-fold, 50-fold, 100-fold, 200-fold, 500-fold, 1000-fold, or 10,000-fold relative to endogenous expression levels of NFIX in the population of hepatocytes.

In some embodiments, the increased expression of NFIC comprises an increase of at least 0.1-fold, 0.2-fold, 0.5-fold, 1-fold, 2-fold, 5-fold, 10-fold, 20-fold, 50-fold, 100-fold, 200-fold, 500-fold, 1000-fold, or 10,000-fold relative to endogenous expression levels of NFIC in the population of hepatocytes.

In some embodiments, the population of hepatocytes is a population of immature hepatocytes.

In some embodiments, the population of hepatocytes is a population of mature hepatocytes.

In some embodiments, the composition further comprises non-hepatocyte cells.

In some embodiments, the population of hepatocytes are in the form of organoids.

In some embodiments, the hepatocytes are derived from pluripotent stem cells. In some embodiments, the pluripotent stem cells are embryonic stem cells or induced pluripotent stem cells.

In some embodiments, the population of hepatocytes comprises at least 10⁶ hepatocytes.

In another aspect, the invention provides a pharmaceutical composition comprising the population of hepatocytes of any one or more of the compositions described herein, and a pharmaceutically acceptable carrier.

In some embodiments, NFIX comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence as set forth in SEQ ID NO: 1.

In some embodiments, NFIC comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by any one of the nucleotide sequences of SEQ ID NO: 2 to SEQ ID NO: 6.

In some embodiments, NFIX comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence set forth in SEQ ID NO: 40.

In some embodiments, NFIC comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to any one of the amino acid sequences set forth in SEQ ID NO: 41-SEQ ID NO: 45.

In another aspect, the invention provides a composition comprising a population of pluripotent stem cells comprising an expression vector, wherein the expression vector comprises a nucleic acid encoding at least one transcription factor selected from the group consisting of Nuclear Factor I X (NFIX) and Nuclear Factor I C (NFIC).

In some embodiments, the transcription factor is NFIX.

In some embodiments, the transcription factor is NFIC.

In some embodiments, the transcription factor is NFIX and NFIC.

In some embodiments, the NFIC is at least one alternatively spliced NFIC variant selected from the group consisting of NFIC, transcript variant 1; NFIC, transcript variant 2; NFIC, transcript variant 3; NFIC, transcript variant 4; and NFIC, transcript variant 5. In some embodiments, the alternatively spliced NFIC variant is NFIC, transcript variant 1. In some embodiments, the alternatively spliced NFIC variant is NFIC, transcript variant 3. In some embodiments, the alternatively spliced NFIC variant is NFIC, transcript variant 1 and NFIC, transcript variant 3.

In some embodiments, the pluripotent stem cells further comprise an expression vector comprising a nucleic acid encoding one or more transcription factors selected from the group consisting of RORC, NR0B2, ESR1, THRSP, TBX15, HLF, ATOH8, NR112, CUX2, ZNF662, TSHZ2, ATF5, NFIA, NFIB, NPAS2, FOS, ONECUT2, PROX1, NR1H4, MLXIPL, ETV1, AR, CEBPB, NR1D1, HEY2, ARID3C, KLF9, and DMRTA1.

In some embodiments, the expression vector is a viral vector. In some embodiments, the viral vector is selected from the group consisting of an adeno-associated virus (AAV) vector, an adenovirus vector, a lentivirus vector, a herpes simplex virus vector, a sendai virus vector, and a retrovirus vector.

In some embodiments, the expression vector is a non-viral vector. In some embodiments, the non-viral vector is selected from the group consisting of a plasmid DNA, a linear double-stranded DNA (dsDNA), a linear single-stranded DNA (ssDNA), a nanoplasmid, a minicircle DNA, a single-stranded oligodeoxynucleotides (ssODN), a DDNA oligonucleotide, a single-stranded mRNA (ssRNA), and a double-stranded mRNA (dsRNA). In some embodiments, the non-viral vector comprises a naked nucleic acid, a liposome, a dendrimer, a nanoparticle, a lipid-polymer system, a solid lipid nanoparticle, and/or a liposome protamine/DNA lipoplex (LPD).

In some embodiments, the expression vector is an inducible expression vector.

In some embodiments, the expression vector comprises a promoter operably linked to a nucleic acid encoding the at least one transcription factor. In some embodiments, the promoter is an endogenous promoter. In some embodiments, the promoter is an artificial promoter. In some embodiments, the promoter is an inducible promoter.

In some embodiments, the expression vector comprises a gene switch construct encoding the at least one transcription factor. In some embodiments, the gene switch construct is a transcriptional gene switch construct. In some embodiments, the gene switch construct is a post-transcriptional gene switch construct.

In some embodiments, the expression vector further comprises a self-cleaving sequence. In some embodiments, the self-cleaving sequence is selected from the group consisting of T2A, P2A, E2A and F2A.

In some embodiments, the pluripotent stem cells are embryonic stem cells or induced pluripotent stem cells.

In some embodiments, the population of pluripotent stem cells comprises at least 10⁶ pluripotent stem cells.

In another aspect, the invention provides a method of treating a disease in a subject in need thereof, the method comprising administering to the subject an effective amount of the composition or the pharmaceutical composition of the disclosure, thereby treating the disease in the subject.

In some embodiments, the disease is selected from the group consisting of fulminant hepatic failure due to any cause, viral hepatitis, drug-induced liver injury, cirrhosis, inherited hepatic insufficiency (such as Wilson's disease, Gilbert's syndrome, or al-antitrypsin deficiency), hepatobiliary carcinoma, autoimmune liver disease (such as autoimmune chronic hepatitis or primary biliary cirrhosis), urea cycle disorder, factor VII deficiency, glycogen storage disease type 1, infantile Refsum's disease, phenylketonuria, severe infantile oxalosis, cirrhosis, liver injury, acute liver failure, hepatobiliary carcinoma, hepatocellular carcinoma, genetic cholestasis (PFIC and alagille syndrome), hereditary hemochromatosis, tyrosinemia type 1, argininosuccinic aciduria (ASL), Crigler-Najjar syndrome, familial amyloid polyneuropathy, atypical haemolytic uremic syndrome-1, primary hyperoxaluria type 1, maple syrup urine disease (MSUD), acute intermittent porphyria, coagulation defects, GSD type Ia (in metabolic control), homozygous familial hypercholesterolemia, organic acidurias, and any other condition that results in impaired hepatic function.

In another aspect, the invention provides a kit comprising the composition or the pharmaceutical composition described herein.

In some embodiments, NFIX comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence as set forth in SEQ ID NO: 1.

In some embodiments, NFIC comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by any one of the nucleotide sequences of SEQ ID NO: 2 to SEQ ID NO: 6.

In some embodiments, NFIX comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence set forth in SEQ ID NO: 40.

In some embodiments, NFIC comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to any one of the amino acid sequences set forth in SEQ ID NO: 41-SEQ ID NO: 45.

In another aspect, the invention provides a kit comprising an expression vector, wherein the expression vector comprises a nucleic acid encoding at least one transcription factor selected from the group consisting of Nuclear Factor I X (NFIX) and Nuclear Factor I C (NFIC).

In some embodiments, the transcription factor is NFIX.

In some embodiments, the transcription factor is NFIC.

In some embodiments, the transcription factor is NFIX and NFIC.

In some embodiments, the NFIC is at least one alternatively spliced NFIC variant selected from the group consisting of NFIC, transcript variant 1; NFIC, transcript variant 2; NFIC, transcript variant 3; NFIC, transcript variant 4; and NFIC, transcript variant 5. In some embodiments, the alternatively spliced NFIC variant is NFIC, transcript variant 1. In some embodiments, the alternatively spliced NFIC variant is NFIC, transcript variant 3. In some embodiments, the alternatively spliced NFIC variant is NFIC, transcript variant 1 and NFIC, transcript variant 3.

In some embodiments, the kit further comprises an expression vector comprising a nucleic acid encoding one or more transcription factors selected from the group consisting of RORC, NR0B2, ESR1, THRSP, TBX15, HLF, ATOH8, NR112, CUX2, ZNF662, TSHZ2, ATF5, NFIA, NFIB, NPAS2, FOS, ONECUT2, PROX1, NR1H4, MLXIPL, ETV1, AR, CEBPB, NR1D1, HEY2, ARID3C, KLF9, and DMRTA1.

In some embodiments, NFIX comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence as set forth in SEQ ID NO: 1.

In some embodiments, NFIC comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by any one of the nucleotide sequences of SEQ ID NO: 2 to SEQ ID NO: 6.

In some embodiments, NFIX comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence set forth in SEQ ID NO: 40.

In some embodiments, NFIC comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to any one of the amino acid sequences set forth in SEQ ID NO: 41-SEQ ID NO: 45.

The present invention is further illustrated by the following detailed description and FIGURES.

### BRIEF DESCRIPTION OF THE DRAWINGS

FIG. 1 shows a schematic representation of the selection of transcription factors (TFs) of the invention.

FIG. 2A shows a schematic representation of the case of use versus physiological relevance of cancer cell lines (HepG2, HuH7 and HepaRG), stem cell derived hepatocytes (Stem Cell/iPSC-Heps) and primary human hepatocytes (PHH).

FIG. 2B shows a principal component analysis of the cells depicted in FIG. 2A. PHH-AQL, PHH-TLY and PHH-NES are adult hepatocytes. PHH-BVI are stillborn hepatocytes. Fetal correspond to human fetal primary hepatocytes. HuH7 cells cluster with hepatocytes differentiated from GMP1 iPSC that were not further treated with Br-cAMP and dexamethasone (“GMP1 control”) and that were further treated with Br-cAMP and dexamethasone for 5 days (“GMPDex”) and therefore are used for the construction of an HuH7 cell line for screening of transcription factors.

FIG. 2C shows a schematic representation of the construction of an HuH7 cell line (HuH7-Tet-On3G) used for screening of transcription factors of the invention.

FIG. 2D shows that the HuH7-Tet-On3G cell line is responsive to doxycycline induction.

FIG. 3 is a panel of bar-graphs showing the expression of mature hepatocyte markers CYP1A2 (FIG. 3A) and CYP3A4 (FIG. 3B) upon increasing expression of different transcription factors in HuH7-Tet-On3G cells. Transduction of the transcription factors was performed at a multiplicity of infection (MOI) of 10. Arrows indicate the transcription factors which upregulated the expression levels of CYP1A2 and CYP3A4. NFIC, transcript variants 1 and 3 (NFIC-1+3) refers to a mixture of alternatively spliced variants of transcription factor NFIC, NFIC, transcript variant 1 (NFIC-1) (NCBI Reference Sequence No.: NM_001245002) and NFIC, transcript variant 3 (NFIC-3) (NCBI Reference Sequence No.: NM_001245004), respectively, which were transduced at an MOI of 5, each for NFIC, transcript variant 1 (NFIC-1) and NFIC, transcript variant 3 (NFIC-3).

FIG. 4A is a schematic representation of alternatively spliced NFIC variants, NFIC, transcript variant 1 (NFIC-1); and NFIC, transcript variant 3 (NFIC-3).

FIG. 4B is a panel of bar-graphs showing the increase in the expression of mature hepatocyte markers CYP1A2 and CYP3A4 upon increasing expression of alternatively spliced NFIC variants, NFIC, transcript variant 1 (NFIC-1), NFIC, transcript variant 3 (NFIC-3), and a combination thereof (NFIC, transcript variants 1 and 3 (NFIC-1+3)) in HuH7-Tet-On3G cells. HuH7-Tet-On3G cells were transduced with lentivirus particles for NFIC, transcript variants 1 and 3 (NFIC-1+3), NFIC, transcript variant 1 (NFIC-1), and NFIC, transcript variant 3 (NFIC-3) at an MOI of 5.

FIG. 5 is a panel of bar-graphs showing that culturing of HuH7-Tet-On3G cells in a culture media comprising dexamethasone and 8-Bromoadenosine 3′, 5′-cyclic monophosphate (8-Br-cAMP) further increases the expression of mature hepatocyte markers CYP1A2 (FIG. 5A), TAT (FIG. 5B) and UGT1A1 (FIG. 5C) upon increasing expression of NFIC, transcript variant 1 (NFIC-1).

FIG. 6 is a panel of bar-graphs showing the expression of immature hepatocyte marker AFP (FIG. 6A), and mature hepatocyte markers CYP1A2 (FIG. 6B), TAT (FIG. 6C), and CYP3A4 (FIG. 6D) upon increasing expression of different transcription factors in HuH7-Tet-On3G cells. Cells were transduced with NFIC, transcript variant 1 (NFIC-1) (MOI of 10), and individual lentiviruses encoding the different transcription factors at an MOI of 10. After transduction, cells were cultured in culture media comprising 1 mM 8-Br-cAMP and 100 nM dexamethasone.

FIG. 7A shows a schematic representation of a four stage, step-wise differentiation of induced pluripotent stem cells (iPSCs) to hepatocyte-like cells. Transductions were performed with Tet-On3G at an MOI of 5, and for each transcription factor (TF) at an MOI of 3, at day 15 of differentiation towards hepatocyte-like cells. Cells were subsequently cultured for 5 days in a culture media in the absence or presence of 1 mM 8-Br-cAMP and 100 nM dexamethasone.

FIG. 7B is a panel of bar-graphs showing increase in the expression of mature hepatocyte markers CYP1A2 and TAT upon increasing expression of NFIC, transcript variant 1 (NFIC-1), NFIX and a combination thereof in iPSC derived immature hepatocytes.

FIG. 8A shows a schematic representation of a four stage, step-wise differentiation of induced pluripotent stem cells (iPSCs) to hepatocyte-like cells. Transductions were performed with Tet-On3G at an MOI of 5, and for each transcription factor (TF) at an MOI of 3, at day 15 of differentiation towards hepatocyte-like cells. Cells were subsequently cultured in a culture media in the absence or presence of 1 mM 8-Br-cAMP and 100 nM dexamethasone, and harvested at day 20 and day 24 of cell culture.

FIG. 8B is a panel of bar-graphs showing decrease in the expression of immature hepatocyte marker AFP and increase in the expression of mature hepatocyte marker CYP1A2 upon increasing expression of NFIC, transcript variant 1 (NFIC-1), NFIX and a combination thereof in iPSC derived immature hepatocytes.

FIG. 9A is graph showing a shift in the transcriptome of iPSC derived immature hepatocytes towards the transcriptome of mature hepatocytes by 30-34% upon increasing expression of NFIC, transcript variant 1 (NFIC-1), NFIX and a combination thereof in iPSC derived immature hepatocytes.

FIG. 9B is a graph showing an expanded view of Bracket 1 of the graph of FIG. 9A.

FIG. 9C is a list of the samples presented in FIGS. 9A-B.

FIG. 10 is a panel of bar-graphs showing results of functional assays for identifying CYP1A2 activity (FIG. 10A), albumin (ALB) secretion (FIG. 10B), alpha fetoprotein (AFP) secretion (FIG. 10C) and urea secretion (FIG. 10D) upon increasing expression of NFIC, transcript variant 1 (NFIC-1), NFIX and a combination thereof in iPSC derived immature hepatocytes. Transductions were performed with Tet-On3G at an MOI of 5, and for each transcription factor at an MOI of 3, at day 15 of differentiation. Cells were subsequently cultured in a culture media in the absence or presence of 1 mM 8-Br-cAMP and 100 nM dexamethasone. Functional assays were performed at day 20 (20 d) and day 24 (24 d) of cell culture.

FIG. 11A shows the transcription factors used in combination experiments.

FIG. 11B a panel of bar-graphs showing the expression of mature hepatocyte markers CYP1A2 and CYP3A4 upon increasing expression of different transcription factors in HuH7-Tet-On3G cells.

FIG. 12 shows a time course analysis of expression of mature hepatocyte markers ALB (FIG. 12A), CYP3A4 (FIG. 12B) and UGT1A1 (FIG. 12C) after forced expression of NFIC, transcript variant 1 (NFIC-1); NFIX; and a combination thereof in iPSC derived immature hepatocytes.

### DETAILED DESCRIPTION

The present invention provides efficient and effective methods of generating mature hepatocytes. The methods include increasing expression of at least one transcription factor selected from the group consisting of Nuclear Factor I X (NFIX) and Nuclear Factor I C (NFIC), in immature hepatocytes, thereby generating mature hepatocytes. Compositions generated by these methods are also provided by the present invention as are methods of using these compositions.

In one aspect, the invention provides methods for generating mature hepatocytes cells from pluripotent stem cells, such as human embryonic stem (hES) cells, embryo-derived cells, and induced pluripotent stem cells (iPS cells). The methods of the invention are efficient and effective, and result in the production of mature hepatocytes that can be used for a variety of applications disclosed herein, for example, treatment of liver diseases.

The following detailed description discloses how to make and use the present invention.

In order that the present invention may be more readily understood, certain terms are first defined. It should also be noted that whenever a value or range of values of a parameter are recited, it is intended that values and ranges intermediate to the recited values are also part of this invention.

In the following description, for purposes of explanation, specific numbers, materials, and configurations are set forth in order to provide a thorough understanding of the invention. It will be apparent, however, to one having ordinary skill in the art that the invention may be practiced without these specific details. In some instances, well-known features may be omitted or simplified so as not to obscure the present invention. Furthermore, reference in the specification to phrases such as “one embodiment” or “an embodiment” mean that a particular feature, structure, or characteristic described in connection with the embodiment is included in at least one embodiment of the invention. The appearances of phrases such as “in one embodiment” in various places in the specification are not necessarily all referring to the same embodiment.

### Definitions

Unless otherwise specified, each of the following terms have the meaning set forth in this section.

The indefinite articles “a” and “an” refer to at least one of the associated noun, and are used interchangeably with the terms “at least one” and “one or more.”

The conjunctions “or” and “and/or” are used interchangeably as non-exclusive disjunctions.

The term “hepatocyte” as used herein refers to a parenchymal liver cell. Hepatocytes make up the majority of the liver's cytoplasmic mass and are involved in protein synthesis and storage, carbohydrate metabolism, cholesterol, bile salt and phospholipid synthesis, and the detoxification, modification and excretion of exogenous and endogenous substances. Hepatocytes include immature hepatocytes that exhibit some but not all characteristics of mature hepatocytes, as well as mature and fully functional hepatocytes which have all characteristics of hepatocytes as determined by morphology, marker expression, and in vitro and in vivo functional assays.

The term “primary hepatocyte” as used herein is a hepatocyte that has been taken directly from living tissue, e.g., liver tissue. In some embodiments, the functionality of primary hepatocytes may be indicated by, for example, albumin production, urea production, and a variety of metabolic enzyme activities, and possess characteristics of mature hepatocytes. In some embodiments, the primary hepatocytes are primary human hepatocytes (“PHH”).

The term “immature hepatocyte”, as used herein, refers to a hepatocyte or hepatic progenitor cell that must undergo maturation to acquire the characteristics and/or functionality of a mature hepatocyte. In some embodiments, an immature hepatocyte is a hepatocyte-like cell that exhibits some but not all characteristics of a mature hepatocyte. In some embodiments, an immature hepatocyte does not express detectable levels of one or more of albumin (ALB), cytochrome P450 enzyme 3A4 (CYP3A4), cytochrome P450 enzyme 1A2 (CYP1A2), tyrosine aminotransferase (TAT), and UDP-glucuronosyltransferase 1A-1 (UGT1A1). In some embodiments, an immature hepatocyte expresses detectable levels of alpha fetoprotein (AFP). In some embodiments, an immature hepatocyte exhibits a decreased secretion of albumin (ALB), an increased secretion of AFP, and/or a decreased activity of CYP1A2, relative to mature hepatocytes or primary hepatocytes. In some embodiments, immature hepatocytes comprise hepatic stem cells and/or hepatic progenitor cells.

The term “hepatic progenitor,” “hepatic progenitor cell,” “hepatoblast” or “hepatoblast cell”, as used herein, refers to a cell which has the capacity to differentiate into a hepatocyte or a cholangiocyte. In some embodiments, hepatic progenitor cells are defined by expression of at least one liver-associated marker such as Hex, HNF4, alpha-fetoprotein (AFP), cytokeratin 19 (CK18), cytokeratin 19 (CK19), hepatocyte nuclear factor 6 (HNF6), and albumin (ALB). In some embodiments, hepatic progenitor cells have a decreased expression level of stem cell genes, such as Nanog, Oct4, and ckit.

The term “hepatic stem cell”, as used herein, refers to a cell that is capable in vivo or in vitro of self renewal and differentiating into hepatocytes and cholangiocytes. In an embodiment, a hepatic stem cell expresses leucine rich repeat containing G-protein-coupled receptor 5 (LGR5) and/or epithelial cell adhesion molecule (EpCAM).

A “mature hepatocyte”, as used herein, refers to a hepatocyte that (i) comprises a gene expression profile that is more similar to a primary hepatocyte or a known mature hepatocyte than a gene expression profile of an immature hepatocyte, and/or (ii) exhibits one or more characteristics of a mature hepatocyte. Non-limiting examples of cell markers useful in distinguishing mature hepatocytes include albumin, asialoglycoprotein receptor, α1-antitrypsin, α-fetoprotein, apoE, arginase I, apoAI, apoAII, apoB, apoCIII, apoCII, aldolase B, alcohol dehydrogenase 1, catalase, CYP3A4, glucokinase, glucose-6-phosphatase, insulin growth factors 1 and 2, IGF-1 receptor, insulin receptor, leptin, liver-specific organic anion transporter (LST-1), L-type fatty acid binding protein, phenylalanine hydroxylase, transferrin, retinol binding protein, erythropoietin (EPO, albumin, al-antitrypsin, asialoglycoprotein receptor, cytokeratin 8 (CK8), cytokeratin 18 (CK18), CYP3A4, fumaryl acetoacetate hydrolase (FAH), glucose-6-phosphates, tyrosine aminotransferase, phosphoenolpyruvate carboxykinase, and tryptophan 2,3-dioxygenase.

In some embodiments, mature hepatocytes exhibit an increased expression of albumin (ALB), cytochrome P450 enzyme 1A2 (CYP1A2), cytochrome P450 enzyme 3A4 (CYP3A4), tyrosine aminotransferase (TAT), and/or UDP-glucuronosyltransferase 1A-1 (UGT1A1) relative to immature hepatocytes. In some embodiments, the mature hepatocytes exhibit a decreased expression of alpha fetoprotein (AFP) relative to immature hepatocytes.

In some embodiments, the mature hepatocytes exhibit an increased secretion of albumin (ALB), a decreased secretion of AFP, and/or an increased activity of CYP1A2, relative to immature hepatocytes.

In some embodiments, the mature hepatocytes comprise increased expression of at least 1, 2, 3, 4, 5, 6, 7, 8, 9, 10 or more genes or proteins selected from the group consisting of ALB, CPS1, G6P, TDO, CYP2C9, CYP2D6, CYP7A1, CYP3A7, CYP1A2, CYP3A4, CYP2B6, NAT2, TAT, ASGPR-1 and UGT1A1 compared to a cell population comprising immature hepatocytes.

In yet another embodiment, mature hepatocytes display a global gene expression profile that is indicative of hepatocyte maturation. Global gene expression profiles may be compared to those of primary hepatocytes or known mature hepatocytes and may be obtained by any method known in the art, for example transcriptomic analysis or microarray analysis.

In an embodiment, one or more characteristics of a mature hepatocyte includes, but is not limited to, epithelial morphology, polarization, polyploidization, gene expression, CYP activities, transferase activities, transporter activities, bile acid synthesis, glycogen storage, serum protein synthesis, cholesterol metabolism, lipid uptake, urea metabolism, coagulation factors, engraftment and repopulation, restoration of liver function, and tumorigenicity. See, e.g., Chen et al, Gastroenterology 2018; 154:1258-1272, which is incorporated in its entirety herein by reference.

The term “increasing expression”, as used herein, refers to increasing the level and/or activity of a nucleic acid, e.g., an RNA or DNA, encoding a transcription factor disclosed herein and/or increasing the level and/or activity of a transcription factor disclosed herein, relative to the endogenous nuclei acid levels and/or protein levels of the transcription factor. In some embodiments, increasing expression of the at least one transcription factor comprises contacting a cell (for example, an immature hepatocyte, a hepatic progenitor cell, or a pluripotent stem cell, e.g., an embryonic stem cell or an induced pluripotent stem cell), with the at least one transcription factor. In some embodiments, increasing expression of the at least one transcription factor comprises transduction of a cell (for example, an immature hepatocyte, a hepatic progenitor cell, or a pluripotent stem cell, e.g., an embryonic stem cell or an induced pluripotent stem cell) with a viral vector encoding the at least one transcription factor. In some embodiments, increasing expression of the at least one transcription factor comprises transfection of a cell (for example, an immature hepatocyte, a hepatic progenitor cell, or a pluripotent stem cell, e.g., an embryonic stem cell or an induced pluripotent stem cell) with an expression vector encoding the at least one transcription factor.

In some embodiments, increasing expression of the at least one transcription factor comprises an increase of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to endogenous expression levels of the at least one transcription factor in a cell (for example, an immature hepatocyte, a hepatic progenitor cell, or a pluripotent stem cell, e.g., an embryonic stem cell or an induced pluripotent stem cell). In some embodiments, increasing expression of the at least one transcription factor comprises an increase of at least 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or 1000% relative to endogenous expression levels of the at least one transcription factor in a cell (for example, an immature hepatocyte, a hepatic progenitor cell, or a pluripotent stem cell, e.g., an embryonic stem cell or an induced pluripotent stem cell).

The term “endogenous” as used herein refers to the native form of a nucleic acid, polynucleotide, oligonucleotide, DNA, RNA, gene, peptide or polypeptide in its natural location in a cell or in the genome of a cell.

The term “maturation”, as used herein, refers to a process that is required for a cell, e.g., an immature hepatocyte, to become more specialized and/or functional, for example, similar to its functional and/or phenotypic state in vivo or similar to a functional and/or phenotypic state of a known mature hepatocyte or primary hepatocyte. In one embodiment, the process by which immature hepatocytes become mature hepatocytes is referred to as maturation.

As used herein, the term “pluripotent stem cells”, “PS cells”, or “PSCs” includes embryonic stem cells, induced pluripotent stem cells, and embryo-derived pluripotent stem cells, regardless of the method by which the pluripotent stem cells are derived. Pluripotent stem cells are defined functionally as stem cells that: (a) are capable of inducing teratomas when transplanted in immunodeficient (SCID) mice; (b) are capable of differentiating to cell types of all three germ layers (e.g., can differentiate to ectodermal, mesodermal, and endodermal cell types); (c) express one or more markers of embryonic stem cells (e.g., express OCT4, alkaline phosphatase, SSEA-3 surface antigen, SSEA-4 surface antigen, NANOG, TRA-1-60, TRA-1-81, SOX2, REX1, etc.); and d) are capable of self-renewal. The term “pluripotent” refers to the ability of a cell to form all lineages of the body or soma (i.e., the embryo proper). For example, embryonic stem cells and induced pluripotent stem cells are a type of pluripotent stem cells that are able to form cells from each of the three germs layers: the ectoderm, the mesoderm, and the endoderm. Pluripotency is a continuum of developmental potencies ranging from the incompletely or partially pluripotent cell which is unable to give rise to a complete organism to the more primitive, more pluripotent cell, which is able to give rise to a complete organism (e.g., an embryonic stem cell). Exemplary pluripotent stem cells can be generated using, for example, methods known in the art. Exemplary pluripotent stem cells include, but are not limited to, embryonic stem cells derived from the inner cell mass of blastocyst stage embryos, embryonic stem cells derived from one or more blastomeres of a cleavage stage or morula stage embryo (optionally without destroying the remainder of the embryo), induced pluripotent stem cells produced by reprogramming of somatic cells into a pluripotent state, and pluripotent cells produced from embryonic germ (EG) cells (e.g., by culturing in the presence of FGF-2, LIF and SCF). Such embryonic stem cells can be generated from embryonic material produced by fertilization or by asexual means, including somatic cell nuclear transfer (SCNT), parthenogenesis, and androgenesis.

In an embodiment, pluripotent stem cells may be genetically engineered or otherwise modified, for example, to increase longevity, potency, homing, to prevent or reduce immune responses, or to deliver a desired factor in cells that are obtained from such pluripotent cells (for example, hepatocytes). For example, the pluripotent stem cell and therefore, the resulting differentiated cell, can be engineered or otherwise modified to lack or have reduced expression of beta 2 microglobulin, HLA-A, HLA-B, HLA-C, TAP1, TAP2, Tapasin, CTIIA, RFX5, TRAC, and/or TRAB genes. As described in WO2012145384 and WO2013158292, which are herein incorporated by reference in their entireties, in some embodiments, the cell, such as a pluripotent stem cell and the resulting differentiated cell such as a hepatocyte, comprises a genetically engineered disruption in a beta-2 microglobulin (B2M) gene. In some embodiments, the cell further comprises a polynucleotide capable of encoding a single chain fusion human leukocyte antigen (HLA) class I protein comprising at least a portion of the B2M protein covalently linked, either directly or via a linker sequence, to at least a portion of an HLA-1α chain. In some embodiments, the HLA-1α chain is selected from HLA-A, HLA-B, HLA-C, HLA-E, HLA-F, and HLA-G. In some embodiments, the cell comprises a genetically engineered disruption in a human leukocyte antigen (HLA) class II-related gene. In some embodiments, the HLA class II-related gene is selected from regulatory factor X-associated ankyrin-containing protein (RFXANK), regulatory factor 5 (RFX5), regulatory factor X associated protein (RFXAP), class II transactivator (CIITA), HLA-DPA (α chain), HLA-DPB (β chain), HLA-DQA, HLA-DQB, HLA-DRA, HLA-DRB, HLA-DMA, HLA-DMB, HLA-DOA, and HLA-DOB. In some embodiments, the cell comprises one or more polynucleotides encoding a single chain fusion HLA class II protein or an HLA class II protein.

The pluripotent stem cell and the resulting differentiated cell may be engineered or otherwise modified to increase expression of a gene. In an embodiment, the pluripotent stem cell may be engineered to express or increase expression of one or more of the transcription factors of the invention. There are a variety of techniques for engineering cells to modulate the expression of one or more genes (or proteins), including the use of viral vectors such as AAV vectors, zinc-finger nucleases (ZFNs), transcription activator-like effector nucleases (TALENs), and CRISPR/Cas-based methods for genome engineering, as well as the use of transcriptional and translational inhibitors such as antisense and RNA interference (which can be achieved using stably integrated vectors and episomal vectors).

The term “embryo” or “embryonic” is meant a developing cell mass that has not been implanted into the uterine membrane of a maternal host. An “embryonic cell” is a cell isolated from or contained in an embryo. This also includes blastomeres, obtained as early as the two-cell stage, or aggregated blastomeres after extraction.

The term “embryo-derived cells” (EDC), as used herein, refers broadly to morula-derived cells, blastocyst-derived cells including those of the inner cell mass, embryonic shield, or epiblast, or other pluripotent stem cells of the early embryo, including primitive endoderm, ectoderm, and mesoderm and their derivatives. “EDC” also including blastomeres and cell masses from aggregated single blastomeres or embryos from varying stages of development, but excludes human embryonic stem cells that have been passaged as cell lines.

The term “embryonic stem cells”, “ES cells,” or “ESCs” as used herein, refer broadly to cells isolated from the inner cell mass of blastocysts or morulae and that have been serially passaged as cell lines. The term also includes cells isolated from one or more blastomeres of an embryo, preferably without destroying the remainder of the embryo (see, e.g., Chung et al., Cell Stem Cell. 2008 Feb. 7; 2(2): 1 13-7; U.S. Pub No. 20060206953; U.S. Pub No. 2008/0057041, each of which is hereby incorporated by reference in its entirety). The ES cells may be derived from fertilization of an egg cell with sperm or DNA, nuclear transfer, parthenogenesis, or by any means to generate ES cells with homozygosity in the HLA region. ES cells may also refer to cells derived from a zygote, blastomeres, or blastocyst-staged mammalian embryo produced by the fusion of a sperm and egg cell, nuclear transfer, parthenogenesis, or the reprogramming of chromatin and subsequent incorporation of the reprogrammed chromatin into a plasma membrane to produce a cell. In an embodiment, the embryonic stem cell may be a human embryonic stem cell (or “hES cells”). In an embodiment, human embryonic stem cells are not derived from embryos over 14 days from fertilization. In another embodiment, human embryonic stem cells are not derived from embryos that have been developed in vivo. In another embodiment, human embryonic stem cells are derived from preimplantation embryos produced by in vitro fertilization.

“Induced pluripotent stem cells” or “iPS cells,” as used herein, generally refer to pluripotent stem cells obtained by reprogramming a somatic cell into a less differentiated state. An iPS cell may be generated by expressing or inducing expression of a combination of factors (“reprogramming factors”), for example, OCT4 (sometimes referred to as OCT 3/4), SOX2, MYC (e.g., c-MYC or any MYC variant), NANOG, LIN28, and KLF4, in a somatic cell. In an embodiment, the reprogramming factors comprise OCT4, SOX2, c-MYC, and KLF4. In another embodiment, reprogramming factors comprise OCT4, SOX2, NANOG, and LIN28. In certain embodiments, at least two reprogramming factors are expressed in a somatic cell to successfully reprogram the somatic cell. In other embodiments, at least three reprogramming factors are expressed in a somatic cell to successfully reprogram the somatic cell. In other embodiments, at least four reprogramming factors are expressed in a somatic cell to successfully reprogram the somatic cell. In another embodiment, at least five reprogramming factors are expressed in a somatic cell to successfully reprogram the somatic cell. In yet another embodiment, at least six reprogramming factors are expressed in the somatic cell, for example, OCT4, SOX2, c-MYC, NANOG, LIN28, and KLF4. In other embodiments, additional reprogramming factors are identified and used alone or in combination with one or more known reprogramming factors to reprogram a somatic cell to a pluripotent stem cell.

iPS cells may be generated using fetal, postnatal, newborn, juvenile, or adult somatic cells. Somatic cells may include, but are not limited to, fibroblasts, keratinocytes, adipocytes, muscle cells, organ and tissue cells, and various blood cells including, but not limited to, hematopoietic cells (e.g., hematopoietic stem cells). In an embodiment, the somatic cells are fibroblast cells, such as a dermal fibroblast, synovial fibroblast, or lung fibroblast, or a non-fibroblastic somatic cell.

iPS cells may be obtained from a cell bank. Alternatively, iPS cells may be newly generated by methods known in the art. iPS cells may be specifically generated using material from a particular patient or matched donor with the goal of generating tissue-matched cells. In an embodiment, iPS cells may be universal donor cells that are not substantially immunogenic.

The induced pluripotent stem cell may be produced by expressing or inducing the expression of one or more reprogramming factors in a somatic cell. Reprogramming factors may be expressed in the somatic cell by infection using a viral vector, such as a retroviral vector or other gene editing technologies, such as CRISPR, Talen, zinc-finger nucleases (ZFNs). Also, reprogramming factors may be expressed in the somatic cell using a non-integrative vector, such as an episomal plasmid, or RNA, such as synthetic mRNA or via an RNA virus such as Sendai virus. When reprogramming factors are expressed using non-integrative vectors, the factors may be expressed in the cells using electroporation, transfection, or transformation of the somatic cells with the vectors. For example, in mouse cells, expression of four factors (OCT3/4, SOX2, c-MYC, and KLF4) using integrative viral vectors is sufficient to reprogram a somatic cell. In human cells, expression of four factors (OCT3/4, SOX2, NANOG, and LIN28) using integrative viral vectors is sufficient to reprogram a somatic cell.

Expression of the reprogramming factors may be induced by contacting the somatic cells with at least one agent, such as a small organic molecule agents, that induce expression of reprogramming factors.

The somatic cell may also be reprogrammed using a combinatorial approach wherein the reprogramming factor is expressed (e.g., using a viral vector, plasmid, and the like) and the expression of the reprogramming factor is induced (e.g., using a small organic molecule).

Once the reprogramming factors are expressed or induced in the cells, the cells may be cultured. Over time, cells with ES characteristics appear in the culture dish. The cells may be chosen and subcultured based on, for example, ES cell morphology, or based on expression of a selectable or detectable marker. The cells may be cultured to produce a culture of cells that resemble ES cells.

To confirm the pluripotency of the iPS cells, the cells may be tested in one or more assays of pluripotency. For examples, the cells may be tested for expression of ES cell markers; the cells may be evaluated for ability to produce teratomas when transplanted into SCID mice; the cells may be evaluated for ability to differentiate to produce cell types of all three germ layers.

iPS cells may be from any species. These iPS cells have been successfully generated using mouse and human cells. Furthermore, iPS cells have been successfully generated using embryonic, fetal, newborn, and adult tissue. Accordingly, one may readily generate iPS cells using a donor cell from any species. Thus, one may generate iPS cells from any species, including but not limited to, human, non-human primates, rodents (mice, rats), ungulates (cows, sheep, etc.), dogs (domestic and wild dogs), cats (domestic and wild cats such as lions, tigers, cheetahs), rabbits, hamsters, goats, elephants, panda (including giant panda), pigs, raccoon, horse, zebra, marine mammals (dolphin, whales, etc.) and the like.

The term “contacting” (e.g., contacting a cell, such as an immature hepatocyte, a hepatic progenitor cell, or a pluripotent stem cell, e.g., an embryonic stem cell or an induced pluripotent stem cell) with a transcription factor(s) according to the invention) is intended to include any way of introducing into a cell a transcription factor(s) and/or incubating the transcription factor(s) and the cell together in vitro (e.g., adding the transcription factor(s) to cells in culture). In some embodiments, the term “contacting” is not intended to include the in vivo exposure of the cell to the transcription factor(s) as disclosed herein that may occur naturally in a subject. The step of contacting a cell with a transcription factor(s) as disclosed herein can be conducted in any suitable manner. The cells may be treated in adherent culture, or in suspension culture, and the transcription factors(s) can be added substantially simultaneously (e.g., together in a cocktail) or sequentially (e.g., within 1 hour, 1 day or more from an addition of a first transcription factor). It is understood that the cells contacted with a transcription factor(s) as disclosed herein can also be simultaneously or subsequently contacted with another agent, such as a growth factor or other differentiation agent or environment to stabilize the cells, or to differentiate the cells further. In an embodiment, contacting the cell with a transcription factor includes transduction of the cell with a vector comprising a nucleic acid encoding the transcription factor(s) or transfection of the cell with an expression vector comprising a nucleic acid encoding the transcription factor(s), and may include culturing the cell under conditions known in the art, for example, for culturing the pluripotent and/or differentiated cells, for example, as further described in the Examples.

As used herein, the term “differentiation” is the process by which an unspecialized (“uncommitted”) or less specialized cell acquires the features of a specialized cell such as, for example, a hepatocyte. A differentiated cell is one that has taken on a more specialized position within the lineage of a cell. For example, an hES cell can be differentiated into various more differentiated cell types, including an hepatocyte. In certain embodiments, differentiation of a cell is performed in vitro, and excludes in vivo differentiation.

As used herein, the term “cultured” or “culturing” refers to the placing of cells in a medium containing, among other things nutrients needed to sustain the life of the cultured cells, any specified added substances. Cells are cultured “in the presence of” a specified substance when the medium in which such cells are maintained contains such specified substance. Culturing can take place in any vessel or apparatus in which the cells can be maintained exposed to the medium, including without limitation petri dishes, culture dishes, blood collection bags, roller bottles, flasks, test tubes, microtiter wells, hollow fiber cartridges or any other apparatus known in the art.

As used herein, the term “subculturing” or “passaging,” refers to transferring some or all cells from a previous culture to fresh growth medium and/or plating onto a new culture dish and further culturing the cells. Subculturing may be done, e.g., to prolong the life, enrich for a desired cell population, and/or expand the number of cells in the culture. For example, the term includes transferring, culturing, or plating some or all cells to a new culture vessel at a lower cell density to allow proliferation of the cells.

As used herein, “administration”, “administering” and variants thereof refers to introducing a composition or agent into a subject and includes concurrent and sequential introduction of a composition or agent. “Administration” can refer, e.g., to therapeutic, pharmacokinetic, diagnostic, research, placebo, and experimental methods. “Administration” also encompasses in vitro and ex vivo treatments. Administration includes self-administration and the administration by another. Administration can be carried out by any suitable route. A suitable route of administration allows the composition or the agent to perform its intended function. For example, if a suitable route is intravenous, the composition is administered by introducing the composition or agent into a vein of the subject.

As used herein, the terms “subject”, “individual”, “host”, and “patient” are used interchangeably herein and refer to any mammalian subject for whom diagnosis, treatment, or therapy is desired, particularly humans. The methods described herein are applicable to both human therapy and veterinary applications. In some embodiments, the subject is a mammal, and in particular embodiments the subject is a human.

As used herein, the terms “therapeutic amount”, “therapeutically effective amount”, an “amount effective”, or “pharmaceutically effective amount” of an active agent (e.g., an hepatocyte) are used interchangeably to refer to an amount that is sufficient to provide the intended benefit of treatment. However, dosage levels are based on a variety of factors, including the type of injury, the age, weight, sex, medical condition of the patient, the severity of the condition, the route of administration, anticipated cell engraftment, long term survival, and/or the particular active agent employed. Thus the dosage regimen may vary widely, but can be determined routinely by a physician using standard methods. Additionally, the terms “therapeutic amount”, “therapeutically effective amounts” and “pharmaceutically effective amounts” include prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For the therapeutic agents described herein (e.g., hepatocytes), a therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan.

Pharmacokinetic principles provide a basis for modifying a dosage regimen to obtain a desired degree of therapeutic efficacy with a minimum of unacceptable adverse effects. In situations where the agent's plasma concentration can be measured and related to therapeutic window, additional guidance for dosage modification can be obtained.

As used herein, the terms “treat”, “treating”, and/or “treatment” include abrogating, substantially inhibiting, slowing or reversing the progression of a condition, substantially ameliorating clinical symptoms of a condition, or substantially preventing the appearance of clinical symptoms of a condition (e.g., a pathological condition), obtaining beneficial or desired clinical results. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (c) limiting recurrence of symptoms in patients that were previously asymptomatic for the disorder(s).

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

### I. Methods of the Invention

The present invention is based on the discovery of methods which include increasing expression of at least one transcription factor selected from the group consisting of NFIC and NFIX, to promote the maturation of hepatocytes, and thereby allow the production of mature and functional hepatocytes. The methods of the invention are efficient and effective, and result in production of mature hepatocytes, for example, from pluripotent stem cells, that can be used for a variety of applications disclosed herein, for example, treatment of liver diseases.

In some embodiments, increasing the expression of NFIX comprises an increase of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to endogenous expression levels of NFIX in the immature hepatocytes.

In some embodiments, increasing the expression of NFIC comprises an increase of at least 0.1-fold, 0.2-fold, 0.5-fold, 1-fold, 2-fold, 5-fold, 10-fold, 20-fold, 50-fold, 100-fold, 200-fold, 500-fold, 1000-fold, or 10,000-fold relative to endogenous expression levels of NFIC in the immature hepatocytes.

In some embodiments, the methods further comprise culturing the immature hepatocytes in a culture media comprising dexamethasone, 8-Bromoadenosine 3′, 5′-cyclic monophosphate (8-Br-cAMP), or a combination thereof.

In some embodiments, the immature hepatocytes comprise an expression vector comprising a nucleic acid encoding the at least one transcription factor.

In some embodiments, increasing the expression of the at least one transcription factor in the immature hepatocytes comprises inducing expression of the at least one transcription factor in the immature hepatocytes.

In some embodiments, the immature hepatocytes are derived from pluripotent stem cells, e.g., embryonic stem cells or induced pluripotent stem cells. Any method for differentiating pluripotent cells into immature hepatocytes may be used. For example, immature hepatocytes may be obtained by differentiating pluripotent stem cells as described herein.

In some embodiments, the pluripotent stem cells may be engineered to comprise an expression vector comprising a nucleic acid encoding the at least one transcription factor. In some embodiments, the expression vector comprises a promoter, e.g., an endogenous promoter, an artificial promoter or an inducible promoter, operably linked to a nucleic acid encoding the at least one transcription factor.

### Cells for Generating Hepatocytes

In certain embodiments of the invention, there are disclosed methods and compositions for producing mature hepatocytes by increasing expression of at least one transcription factor selected from the group consisting of Nuclear Factor I X (NFIX) and Nuclear Factor I C (NFIC) in immature hepatocytes. In some embodiments, the mature and immature hepatocytes are derived from pluripotent stem cells, for example, embryonic stem cells, induced pluripotent stem cells, fetal stem cells, and/or adult stem cells. In further embodiments, the mature and immature hepatocytes may be derived from somatic cells.

### A. Stem Cells

In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues.

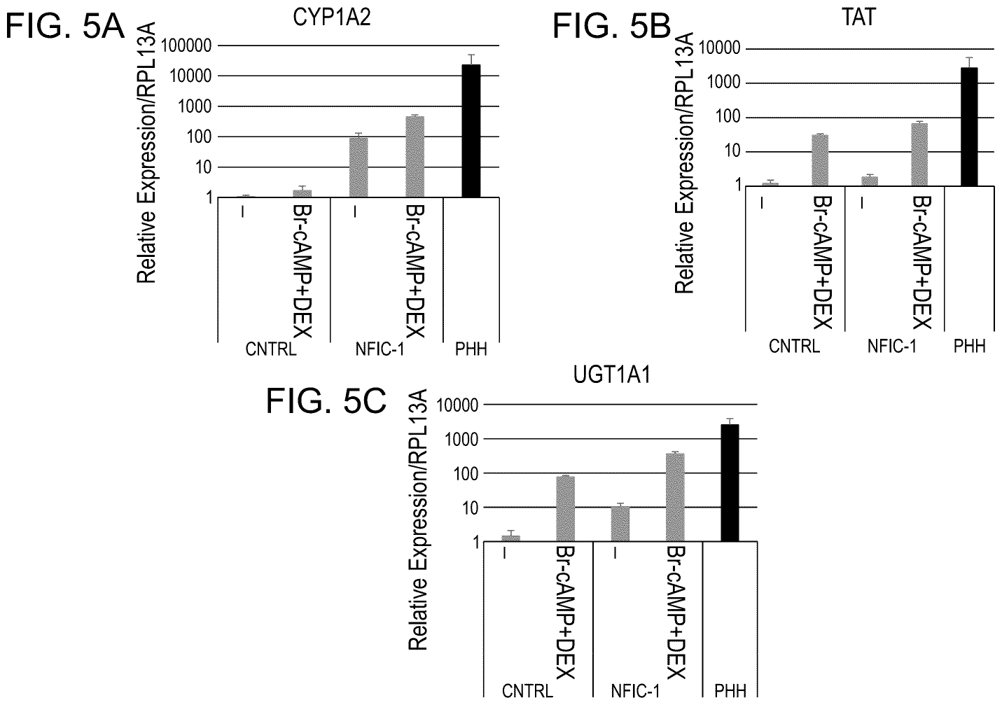

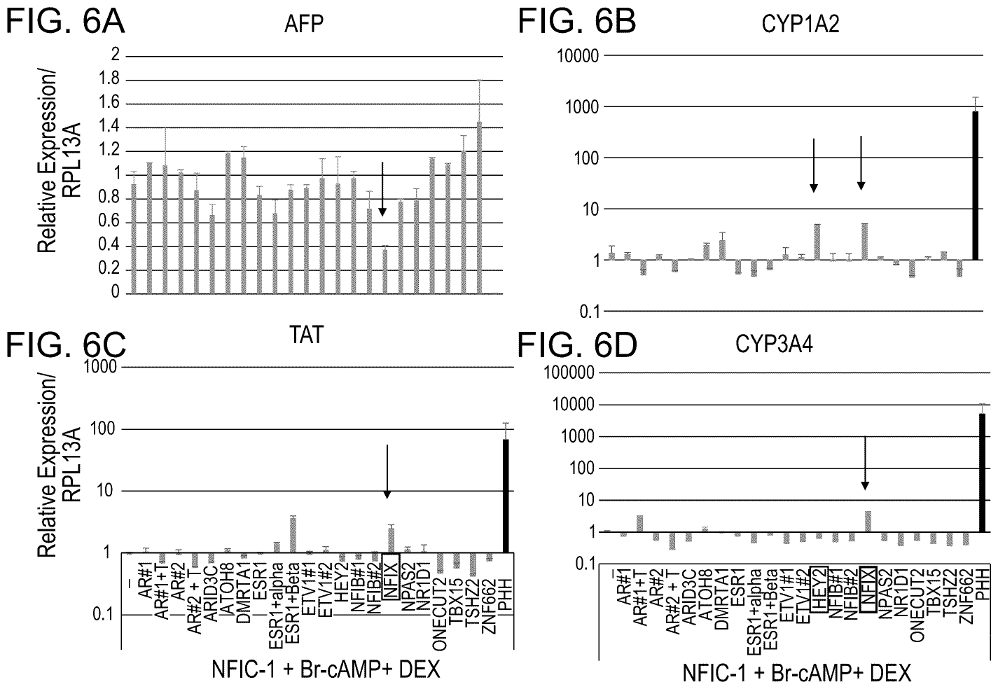

Pluripotent stem cells, such as human embryonic stem cells (ESCs) and induced pluripotent stem cells (iPSC) are capable of long-term proliferation in vitro, while retaining the potential to differentiate into all cell types of the body, including immature hepatocytes. Thus these cells could potentially provide an unlimited supply of patient-specific functional hepatocytes for both drug development and transplantation therapies. The differentiation of pluripotent stem cells to hepatocytes in vitro may involve the addition of different growth factors at different stages of differentiation, and may require about 15-20 days of differentiation (see e.g. FIGS. 5A and 6A). One of the challenges with differentiating pluripotent stem cells into hepatocytes in vitro is the hepatocytes appear more functionally like fetal hepatocytes, e.g., immature hepatocytes, and do not yet exhibit the full functional spectrum of mature hepatocytes, e.g., primary human hepatocytes (PHH). Pluripotent stem cells, such as human ESC/iPSCs, with their unlimited proliferation ability, provide an advantage over somatic cells as the starting cell population for hepatocyte differentiation.

Pluripotent stem cells, e.g., embryonic stem (ES) cells or iPS cells, may be the starting material of the disclosed method. In any of the embodiments herein, the pluripotent stem cell may be human pluripotent stem cells (hPSCs). Pluripotent stem cells (PSCs) may be cultured in any way known in the art, such as in the presence or absence of feeder cells. Additionally, PSCs produced using any method can be used as the starting material to produce hepatocytes. For example, the hES cells may be derived from blastocyst stage embryos that were the product of in vitro fertilization of egg and sperm. Alternatively, the hES cells may be derived from one or more blastomeres removed from an early cleavage stage embryo, optionally, without destroying the remainder of the embryo. In still other embodiments, the hES cells may be produced using nuclear transfer. In a further embodiment, iPSCs may be used. As a starting material, previously cryopreserved PSCs may be used. In another embodiment, PSCs that have never been cryopreserved may be used.

In one aspect of the present invention, PSCs are plated onto an extracellular matrix under feeder or feeder-free conditions. In an embodiment, the PSCs can be cultured on an extracellular matrix, including, but not limited to, laminin, fibronectin, vitronectin, Matrigel, CellStart, collagen, or gelatin. In some embodiments, the extracellular matrix is laminin with or without e-cadherin. In some embodiments, laminin may be selected from the group comprising laminin 521, laminin 511, or iMatrix511. In some embodiments, the feeder cells are human feeder cells, such as human dermal fibroblasts (HDF). In other embodiments, the feeder cells are mouse embryo fibroblasts (MEF).

In certain embodiments, the media used when culturing the PSCs may be selected from any media appropriate for culturing PSCs. In some embodiments, any media that is capable of supporting PSC cultures may be used. For example, one of skill in the art may select amongst commercially available or proprietary media.

The medium that supports pluripotency may be any such medium known in the art. In some embodiments, the medium that supports pluripotency is Nutristem™. In some embodiments, the medium that supports pluripotency is TeSR™. In some embodiments, the medium that supports pluripotency is StemFit™. In other embodiments, the medium that supports pluripotency is Knockout™ DMEM (Gibco), which may be supplemented with Knockout™ Serum Replacement (Gibco), LIF, bFGF, or any other factors. Each of these exemplary media is known in the art and commercially available. In further embodiments, the medium that supports pluripotency may be supplemented with bFGF or any other factors. In an embodiment, bFGF may be supplemented at a low concentration (e.g., 4 ng/ml). In another embodiment, bFGF may be supplemented at a higher concentration (e.g., 100 ng/mL), which may prime the PSCs for differentiation.

The concentration of PSCs to be used in the production method of the present invention is not particularly limited. For example, when a 10 cm dish is used, 1+10⁴-1×10⁸ cells per dish, preferably 5+10⁴-5×10⁶ cells per dish, more preferably 1×10⁵-1×10⁷ cells, per dish are used.

In some embodiments, the PSCs are plated with a cell density of about 1,000-100,000 cells/cm². In some embodiments, the PSCs are plated with a cell density of about 5000-100,000 cells/cm², about 5000-50,000 cells/cm², or about 5000-15,000 cells/cm². In other embodiments, the PSCs are plated at a density of about 10,000 cells/cm².

In some embodiments, the medium that supports pluripotency, e.g., StemFit™ or other similar medium, is replaced with a differentiation medium to differentiate the cells into immature hepatocytes. In some embodiments, replacement of the media from the medium that supports pluripotency to a differentiation medium may be performed at different time points during the cell culture of PSCs and may also depend on the initial plating density of the PSCs. In some embodiments, replacement of the media can be performed after 3-14 days of culture of the PSCs in the pluripotency medium. In some embodiments, replacement of the media may be performed at day 3, 4, 5, 6, 7, 8, 9, 10, 11, 12, 13, or 14.

In some embodiments, the stem cells useful for the method described herein include but not limited to embryonic stem cells, induced pluripotent stem cells, mesenchymal stem cells, bone-marrow derived stem cells, hematopoietic stem cells, chondrocyte progenitor cells, epidermal stem cells, gastrointestinal stem cells, neural stem cells, hepatic stem cells, adipose-derived mesenchymal stem cells, pancreatic progenitor cells, hair follicular stem cells, endothelial progenitor cells and smooth muscle progenitor cells.

In some embodiments, the stem cells used for the method described herein is isolated from umbilical cord, placenta, amniotic fluid, chorion villi, blastocysts, bone marrow, adipose tissue, brain, peripheral blood, the gastrointestinal tract, cord blood, blood vessels, skeletal muscle, skin, liver and menstrual blood.

The detailed procedures for the isolation of human stem cells from various sources are described in Current Protocols in Stem Cell Biology (2007), which is incorporated by reference in its entirety herein. Methods of isolating and culturing stem cells from various sources are also described in U.S. Pat. Nos. 5,486,359, 6,991,897, 7,015,037, 7,422,736, 7,410,798, 7,410,773, 7,399,632; each of which is incorporated by reference in its entirety herein.

### B. Somatic Cells

In certain aspects of the invention, there may also be provided methods of transdifferentiation, i.e., the direct conversion of one somatic cell type into another, e.g., deriving hepatocytes from other somatic cells. Transdifferentiation may involve the use of hepatocyte differentiation transcription factor genes or gene products to increase expression levels of such genes in somatic cells for production of hepatocytes.

However, human somatic cells may be limited in supply, especially those from living donors. In order to provide an unlimited supply of starting cells for hepatocyte differentiation, somatic cells may be immortalized by introduction of immortalizing genes or proteins, such as hTERT and/or other oncogenes. The immortalization of cells may be reversible (e.g., using removable expression cassettes) or inducible (e.g., using inducible promoters).

Somatic cells in certain aspects of the invention may be primary cells (non-immortalized cells), such as those freshly isolated from an animal, or may be derived from a cell line (immortalized cells). The cells may be maintained in cell culture following their isolation from a subject. In certain embodiments the cells are passaged once or more than once (e.g., between 2-5,5-10, 10-20, 20-50, 50-100 times, or more) prior to their use in a method of the invention. In some embodiments the cells will have been passaged no more than 1, 2, 5, 10, 20, or 50 times prior to their use in a method of the invention.

The somatic cells used or described herein may be native somatic cells, or engineered somatic cells, i.e., somatic cells which have been genetically altered. Somatic cells of the present invention are typically mammalian cells, such as, for example, human cells, primate cells or mouse cells. They may be obtained by well-known methods and can be obtained from any organ or tissue containing live somatic cells, e.g., blood, bone marrow, skin, lung, pancreas, liver, stomach, intestine, heart, reproductive organs, bladder, kidney, urethra and other urinary organs, etc.

Mammalian somatic cells useful in the present invention include, but are not limited to, Sertoli cells, endothelial cells, granulosa epithelial cells, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, cardiac muscle cells, and other muscle cells, etc.

Methods described herein may be used to program one or more somatic cells, e.g., colonies or populations of somatic cells into hepatocytes. In some embodiments a population of cells of the present invention is substantially uniform in that at least 90% of the cells display a phenotype or characteristic of interest. In some embodiments at least 95%, 96%, 97%, 98%, 99%, 99.5%, 99.8%, 99.9, 99.95% or more of the cells display a phenotype or characteristic of interest. In certain embodiments of the invention the somatic cells have the capacity to divide, i.e., the somatic cells are not post-mitotic.

Somatic cells may be partially or completely differentiated. As described herein, both partially differentiated somatic cells and fully differentiated somatic cells can be differentiated to produce hepatocytes.

### Transcription Factors for Use in the Methods of the Invention

Mature hepatocytes can be generated by increasing the expression in immature hepatocytes of at least one transcription factor described herein. Any transcription factor important for promoting hepatocyte differentiation, maturation or function may be used, for example, at least one transcription factor selected from the transcription factors described in Table 1. All the isoforms and variants of the transcription factors listed in Table 1 may be included in this invention. Non-limiting examples of accession numbers for certain isoforms or variants of the transcription factors of the invention are described in Table 1.

TABLE 1
Transcription Factors for Generating Mature Hepatocytes

| | | SEQ |
|---|---|---|
| Transcription Factor | Accession No. | ID NO. |
| NFIX | NM_002501.4 | 1 |
| NFIC, transcript variant 1 (NFIC-1) | NM_001245002 | 2 |
| NFIC, transcript variant 2 (NFIC-2) | NM 205843 | 3 |
| NFIC, transcript variant 3 (NFIC-3) | NM_001245004 | 4 |
| NFIC, transcript variant 4 (NFIC-4) | NM_001245005 | 5 |
| NFIC, transcript variant 5 (NFIC-5) | NM 005597 | 6 |
| RORC | NM_005060.3 | 7 |
| NR0B2 | NM_021969.2 | 8 |
| ESR1 | NM_001291230.1 | 9 |
| THRSP | NM_003251.3 | 10 |
| TBX15 | NM_152380 | 11 |
| HLF | NM_002126.4 | 12 |
| ATOH8 | NM_032827.7 | 13 |
| NR1I2 | NM_003889.3 | 14 |
| CUX2 | NM_015267.3 | 15 |
| ZNF662 | NM_001134656.1 | 16 |
| TSHZ2 | NM_173485.5 | 17 |
| ATF5 | NM_001193646.1 | 18 |
| NFIA | NM_001134673.3 | 19 |
| NFIB | NM_005596.3 | 20 |
| NPAS2 | XM_005263953.2 | 21 |
| FOS | NM_005252.3 | 22 |
| ONECUT2 | NM_004852.2 | 23 |
| PROX1, transcript variant 1 | NM_001270616.2 | 24 |
| PROX1, transcript variant 2 | NM_002763.5 | 39 |
| NR1H4 | NM_001206979.1 | 25 |
| MLXIPL | NM_032951.2 | 26 |
| ETV1 | NM_001163147 | 27 |
| AR | NM_000044.3 | 28 |
| CEBPB | NM_005194.3 | 29 |
| NR1D1 | NM_021724.4 | 30 |
| HEY2 | NM_012259.2 | 31 |
| ARID3C | NM_001017363.1 | 32 |
| KLF9 | NM_001206.2 | 33 |
| DMRTA1 | NM_022160.2 | 34 |

In some embodiments, the at least one transcription factor is selected from the group consisting of NFIX, NFIC, RORC, NR0B2, ESR1, THRSP, TBX15, HLF, ATOH8, NR112, CUX2, ZNF662, TSHZ2, ATF5, NFIA, NFIB, NPAS2, FOS, ONECUT2, PROX1, NR1H4, MLXIPL, ETV1, AR, CEBPB, NR1D1, HEY2, ARID3C, KLF9, and DMRTA1.

In some embodiments, the transcription factor is Nuclear Factor I X (NFIX). As used herein, “NFIX” refers to the well-known gene and protein. NFIX is also known as Nuclear Factor I X, Nuclear Factor 1 X-Type, NF1-X, or NF-I/X. The protein encoded by the NFIX gene is a transcription factor that binds the palindromic sequence 5′-TTGGCNNNNNGCCAA-3 (SEQ ID NO: 46) in viral and cellular promoters and in the origin of replication of adenovirus type 2. The NFIX protein is individually capable of activating transcription and replication. The sequence of a human NFIX mRNA transcript can be found at National Center for Biotechnology Information (NCBI) RefSeq accession number NM_002501.4 (SEQ ID NO: 1). Additional examples of NFIX mRNA sequences are readily available using publicly available databases, e.g., GenBank, UniProt, and OMIM.

An exemplary sequence of NFIX comprises the nucleotide sequence of SEQ ID NO: 1, or an amino acid sequence encoded therefrom. In some embodiments, NFIX comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 1. In some embodiments, NFIX comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 1.

In some embodiments, the methods of the invention are directed to increasing the expression of NFIX by at least 0.1-fold, 0.2-fold, 0.5-fold, 1-fold, 2-fold, 5-fold, 10-fold, 20-fold, 50-fold, 100-fold, 200-fold, 500-fold, 1000-fold, or 10,000-fold relative to endogenous expression levels of NFIX in immature hepatocytes. In some embodiments, the increased expression of NFIX comprises an increase of at least 0.1-fold relative to endogenous expression levels of NFIX in the immature hepatocytes. In some embodiments, the increased expression of NFIX comprises an increase of at least 0.2-fold relative to endogenous expression levels of NFIX in the immature hepatocytes. In some embodiments, the increased expression of NFIX comprises an increase of at least 0.5-fold relative to endogenous expression levels of NFIX in the immature hepatocytes. In some embodiments, the increased expression of NFIX comprises an increase of at least 1-fold relative to endogenous expression levels of NFIX in the immature hepatocytes. In some embodiments, the increased expression of NFIX comprises an increase of at least 2-fold relative to endogenous expression levels of NFIX in the immature hepatocytes. In some embodiments, the increased expression of NFIX comprises an increase of at least 5-fold relative to endogenous expression levels of NFIX in the immature hepatocytes. In some embodiments, the increased expression of NFIX comprises an increase of at least 10-fold relative to endogenous expression levels of NFIX in the immature hepatocytes. In some embodiments, the increased expression of NFIX comprises an increase of at least 20-fold relative to endogenous expression levels of NFIX in the immature hepatocytes. In some embodiments, the increased expression of NFIX comprises an increase of at least 50-fold relative to endogenous expression levels of NFIX in the immature hepatocytes. In some embodiments, the increased expression of NFIX comprises an increase of at least 100-fold relative to endogenous expression levels of NFIX in the immature hepatocytes. In some embodiments, the increased expression of NFIX comprises an increase of at least 200-fold relative to endogenous expression levels of NFIX in the immature hepatocytes. In some embodiments, the increased expression of NFIX comprises an increase of at least 500-fold relative to endogenous expression levels of NFIX in the immature hepatocytes. In some embodiments, the increased expression of NFIX comprises an increase of at least 1,000-fold relative to endogenous expression levels of NFIX in the immature hepatocytes. In some embodiments, the increased expression of NFIX comprises an increase of at least 10,000-fold relative to endogenous expression levels of NFIX in the immature hepatocytes.

In some embodiments, the transcription factor is Nuclear Factor I C (NFIC). As used herein, “NFIC” refers to the well-known gene and protein. The term NFIC includes alternatively spliced or transcript variants (e.g., NFIC transcript variants 1-5) and protein isoforms. NFIC is also known as Nuclear Factor I C, CTF, Nuclear Factor 1 C-Type, NF1-C, or NF-I/C. The protein encoded by the NFIC gene belongs to the CTF/NF-I family. These are dimeric DNA-binding proteins, and function as cellular transcription factors and as replication factors for adenovirus DNA replication. The NFIC protein recognizes and binds the palindromic sequence 5′-TTGGCNNNNNGCCAA-3′ (SEQ ID NO: 46) present in viral and cellular promoters and in the origin of replication of adenovirus type 2. The NFIC protein is individually capable of activating transcription and replication. The NFIC gene encodes alternatively spliced variants. In some embodiments, NFIC is NFIC, transcript variant 1. The sequence of a human NFIC, transcript variant 1 mRNA transcript can be found at NCBI RefSeq accession number NM_001245002 (SEQ ID NO: 2). In some embodiments, NFIC is NFIC, transcript variant 2. The sequence of a human NFIC, transcript variant 2 mRNA transcript can be found at NCBI RefSeq accession number NM_205843 (SEQ ID NO: 3). In some embodiments, NFIC is NFIC, transcript variant 3. The sequence of a human NFIC, transcript variant 3 mRNA transcript can be found at NCBI RefSeq accession number NM_001245004 (SEQ ID NO: 4). In some embodiments, NFIC is NFIC, transcript variant 4. The sequence of a human NFIC, transcript variant 4 mRNA transcript can be found at NCBI RefSeq accession number NM_001245005 (SEQ ID NO: 5). In some embodiments, NFIC is NFIC, transcript variant 5. The sequence of a human NFIC, transcript variant 5 mRNA transcript can be found at NCBI RefSeq accession number NM_005597 (SEQ ID NO: 6). In some embodiments, NIFIC is any combination of NFIC, transcript variants 1-5. In some embodiments, NFIC is NFIC, transcript variant 1 and NFIC, transcript variant 3. Additional examples of NFIC mRNA sequences are readily available using publicly available databases, e.g., GenBank, UniProt, and OMIM.

An exemplary sequence of NFIC, transcript variant 1 comprises the nucleotide sequence of SEQ ID NO: 2, or an amino acid sequence encoded therefrom. In some embodiments, NFIC, transcript variant 1 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 2. In another embodiment, NIFC, transcript variant 1 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 2.

An exemplary sequence of NFIC, transcript variant 2 comprises the nucleotide sequence of SEQ ID NO: 3, or an amino acid sequence encoded therefrom. In some embodiments, NFIC, transcript variant 2 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 3. In an embodiment, NFIC, transcript variant 2 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 3.

An exemplary sequence of NFIC, transcript variant 3 comprises the nucleotide sequence of SEQ ID NO: 4, or an amino acid sequence encoded therefrom. In some embodiments, NFIC, transcript variant 3 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 4. In an embodiment, NFIC, transcript variant 3 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 4.

An exemplary sequence of NFIC, transcript variant 4 comprises the nucleotide sequence of SEQ ID NO: 5, or an amino acid sequence encoded therefrom. In some embodiments, NFIC, transcript variant 4 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 5. In an embodiment, NFIC, transcript variant 4 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 5.

An exemplary sequence of NFIC, transcript variant 5 comprises the nucleotide sequence of SEQ ID NO: 6, or an amino acid sequence encoded therefrom. In some embodiments, NFIC, transcript variant 5 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 6. In an embodiment, NFIC, transcript variant 5 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 6.

In some embodiments, the methods of the invention are directed to increasing the expression of NFIC by at least 0.1-fold, 0.2-fold, 0.5-fold, 1-fold, 2-fold, 5-fold, 10-fold, 20-fold, 50-fold, 100-fold, 200-fold, 500-fold, 1000-fold, or 10,000-fold relative to endogenous expression levels of NFIC in the immature hepatocytes. In some embodiments, the increased expression of NFIC comprises an increase of at least 0.1-fold relative to endogenous expression levels of NFIC in the immature hepatocytes. In some embodiments, the increased expression of NFIC comprises an increase of at least 0.2-fold relative to endogenous expression levels of NFIC in the immature hepatocytes. In some embodiments, the increased expression of NFIC comprises an increase of at least 0.5-fold relative to endogenous expression levels of NFIC in the immature hepatocytes. In some embodiments, the increased expression of NFIC comprises an increase of at least 1-fold relative to endogenous expression levels of NFIC in the immature hepatocytes. In some embodiments, the increased expression of NFIC comprises an increase of at least 2-fold relative to endogenous expression levels of NFIC in the immature hepatocytes. In some embodiments, the increased expression of NFIC comprises an increase of at least 5-fold relative to endogenous expression levels of NFIC in the immature hepatocytes. In some embodiments, the increased expression of NFIC comprises an increase of at least 10-fold relative to endogenous expression levels of NFIC in the immature hepatocytes. In some embodiments, the increased expression of NFIC comprises an increase of at least 20-fold relative to endogenous expression levels of NFIC in the immature hepatocytes. In some embodiments, the increased expression of NFIC comprises an increase of at least 50-fold relative to endogenous expression levels of NFIC in the immature hepatocytes. In some embodiments, the increased expression of NFIC comprises an increase of at least 100-fold relative to endogenous expression levels of NFIC in the immature hepatocytes. In some embodiments, the increased expression of NFIC comprises an increase of at least 200-fold relative to endogenous expression levels of NFIC in the immature hepatocytes. In some embodiments, the increased expression of NFIC comprises an increase of at least 500-fold relative to endogenous expression levels of NFIC in the immature hepatocytes. In some embodiments, the increased expression of NFIC comprises an increase of at least 1,000-fold relative to endogenous expression levels of NFIC in the immature hepatocytes. In some embodiments, the increased expression of NFIC comprises an increase of at least 10,000-fold relative to endogenous expression levels of NFIC in the immature hepatocytes.

In some embodiments, the transcription factor is RORC. The sequence of a human RORC mRNA transcript can be found at NCBI RefSeq accession number NM_005060.3 (SEQ ID NO: 7). An exemplary sequence of RORC comprises the nucleotide sequence of SEQ ID NO: 7, or an amino acid sequence encoded therefrom. In some embodiments, RORC comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 7. In an embodiment, RORC comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 7.

In some embodiments, the transcription factor is NR0B2. The sequence of a human NR0B2 mRNA transcript can be found at NCBI RefSeq accession number NM_021969.2 (SEQ ID NO: 8). An exemplary sequence of NR0B2 comprises the nucleotide sequence of SEQ ID NO: 8, or an amino acid sequence encoded therefrom. In some embodiments, NR0B2 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 8. In an embodiment, NR0B2 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 8.

In some embodiments, the transcription factor is ESR1. The sequence of a human ESR1 mRNA transcript can be found at NCBI RefSeq accession number NM_001291230.1 (SEQ ID NO: 9). An exemplary sequence of ESR1 comprises the nucleotide sequence of SEQ ID NO: 9, or an amino acid sequence encoded therefrom. In some embodiments, ESR1 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 9. In an embodiment, ESR1 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 9.

In some embodiments, the transcription factor is THRSP. The sequence of a human THRSP mRNA transcript can be found at NCBI RefSeq accession number NM_003251.3 (SEQ ID NO: 10). An exemplary sequence of THRSP comprises the nucleotide sequence of SEQ ID NO: 10, or an amino acid sequence encoded therefrom. In some embodiments, THRSP comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 10. In an embodiment, THRSP comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 10.

In some embodiments, the transcription factor is TBX15. The sequence of a human TBX15 mRNA transcript can be found at NCBI RefSeq accession number NM_152380 (SEQ ID NO: 11). An exemplary sequence of TBX15 comprises the nucleotide sequence of SEQ ID NO: 11, or an amino acid sequence encoded therefrom. In some embodiments, TBX15 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 11. In an embodiment, TBX15 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 11.

In some embodiments, the transcription factor is HLF. The sequence of a human HLF mRNA transcript can be found at NCBI RefSeq accession number NM_002126.4 (SEQ ID NO: 12). An exemplary sequence of HLF comprises the nucleotide sequence of SEQ ID NO: 12, or an amino acid sequence encoded therefrom. In some embodiments, HLF comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 12. In an embodiment, HLF comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 12.

In some embodiments, the transcription factor is ATOH8. The sequence of a human ATOH8 mRNA transcript can be found at NCBI RefSeq accession number NM_032827.7 (SEQ ID NO: 13). An exemplary sequence of ATOH8 comprises the nucleotide sequence of SEQ ID NO: 13, or an amino acid sequence encoded therefrom. In some embodiments, ATOH8 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 13. In an embodiment, ATOH8 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 13.

In some embodiments, the transcription factor is NR112. The sequence of a human NR112 mRNA transcript can be found at NCBI RefSeq accession number NM_003889.3 (SEQ ID NO: 14). An exemplary sequence of NR112 comprises the nucleotide sequence of SEQ ID NO: 14, or an amino acid sequence encoded therefrom. In some embodiments, NR112 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 14. In an embodiment, NR1I2 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 14.

In some embodiments, the transcription factor is CUX2. The sequence of a human CUX2 mRNA transcript can be found at NCBI RefSeq accession number NM_015267.3 (SEQ ID NO: 15). An exemplary sequence of CUX2 comprises the nucleotide sequence of SEQ ID NO: 15, or an amino acid sequence encoded therefrom. In some embodiments, CUX2 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 15. In an embodiment, CUX2 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 15.

In some embodiments, the transcription factor is ZNF662. The sequence of a human ZNF662 mRNA transcript can be found at NCBI RefSeq accession number NM_001134656.1 (SEQ ID NO: 16). An exemplary sequence of ZNF662 comprises the nucleotide sequence of SEQ ID NO: 16, or an amino acid sequence encoded therefrom. In some embodiments, ZNF662 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 16. In an embodiment, ZNF662 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 16.

In some embodiments, the transcription factor is TSHZ2. The sequence of a human TSHZ2 mRNA transcript can be found at NCBI RefSeq accession number NM_173485.5 (SEQ ID NO: 17). An exemplary sequence of TSHZ2 comprises the nucleotide sequence of SEQ ID NO: 17, or an amino acid sequence encoded therefrom. In some embodiments, TSHZ2 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 17. In an embodiment, TSHZ2 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 17.

In some embodiments, the transcription factor is ATF5. The sequence of a human ATF5 mRNA transcript can be found at NCBI RefSeq accession number NM_001193646.1 (SEQ ID NO: 18). An exemplary sequence of ATF5 comprises the nucleotide sequence of SEQ ID NO: 18, or an amino acid sequence encoded therefrom. In some embodiments, ATF5 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 18. In an embodiment, ATF5 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 18.

In some embodiments, the transcription factor is NFIA. The sequence of a human NFIA mRNA transcript can be found at NCBI RefSeq accession number NM_001134673.3 (SEQ ID NO: 19). An exemplary sequence of NFIA comprises the nucleotide sequence of SEQ ID NO: 19, or an amino acid sequence encoded therefrom. In some embodiments, NFIA comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 19. In an embodiment, NFIA comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 19.

In some embodiments, the transcription factor is NFIB. The sequence of a human NFIB mRNA transcript can be found at NCBI RefSeq accession number NM_005596.3 (SEQ ID NO: 20). An exemplary sequence of NFIB comprises the nucleotide sequence of SEQ ID NO: 20, or an amino acid sequence encoded therefrom. In some embodiments, NFIB comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 20. In an embodiment, NFIB comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 20.

In some embodiments, the transcription factor is NPAS2. The sequence of a human NPAS2 mRNA transcript can be found at NCBI RefSeq accession number XM_005263953.2 (SEQ ID NO: 21). An exemplary sequence of NPAS2 comprises the nucleotide sequence of SEQ ID NO: 21, or an amino acid sequence encoded therefrom. In some embodiments, NPAS2 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 21. In an embodiment, NPAS2 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 21.

In some embodiments, the transcription factor is FOS. The sequence of a human FOS mRNA transcript can be found at NCBI RefSeq accession number NM_005252.3 (SEQ ID NO: 22). An exemplary sequence of FOS comprises the nucleotide sequence of SEQ ID NO: 22, or an amino acid sequence encoded therefrom. In some embodiments, FOS comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 22. In an embodiment, FOS comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 22.

In some embodiments, the transcription factor is ONECUT2. The sequence of a human ONECUT2 mRNA transcript can be found at NCBI RefSeq accession number NM_004852.2 (SEQ ID NO: 23). An exemplary sequence of ONECUT2 comprises the nucleotide sequence of SEQ ID NO: 23, or an amino acid sequence encoded therefrom. In some embodiments, ONECUT2 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 23. In an embodiment, ONECUT2 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 23.

In some embodiments, the transcription factor is PROX1. The sequence of a human PROX1 mRNA transcript can be found at NCBI RefSeq accession number NM_001270616.2 (PROX1, transcript variant 1; SEQ ID NO: 24) or NM_002763.5 (PROX1, transcript variant 2; SEQ ID NO: 39). An exemplary sequence of PROX1 comprises the nucleotide sequence of SEQ ID NO: 24, or an amino acid sequence encoded therefrom. In some embodiments, PROX1 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 24. In an embodiment, PROX1 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 24. An exemplary sequence of PROX1 comprises the nucleotide sequence of SEQ ID NO: 39, or an amino acid sequence encoded therefrom. In some embodiments, PROX1 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 39. In an embodiment, PROX1 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 39.

In some embodiments, the transcription factor is NR1H4. The sequence of a human NR1H4 mRNA transcript can be found at NCBI RefSeq accession number NM_001206979.1 (SEQ ID NO: 25). An exemplary sequence of NR1H4 comprises the nucleotide sequence of SEQ ID NO: 25, or an amino acid sequence encoded therefrom. In some embodiments, NR1H4 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 25. In an embodiment, NR1H4 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 25.

In some embodiments, the transcription factor is MLXIPL. The sequence of a human MLXIPL mRNA transcript can be found at NCBI RefSeq accession number NM_032951.2 (SEQ ID NO: 26). An exemplary sequence of MLXIPL comprises the nucleotide sequence of SEQ ID NO: 26, or an amino acid sequence encoded therefrom. In some embodiments, MLXIPL comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 26. In an embodiment, MLXIPL comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 26.

In some embodiments, the transcription factor is ETV1. The sequence of a human ETV1 mRNA transcript can be found at NCBI RefSeq accession number NM_001163147 (SEQ ID NO: 27). An exemplary sequence of ETV1 comprises the nucleotide sequence of SEQ ID NO: 27, or an amino acid sequence encoded therefrom. In some embodiments, ETV1 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 27. In an embodiment, ETV1 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 27.

In some embodiments, the transcription factor is AR. The sequence of a human AR mRNA transcript can be found at NCBI RefSeq accession number NM_000044.3 (SEQ ID NO: 28). An exemplary sequence of AR comprises the nucleotide sequence of SEQ ID NO: 28, or an amino acid sequence encoded therefrom. In some embodiments, AR comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 28. In an embodiment, AR comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 28.

In some embodiments, the transcription factor is CEBPB. The sequence of a human CEBPB mRNA transcript can be found at NCBI RefSeq accession number NM_005194.3 (SEQ ID NO: 29). An exemplary sequence of CEBPB comprises the nucleotide sequence of SEQ ID NO: 29, or an amino acid sequence encoded therefrom. In some embodiments, CEBPB comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 29. In an embodiment, CEBPB comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 29.

In some embodiments, the transcription factor is NR1D1. The sequence of a human NR1D1 mRNA transcript can be found at NCBI RefSeq accession number NM_021724.4 (SEQ ID NO: 30). An exemplary sequence of NR1D1 comprises the nucleotide sequence of SEQ ID NO: 30, or an amino acid sequence encoded therefrom. In some embodiments, NR1D1 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 30. In an embodiment, NR1D1 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 30.

In some embodiments, the transcription factor is HEY2. The sequence of a human HEY2 mRNA transcript can be found at NCBI RefSeq accession number NM_012259.2 (SEQ ID NO: 31). An exemplary sequence of HEY2 comprises the nucleotide sequence of SEQ ID NO: 31, or an amino acid sequence encoded therefrom. In some embodiments, HEY2 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 31. In an embodiment, HEY2 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 31.

In some embodiments, the transcription factor is ARID3C. The sequence of a human ARID3C mRNA transcript can be found at NCBI RefSeq accession number NM_001017363.1 (SEQ ID NO: 32). An exemplary sequence of ARID3C comprises the nucleotide sequence of SEQ ID NO: 32, or an amino acid sequence encoded therefrom. In some embodiments, ARID3C comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 32. In an embodiment, ARID3C comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 32.

In some embodiments, the transcription factor is KLF9. The sequence of a human KLF9 mRNA transcript can be found at NCBI RefSeq accession number NM_001206.2 (SEQ ID NO: 33). An exemplary sequence of KLF9 comprises the nucleotide sequence of SEQ ID NO: 33, or an amino acid sequence encoded therefrom. In some embodiments, KLF9 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 33. In an embodiment, KLF9 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 33.

In some embodiments, the transcription factor is DMRTA1. The sequence of a human DMRTA1 mRNA transcript can be found at NCBI RefSeq accession number NM_022160.2 (SEQ ID NO: 34). An exemplary sequence of DMRTA1 comprises the nucleotide sequence of SEQ ID NO: 34, or an amino acid sequence encoded therefrom. In some embodiments, DMRTA1 comprises a nucleotide sequence that is at least 80%, at least 85%, at least 90%, at least 91%, at least 92%, at least 93%, at least 94%, at least 95%, at least 96%, at least 97%, at least 98%, at least 99% or 100% identical to the nucleotide sequence of SEQ ID NO: 34. In an embodiment, DMRTA1 comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence of SEQ ID NO: 34.

### Increasing Expression of Transcription Factors

Vectors for delivery of nucleic acids encoding the transcription factor(s) of the invention may be constructed to express the transcription factor(s) in the cells of the disclosure, for example, an immature hepatocyte, a hepatic progenitor cell, or a pluripotent stem cell, e.g., an embryonic stem cell or an induced pluripotent stem cell. In some embodiments, the nucleic acid is DNA. In some embodiments, the nucleic acid is RNA. In some embodiments, the nucleic acid is modified DNA. In some embodiments, the nucleic acid is modified RNA.

In addition, protein transduction compositions or methods may also be used to effect expression of the transcription factor(s) in the methods of the invention.

### A. Nucleic Acid Delivery Systems

One of skill in the art would be well equipped to construct a vector through standard recombinant techniques (see, for example, Sambrook et al., 2001; Ausubel et al., 1996; Maniatis et al., 1988; and Ausubel et al., 1994; each of which is incorporated in its entirety herein by reference). Vectors comprising a nucleic acid encoding the at least one transcription factor of the disclosure include, but are not limited to, viral vectors, non-viral vectors and/or inducible expression vectors.

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide.

Such components also might include markers, such as detectable and/or selection markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

### 1. Viral Vectors

Viral vectors encoding at least one transcription factor of the invention may be provided in certain aspects of the present disclosure. A viral vector is a kind of an expression construct that utilizes viral sequences to introduce nucleic acid and possibly proteins into a cell. Non-limiting examples of viral vectors that may be used to deliver a nucleic acid of certain aspects of the present invention are described below.

In some embodiments, the viral vector is a non-integrating viral vector. An exemplary non-integrating viral vector of the disclosure is selected from the group consisting of an adeno-associated virus (AAV) vector, e.g., AAV1, AAV2, AAV3, AAV4, AAV5, AAV6, AAV7, AAV8, AAV9, AAV10, AAV3B, AAV-218, Rh1O, Rh74 etc.; an adenovirus (Ad) vector, including replication competent, replication deficient and gutless forms thereof, e.g., Ad7, Ad4, Ad2, Ad5 etc.; a simian virus 40 (SV-40) vector, a bovine papilloma virus vector, an Epstein-Barr virus vector, a herpes virus vector, a vaccinia virus vector, a Harvey murine sarcoma virus vector, a murine mammary tumor virus vector, or a Rous sarcoma virus vector.

In some embodiments, the viral vector is an integrating viral vector, e.g., a retroviral vector. Retroviruses have promise as gene delivery vectors due to their ability to integrate their genes into the host genome, transferring a large amount of foreign genetic material, infecting a broad spectrum of species and cell types and of being packaged in special cell lines.

In some embodiments, integrating viral vectors are derived from retroviral vectors (e.g., Moloney murine leukemia virus vectors (MoMLV), MSCV, SFFV, MPSV, SNV, etc.), lentiviral vectors (e.g. derived from HIV-1, HIV-2, SIV, BIV, FIV etc.), or vectors derived therefrom.

Recombinant vectors are also capable of infecting non-dividing cells, and can be used in the methods of the invention for both in vivo and ex vivo gene transfer and expression of nucleic acid sequences. For example, recombinant lentiviruses capable of infecting a non-dividing cell, wherein a suitable host cell (i.e., the virus producing cell, and not a hepatocyte of the disclosure), is transfected with two or more vectors carrying the packaging functions, namely gag, pol and env, as well as rev and tat is described in U.S. Pat. No. 5,994,136, incorporated in its entirety herein by reference.

### 2. Episomal Vectors and Other Non-Viral Vectors

The use of plasmid- or liposome-based extra-chromosomal (i.e., episomal) vectors may be also provided in certain aspects of the invention. Such episomal vectors may include, e.g., oriP-based vectors, and/or vectors encoding a derivative of EBNA-1. These vectors may permit large fragments of DNA to be introduced to a cell and maintained extra-chromosomally, replicated once per cell cycle, partitioned to daughter cells efficiently, and elicit substantially no immune response.

Other extra-chromosomal vectors include other lymphotrophic herpes virus-based vectors. Exemplary lymphotrophic herpes viruses include, but are not limited to EBV, Kaposi's sarcoma herpes virus (KSHV); Herpes virus saimiri (HS) and Marek's disease virus (MDV). Also other sources of episome-base vectors are contemplated, such as yeast ARS, adenovirus, SV40, or BPV.

In some embodiments, the vector is a non-viral vector. In some embodiments, the non-viral vector is selected from the group consisting of a plasmid DNA, a linear double-stranded DNA (dsDNA), a linear single-stranded DNA (ssDNA), a nanoplasmid, a minicircle DNA, a single-stranded oligodeoxynucleotides (ssODN), a DDNA oligonucleotide, a single-stranded mRNA (ssRNA), and a double-stranded mRNA (dsRNA).

In some embodiments, the non-viral vector comprises a naked nucleic acid, a liposome, a dendrimer, a nanoparticle, a lipid-polymer system, a solid lipid nanoparticle, and/or a liposome protamine/DNA lipoplex (LPD).

In some embodiments, the non-viral vector comprises an mRNA. In some embodiments, the mRNA may be delivered as naked modified mRNA, for example, in a sucrose-citrate buffer or saline solution. In other embodiments, a non-viral vector comprises an mRNA complexed with a transfection reagent, such as Lipofectamine 2000, jetPEI, RNAiMAX, and/or Invivofectamine. To protect mRNA against degradation by nucleases and shield its negative charge, amine-containing materials are also commonly used as non-viral vectors. One of the most developed methods for mRNA delivery is co-formulation into lipid nanoparticles (LNPs). LNP formulations are typically composed of (1) an ionizable or cationic lipid or polymeric material, bearing tertiary or quaternary amines to encapsulate the polyanionic mRNA; (2) a zwitterionic lipid (e.g., 1,2-diolcoyl-sn-glycero-3-phosphoethanolamine [DOPE]) that resembles the lipids in the cell membrane; (3) cholesterol to stabilize the lipid bilayer of the LNP; and (4) a polyethylene glycol (PEG)-lipid to lend the nanoparticle a hydrating layer, improve colloidal stability, and reduce protein absorption. Exemplary non-viral vectors comprising an mRNA are described in Kowalksi et al., 2019, Mol Ther.; 27(4): 710-728; incorporated in its entirety herein by reference.

### 3. Transposon-Based System

According to a particular embodiment the introduction of nucleic acids may use a transposo—transposase system. The used transposon—transposase system could be the well known Sleeping Beauty, the Frog Prince transposon—transposase system (for the description of the latter see e.g. EP1507865), or the TTAA-specific transposon piggyBac system.

Transposons are sequences of DNA that can move around to different positions within the genome of a single cell, a process called transposition. In the process, they can cause mutations and change the amount of DNA in the genome. There are a variety of mobile genetic elements, and they can be grouped based on their mechanism of transposition. Class I mobile genetic elements, or retrotransposons, copy themselves by first being transcribed to RNA, then reverse transcribed back to DNA by reverse transcriptase, and then being inserted at another position in the genome. Class II mobile genetic elements move directly from one position to another using a transposase to “cut and paste” them within the genome.

### 4. Homologous Recombination

Homologous recombination (HR) is a targeted genome modification technique that has been the standard method for genome engineering in mammalian cells since the mid 1980s. The use of meganucleases, or homing endonucleases, such as I-SceI have been used to increase the efficiency of HR. Both natural meganucleases as well as engineered meganucleases with modified targeting specificities have been utilized to increase HR efficiency. Another path toward increasing the efficiency of HR has been to engineer chimeric endonucleases with programmable DNA specificity domains. Zinc-finger nucleases (ZFN) are one example of such a chimeric molecule in which Zinc-finger DNA binding domains are fused with the catalytic domain of a Type IIS restriction endonuclease such as FokI. Another class of such specificity molecules includes Transcription Activator Like Effector (TALE) DNA binding domains fused to the catalytic domain of a Type IIS restriction endonuclease such as FokI. Another class of such molecules that facilitate targeted genome modification include the CRISPR/Cas system, for example, as described in Ran et al., 2013; Nature Protocols 8:2281-2308; which is incorporated in its entirety herein by reference.

### B. Regulatory Elements

Eukaryotic expression cassettes included in the vectors preferably contain (in a 5′-to-3′ direction) a eukaryotic transcriptional promoter operably linked to a protein-coding sequence, splice signals including intervening sequences, and a transcriptional termination/polyadenylation sequence.

### 1. Promoter/Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “operably linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. Additional promoter elements regulate the frequency of transcriptional initiation. Typically, these are located in the region 30-110 by upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 by apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each of which is incorporated herein by reference in its entirety). Furthermore, it is contemplated the control sequences that direct transcription and/or expression of sequences within non-nuclear organelles such as mitochondria, chloroplasts, and the like, can be employed as well.

The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be artificial or endogenous.

In some embodiments, the promoter is an inducible promoter. The term “inducible promoter” is known in the art and refers to promoters that are active only in response to a stimulus. Inducible promoters selectively express a nucleic acid molecule in response to the presence of an endogenous or exogenous stimulus, for example a chemical compound (a chemical inducer) or in response to environmental, hormonal, chemical, and/or developmental signals. Inducible promoters include, for example, promoters induced or regulated by light, heat, stress, (e.g., salt stress, or osmotic stress), phytohormones, wounding, or chemicals such as ethanol, abscisic acid (ABA), jasmonate, salicylic acid, or safeners. In some embodiments, the inducible promoter is an EF1a promoter. In some embodiments, the inducible promoter is a PGK promoter.

Additionally any promoter/enhancer combination (as per, for example, the Eukaryotic Promoter Data Base EPDB, through world wide web at epd.isb-sib.ch/) could also be used to drive expression. Non-limiting examples of promoters include a constitutive EF1 alpha promoter; early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box.

Several enhancer sequences for liver-specific genes have been documented. For example, PCT Publication No. WO2009130208 describes several liver-specific regulatory enhancer sequences, and is incorporated herein in its entirety by reference. PCT Publication No. WO95/011308 describes a gene therapy vector comprising a hepatocyte-specific control region (HCR) enhancer linked to a promoter and a transgene, and is incorporated herein in its entirety by reference. PCT Publication No. WO01/098482 teaches a combination of specific ApoE enhancer sequences or a truncated version thereof with hepatic promoters, which is incorporated herein in its entirety by reference.

### 2. Initiation Signals, Internal Ribosome Binding Sites and Self-Cleaving Sequences

A specific initiation signal also may be used for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819; each of which is incorporated in its entirety herein by reference).

In some embodiments, self-cleaving sequences can be used to co-express genes. The term “self-cleaving sequence” as used herein refers to a sequence that links open reading frames to form a single cistron, and induces ribosomal skipping during translation. Ribosomal skipping causes the two coding sequences connected by the self-cleaving sequence to be translated into two separate peptides. For example, 2A self-cleaving sequences can be used to create linked- or co-expression of genes in the constructs provided in the present disclosure. Exemplary self-cleaving sequences include, but are not limited to, T2A, P2A, E2A and F2A, as described in Table 2.

TABLE 2
Exemplary 2A Sequences

| | | |
|---|---|---|
| T2A | GSGEGRGSLLTCGDVEENPGP | SEQ ID NO: 35 |
| P2A | GSGATNFSLLKQAGDVEENPGP | SEQ ID NO: 36 |
| E2A | GSGQCTNYALLKLAGDVESNPGP | SEQ ID NO: 37 |
| F2A | GSGVKQTLNFDLLKLAGDVESNPGP | SEQ ID NO: 38 |

In some embodiments, T2A comprises an amino acid sequence having at least 90%, 91%, 92%, 93%, 94%, 95%, 96%, 97%, 98%, 99% or 100% identity with the amino acid sequence of SEQ ID NO: 35, or a nucleic acid encoding such amino acid sequence.

In some embodiments, P2A comprises an amino acid sequence having at least 90%, 91%, 92%, 93%, 94%, 95%, 96%, 97%, 98%, 99% or 100% identity with the amino acid sequence of SEQ ID NO: 36, or a nucleic acid encoding such amino acid sequence.

In some embodiments, E2A comprises an amino acid sequence having at least 90%, 91%, 92%, 93%, 94%, 95%, 96%, 97%, 98%, 99% or 100% identity with the amino acid sequence of SEQ ID NO: 37, or a nucleic acid encoding such amino acid sequence.

In some embodiments, F2A comprises an amino acid sequence having at least 90%, 91%, 92%, 93%, 94%, 95%, 96%, 97%, 98%, 99% or 100% identity with the amino acid sequence of SEQ ID NO: 38, or a nucleic acid encoding such amino acid sequence.

### 3. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively, a replication origin of other extra-chromosomally replicating virus as described above or an autonomously replicating sequence (ARS) can be employed.

### 4. Selection and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated.

Alternatively, screenable enzymes such as negative selection markers may be utilized. In certain embodiments, the negative selection marker comprises one or more suicide genes, which upon administration of a prodrug, effects transition of a gene product to a compound which kills its host cell. Exemplary suicide genes of the disclosure include, but are not limited to, inducible caspase 9 (or caspase 3 or 7), CD20, CD52, EGFRt, thymidine kinase, cytosine deaminase, HER1 and any combination thereof. Further suicide genes known in the art that may be used in the present disclosure include purine nucleoside phosphorylase (PNP), cytochrome p450 enzymes (CYP), carboxypeptidases (CP), carboxylesterase (CE), nitroreductase (NTR), guanine ribosyltransferase (XGRTP), glycosidase enzymes, and thymidine phosphorylase (TP).

One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art. One feature of the present invention includes using selection and screenable markers to select for hepatocytes after the transcription factors have effected a desired change in those cells.

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated.

### C. Nucleic Acid Delivery

In certain embodiments, increasing the expression of the at least one transcription factor in the immature hepatocytes comprises contacting a cell, e.g., a pluripotent stem cell, an immature hepatocyte, or a hepatic progenitor cell, with the at least one transcription factor. In some embodiments, the cell e.g., a pluripotent stem cell, an immature hepatocyte, or a hepatic progenitor cell, comprises an expression vector comprising a nucleic acid encoding the at least one transcription factor.

Introduction of a nucleic acid, such as DNA, RNA, modified DNA or modified RNA into cells of the current invention, e.g., a pluripotent stem cell, an immature hepatocyte, or a hepatic progenitor cell, may use any suitable methods for nucleic acid delivery for transformation of a cell, as described herein or as would be known to one of ordinary skill in the art. Such methods include, but are not limited to, direct delivery of DNA such as by ex vivo transfection (Wilson et al., 1989, Nabel et al, 1989; each of which is incorporated in its entirety herein by reference), by injection (U.S. Pat. Nos. 5,994,624, 5,981,274, 5,945,100, 5,780,448, 5,736,524, 5,702,932, 5,656,610, 5,589,466 and 5,580,859; each of which is incorporated in its entirety herein by reference), including microinjection (Harland and Weintraub, 1985; U.S. Pat. No. 5,789,215; each of which is incorporated in its entirety herein by reference); by electroporation (U.S. Pat. No. 5,384,253; Tur-Kaspa et al., 1986; Potter et al., 1984; each of which is incorporated in its entirety herein by reference); by calcium phosphate precipitation (Graham and Van Der Eb, 1973; Chen and Okayama, 1987; Rippe et al., 1990; each of which is incorporated in its entirety herein by reference); by using DEAE-dextran followed by polyethylene glycol; by direct sonic loading (Fechheimer et al., 1987; which is incorporated in its entirety herein by reference); by liposome mediated transfection (Nicolau and Sene, 1982; Fraley et al., 1979; Nicolau et al., 1987; Wong et al., 1980; Kaneda et al., 1989; Kato et al., 1991; each of which is incorporated in its entirety herein by reference) and receptor-mediated transfection (Wu and Wu, 1987; Wu and Wu, 1988; each of which is incorporated in its entirety herein by reference); by microprojectile bombardment (PCT Application Nos. WO 94/09699 and 95/06128; U.S. Pat. Nos. 5,610,042; 5,322,783 5,563,055, 5,550,318, 5,538,877 and 5,538,880; each of which is incorporated in its entirety herein by reference); by agitation with silicon carbide fibers (Kaeppler et al., 1990; U.S. Pat. Nos. 5,302,523 and 5,464,765; each of which is incorporated in its entirety herein by reference); by Agrobacterium-mediated transformation (U.S. Pat. Nos. 5,591,616 and 5,563,055; each of which is incorporated in its entirety herein by reference); by desiccation/inhibition-mediated DNA uptake (Potrykus et al., 1985; which is incorporated in its entirety herein by reference), and any combination of such methods. Through the application of techniques such as these, organelle(s), cell(s), tissue(s) or organism(s) may be stably or transiently transformed.

In a certain embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated is an nucleic acid complexed with Lipofectamine (Gibco BRL) or Superfect (Qiagen). The amount of liposomes used may vary upon the nature of the liposome as well as the cell used, for example, about 5 to about 20 μg vector DNA per 1 to 10 million of cells may be contemplated.

In certain embodiments of the present invention, a nucleic acid is introduced into an organelle, a cell, a tissue or an organism via electroporation. Electroporation involves the exposure of a suspension of cells and DNA to a high-voltage electric discharge. Recipient cells can be made more susceptible to transformation by mechanical wounding. Also the amount of vectors used may vary upon the nature of the cells used, for example, about 5 to about 20 μg vector DNA per 1 to 10 million of cells may be contemplated.

In other embodiments of the present invention, a nucleic acid is introduced to the cells using calcium phosphate precipitation.

In another embodiment, a nucleic acid is delivered into a cell using DEAE-dextran followed by polyethylene glycol.

Additional embodiments of the present invention include the introduction of a nucleic acid by direct sonic loading.

Microprojectile bombardment techniques can also be used to introduce a nucleic acid into at least one, organelle, cell, tissue or organism (U.S. Pat. Nos. 5,550,318; 5,538,880; 5,610,042; and PCT Application WO 94/09699; each of which is incorporated herein by reference). This method depends on the ability to accelerate DNA-coated microprojectiles to a high velocity allowing them to pierce cell membranes and enter cells without killing them (Klein et al., 1987; which is incorporated in its entirety herein by reference). There are a wide variety of microprojectile bombardment techniques known in the art, which are suitable for use in the methods of the invention.

### D. Gene Switches

In some embodiments, the cells of the disclosure, e.g., pluripotent stem cells or immature hepatocytes, are engineered to comprise a gene switch construct encoding the transcription factor(s) of the invention. Gene switch constructs provide basic building blocks for the construction of complex gene circuits that transform cells into useful cell-based machines for biomedical applications. Ligand-responsive gene switch constructs are cellular sensors that are able to process specific signals to generate gene product responses. Their involvement in complex gene circuits results in sophisticated circuit topologies that are reminiscent of electronics and that are capable of providing engineered cells with the ability to memorize events, oscillate protein production, and perform complex information-processing tasks (see, Ausländer et al., 2016; Cold Spring Harb Perspect Biol.; 8(7): a023895; incorporated in its entirety herein by reference). Based on the gene switch construct design strategy, the cells of the disclosure, e.g., pluripotent stem cells or immature hepatocytes, can be engineered to comprise a gene switch construct encoding the transcription factor of the disclosure, along with various synthetic systems to sense different ligand inputs that in turn mediate expression of the gene switch construct encoding the transcription factor of the disclosure.

### 1. Transcriptional Gene Switches

In some embodiments, the gene switch construct is a transcriptional gene switch construct. In some embodiments, the transcriptional gene switch construct comprises use of prokaryotic regulator proteins fused to transcriptional regulator proteins, which bind to DNA operator sequences to control the expression of the gene switch construct in a ligand-responsive manner. In some embodiments, the transcriptional gene switch construct comprises use of combining prokaryotic regulator proteins with ligand- or light-induced dimerization systems (DSs) enables the signal dependent recruitment of transcriptional regulator proteins. In some embodiments, the transcriptional gene switch construct comprises use of eukaryotic cell-surface-located G protein-coupled receptors (GPCRs) that sense extracellular signals and trigger signal transduction via signaling pathways to control expression of the gene switch construct. In some embodiments, the transcriptional gene switch construct comprises use of an engineered diguanylate cyclase (DGCL) that synthesizes the second messenger cyclic-di-GMP in a red-light-responsive manner, triggering a downstream signaling pathway and leading to the transcriptional activation of the gene switch construct. In some embodiments, the transcriptional gene switch construct comprises use of any of the synthetic systems described in Ausländer et al., 2016; incorporated in its entirety herein by reference.

### 2. Post-Transcriptional Gene Switches

In some embodiments, the gene switch construct is a post-transcriptional gene switch construct. In some embodiments, the post-transcriptional gene switch construct comprises use of aptazymes fused to primary microRNA (pri-miRNA) molecules, enabling the ligand-responsive control of pri-miRNA processing and posttranscriptional target gene control. In some embodiments, the post-transcriptional gene switch construct comprises use of protein responsive aptazymes integrated into messenger RNAs (mRNAs) to regulate their stability, depending on the presence or absence of the protein ligand. In some embodiments, the post-transcriptional gene switch construct comprises use of protein binding to protein-binding aptamers that are integrated into small hairpin RNAs (shRNAs) and inhibit shRNA processing and allows for protein-controlled expression of the gene switch construct. In some embodiments, the post-transcriptional gene switch construct comprises use of protein-binding aptamers integrated into the 5′ untranslated regions (UTRs) of mRNAs to control translational initiation in a protein-dependent manner. In some embodiments, the post-transcriptional gene switch construct comprises use of integration of protein-binding aptamers into close proximity of splicing sites to allow protein-responsive alternative splicing regulation. In some embodiments, the post-transcriptional gene switch construct comprises use of an ATetR-binding aptamer combined with a theophylline-responsive aptamer to enable the theophylline-dependent folding of the TetR-binding aptamer. When bound to its cognate aptamer, the TetR protein loses its DNA operator binding ability and influences gene expression at the transcriptional level.

Integrases can also act as functional genetic switch controllers, activating the coding sequence or the promoter switches designed to be turned on in eukaryotic cells. Integrases show accuracy in their site recognition and recombination process, and are not cytotoxic. In some embodiments, the gene switch construct comprises use of genetic switches controlled by serine integrases, as described in Gomide et al., 2020, Commun Biol.; 3(1):255; incorporated in its entirety herein by reference.

### E. Protein Transduction

In certain embodiments, the cells of the disclosure, e.g., immature hepatocytes, may be contacted with transcription factor(s) comprising polypeptides at a sufficient amount for generating mature hepatocytes. Protein transduction has been used as a method for enhancing the delivery of macromolecules into cells. Protein transduction domains may be used to introduce transcription factor polypeptides or functional fragments thereof directly into cells.

A “protein transduction domain” or “PTD” is an amino acid sequence that can cross a biological membrane, particularly a cell membrane. When attached to a heterologous polypeptide, a PTD can enhance the translocation of the heterologous polypeptide across a biological membrane. The PTD is typically covalently attached (e.g., by a peptide bond) to the heterologous DNA binding domain. For example, the PTD and the heterologous DNA binding domain can be encoded by a single nucleic acid, e.g., in a common open reading frame or in one or more exons of a common gene. An exemplary PTD can include between 10-30 amino acids and may form an amphipathic helix. Many PTD's are basic in character. For example, a basic PTD can include at least 4, 5, 6 or 8 basic residues (e.g., arginine or lysine). A PTD may be able to enhance the translocation of a polypeptide into a cell that lacks a cell wall or a cell from a particular species, e.g., a mammalian cell, such as a human, simian, murine, bovine, equine, feline, or ovine cell.

A PTD can be linked to an artificial transcription factor, for example, using a flexible linker. Flexible linkers can include one or more glycine residues to allow for free rotation. For example, the PTD can be spaced from a DNA binding domain of the transcription factor by at least 10, 20, or 50 amino acids. A PTD can be located N- or C-terminal relative to a DNA binding domain. Being located N- or C-terminal to a particular domain does not require being adjacent to that particular domain. For example, a PTD N-terminal to a DNA binding domain can be separated from the DNA binding domain by a spacer and/or other types of domains. A PTD can be chemically synthesized then conjugated chemically to separately prepared DNA binding domain with or without linker peptide. An artificial transcription factor can also include a plurality of PTD's, e.g., a plurality of different PTD's or at least two copies of one PTD.

Several proteins and small peptides have the ability to transduce or travel through biological membranes independent of classical receptor- or endocytosis-mediated pathways. Examples of these proteins include the HIV-1 TAT protein, the herpes simplex virus 1 (HSV-1) DNA-binding protein VP22, and the Drosophila Antennapedia (Antp) homeotic transcription factor. The small protein transduction domains (PTDs) from these proteins can be fused to other macromolecules, peptides or proteins to successfully transport them into a cell. Sequence alignments of the transduction domains from these proteins show a high basic amino acid content (Lys and Arg) which may facilitate interaction of these regions with negatively charged lipids in the membrane. Secondary structure analyses show no consistent structure between all three domains.

The advantages of using fusions of these transduction domains is that protein entry is rapid, concentration-dependent and appears to work with difficult cell types. PTDs are further described in U.S. 2003/0082561; U.S. 2002/0102265; U.S. 2003/0040038; each of which is incorporated in its entirety herein by reference.

In addition to PTDs, cellular uptake signals can be used. Such signals include amino acid sequences which are specifically recognized by cellular receptors or other surface proteins. Interaction between the cellular uptake signal and the cell cause internalization of the artificial transcription factor that includes the cellular uptake signal. Some PTDs may also function by interaction with cellular receptors or other surface proteins.

### Cell Culturing

Generally, cells of the present invention are cultured in a culture medium, which is a nutrient-rich buffered solution capable of sustaining cell growth.

Hepatocytes of the invention can be made by culturing pluripotent stem cells or other cells, e.g., immature hepatocytes in a medium under conditions that increase the intracellular level of transcription factors described herein to be sufficient to promote generation of mature hepatocytes. The medium may also contain one or more hepatocyte differentiation agents, like various kinds of growth factors. These agents may either help induce cells to commit to a more mature phenotype—or preferentially promote survival of the mature cells—or have a combination of both these effects.

Hepatocyte differentiation agents illustrated in this disclosure may include soluble growth factors (peptide hormones, cytokines, ligand-receptor complexes, and other compounds) that are capable of promoting the growth of cells of the hepatocyte lineage. Non-limiting examples of such agents include but are not limited to epidermal growth factor (EGF), insulin, TGF-α, TGF-β, fibroblast growth factor (FGF), heparin, hepatocyte growth factor (HGF), Oncostatin M (OSM), IL-1, IL-6, insulin-like growth factors I and II (IGF-I, IGF-2), heparin binding growth factor 1 (HBGF-1), Wnt Family Member 3A (WNT3A), A83, CHIR, and glucagon. The skilled artisan will already appreciate that Oncostatin M is structurally related to Leukemia inhibitory factor (LIF), Interleukin-6 (IL-6), and ciliary neurotrophic factor (CNTF).

In some embodiments, the methods of the invention comprise increasing expression of at least one transcription factor selected from the group consisting of Nuclear Factor I X (NFIX) and Nuclear Factor I C (NFIC), in immature hepatocytes and culturing the immature hepatocytes in a culture media comprising dexamethasone, 8-Bromoadenosine 3′, 5′-cyclic monophosphate (8-Br-cAMP), or a combination thereof.

In some embodiments, the culturing the immature hepatocytes in a culture media comprising dexamethasone, 8-Br-cAMP, or a combination thereof is performed for at least 1, 2, 3, 4, 5, 6, 7, 8, 9, 10, 12, 15 or 20 days. In some embodiments, the culturing the immature hepatocytes in a culture media comprising dexamethasone, 8-Br-cAMP, or a combination thereof is performed for at least 1-3 days. In some embodiments, the culturing the immature hepatocytes in a culture media comprising dexamethasone, 8-Br-cAMP, or a combination thereof is performed for at least 2-5 days. In some embodiments, the culturing the immature hepatocytes in a culture media comprising dexamethasone, 8-Br-cAMP, or a combination thereof is performed for at least 3-7 days. In some embodiments, the culturing the immature hepatocytes in a culture media comprising dexamethasone, 8-Br-cAMP, or a combination thereof is performed for at least 5-9 days.

In some embodiments, the concentration of 8-Br-CAMP is at least 0.1 mM, 0.2 mM, 0.4 mM, 0.6 mM, 0.8 nM, 1 mM, 1.5 mM, 2 mM, 3 mM, 5 mM, 10 mM, 20 mM, 30 mM, 40 mM or 50 mM. In some embodiments, the concentration of 8-Br-CAMP is about 0.1-0.5 mM, 0.2-0.7 mM, 0.3-0.9 mM, 0.5-1 mM, 1-5 mM, 5-10 mM, or 10-50 mM. In some embodiments, the concentration of 8-Br-CAMP is at least 0.1 mM. In some embodiments, the concentration of 8-Br-CAMP is at least 0.2 mM. In some embodiments, the concentration of 8-Br-CAMP is at least 0.5 mM. In some embodiments, the concentration of 8-Br-CAMP is at least 1 mM. In some embodiments, the concentration of 8-Br-CAMP is at least 5 mM. In some embodiments, the concentration of 8-Br-CAMP is at least 10 mM.

In some embodiments, the concentration of dexamethasone is at least 5 nM, 10, nM, 20 nM, 40 nM, 60 nM, 80 nM, 100 nM, 200 nM, 300 nM, 500 nM, 1 mM, 5 mM or 10 mM. In some embodiments, the concentration of dexamethasone is about 5-10 nM, 20-50 nM, 30-90 nM, 50-100 nM, 200-500 nM, 1-3 mM, 2-5 mM or 5-10 mM. In some embodiments, the concentration of dexamethasone is at least 5 nM. In some embodiments, the concentration of dexamethasone is at least 10 nM. In some embodiments, the concentration of dexamethasone is at least 20 nM. In some embodiments, the concentration of dexamethasone is at least 50 nM. In some embodiments, the concentration of dexamethasone is at least 100 nM. In some embodiments, the concentration of dexamethasone is at least 200 nM. In some embodiments, the concentration of dexamethasone is at least 500 nM. In some embodiments, the concentration of dexamethasone is at least 1 mM. In some embodiments, the concentration of dexamethasone is at least 5 mM. In some embodiments, the concentration of dexamethasone is at least 10 mM.

In some embodiments, the immature hepatocytes are cultured for at least 1, 2, 3, 4, 5, 6, 7, 8, 9 or 10 days before increasing the expression of at least one transcription factor disclosed herein. In some embodiments, the immature hepatocytes are cultured for at least 2 days before increasing the expression of the at least one transcription factor. In some embodiments, the immature hepatocytes are cultured for at least 5 days before increasing the expression of the at least one transcription factor. In some embodiments, the immature hepatocytes are cultured for at least 10 days before increasing the expression of the at least one transcription factor.

In some embodiments, the immature hepatocytes are cultured for at least 1, 2, 3, 4, 5, 6, 7, 8, 9 or 10 days after increasing the expression of at least one transcription factor disclosed herein. In some embodiments, the immature hepatocytes are cultured for at least 2 days after increasing the expression of the at least one transcription factor. In some embodiments, the immature hepatocytes are cultured for at least 5 days after increasing the expression of the at least one transcription factor. In some embodiments, the immature hepatocytes are cultured for at least 10 days after increasing the expression of the at least one transcription factor.

In some embodiments, the immature hepatocytes are derived from pluripotent stem cells. Culture media suitable for isolating, expanding and differentiating pluripotent stem cells into immature hepatocytes according to the method described herein include but not limited to high glucose Dulbecco's Modified Eagle's Medium (DMEM), DMEM/F-15, Leibovitz L-15, RPMI 1640, Iscove's modified Dubelcco's media (IMDM), and Opti-MEM SFM (Invitrogen Inc.). Chemically Defined Medium comprises a minimum essential medium such as Iscove's Modified Dulbecco's Medium (IMDM) (Gibco), supplemented with human serum albumin, human Ex Cyte lipoprotein, transferrin, insulin, vitamins, essential and non essential amino acids, sodium pyruvate, glutamine and a mitogen is also suitable. As used herein, a mitogen refers to an agent that stimulates cell division of a cell. An agent can be a chemical, usually some form of a protein that encourages a cell to commence cell division, triggering mitosis. In one embodiment, serum free media (U.S. application Ser. No. 08/464,599 and PCT Publication No. WO96/39487; each of which is incorporated in its entirety herein by reference) and complete media (U.S. Pat. No. 5,486,359, incorporated in its entirety herein by reference), are contemplated for use with the methods described herein. In some embodiments, the culture medium is supplemented with 10% Fetal Bovine Serum (FBS), human autologous serum, human AB serum or platelet rich plasma supplemented with heparin (2 U/ml). Cell cultures may be maintained in a CO2 atmosphere, e.g., 5% to 12%, to maintain pH of the culture fluid, incubated at 37º C, in a humid atmosphere and passaged to maintain a confluence below 85%.

Pluripotent stem cells to be differentiated into immature hepatocytes may be cultured in a medium sufficient to maintain the pluripotency. Culturing of induced pluripotent stem (iPS) cells generated in certain aspects of this invention can use various medium and techniques developed to culture primate pluripotent stem cells, more specially, embryonic stem cells (U.S. Patent Application No. 20070238170 and U.S. Patent Application No. 20030211603; each of which is incorporated in its entirety herein by reference). For example, like human embryonic stem (hES) cells, iPS cells can be maintained in 80% DMEM (Gibco #10829-018 or #11965-092), 20% defined fetal bovine serum (FBS) not heat inactivated, 1% non-essential amino acids, 1 mM L-glutamine, and 0.1 mM .beta.-mercaptoethanol. Alternatively, ES cells can be maintained in serum-free medium, made with 80% Knock-Out DMEM (Gibco #10829-018), 20% serum replacement (Gibco #10828-028), 1% non-essential amino acids, 1 mM L-glutamine, and 0.1 mM .beta.-mercaptoethanol.

In some embodiments, methods of culturing pluripotent stem cells and inducing formation of immature hepatocytes comprise culturing the pluripotent stem cells in a first differentiation media comprising Activin A, a second differentiation media comprising at least one of BMP4 and FGF2, and a third differentiation media comprising HGF, thereby generating the immature hepatocytes.

In some embodiments, the first differentiation media, the second differentiation media and the third differentiation media are each cultured for at least 1, 2, 3, 4, 5, 6, 7, 8, 9 or 10 days. In some embodiments, the first differentiation media is cultured for at least 1, 2, 3, 4, 5, 6, 7, 8, 9 or 10 days. In some embodiments, the second differentiation media is cultured for at least 1, 2, 3, 4, 5, 6, 7, 8, 9 or 10 days. In some embodiments, the third differentiation media is cultured for at least 1, 2, 3, 4, 5, 6, 7, 8, 9 or 10 days.

In some embodiments, the immature hepatocytes are cultured for at least 1, 2, 3, 4, 5, 6, 7, 8, 9 or 10 days before increasing the expression of at least one transcription factor disclosed herein. In some embodiments, the immature hepatocytes are cultured for at least 2 days before increasing the expression of the at least one transcription factor. In some embodiments, the immature hepatocytes are cultured for at least 5 days before increasing the expression of the at least one transcription factor. In some embodiments, the immature hepatocytes are cultured for at least 10 days before increasing the expression of the at least one transcription factor. In some embodiments, the immature hepatocytes are cultured in a culture media comprising hepatocyte growth factor (HGF) before increasing the expression of the at least one transcription factor.

In some embodiments, the immature hepatocytes are cultured for at least 1, 2, 3, 4, 5, 6, 7, 8, 9 or 10 days after increasing the expression of at least one transcription factor disclosed herein. In some embodiments, the immature hepatocytes are cultured for at least 2 days before increasing the expression of the at least one transcription factor. In some embodiments, the immature hepatocytes are cultured for at least 5 days before increasing the expression of the at least one transcription factor. In some embodiments, the immature hepatocytes are cultured for at least 10 days before increasing the expression of the at least one transcription factor. In some embodiments, the immature hepatocytes are cultured in a culture media comprising hepatocyte growth factor (HGF) before increasing the expression of the at least one transcription factor.

In some embodiments, the immature hepatocytes are cultured for at least 1, 2, 3, 4, 5, 6, 7, 8, 9 or 10 days after increasing the expression of at least one transcription factor disclosed herein. In some embodiments, the immature hepatocytes are cultured for at least 2 days after increasing the expression of the at least one transcription factor. In some embodiments, the immature hepatocytes are cultured for at least 5 days after increasing the expression of the at least one transcription factor. In some embodiments, the immature hepatocytes are cultured for at least 10 days after increasing the expression of the at least one transcription factor. In some embodiments, the immature hepatocytes are cultured in a culture media comprising oncostatin-M (OSM) after increasing the expression of the at least one transcription factor.

In order to generate pluripotent stem cell derived immature hepatocytes, in some embodiments, monolayers of pluripotent cells are harvested and plated, e.g., at a density of 2×10⁵ cells/cm². Stage 1 of the differentiation process is initiated by culturing pluripotent stem cells for at least 1, 2 or 3 days in a culture media comprising one or more of Activin A, BMP4, FGF-2, or B27. This is followed by culturing the cells for at least 1, 2, or 3 days in a culture media comprising one or more of Activin A and B27. Stage 2 of the differentiation process comprised culturing the cells derived from stage 1 for at least 1, 2, 3, 4, or 5 days in a culture media comprising one or more of BMP4, FGF-2 or B27. Stage 3 is initiated by culturing the cells derived from stage 2 for at least 1, 2, 3, 4, or 5 days in a culture media comprising one or more of HGF, or B27 (e.g., supplemented with insulin). Finally, stage 4 comprises culturing the cells derived from stage 3 for at least 1, 2, 3, 4, or 5 days in a culture media comprising one or more of oncostatin-M, or SingleQuots (without EGF).

In some embodiments, pluripotent stem cell derived hepatocytes are derived from culture dishes using a four stage, twenty-day protocol, as previously described by Mallanna et al., 2013 (Curr Protoc Stem Cell Biol.; 26:1G.4.1-1G.4.13; which is incorporated by reference in its entirety herein).

### Hepatocyte Characteristics

Cells can be characterized according to a number of phenotypic and/or functional criteria. The criteria include but are not limited to the detection or quantitation of expressed cell markers, enzymatic activity, and the characterization of morphological features and intercellular signaling.

Hepatocytes, e.g., mature hepatocytes embodied in certain aspects of this invention have morphological features characteristic of hepatocytes in the nature, such as primary hepatocytes from organ sources. The features are readily appreciated by those skilled in the art, and include any or all of the following: a polygonal cell shape, a binucleate phenotype, the presence of rough endoplasmic reticulum for synthesis of secreted protein, the presence of Golgi-endoplasmic reticulum lysosome complex for intracellular protein sorting, the presence of peroxisomes and glycogen granules, relatively abundant mitochondria, and the ability to form tight intercellular junctions resulting in creation of bile canalicular spaces. A number of these features present in a single cell are consistent with the cell being a member of the hepatocyte lineage.

Mature hepatocytes of the invention can also be characterized according to whether they express phenotypic markers characteristic of cells of the hepatocyte lineage. Non-limiting examples of cell markers useful in distinguishing mature hepatocytes include albumin, asialoglycoprotein receptor, al-antitrypsin, α-fetoprotein, apoE, arginase I, apoAI, apoAII, apoB, apoCIII, apoCII, aldolase B, alcohol dehydrogenase 1, catalase, CYP3A4, glucokinase, glucose-6-phosphatase, insulin growth factors 1 and 2, IGF-1 receptor, insulin receptor, leptin, liver-specific organic anion transporter (LST-1), L-type fatty acid binding protein, phenylalanine hydroxylase, transferrin, retinol binding protein, erythropoietin (EPO, albumin, al-antitrypsin, asialoglycoprotein receptor, cytokeratin 8 (CK8), cytokeratin 18 (CK18), CYP3A4, fumaryl acetoacetate hydrolase (FAH), glucose-6-phosphates, tyrosine aminotransferase, phosphoenolpyruvate carboxykinase, and tryptophan 2,3-dioxygenase.

Mature hepatocytes may also display a global gene expression profile that is indicative of hepatocyte maturation. Global gene expression profiles may be compared to those of primary hepatocytes or known mature hepatocytes and may be obtained by any method known in the art, for example transcriptomic analysis, microarray analysis, or as described in the Examples. In some embodiments, increasing the expression of the at least one transcription factor shifts the transcriptome of immature hepatocytes towards the transcriptome of mature hepatocytes by at least 1%, 5%, 10%, 20%, 30%, 40%, or 50%. In some embodiments, increasing the expression of the at least one transcription factor shifts the transcriptome of immature hepatocytes towards the transcriptome of mature hepatocytes by at least 1%. In some embodiments, increasing the expression of the at least one transcription factor shifts the transcriptome of immature hepatocytes towards the transcriptome of mature hepatocytes by at least 5%. In some embodiments, increasing the expression of the at least one transcription factor shifts the transcriptome of immature hepatocytes towards the transcriptome of mature hepatocytes by at least 10%. In some embodiments, increasing the expression of the at least one transcription factor shifts the transcriptome of immature hepatocytes towards the transcriptome of mature hepatocytes by at least 20%. In some embodiments, increasing the expression of the at least one transcription factor shifts the transcriptome of immature hepatocytes towards the transcriptome of mature hepatocytes by at least 30%. In some embodiments, increasing the expression of the at least one transcription factor shifts the transcriptome of immature hepatocytes towards the transcriptome of mature hepatocytes by at least 40%. In some embodiments, increasing the expression of the at least one transcription factor shifts the transcriptome of immature hepatocytes towards the transcriptome of mature hepatocytes by at least 50%.

Assessment of the level of expression of such markers in mature hepatocytes can be determined in comparison with other cells, e.g., immature hepatocytes. Positive controls for the markers of mature hepatocytes include adult hepatocytes of the species of interest, e.g., primary human hepatocytes (PHH).

Tissue-specific (e.g., hepatocyte-specific) protein and oligosaccharide determinants listed in this disclosure can be detected using any suitable immunological technique-such as flow immunocytochemistry for cell-surface markers, immunohistochemistry (for example, of fixed cells or tissue sections) for intracellular or cell-surface markers, Western blot analysis of cellular extracts, and enzyme-linked immunoassay, for cellular extracts or products secreted into the medium. Expression of an antigen by a cell is said to be “antibody-detectable” if a significantly detectable amount of antibody will bind to the antigen in a standard immunocytochemistry or flow cytometry assay, optionally after fixation of the cells, and optionally using a labeled secondary antibody or other conjugate (such as a biotin-avidin conjugate) to amplify labeling.

The expression of tissue-specific (e.g., mature hepatocyte-specific) markers can also be detected at the mRNA level by Northern blot analysis, dot-blot hybridization analysis, or by real time polymerase chain reaction (RT-PCR) using sequence-specific primers in standard amplification methods (U.S. Pat. No. 5,843,780). Sequence data for the particular markers listed in this disclosure can be obtained from public databases such as GenBank. Expression at the mRNA level is said to be “detectable” according to one of the assays described in this disclosure if the performance of the assay on cell samples according to standard procedures in a typical controlled experiment results in clearly discernable hybridization or amplification product within a standard time window. Unless otherwise required, expression of a particular marker is indicated if the corresponding mRNA is detectable by RT-PCR. Expression of tissue-specific markers as detected at the protein or mRNA level is considered positive if the level is at least 2-fold, and preferably more than 10- or 50-fold above that of a control cell, such as an undifferentiated pluripotent stem cell, a fibroblast, or other unrelated cell type.

Mature hepatocytes can also be characterized according to whether they display enzymatic activity that is characteristic of mature hepatocytes. For example, assays for glucose-6-phosphatase activity are described by Bublitz (1991); Yasminch et al. (1992); and Ockerman (1968); each of which is incorporated in its entirety herein by reference. Assays for alkaline phosphatase (ALP) and 5-nucleotidase (5′-Nase) in liver cells are described by Shiojiri (1981); which is incorporated in its entirety herein by reference.

In other embodiments, mature hepatocytes of the invention are assayed for activity indicative of xenobiotic detoxification. Cytochrome p450 is a key catalytic component of the mono-oxygenase system. It constitutes a family of hemoproteins responsible for the oxidative metabolism of xenobiotics (administered drugs), and many endogenous compounds. Different cytochromes present characteristic and overlapping substrate specificity. Most of the biotransforming ability is attributable by the cytochromes designated 1A2, 2A6, 2B6, 3A4, 2C 9-11, 2D6, and 2E1 (Gomes-Lechon et al., 1997); which is incorporated in its entirety herein by reference.

A number of assays are known in the art for measuring xenobiotic detoxification by cytochrome p450 enzyme activity. Detoxification by CYP3 A4 is demonstrated using the P450-Glo™ CYP3A4 DMSO-tolerance assay (Luciferin-PPXE) and the P450-Glo™ CYP3A4 cell-based/biochemical assay (Luciferin-PFBE) (Promega Inc, #V8911 and #V8901). Detoxification by CYP1A1 and or CYP1B1 is demonstrated using the P450-Glo™ assay (Luciferin-CEE) (Promega Inc., #V8762). Detoxification by CYP1A2 and or CYP4A is demonstrated using the P450-Glo™ assay (Luciferin-ME) (Promega Inc., #V8772) Detoxification by CYP2C9 is demonstrated using the P450-Glo™ CYP2C9 assay (Luciferin-H) (Promega Inc., #V8791).

In another aspect, the biological function of a mature hepatocyte of the invention is evaluated, for example, by analysing glycogen storage. Glycogen storage is characterized by assaying Periodic Acid Schiff (PAS) functional staining for glycogen granules. The cells are first oxidized by periodic acid. The oxidative process results in the formation of aldehyde groupings through carbon-to-carbon bond cleavage. Free hydroxyl groups should be present for oxidation to take place. Oxidation is completed when it reaches the aldehyde stage. The aldehyde groups are detected by the Schiff reagent. A colorless, unstable dialdehyde compound is formed and then transformed to the colored final product by restoration of the quinoid chromophoric grouping (Thompson, 1966; Shechan and Hrapchak, 1987; each of which is incorporated in its entirety herein by reference). PAS staining can be performed according the protocol described at world wide web at jhu.edu/˜iic/PDF jrotocols/LM/Glycogen Staining pdf and library.med.utah.edu/WebPath/HISTHTML/MANUALS/PAS.PDF with some modifications for an in vitro culture of hepatocyte-like cells. One of ordinary skill in the art should be able to make the appropriate modifications.

In another aspect, a mature hepatocyte of the invention is characterized for urea production. Urea production can be assayed colorimetrically using kits from Sigma Diagnostic (Miyoshi et al, 1998; which is incorporated in its entirety herein by reference) based on the biochemical reaction of urease reduction to urea and ammonia and the subsequent reaction with 2-oxoglutarate to form glutamate and NAD.

In another aspect, bile secretion is analyzed. Biliary secretion can be determined by fluorescein diacetate time lapse assay. Briefly, monolayer cultures of cells, e.g., mature hepatocytes, are rinsed with phosphate buffered saline (PBS) three times and incubated with serum-free hepatocyte growth media supplemented with doxycycline and fluorescein diacetate (20 μg/ml) (Sigma-Aldrich) at 37° C. for 35 minutes. The cells are washed with PBS three times and fluorescence imaging is carried out. Fluorescein diacetate is a non fluorescent precursor of fluorescein. The image is evaluated to determine that the compound had been taken up and metabolized in the hepatocyte-like cell to fluorescein. In some embodiments, the compound is secreted into intercellular clefts of the monolayer of cells. Alternatively, bile secretion is determined by a method using sodium fluorescein described by Gebhart and Wang (1982); which is incorporated in its entirety herein by reference.

In yet another aspect, lipid synthesis is analyzed. Lipid synthesis in the mature hepatcytes can be determined by oil red O staining Oil Red O (Solvent Red 27, Sudan Red 5B, C.I. 26125, C26H24N4O) is a lysochrome (fat-soluble dye) diazo dye used for staining of neutral triglycerides and lipids on frozen sections and some lipoproteins on paraffin sections. It has the appearance of a red powder with maximum absorption at 518(359) nm. Oil Red O is one of the dyes used for Sudan staining Similar dyes include Sudan III, Sudan IV, and Sudan Black B. The staining has to be performed on fresh samples and/or formalin fixed samples. Hepatocyte-like cells are cultured on microscope slides, rinsed in PBS three times, the slides are air dried for 30-60 minutes at room temperature, fixed in ice cold 10% formalin for 5-10 minutes, and then rinse immediately in 3 changes of distilled water. The slide is then placed in absolute propylene glycol for 2-5 minutes to avoid carrying water into Oil Red O and stained in pre-warmed Oil Red O solution for 8 minutes in 600° C. oven. The slide is then placed in 85% propylene glycol solution for 2-5 minutes and rinsed in 2 changes of distilled water. Oil red O staining can also be performed according the protocol described at library.med.utah.edu/WebPath/HISTHTML/MANUALS/OILRED.PDF with some modifications for an in vitro culture of hepatocyte-like cell by one of ordinary skill in the art.

In still another aspect, the mature hepatcytes are assayed for glycogen synthesis. Glycogen assays are well known to one of ordinary skill in the art, for example, in Passonneau and Lauderdale (1974). Alternatively, commercial glycogen assays can be used, for example, from BioVision, Inc. catalog #K646-100.

Mature hepatocytes can also be evaluated by their ability to store glycogen. A suitable assay uses Periodic Acid Schiff (PAS) stain, which does not react with mono- and disaccharides, but stains long-chain polymers such as glycogen and dextran. PAS reaction provides quantitative estimations of complex carbohydrates as well as soluble and membrane-bound carbohydrate compounds. Kirkeby et al. (1992) describe a quantitative PAS assay of carbohydrate compounds and detergents. van der Laarse et al. (1992) describe a microdensitometric histochemical assay for glycogen using the PAS reaction. Evidence of glycogen storage is determined if the cells are PAS-positive at a level that is at least 2-fold, and preferably more than 10-fold above that of a control cell, such as a fibroblast The cells can also be characterized by karyotyping according to standard methods.

Assays are also available for enzymes involved in the conjugation, metabolism, or detoxification of small molecule drugs. For example, mature hepatocytes can be characterized by an ability to conjugate bilirubin, bile acids, and small molecule drugs, for excretion through the urinary or biliary tract. Cells are contacted with a suitable substrate, incubated for a suitable period, and then the medium is analyzed (by GCMS or other suitable technique) to determine whether a conjugation product has been formed. Drug metabolizing enzyme activities include de-ethylation, dealkylation, hydroxylation, demethylation, oxidation, glucuroconjugation, sulfoconjugation, glutathione conjugation, and N-acetyl transferase activity (A. Guillouzo, pp 411-431 in In vitro Methods in Pharmaceutical Research, Academic Press, 1997; which is incorporated in its entirety herein by reference). Assays include phenacetin de-ethylation, procainamide N-acetylation, paracetamol sulfoconjugation, and paracetamol glucuronidation (Chesne et al., 1988; which is incorporated in its entirety herein by reference).

A further feature of certain cell populations, e.g., mature hepatcytes of this invention is that they are susceptible under appropriate circumstances to pathogenic agents that are tropic for primate liver cells. Such agents include hepatitis A, B, C, and delta, Epstein-Barr virus (EBV), cytomegalovirus (CMV), tuberculosis, and malaria. For example, infectivity by hepatitis B can be determined by combining cultured mature hepatocytes with a source of infectious hepatitis B particles (such as serum from a human HBV carrier). The liver cells can then be tested for synthesis of viral core antigen (HBcAg) by immunohistochemistry or RT-PCR.

In still another aspect, the mature hepatocytes can be assessed for their ability to engraft and/or exhibit long-term survival in a subject. In an embodiment, in order to determine whether the hepatocytes survive and maintain their phenotype in vivo, hepatocytes are administered to an animal (such as SCID mice) at a site amenable for further observation, such as under the kidney capsule, into the spleen, or into a liver lobule. Tissues are harvested after a period of a few days to several weeks or more, to assess the presence and phenotype of the administered cells, e.g., by immunohistochemistry or ELISA using human-specific antibody, or by RT-PCR analysis. Suitable markers for assessing gene expression at the mRNA or protein level are provided in this disclosure. Effects on hepatic function can also be determined by evaluating markers expressed in liver tissue, e.g., cytochrome p450 activity, and blood indicators, such as alkaline phosphatase activity, bilirubin conjugation, and prothrombin time.

Assays for determining the ability of mature hepatocytes to engraft and/or exhibit long-term survival in a subject are described in, for example, U.S. Pat. No. 9,260,722; and US Publication No. 2020/0216823; each of which is incorporated in its entirety herein by reference.

In some embodiments, the mature hepatocytes engraft into a target tissue of the subject. In some embodiments, the mature hepatocytes comprise a population of mature hepatocytes, wherein at least 0.1%, 0.2%, 0.5%, 1%, 2%, 3%, 4%, 5%, 10%, 20%, 30%, 40%, 50%, 60%, 70%, 80%, 90%, or 100% of the mature hepatocytes engraft into the target tissue of the subject. In some embodiments, the target tissue is a liver.

The skilled artisan will readily appreciate that an advantage of mature hepatocytes is that they will be essentially free of other cell types that typically contaminate primary hepatocyte cultures isolated from adult or fetal liver tissue. Mature hepatocytes provided according to certain aspects of this invention can have a number of the features of the stage of cell they are intended to represent. The more of these features that are present in a particular cell, the more it can be characterized as a cell of the hepatocyte lineage. Cells having at least 2, 3, 5, 7, or 9 of these features are increasingly more preferred. In reference to a particular cell population as may be present in a culture vessel or a preparation for administration, uniformity between cells in the expression of these features is often advantageous. In this circumstance, populations in which at least about 10%, 20%, 30%, 40%, 60%, 80%, 90%, 95%, 98%, 99%, or 100% of the cells have the desired features are increasingly more preferred.

Other desirable features of hepatocytes provided in certain aspects of this invention are an ability to act as target cells in drug screening assays, and an ability to reconstitute liver function, both in vivo, and as part of an extracorporeal device. These features are described further in sections that follow.

### II. Cells and Compositions of the Invention

A further aspect of the invention provides a composition comprising a population of hepatocytes, for example, produced according to any of the methods described herein. In some embodiments, the composition comprises an enriched, purified or isolated population of hepatocytes, for example, produced according to any of the methods described herein. The enriched, purified or isolated population of hepatocytes can be single cell suspensions, aggregates, chimeric aggregates, and/or structures, including branched structures and/or cysts.

In some embodiments, the population of hepatocytes comprise increased expression levels of at least one transcription factor selected from the group consisting of Nuclear Factor I X (NFIX) and Nuclear Factor I C (NFIC), relative to endogenous expression levels of the transcription factor in the population of hepatocytes.

In some embodiments, the increased expression of NFIX comprises an increase of at least 0.1-fold, 0.2-fold, 0.5-fold, 1-fold, 2-fold, 5-fold, 10-fold, 20-fold, 50-fold, 100-fold, 200-fold, 500-fold, 1000-fold, or 10,000-fold relative to endogenous expression levels of NFIX in the population of hepatocytes. In some embodiments, the increased expression of NFIX comprises an increase of at least 0.1-fold relative to endogenous expression levels of NFIX in the population of hepatocytes. In some embodiments, the increased expression of NFIX comprises an increase of at least 0.2-fold relative to endogenous expression levels of NFIX in the population of hepatocytes. In some embodiments, the increased expression of NFIX comprises an increase of at least 0.5-fold relative to endogenous expression levels of NFIX in the population of hepatocytes. In some embodiments, the increased expression of NFIX comprises an increase of at least 1-fold relative to endogenous expression levels of NFIX in the population of hepatocytes. In some embodiments, the increased expression of NFIX comprises an increase of at least 2-fold relative to endogenous expression levels of NFIX in the population of hepatocytes. In some embodiments, the increased expression of NFIX comprises an increase of at least 5-fold relative to endogenous expression levels of NFIX in the population of hepatocytes. In some embodiments, the increased expression of NFIX comprises an increase of at least 10-fold relative to endogenous expression levels of NFIX in the population of hepatocytes. In some embodiments, the increased expression of NFIX comprises an increase of at least 20-fold relative to endogenous expression levels of NFIX in the population of hepatocytes. In some embodiments, the increased expression of NFIX comprises an increase of at least 50-fold relative to endogenous expression levels of NFIX in the population of hepatocytes. In some embodiments, the increased expression of NFIX comprises an increase of at least 100-fold relative to endogenous expression levels of NFIX in the population of hepatocytes. In some embodiments, the increased expression of NFIX comprises an increase of at least 200-fold relative to endogenous expression levels of NFIX in the population of hepatocytes. In some embodiments, the increased expression of NFIX comprises an increase of at least 500-fold relative to endogenous expression levels of NFIX in the population of hepatocytes. In some embodiments, the increased expression of NFIX comprises an increase of at least 1,000-fold relative to endogenous expression levels of NFIX in the population of hepatocytes. In some embodiments, the increased expression of NFIX comprises an increase of at least 10,000-fold relative to endogenous expression levels of NFIX in the population of hepatocytes.

In some embodiments, the increased expression of NFIC comprises an increase of at least 0.1-fold, 0.2-fold, 0.5-fold, 1-fold, 2-fold, 5-fold, 10-fold, 20-fold, 50-fold, 100-fold, 200-fold, 500-fold, 1000-fold, or 10,000-fold relative to endogenous expression levels of NFIC in the population of hepatocytes. In some embodiments, the increased expression of NFIC comprises an increase of at least 0.1-fold relative to endogenous expression levels of NFIC in the population of hepatocytes. In some embodiments, the increased expression of NFIC comprises an increase of at least 0.2-fold relative to endogenous expression levels of NFIC in the population of hepatocytes. In some embodiments, the increased expression of NFIC comprises an increase of at least 0.5-fold relative to endogenous expression levels of NFIC in the population of hepatocytes. In some embodiments, the increased expression of NFIC comprises an increase of at least 1-fold relative to endogenous expression levels of NFIC in the population of hepatocytes. In some embodiments, the increased expression of NFIC comprises an increase of at least 2-fold relative to endogenous expression levels of NFIC in the population of hepatocytes. In some embodiments, the increased expression of NFIC comprises an increase of at least 5-fold relative to endogenous expression levels of NFIC in the population of hepatocytes. In some embodiments, the increased expression of NFIC comprises an increase of at least 10-fold relative to endogenous expression levels of NFIC in the population of hepatocytes. In some embodiments, the increased expression of NFIC comprises an increase of at least 20-fold relative to endogenous expression levels of NFIC in the population of hepatocytes. In some embodiments, the increased expression of NFIC comprises an increase of at least 50-fold relative to endogenous expression levels of NFIC in the population of hepatocytes. In some embodiments, the increased expression of NFIC comprises an increase of at least 100-fold relative to endogenous expression levels of NFIC in the population of hepatocytes. In some embodiments, the increased expression of NFIC comprises an increase of at least 200-fold relative to endogenous expression levels of NFIC in the population of hepatocytes. In some embodiments, the increased expression of NFIC comprises an increase of at least 500-fold relative to endogenous expression levels of NFIC in the population of hepatocytes. In some embodiments, the increased expression of NFIC comprises an increase of at least 1,000-fold relative to endogenous expression levels of NFIC in the population of hepatocytes. In some embodiments, the increased expression of NFIC comprises an increase of at least 10,000-fold relative to endogenous expression levels of NFIC in the population of hepatocytes.

In some embodiments, the population of hepatocytes further comprise increased expression levels of one or more transcription factors selected from the group consisting of RORC, NR0B2, ESR1, THRSP, TBX15, HLF, ATOH8, NR112, CUX2, ZNF662, TSHZ2, ATF5, NFIA, NFIB, NPAS2, FOS, ONECUT2, PROX1, NR1H4, MLXIPL, ETV1, AR, CEBPB, NR1D1, HEY2, ARID3C, KLF9, and DMRTA1 relative to endogenous expression levels of the one or more transcription factors in the population of hepatocytes. In some embodiments, the one or more transcription factor is RORC. In some embodiments, the one or more transcription factor is NR0B2. In some embodiments, the one or more transcription factor is ESR1. In some embodiments, the one or more transcription factor is THRSP. In some embodiments, the one or more transcription factor is TBX15. In some embodiments, the one or more transcription factor is HLF. In some embodiments, the one or more transcription factor is ATOH8. In some embodiments, the one or more transcription factor is NR112. In some embodiments, the one or more transcription factor is CUX2. In some embodiments, the one or more transcription factor is ZNF662. In some embodiments, the one or more transcription factor is TSHZ2. In some embodiments, the one or more transcription factor is ATF5. In some embodiments, the one or more transcription factor is NFIA. In some embodiments, the one or more transcription factor is NFIB. In some embodiments, the one or more transcription factor is NPAS2. In some embodiments, the one or more transcription factor is FOS. In some embodiments, the one or more transcription factor is ONECUT2. In some embodiments, the one or more transcription factor is PROX1. In some embodiments, the one or more transcription factor is NR1H4. In some embodiments, the one or more transcription factor is MLXIPL. In some embodiments, the one or more transcription factor is ETV1. In some embodiments, the one or more transcription factor is AR. In some embodiments, the one or more transcription factor is CEBPB. In some embodiments, the one or more transcription factor is NR1D1. In some embodiments, the one or more transcription factor is HEY2. In some embodiments, the one or more transcription factor is ARID3C. In some embodiments, the one or more transcription factor is KLF9. In some embodiments, the one or more transcription factor is DMRTA1.

In some embodiments, the population of hepatocytes is a population of immature hepatocytes. In some embodiments, the population of hepatocytes is a population of mature hepatocytes. In some embodiments, the population of hepatocytes comprises both mature and immature hepatocytes.

In some embodiments, the mature hepatocytes exhibit an increased expression of albumin (ALB), cytochrome P450 enzyme 1A2 (CYP1A2), cytochrome P450 enzyme 3A4 (CYP3A4), tyrosine aminotransferase (TAT), and/or UDP-glucuronosyltransferase 1A-1 (UGT1A1) relative to immature hepatocytes.

In some embodiments, the increased expression of CYP1A2 comprises an increase of at least 2-fold, 5-fold, 10-fold, 50-fold, 100-fold, 200-fold, 500-fold, 1,000-fold, 2,000-fold, 5,000-fold, or 10,000-fold relative to immature hepatocytes. In some embodiments, the increased expression of CYP3A4 comprises an increase of at least 2-fold, 5-fold, 10-fold, 50-fold, 100-fold, 200-fold, 500-fold, 1,000-fold, 2,000-fold, 5,000-fold, or 10,000-fold relative to immature hepatocytes. In some embodiments, the increased expression of TAT comprises an increase of at least 2-fold, 5-fold, 10-fold, 50-fold, 100-fold, 200-fold, 500-fold, 1,000-fold, 2,000-fold, 5,000-fold, or 10,000-fold relative to immature hepatocytes. In some embodiments, the increased expression of UGT1A1 comprises an increase of at least 2-fold, 5-fold, 10-fold, 50-fold, 100-fold, 500-fold, 1,000-fold, 2,000-fold, 5,000-fold, or 10,000-fold relative to immature hepatocytes.

In some embodiments, the mature hepatocytes exhibit a decreased expression of alpha fetoprotein (AFP) relative to immature hepatocytes. In some embodiments, the decreased expression of AFP comprises a decrease of at least 0.1-fold, 0.2-fold, 0.5-fold, 1-fold, 2-fold, 3-fold, or 4-fold relative to immature hepatocytes.

In some embodiments, the mature hepatocytes exhibit an increased secretion of albumin, a decreased secretion of AFP, and/or an increased activity of CYP1A2, relative to immature hepatocytes. In some embodiments, the increased secretion of ALB comprises an increase of at least 5%, 10%, 15%, 20% or 25% relative to immature hepatocytes. In some embodiments, the decreased secretion of AFP comprises a decrease of at least 5%, 10%, 20%, 40%, or 60% relative to immature hepatocytes. In some embodiments, the increased activity of CYP1A2 comprises an increase of at least 2-fold, 5-fold, 10-fold, 50-fold, 100-fold, 200-fold, or 400-fold relative to immature hepatocytes.

In some embodiments, the composition of a population of hepatocytes comprises about 1×10⁶ hepatocytes to about 1×10¹² hepatocytes. In some embodiments, the composition of a population of hepatocytes comprises at least 1×10⁵, 1×10⁶, 1×10⁷, 1×10⁸, 1×10⁹, 1×10¹⁰, 1×10¹¹, or 1×10¹² hepatocytes.

Also provided herein are pharmaceutical compositions and formulations comprising hepatocytes, e.g., mature or immature hepatocytes, and a pharmaceutically acceptable carrier.

In some embodiments, the pharmaceutical composition comprises a dose ranging from about 1×10⁶ hepatocytes to about 1×10¹² hepatocytes. In some embodiments, the dose is about 1×10⁵, 1×10⁶, 1×10⁷, 1×10⁸, 1×10⁹, 1×10¹⁰, 1×10¹¹, or 1×10¹² hepatocytes. In some embodiments, a pharmaceutical composition comprises a dose ranging from about 1×10⁶ hepatocytes to about 1×10¹² hepatocytes.

A further aspect of the invention provides a composition comprising a population of pluripotent stem cells comprising an expression vector, wherein the expression vector comprises a nucleic acid encoding at least one transcription factor of the disclosure.

In some embodiments, the transcription factor is NFIX. In some embodiments, the transcription factor is NFIC. In some embodiments, the transcription factor is NFIX and NFIC.

In some embodiments, the population of pluripotent stem cells further comprise an expression vector comprising a nucleic acid encoding one or more transcription factors selected from the group consisting of RORC, NR0B2, ESR1, THRSP, TBX15, HLF, ATOH8, NR112, CUX2, ZNF662, TSHZ2, ATF5, NFIA, NFIB, NPAS2, FOS, ONECUT2, PROX1, NR1H4, MLXIPL, ETV1, AR, CEBPB, NR1D1, HEY2, ARID3C, KLF9, and DMRTA1. In some embodiments, the one or more transcription factor is RORC. In some embodiments, the one or more transcription factor is NR0B2. In some embodiments, the one or more transcription factor is ESR1. In some embodiments, the one or more transcription factor is THRSP. In some embodiments, the one or more transcription factor is TBX15. In some embodiments, the one or more transcription factor is HLF. In some embodiments, the one or more transcription factor is ATOH8. In some embodiments, the one or more transcription factor is NR112. In some embodiments, the one or more transcription factor is CUX2. In some embodiments, the one or more transcription factor is ZNF662. In some embodiments, the one or more transcription factor is TSHZ2. In some embodiments, the one or more transcription factor is ATF5. In some embodiments, the one or more transcription factor is NFIA. In some embodiments, the one or more transcription factor is NFIB. In some embodiments, the one or more transcription factor is NPAS2. In some embodiments, the one or more transcription factor is FOS. In some embodiments, the one or more transcription factor is ONECUT2. In some embodiments, the one or more transcription factor is PROX1. In some embodiments, the one or more transcription factor is NR1H4. In some embodiments, the one or more transcription factor is MLXIPL. In some embodiments, the one or more transcription factor is ETV1. In some embodiments, the one or more transcription factor is AR. In some embodiments, the one or more transcription factor is CEBPB. In some embodiments, the one or more transcription factor is NR1D1. In some embodiments, the one or more transcription factor is HEY2. In some embodiments, the one or more transcription factor is ARID3C. In some embodiments, the one or more transcription factor is KLF9. In some embodiments, the one or more transcription factor is DMRTA1.

In some embodiments, the composition comprising a population of pluripotent stem cells comprises about 1×10⁶ pluripotent stem cells to about 1×10¹² pluripotent stem cells. In some embodiments, the composition comprising a population of pluripotent stem cells comprises at least 1×10⁵, 1×10⁶, 1×10⁷, 1×10⁸, 1×10⁹, 1×10¹⁰, 1×10¹¹, or 1×10¹² pluripotent stem cells.

In some embodiments, the pluripotent stem cells are embryonic stem cells. In some embodiments, the pluripotent stem cells are induced pluripotent stem cells.

A further aspect of the invention provides a composition comprising a population of immature hepatocytes comprising an expression vector, wherein the expression vector comprises a nucleic acid encoding at least one transcription factor of the disclosure.

In some embodiments, the transcription factor is NFIX. In some embodiments, the transcription factor is NFIC. In some embodiments, the transcription factor is NFIX and NFIC.

In some embodiments, the population of immature hepatocytes further comprise an expression vector comprising a nucleic acid encoding one or more transcription factors selected from the group consisting of RORC, NR0B2, ESR1, THRSP, TBX15, HLF, ATOH8, NR112, CUX2, ZNF662, TSHZ2, ATF5, NFIA, NFIB, NPAS2, FOS, ONECUT2, PROX1, NR1H4, MLXIPL, ETV1, AR, CEBPB, NR1D1, HEY2, ARID3C, KLF9, and DMRTA1. In some embodiments, the one or more transcription factor is RORC. In some embodiments, the one or more transcription factor is NR0B2. In some embodiments, the one or more transcription factor is ESR1. In some embodiments, the one or more transcription factor is THRSP. In some embodiments, the one or more transcription factor is TBX15. In some embodiments, the one or more transcription factor is HLF. In some embodiments, the one or more transcription factor is ATOH8. In some embodiments, the one or more transcription factor is NR112. In some embodiments, the one or more transcription factor is CUX2. In some embodiments, the one or more transcription factor is ZNF662. In some embodiments, the one or more transcription factor is TSHZ2. In some embodiments, the one or more transcription factor is ATF5. In some embodiments, the one or more transcription factor is NFIA. In some embodiments, the one or more transcription factor is NFIB. In some embodiments, the one or more transcription factor is NPAS2. In some embodiments, the one or more transcription factor is FOS. In some embodiments, the one or more transcription factor is ONECUT2. In some embodiments, the one or more transcription factor is PROX1. In some embodiments, the one or more transcription factor is NR1H4. In some embodiments, the one or more transcription factor is MLXIPL. In some embodiments, the one or more transcription factor is ETV1. In some embodiments, the one or more transcription factor is AR. In some embodiments, the one or more transcription factor is CEBPB. In some embodiments, the one or more transcription factor is NR1D1. In some embodiments, the one or more transcription factor is HEY2. In some embodiments, the one or more transcription factor is ARID3C. In some embodiments, the one or more transcription factor is KLF9. In some embodiments, the one or more transcription factor is DMRTA1.

In some embodiments, the composition comprising a population of immature hepatocytes comprises about 1×10⁶ immature hepatocytes to about 1×10¹² immature hepatocytes. In some embodiments, the composition comprising a population of immature hepatocytes comprises at least 1×10⁵, 1×10⁶, 1×10⁷, 1×10⁸, 1×10⁹, 1×10¹⁰, 1×10¹¹, or 1×10¹² immature hepatocytes.

Also provided herein are pharmaceutical compositions and formulations comprising immature hepatocytes, and a pharmaceutically acceptable carrier.

In some embodiments, the pharmaceutical composition comprises a dose ranging from about 1×10⁶ immature hepatocytes to about 1×10¹² immature hepatocytes. In some embodiments, the dose is about 1×10⁵, 1×10⁶, 1×10⁷, 1×10⁸, 1×10⁹, 1×10¹⁰, 1×10¹¹, or 1×10¹² immature hepatocytes. In some embodiments, a pharmaceutical composition comprises a dose ranging from about 1×10⁶ immature hepatocytes to about 1×10¹² immature hepatocytes.

Pharmaceutical compositions and formulations as described herein can be prepared by mixing the cells of the disclosure, e.g., mature hepatocytes with one or more optional pharmaceutically acceptable carriers (Remington's Pharmaceutical Sciences 22nd edition, 2012; incorporated in its entirety herein reference), in the form of aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968; each of which is incorporated in its entirety herein reference. In one aspect, a sHASEGP is combined with one or more additional glycosaminoglycanases such as chondroitinases.

In certain embodiments, the composition and pharmaceutical composition comprising hepatocytes comprise a substantially purified population of hepatocytes. For example, the composition of hepatocytes may contain less than 25%, 20%, 15%, 10%, 9%, 8%, 7%, 6%, 5%, 4%, 3%, 2%, or less than 1% of cells other than hepatocytes. In some embodiments, the composition of hepatocytes contains less than 25%, 20%, 15%, 10%, 9%, 8%, 7%, 6%, 5%, 4%, 3%, 2%, or less than 1% of pluripotent stem cells. In another embodiment, the composition of hepatocytes is devoid of or is undetectable for pluripotent stem cells. In some embodiments, the composition comprising a substantially purified population of hepatocytes is one in which the hepatocytes comprise at least about 75% of the cells in the composition. In other embodiments, a substantially purified population of hepatocytes is one in which the hepatocytes comprise at least about 80%, 85%, 90%, 91%, 92%, 93%, 94%, 95%, 96%, 97%, 97.5%, 98%, 99%, or even greater than 99% of the cells in the population. In any of the embodiments, the hepatocytes may be mature hepatocytes.

In another embodiment, the composition and pharmaceutical composition comprising hepatocytes may comprise cells other than hepatocytes that may be useful in augmenting or complementing the function of the hepatocytes, including, but not limited to mesenchymal stem cells, endothelial cells, cholangiocytes, stellate cells, and/or Kupffer cells. In other embodiments, the composition and pharmaceutical composition comprising hepatocytes comprise organoids, which are three-dimensional structures of cells that are often capable of self-organization and provide an environment for high cell-extracellular matrix and cell-cell interactions in vivo. See e.g., Olgasi et al., International Journal of Molecular Sciences 21:6215 (2020), which is incorporated in its entirety herein by reference. The organoids comprise hepatocytes and may further comprise other cells, such as mesenchymal stem cells, endothelial cells, cholangiocytes, stellate cells, and Kupffer cells. In any of the embodiments, the hepatocytes may be mature hepatocytes.

### III. Methods of Use of Hepatocytes

Hepatocytes and pharmaceutical compositions produced by the methods described herein may be used for cell-based treatments in which hepatocytes are needed or would improve treatment. Methods of using hepatocytes provided by the present invention for treating various conditions that may benefit from hepatocyte-based therapies are described herein. The particular treatment regimen, route of administration, and any adjuvant therapy will be tailored based on the particular condition, the severity of the condition, and the patient's overall health. Additionally, in certain embodiments, administration of hepatocytes may be effective to fully restore loss of liver function or other symptoms. In other embodiments, administration of hepatocytes may be effective to reduce the severity of the symptoms and/or to prevent further degeneration in the patient's condition. The invention contemplates that administration of a composition comprising hepatocytes can be used to treat (including reducing the severity of the symptoms, in whole or in part) any of the conditions described herein.

The invention contemplates that hepatocytes, including compositions comprising hepatocytes, derived using any of the methods described herein can be used in the treatment of any of the indications described herein. Further, the invention contemplates that any of the compositions comprising hepatocytes described herein can be used in the treatment of any of the indications described herein. In another embodiment, the hepatocytes of the invention may be administered with other therapeutic cells or agents. The hepatocytes may be administered simultaneously in a combined or separate formulation, or sequentially.

In an embodiment, the present invention provides a method of treating a disease or disorder selected from the group consisting of fulminant hepatic failure due to any cause, viral hepatitis, drug-induced liver injury, cirrhosis, inherited hepatic insufficiency (such as Wilson's disease, Gilbert's syndrome, or al-antitrypsin deficiency), hepatobiliary carcinoma, autoimmune liver disease (such as autoimmune chronic hepatitis or primary biliary cirrhosis), urea cycle disorder, factor VII deficiency, glycogen storage disease type 1, infantile Refsum's disease, phenylketonuria, severe infantile oxalosis, cirrhosis, liver injury, acute liver failure, hepatobiliary carcinoma, hepatocellular carcinoma, genetic cholestasis (PFIC and alagille syndrome), hereditary hemochromatosis, tyrosinemia type 1, argininosuccinic aciduria (ASL), Crigler-Najjar syndrome, familial amyloid polyneuropathy, atypical haemolytic uremic syndrome-1, primary hyperoxaluria type 1, maple syrup urine disease (MSUD), acute intermittent porphyria, coagulation defects, GSD type Ia (in metabolic control), homozygous familial hypercholesterolemia, organic acidurias, and any other condition that results in impaired hepatic function.

The hepatocytes provided by methods and compositions of the invention can also be used in a variety of applications. These include but are not limited to transplantation or implantation of the hepatocytes in vivo; screening for cytotoxic compounds, carcinogens, mutagens growth/regulatory factors, or pharmaceutical compounds in vitro; elucidating the mechanism of liver diseases and infections; studying the mechanism by which drugs and/or growth factors operate; diagnosing and monitoring cancer in a patient; gene therapy; and the production of biologically active products. In some embodiments, the hepatocytes comprise mature hepatocytes, immature hepatocytes or a combination thereof.

### Test Compound Screening

The hepatocytes of the invention can be used to screen for factors (such as solvents, small molecule drugs, peptides, and polynucleotides) or environmental conditions (such as culture conditions or manipulation) that affect the characteristics of hepatocytes provided herein.

In some applications, stem cells (differentiated or undifferentiated) are used to screen factors that promote maturation of cells along the hepatocyte lineage, or promote proliferation and maintenance of such cells in long-term culture. For example, candidate hepatocyte maturation factors or growth factors are tested by adding them to stem cells in different wells, and then determining any phenotypic change that results, according to desirable criteria for further culture and use of the cells.

Particular screening applications of this invention relate to the testing of pharmaceutical compounds in drug research, for example, as described in In vitro Methods in Pharmaceutical Research, Academic Press, 1997, and U.S. Pat. No. 5,030,015; each of which is incorporated in its entirety herein by reference. In certain aspects of the invention, hepatocytes play the role of test cells for standard drug screening and toxicity assays, as have been previously performed on hepatocyte cell lines or primary hepatocytes in short-term culture. Assessment of the activity of candidate pharmaceutical compounds generally involves combining the hepatocytes provided in certain aspects of this invention with the candidate compound, determining any change in the morphology, marker phenotype, or metabolic activity of the cells that is attributable to the compound (compared with untreated cells or cells treated with an inert compound), and then correlating the effect of the compound with the observed change. The screening may be done either because the compound is designed to have a pharmacological effect on liver cells, or because a compound designed to have effects elsewhere may have unintended hepatic side effects. Two or more drugs can be tested in combination (by combining with the cells either simultaneously or sequentially), to detect possible drug-drug interaction effects.

In some applications, compounds are screened initially for potential hepatotoxicity (Castell et al., 1997; incorporated in its entirety herein by reference). Cytotoxicity can be determined in the first instance by the effect on cell viability, survival, morphology, and leakage of enzymes into the culture medium. More detailed analysis is conducted to determine whether compounds affect cell function (such as gluconeogenesis, ureogenesis, and plasma protein synthesis) without causing toxicity. Lactate dehydrogenase (LDH) is a good marker because the hepatic isoenzyme (type V) is stable in culture conditions, allowing reproducible measurements in culture supernatants after 12-24 hours of incubation. Leakage of enzymes such as mitochondrial glutamate oxaloacetate transaminase and glutamate pyruvate transaminase can also be used. Gomez-Lechon et al. (1996), which is incorporated in its entirety herein by reference, describes a microassay for measuring glycogen, which can be used to measure the effect of pharmaceutical compounds on hepatocyte gluconeogenesis.

Other current methods to evaluate hepatotoxicity include determination of the synthesis and secretion of albumin, cholesterol, and lipoproteins; transport of conjugated bile acids and bilirubin; ureagenesis; cytochrome p450 levels and activities; glutathione levels; release of α-glutathione s-transferase; ATP, ADP, and AMP metabolism; intracellular K+ and Ca2+ concentrations; the release of nuclear matrix proteins or oligonucleosomes; and induction of apoptosis (indicated by cell rounding, condensation of chromatin, and nuclear fragmentation). DNA synthesis can be measured as [3H]-thymidine or BrdU incorporation. Effects of a drug on DNA synthesis or structure can be determined by measuring DNA synthesis or repair. [3H]-thymidine or BrdU incorporation, especially at unscheduled times in the cell cycle, or above the level required for cell replication, is consistent with a drug effect. Unwanted effects can also include unusual rates of sister chromatid exchange, determined by metaphase spread.

### Liver Therapy and Transplantation

The invention also provides the use of hepatocytes described herein to restore a degree of liver function to a subject in need thereof due to, for example, an acute, chronic, or inherited impairment of liver function.

To determine the suitability of hepatocytes provided herein for therapeutic applications, the cells can first be tested in a suitable animal model. At one level, cells are assessed for their ability to survive and maintain their phenotype in vivo. Hepatocytes provided herein are administered to immunodeficient animals (such as SCID mice, or animals rendered immunodeficient chemically or by irradiation) at a site amenable for further observation, such as under the kidney capsule, into the spleen, or into a liver lobule. Tissues are harvested after a period of a few days to several weeks or more, and assessed. This can be performed by providing the administered cells with a detectable label (such as green fluorescent protein, or β-galactosidase); or by measuring a constitutive marker specific for the administered cells. Where hepatocytes provided herein are being tested in a rodent model, the presence and phenotype of the administered cells can be assessed by immunohistochemistry or ELISA using human-specific antibody, or by RT-PCR analysis using primers and hybridization conditions that cause amplification to be specific for human polynucleotide sequences. Suitable markers for assessing gene expression at the mRNA or protein level are provided herein. General descriptions for determining the fate of hepatocyte-like cells in animal models are described by, for example, Grompe et al. (1999); Peeters et al., (1997); and Ohashi et al. (2000); each of which is incorporated in its entirety herein by reference.

At another level, hepatocytes provided herein are assessed for their ability to restore liver function in an animal lacking full liver function. Braun et al. (2000), which is incorporated in its entirety herein by reference, outline a model for toxin-induced liver disease in mice transgenic for the HSV-tk gene. Rhim et al. (1995) and Lieber et al. (1995), each of which is incorporated in its entirety herein by reference, outline models for liver disease by expression of urokinase. Mignon et al. (1998), which is incorporated in its entirety herein by reference, outline liver disease induced by antibody to the cell-surface marker Fas. Overturf et al. (1998), which is incorporated in its entirety herein by reference, have developed a model for Hereditary Tyrosinemia Type I in mice by targeted disruption of the Fah gene. The animals can be rescued from the deficiency by providing a supply of 2-(2-nitro-4-fluoro-methyl-benzoyl)-1,3-cyclohexanedione (NTBC), but they develop liver disease when NTBC is withdrawn. Acute liver disease can be modeled by 90% hepatectomy, as described by Kobayashi et al., 2000; which is incorporated in its entirety herein by reference. Acute liver disease can also be modeled by treating animals with a hepatotoxin such as galactosamine, CCl4, or thioacetamide.

Chronic liver diseases such as cirrhosis can be modeled by treating animals with a sub-lethal dose of a hepatotoxin long enough to induce fibrosis (Rudolph et al., 2000; which is incorporated in its entirety herein by reference). Assessing the ability of hepatocytes provided herein to reconstitute liver function involves administering the cells to such animals, and then determining survival over a 1 to 8 week period or more, while monitoring the animals for progress of the condition. Effects on hepatic function can be determined by evaluating markers expressed in liver tissue, cytochrome p450 activity, and blood indicators, such as alkaline phosphatase activity, bilirubin conjugation, and prothrombin time), and survival of the host. Any improvement in survival, disease progression, or maintenance of hepatic function according to any of these criteria relates to effectiveness of the therapy, and can lead to further optimization.

Hepatocytes (for example, mature hepatocytes) provided in certain aspects of this invention that demonstrate desirable functional characteristics according to their profile of metabolic enzymes, or efficacy in animal models, may also be suitable for direct administration to human subjects with impaired liver function. For purposes of hemostasis, the cells can be administered at any site that has adequate access to the circulation, typically within the abdominal cavity. For some metabolic and detoxification functions, it is advantageous for the cells to have access to the biliary tract. Accordingly, the cells are administered near the liver (e.g., in the treatment of chronic liver disease) or the spleen (e.g., in the treatment of fulminant hepatic failure). In one method, the cells administered into the hepatic circulation either through the hepatic artery, or through the portal vein, by infusion through an in-dwelling catheter. A catheter in the portal vein can be manipulated so that the cells flow principally into the spleen, or the liver, or a combination of both. In another method, the cells are administered by placing a bolus in a cavity near the target organ, typically in an excipient or matrix that will keep the bolus in place. In another method, the cells are injected directly into a lobe of the liver or the spleen.

The hepatocytes provided in certain aspects of this invention can be used for therapy of any subject in need of having hepatic function restored or supplemented. Human conditions that may be appropriate for such therapy include, but are not limited to, fulminant hepatic failure due to any cause, viral hepatitis, drug-induced liver injury, cirrhosis, inherited hepatic insufficiency (such as Wilson's disease, Gilbert's syndrome, or al-antitrypsin deficiency), hepatobiliary carcinoma, autoimmune liver disease (such as autoimmune chronic hepatitis or primary biliary cirrhosis), urea cycle disorder, factor VII deficiency, glycogen storage disease type 1, infantile Refsum's disease, phenylketonuria, severe infantile oxalosis, cirrhosis, liver injury, acute liver failure, hepatobiliary carcinoma, hepatocellular carcinoma, genetic cholestasis (PFIC and alagille syndrome), hereditary hemochromatosis, tyrosinemia type 1, argininosuccinic aciduria (ASL), Crigler-Najjar syndrome, familial amyloid polyneuropathy, atypical haemolytic uremic syndrome-1, primary hyperoxaluria type 1, maple syrup urine disease (MSUD), acute intermittent porphyria, coagulation defects, GSD type Ia (in metabolic control), homozygous familial hypercholesterolemia, organic acidurias, and any other condition that results in impaired hepatic function. For human therapy, the dose is generally between about 10⁹ and 10¹² cells, and typically between about 5×10⁹ and 5×10¹⁰ cells, making adjustments for the body weight of the subject, nature and severity of the affliction, and the replicative capacity of the administered cells.

### Use in a Liver Assist Device

The invention also provides methods of use of the hepatocytes disclosed herein that are encapsulated or part of a bioartificial liver device. Various forms of encapsulation are described in the art, for example, in Cell Encapsulation Technology and Therapeutics, 1999; which is incorporated in its entirety herein by reference. Hepatocytes provided in certain aspects of this invention can be encapsulated according to such methods for use either in vitro or in vivo.

Bioartificial organs for clinical use are designed to support an individual with impaired liver function—either as a part of long-term therapy, or to bridge the time between a fulminant hepatic failure and hepatic reconstitution or liver transplant. Bioartificial liver devices are described by Macdonald et al., pp. 252-286 of “Cell Encapsulation Technology and Therapeutics”, and exemplified in U.S. Pat. Nos. 5,290,684, 5,624,840, 5,837,234, 5,853,717, and 5,935,849; each of which is incorporated in its entirety herein by reference. Suspension-type bioartificial livers comprise cells suspended in plate dialysers, microencapsulated in a suitable substrate, or attached to microcarrier beads coated with extracellular matrix. Alternatively, hepatocytes can be placed on a solid support in a packed bed, in a multiplate flat bed, on a microchannel screen, or surrounding hollow fiber capillaries. The device has an inlet and outlet through which the subject's blood is passed, and sometimes a separate set of ports for supplying nutrients to the cells.

Hepatocytes are prepared according to the methods described herein, and then plated into the device on a suitable substrate, such as a matrix of Matrigel® or collagen. The efficacy of the device can be assessed by comparing the composition of blood in the afferent channel with that in the efferent channel—in terms of metabolites removed from the afferent flow, and newly synthesized proteins in the efferent flow. Devices of this kind can be used to detoxify a fluid such as blood, wherein the fluid comes into contact with the hepatocytes provided in certain aspects of this invention under conditions that permit the cell to remove or modify a toxin in the fluid. The detoxification will involve removing or altering at least one ligand, metabolite, or other compound (either natural and synthetic) that is usually processed by the liver. Such compounds include but are not limited to bilirubin, bile acids, urea, heme, lipoprotein, carbohydrates, transferrin, hemopexin, asialoglycoproteins, hormones like insulin and glucagon, and a variety of small molecule drugs. The device can also be used to enrich the efferent fluid with synthesized proteins such as albumin, acute phase reactants, and unloaded carrier proteins. The device can be optimized so that a variety of these functions is performed, thereby restoring as many hepatic functions as are needed. In the context of therapeutic care, the device processes blood flowing from a patient in hepatocyte failure, and then the blood is returned to the patient.

The invention also provides methods of use of the hepatocytes disclosed herein, for example, in combination with other cell types, as organoids. Organoids can be established from the hepatocytes and grown for multiple months, while retaining key morphological, functional and gene expression features. See, e.g., Hu et al., 2018, Cell; 175(6): 1591-1606; which is incorporated in its entirety herein by reference.

Further, for purposes of manufacture, distribution, and use, the hepatocytes of the invention may be supplied in the form of a cell culture or suspension in an isotonic excipient or culture medium, optionally frozen to facilitate transportation or storage.

The invention also includes different reagent systems, comprising a set or combination of cells that exist at any time during manufacture, distribution, or use. The cell sets comprise any combination of two or more cell populations described in this disclosure, e.g., mature hepatocytes, their precursors and subtypes, in combination with undifferentiated stem cells, somatic cell-derived hepatocytes, or other differentiated cell types. The cell populations in the set sometimes share the same genome or a genetically modified form thereof.

The invention contemplates that compositions of hepatocytes, for example, obtained from human pluripotent stem cells (e.g., human embryonic stem cells or other pluripotent stem cells) can be used to treat any of the foregoing diseases or conditions. These diseases can be treated with compositions of hepatocytes comprising hepatocytes of varying levels of maturity, as well as with compositions of hepatocytes that are enriched for mature hepatocytes.

### IV. Methods of Administration of Hepatocytes

The hepatocytes of the invention may be administered by any route of administration appropriate for the disease or disorder being treated. In an embodiment, the hepatocytes of the invention may be administered topically, systemically, or locally, such as by injection, or as part of a device or implant (e.g., a sustained release implant). For example, the hepatocytes of the present invention may be transplanted into the hepatocellular space by using surgery when treating a patient with a disorder or disease, such as fulminant hepatic failure due to any cause, viral hepatitis, drug-induced liver injury, cirrhosis, inherited hepatic insufficiency (such as Wilson's disease, Gilbert's syndrome, or al-antitrypsin deficiency), hepatobiliary carcinoma, autoimmune liver disease (such as autoimmune chronic hepatitis or primary biliary cirrhosis), urea cycle disorder, factor VII deficiency, glycogen storage disease type 1, infantile Refsum's disease, phenylketonuria, severe infantile oxalosis, cirrhosis, liver injury, acute liver failure, hepatobiliary carcinoma, hepatocellular carcinoma, genetic cholestasis (PFIC and alagille syndrome), hereditary hemochromatosis, tyrosinemia type 1, argininosuccinic aciduria (ASL), Crigler-Najjar syndrome, familial amyloid polyneuropathy, atypical haemolytic uremic syndrome-1, primary hyperoxaluria type 1, maple syrup urine disease (MSUD), acute intermittent porphyria, coagulation defects, GSD type Ia (in metabolic control), homozygous familial hypercholesterolemia, organic acidurias, and any other condition that results in impaired hepatic function. One skilled in the art would be able to determine the route of administration for the disease or disorder being treated.

Hepatocytes of the invention may be delivered in a pharmaceutically acceptable formulation by injection. Concentrations for injections may be at any amount that is effective and non-toxic, depending upon the factors described herein. In an embodiment, at least 1×10⁶, 2×10⁶, 5×10⁶, 1×10⁷, 1×10⁸, or 1×10¹⁰ hepatocytes may be administered to a patient in need thereof.

Products and systems, such as delivery vehicles, comprising the agents of the invention, especially those formulated as pharmaceutical compositions, as well as kits comprising such delivery vehicles and/or systems, are also envisioned as being part of the present invention.

In certain embodiments, a therapeutic method of the invention includes the step of administering hepatocytes of the invention with an implant or device. In certain embodiments, the device is bioerodible implant for treating a disease or condition described herein.

The volume of composition administered according to the methods described herein is also dependent on factors such as the mode of administration, number of hepatocytes, age of the patient, and type and severity of the disease being treated.

Hepatocytes can be delivered one or more times periodically throughout the life of a patient. For example hepatocytes can be delivered once per year, once every 6-12 months, once every 3-6 months, once every 1-3 months, or once every 1-4 weeks. Alternatively, more frequent administration may be desirable for certain conditions or disorders. If administered by an implant or device, hepatocytes can be administered one time, or one or more times periodically throughout the lifetime of the patient, as necessary for the particular patient and disorder or condition being treated. Similarly contemplated is a therapeutic regimen that changes over time. In certain embodiments, patients are also administered immunosuppressive therapy, either before, concurrently with, or after administration of the hepatocytes. Immunosuppressive therapy may be necessary throughout the life of the patient, or for a shorter period of time. Examples of immunosuppressive therapy include, but are not limited to, one or more of: anti-lymphocyte globulin (ALG) polyclonal antibody, anti-thymocyte globulin (ATG) polyclonal antibody, azathioprine, BASILIXIMAB® (anti-I L-2Ra receptor antibody), cyclosporin (cyclosporin A), DACLIZUMAB® (anti-IL-2Ra receptor antibody), everolimus, mycophenolic acid, RITUXIMAB® (anti-CD20 antibody), sirolimus, tacrolimus (Prograf™), and mycophenolate mofetil (MMF).

In certain embodiments, hepatocytes of the present invention are formulated with a pharmaceutically acceptable carrier. For example, hepatocytes may be administered alone or as a component of a pharmaceutical formulation. The hepatocytes may be formulated for administration in any convenient way for use in human medicine. In certain embodiments, pharmaceutical compositions suitable for parenteral administration may comprise the hepatocytes, in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof. Proper fluidity can be maintained, for example, by the use of coating materials, such as lecithin, by the maintenance of the required particle size in the case of dispersions, and by the use of surfactants.

### V. Kits

In another aspect, the invention provides an article of manufacture or a kit comprising a population of hepatocytes, for example, a population of pluripotent stem cells, immature hepatocytes, mature hepatocytes, and/or a pharmaceutical composition of the disclosure.

In another aspect, the invention provides an article of manufacture or a kit comprising an expression vector, wherein the expression vector comprises a nucleic acid encoding at least one transcription factor selected from the group consisting of Nuclear Factor I X (NFIX) and Nuclear Factor I C (NFIC).

In some embodiments, the transcription factor is NFIX. In some embodiments, the transcription factor is NFIC. In some embodiments, the transcription factor is NFIX and NFIC.

In some embodiments, the NFIC is at least one alternatively spliced NFIC variant selected from the group consisting of NFIC, transcript variant 1; NFIC, transcript variant 2; NFIC, transcript variant 3; NFIC, transcript variant 4; and NFIC, transcript variant 5. In some embodiments, the alternatively spliced NFIC variant is NFIC, transcript variant 1. In some embodiments, the alternatively spliced NFIC variant is NFIC, transcript variant 3. In some embodiments, the alternatively spliced NFIC variant is NFIC, transcript variant 1 and NFIC, transcript variant 3.

The article of manufacture or kit can further comprise a package insert comprising instructions for using the population of hepatocytes or the pharmaceutical composition of the invention, for example, to treat or delay progression of any disease disclosed herein. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent). Suitable containers for the one or more agent include, for example, bottles, vials, bags and syringes.

All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1: Materials and Methods

### Lentivirus Production:

Tet-On 3G viral particles were purchased from TakaraBio (Takarabio, Cat. #0055VCT). pLVX-TRE3G (Takarabio, Cat. #631187) was used as the lentiviral vector to express the genes of interest under a Tet-On inducible promoter and pLVX-TRE3G-Luciferase was used as a positive control. Lentiviral particles were produced using a series of products developed by TakaraBio (www.takarabio.com). Packaging of viruses was performed using a fourth-generation lentivirus packaging system consisting of the Lenti-X 293T Cells (Takarabio, Cat. #632180) and Lenti-X Packaging Single Shots (Takarabio, Cat. #631275 & 631276). Viral concentration and quantity were determined using the Lenti-X™ Concentrator (Takarabio, Cat. #631231 & 631232) and the Lenti-X qRT-PCR Titration Kit (Takarabio, Cat. #631235), respectively. All procedures were performed using manufacturer recommended protocols. Viruses were aliquoted and stored at −80° C. until use.

### Viral Titering by GFP Limiting Dilution:

GFP (GeneCopoeia, Cat #Lv215) under an EF1a promoter was used as a source of GFP viral particles. Viral particles were generated as described above. To determine viral multiplicity of infection (MOI) relationship with copy number per microliter, 1.1×10⁵ cells were plated in a 12 well plate. One day after plating, 1.2 mL of GFP lentiviral serial dilutions were used for transduction. For each concentration of GFP, polybrene (6 μg/μl) (Sigma, Cat. #H9268) and 0.5 mL of viral serial dilutions were placed in duplicate wells and spin-infections were performed for 1 hour at room temperature at 2000 rpm. The media was changed the day after transduction to 1 mL of fresh media. 72 hours after transduction the percentage of GFP positive cells was determined using Flow cytometry (Macsquant). Only wells with 1% to 20% GFP+ were used for transforming unit calculations.

### HuH7 Cell Culture Conditions:

Hepatoma cell line HuH7 was grown in low glucose DMEM (ThermoFisher, Cat. 11885-084) media containing 10% FBS, (ThermoFisher, Cat. #26140-079). HuH7 cells were split twice a week. For dissociation, cells were first washed in PBS−/− (ThermoFisher, Cat. #14190-144) followed with 0.25% Trypsin—0.02% EDTA (Sigma, Cat. #59428C), and incubated for 4 minutes at room temperature. Cells were collected in 9 ml of HuH7 growth medium and centrifuged at 1000 rpm for 5 minutes. The supernatant was removed, and cells were seeded at a 1:4 split ratio.

### Development of an HuH7-Tet-On3G Cell Line:

Hepatoma cell line HuH7 was transduced with lentiviral particles encoding the Tet-On3G transactivator (Tet-On3G) under a constitutive EF1 alpha promoter. Transductions were performed in the presence of 6 μg/μl of polybrene using spin-infections for 1 hour at room temperature at 2000 rpm. The day after transduction, the cell media was changed. The viruses contained a neomycin selectable marker, which allowed selection of pools containing lentiviral integrations. Optimal neomycin (G418), (ThermoFisher, Cat. #10131027) concentration for selection (1.1 mg/ml) was determined empirically based on the minimum dose of neomycin that induced cell death after 4 days. For cell line validation, HuH7 cells with a Tet-On3G integration (HuH7-Tet-On3G) were transduced with TRE-luciferase control lentiviral particles. Media was changed in the presence or absence of doxycycline (1 μg/ml, Sigma Cat. #D3072).

### Transcription Factor Screen in HuH7-Tet-On3G Cell Line:

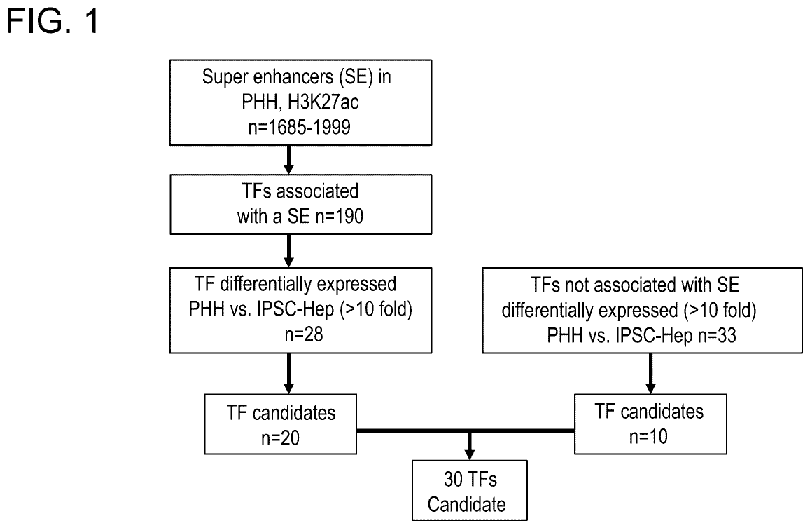

A schematic representation of the selection of transcription factors of the invention is depicted by FIG. 1. Transductions with lentivirus particles encoding the candidate transcription factors were performed in HuH7-Tet-On3G cells using spin-infections for 1 hour at room temperature at 2000 rpm, at an MOI of 10 in the presence of polybrene (6 μg/μl). The day after transduction, the cell media was changed. Media changes in the presence or absence of doxycycline (1 μg/ml), were performed every 2 days for a total of 4-5 days.

### Stem Cell Culture:

Human iPSC cells (“hiPSC-GMP1” or “GMP1 iPSC”) were maintained in mTeSR™ 1 media (STEMCELL Technologies, Cat. #85850) on flasks coated with vitronectin (ThermoFisher, Cat. #A14700) at 1/100 dilution in PBS−/−. Cells were cultured under conditions of 20% O₂/5% CO₂ and passaged every 3-4 days by producing small clumps using EDTA (0.5 mM; ThermoFisher, Cat. #AM9260G).

### Hepatocyte Differentiation Protocol:

The pluripotent stem cell derived hepatocytes were derived from culture dishes using a four stage, twenty-day protocol, as previously described by Mallanna et al., 2013 (Curr Protoc Stem Cell Biol.; 26:1G.4.1-1G.4.13; which is incorporated by reference in its entirety herein). In order to generate hepatocytes, monolayers of pluripotent cells were harvested using accutase (STEMCELL Technologies, Cat. #07920) for 7 minutes at 37° C. and transferred to LN521 (ThermoFisher, Cat. #A29248) pre-coated plates, at a density of 2×10⁵ cells/cm². Cells were cultured using an mTeSR™1 media for 24 hours prior to induction. The base medium for differentiation comprises RPMI (ThermoFisher, Cat. #22400-089) containing 1× Pen Strep (ThermoFisher, Cat. #15140-122) and 1% MEM-NEAA (ThermoFisher, Cat. #11140-050). Stage 1 of the differentiation process was initiated by culturing pluripotent stem cells for 2 days in a culture media comprising 100 ng/ml of Activin A (R&D systems, Cat. #338-AC-010), 20 ng/ml of BMP4 (R&D, Cat. #314BP) and 10 ng/ml of FGF-2 (ThermoFisher, Cat. #PHG0266) in an RPMI media, which may be supplemented with 2% B27 without insulin (ThermoFisher, Cat. #A1895601). This was followed by culturing the cells for 3 days in a culture media comprising 100 ng/ml of Activin A (R&D systems, Cat. #338-AC-010) in an RPMI media, which may be supplemented with 2% B27 without insulin. Stage 2 of the differentiation process comprised culturing the cells derived from stage 1 for 5 days in a culture media comprising 20 ng/ml of BMP4 (R&D, Cat. #314BP) and 10 ng/ml of FGF-2 in an RPMI media, which may be supplemented with 2% B27 with insulin (ThermoFisher, Cat. #A3582801). Stage 3 was initiated by culturing the cells derived from stage 2 for 5 days in a culture media comprising 20 ng/ml of HGF (Peprotech, Cat. #100-39) in an RPMI media, which may be supplemented with 2% B27 with insulin. Finally, stage 4 comprised culturing the cells derived from stage 3 for 5 days in a culture media comprising 20 ng/ml of Oncostatin-M (R&D systems, Cat. #295-OM-010) in a Hepatocyte Culture Media (Lonza, Cat. #CC-3198), which may be supplemented with SingleQuots (without EGF).

Transductions of iPSC-Derived Hepatocytes:

Transductions with lentivirus particles encoding the transcription factors (using an MOI of 3), and Tet-On3G (using an MOI of 5) were performed at the end of stage 3 of the differentiation protocol (at days 15-16 of the cell culture) using spin-infections for 1 hour at room temperature at 2000 rpm, in the presence of polybrene (6 μg/μl). The day after transduction, the culture media was changed. Media changes during stage 4 were performed using culture media comprising doxycycline (1 μg/ml), every day for a total of 5 or 9 days (i.e., cells were harvested at day 20 or day 24 of cell culture).

### Real Time PCR Analysis of Mature Hepatocyte Markers:

Total RNA from cultured cells was isolated using the RNeasy Micro kit (Qiagen, Cat. #74004), and cDNA was generated with High Capacity RNA-to-cDNA Transcription System (ThermoFisher, Cat. #4387406). Real-time quantitative PCR reactions were performed on a QuantStudio 7 Flex machine (ThermoFisher) using Taqman probes and Fast advance mix (ThermoFisher, Cat. #A44360). cDNA levels of target genes were analyzed using comparative Ct methods, where Ct is the cycle threshold number normalized to RPL13A.

### Compounds:

8-Bromoadenosine 3′, 5′-cyclic monophosphate (8-Br-cAMP) (Sigma, Cat. #B7880) was dissolved with PBS−/− (ThermoFisher, Cat. #14190-144) to a concentration of 100 mM (100×), and dexamethasone (sigma, Cat. #D4902) was dissolved in DMSO to a concentration of 100 M (1000×).

### CYP1A2 Functional Assay:

CYP1A2 activity was measured using the Promega kit (Promega, Cat. #V8422) following the manufacturer recommendations. Primary human hepatocytes (150,000 viable cells) were plated in each well of a Collagen I coated 48-well plate in 250 μl of In VitroGRO CP medium (BioIVT, Cat. #Z99029). Cells were maintained for 2 days in In VitroGRO CP medium, and the medium was changed daily. CYP1A2 activity was induced in In VitroGRO HI medium (BioIVT, Cat. #Z99009) for 2 days using 100 μM Omeprazole, and the In VitroGRO HI medium containing Omeprazole was changed daily. After 48-hours of incubation with Omeprazole, cells were washed twice with plain In VitroGRO KHB medium (BioIVT, Cat. #Z99074), and were fed with 150 μl per well of fresh KHB medium containing 6 uM Luciferin-1A2 and 3 mM Salicylamide. A CYP1A2 inhibitor, 5 M α-naphthoflavone, was included along with Luciferin-1A2 substrate in the inhibitor control wells. For background luminescence control, 150 μl of KHB media containing Luciferin-1A2 and 3 mM Salicylamide was included into empty wells. 20 μl of supplied 2M D-Cysteine was added to 10 ml reconstituted Luciferin Detection Reagent. After incubation at 37° C. for 60 minutes, 50 μl of supernatant was transferred to opaque white assay plates (Costar 3912), and 50 μl of Luciferin detection reagent was added. After Incubation at room temperature for 20 minutes, luminescence was read using a luminometer. hiPSC-GMP1 were seeded at a density of 2×10⁵ cells/cm² in 48 well plates. hiPSC-GMP1 were differentiated to hepatocyte like cells as described in detail above. Transductions with Teton, NFIC and NFIX were performed at day 15, as described in detail above. CYP1A2 activity was measured using the manufacturer recommendations. To measure the 20-day timepoint, day 18 hepatocyte like cells were incubated in Hepatocyte Culture Media (Lonza) supplemented with SingleQuots (without EGF) for 2 days with 100 μM Omeprazole. After 48-hours of incubation with Omeprazole, cells were washed twice with plain KHB medium and CYP1A2 activity was measured as described in detail above. For the 24-day time point, hepatocyte like cells at day 22 of differentiation were treated as described in detail above for the 20-day timepoint. CYP1A2 activity was normalized for the number of cells.

### AFP, ALB and Urea Secretion in Media:

Primary human hepatocytes (150,000 viable cells) were plated in each well of a Collagen I coated 48-well plate in 250 μl of In VitroGRO CP medium (BioIVT, Cat. #Z99029). Cells were maintained for 2 days in In VitroGRO CP medium. hiPSC-GMP1 were differentiated to hepatocyte like cells as described in detail above. Transductions with Teton, NFIC and NFIX were performed at day 15, as described in detail above. Supernatants were collected from primary human hepatocytes at day 2 after plating, or from GMP1-derived hepatocyte-like cells at day 20 or day 24 of differentiation. These supernatants were used for performing an ELISA assay of human alpha fetoprotein (AFP) (Abcam, Cat. #ab108838), human albumin (ALB) (Abcam, Cat. #ab108788) or for an enzymatic assay for measuring urea secretion (Sigma, Cat. #MAK006). Procedures were performed following manufacturers recommendations for each of the foregoing assays. AFP, ALB and urea secretion in the culture media was normalized for the number of cells.

### Example 2: A Model System to Screen Transcription Factor Candidates

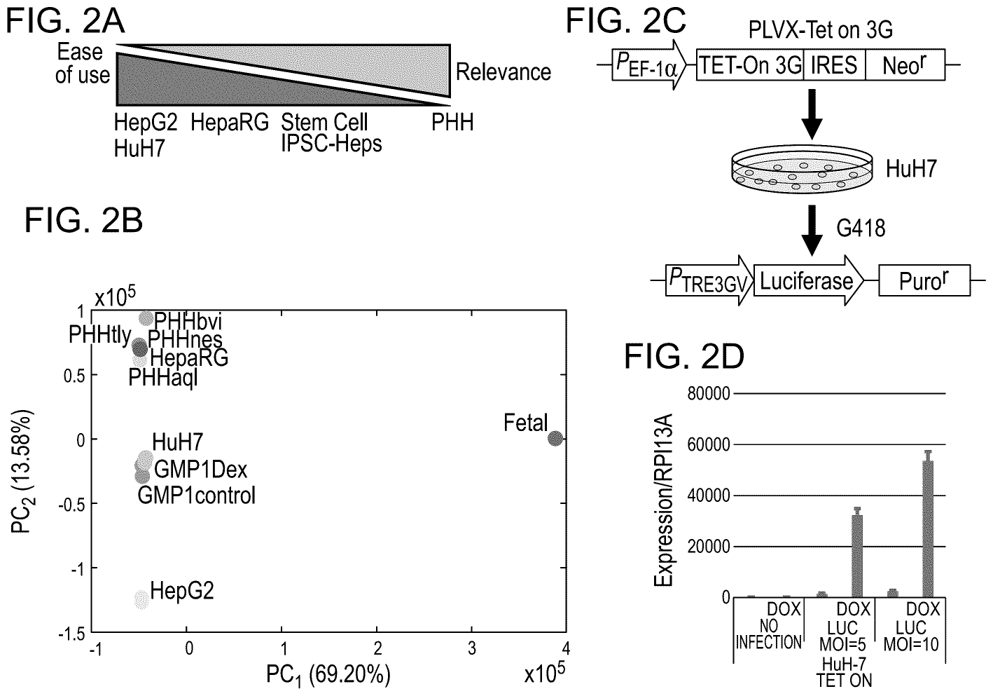

A principal component analysis (PCA) was performed for cancer cell lines (HepG2, HuH7 and HepaRG), stem cell derived hepatocytes (Stem Cell/iPSC-Heps) and primary human hepatocytes (PHH) (FIGS. 2A-B). PHH-AQL, PHH-TLY and PHH-NES are adult hepatocytes. PHH-BVI are stillborn hepatocytes and Fetal correspond to human fetal primary hepatocytes. HuH7 cells cluster with hepatocytes differentiated from GMP1 iPSC that were not further treated with Br-cAMP and dexamethasone (“GMP1 control”) and that were further treated with Br-cAMP and dexamethasone for 5 days (“GMPDex”), and therefore, were used for the construction of a HuH7-Tet-On3G cell line (FIG. 2C), as described in Example 1, for screening of transcription factors of the invention. As depicted in FIG. 2D, the HuH7-Tet-On3G cell line was responsive to doxycycline induction. The HuH7-Tet-On3G cell line was transduced using lentivirus particles containing a Tet response element upstream of luciferase (TRE-Luc), at an MOI of 0, 5 and 10. Cells were grown for 48 hours after transduction in the presence or absence of 1 μg/ml doxycycline. Luciferase expression relative to the house-keeping gene RPL13A was normalized to the non-infected control sample in the absence of doxycycline. This study depicts an exemplary model system that was used to screen transcription factor candidates of the invention.

### Example 3: Increasing Expression of Different Transcription Factors in Immature Hepatocytes

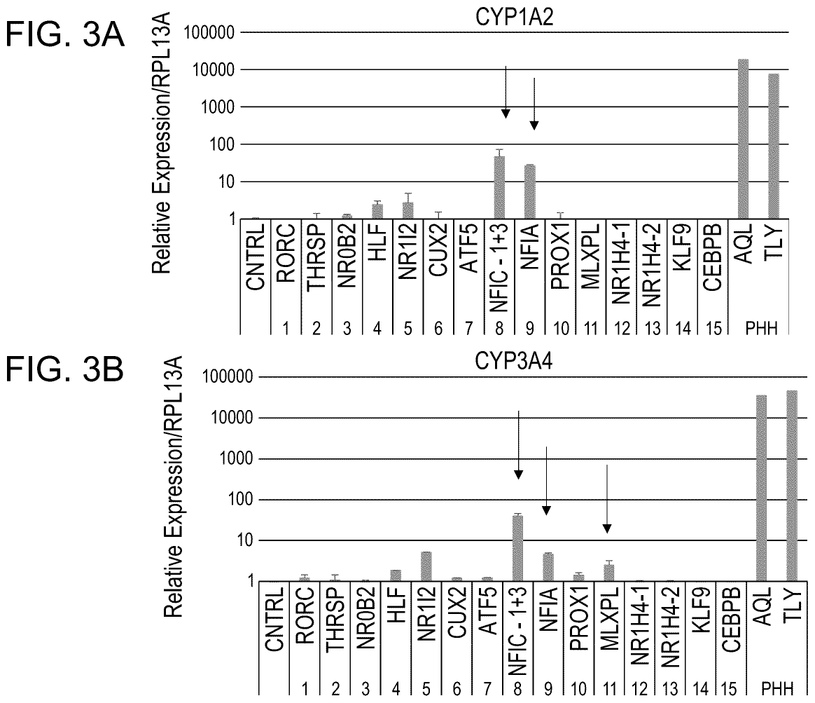

The screening for transcription factors that promote maturation of hepatocytes was performed in a HuH7-Tet-On3G cell line, which was generated as described above in Example 1. The screening for the transcription factors was performed by measuring an increase in the expression of mature hepatocyte markers, CYP1A2 (FIG. 3A) and CYP3A4 (FIG. 3B) after transduction of cells with lentivirus particles comprising the different transcription factor candidates. Transduction of the transcription factors was performed at a multiplicity of infection (MOI) of 10. NFIC, transcript variants 1 and 3 (NFIC-1+3) refers to a mixture of alternatively spliced variants of transcription factor NFIC, NFIC, transcript variant 1 (NFIC-1) (NCBI Reference Sequence No.: NM_001245002) and NFIC, transcript variant 3 (NFIC-3) (NCBI Reference Sequence No.: NM_001245004), respectively, which were transduced at an MOI of 5, each for NFIC, transcript variant 1 (NFIC-1), and NFIC, transcript variant 3 (NFIC-3). After transduction, cells were cultured for 5 days using an HuH7 media comprising 1 μg/ml of doxycycline. The expression of mature hepatocyte markers was plotted relative to the house-keeping gene RPL13A, and was normalized to the non-infected cells. Adult primary human hepatocytes (PHH), lots AQL and TLY, were used as a positive control. PHH cells were obtained from BioIVT and the mRNA was extracted from frozen vials. The arrows in FIGS. 3A-B describe the different transcription factors that upregulated the expression levels of mature hepatocyte markers CYP1A2 and CYP3A4.

### Example 4: Increasing Expression of Transcription Factor NFIC in Immature Hepatocytes Increases Expression of Mature Hepatocyte Markers

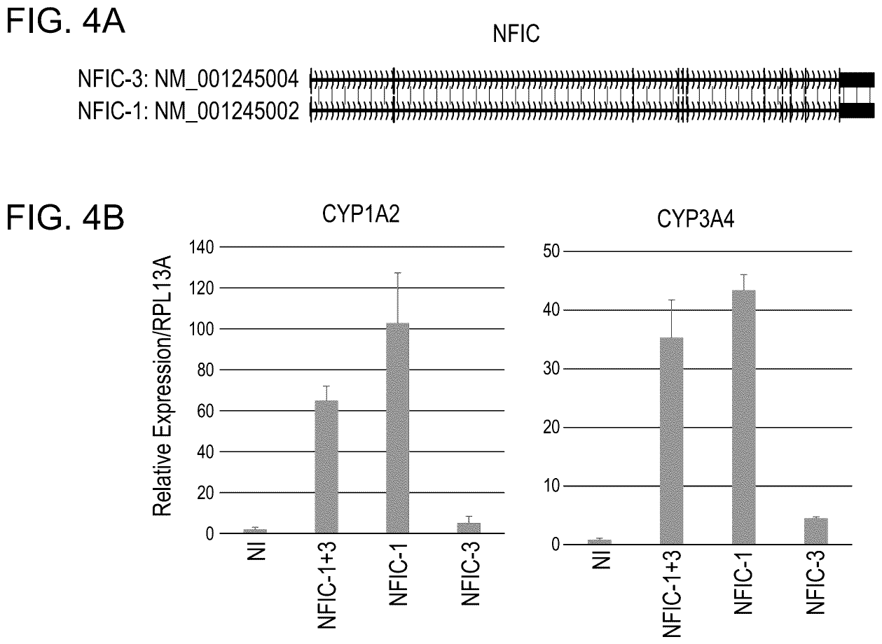

The HuH7-Tet-On3G cells, generated as described above in Example 1, were transduced with lentivirus particles comprising transcription factors NFIC, transcript variants 1 and 3 (NFIC-1+3); NFIC, transcript variant 1 (NFIC-1); or NFIC, transcript variant 3 (NFIC-3) at an MOI of 5. NFIC, transcript variants 1 and 3 (NFIC-1+3) refers to a mixture of alternatively spliced variants of transcription factor NFIC, NFIC, transcript variant 1 (NFIC-1) and NFIC, transcript variant 3 (NFIC-3), respectively (FIG. 4A). After transduction, the cells were cultured for 5 days in a culture media comprising 1 μg/ml of doxycycline. The expression levels of the mature hepatocyte markers CYP1A2 and CYP3A4 were determined relative to the house-keeping gene RPL13A, and were normalized to the non infected (“NI”) cells. The results of the study, as depicted in FIG. 4B, demonstrate that increasing expression of NFIC in immature hepatocytes increases expression levels of mature hepatocyte markers, and thereby promotes the generation of mature hepatocytes.

### Example 5: Increasing Expression of Transcription Factor NFIC in Immature Hepatocytes Cultured in Presence of Dexamethasone and 8-Br-CAMP Increases Expression of Mature Hepatocyte Markers

The HuH7-Tet-On3G cells, generated as described above in Example 1, were transduced with lentivirus particles comprising transcription factor NFIC, transcript variant 1 (NFIC-1) at an MOI of 50. After transduction, the cells were cultured for 5 days in a culture media comprising 1 μg/ml of doxycycline in the presence or absence of 1 mM 8-Bromoadenosine 3′, 5′-cyclic monophosphate (8-Br-cAMP) and 100 nM dexamethasone. The expression levels of the mature hepatocyte markers CYP1A2 (FIG. 5A), TAT (FIG. 5B) and UGT1A1 (FIG. 5C) were determined relative to the house-keeping gene RPL13A, and were normalized to the non-infected negative control sample (in the absence of 8-Br-cAMP and dexamethasone). Primary human hepatocytes (PHH) expression values correspond to an average of the expression values of lots PHH-AQL and PHH-TLY. PHH cells were obtained from BioIVT and the mRNA was extracted from frozen vials. The results of the study, as depicted in FIG. 5, demonstrate that increasing expression of NFIC in immature hepatocytes cultured in presence of dexamethasone and 8-Br-CAMP increases expression levels of mature hepatocyte markers, and thereby promotes the generation of mature hepatocytes.

### Example 6: Increasing Expression of Different Transcription Factors in Immature Hepatocytes

The screening for transcription factors that promote maturation of hepatocytes was performed in a HuH7-Tet-On3G cell line, which was generated as described above in Example 1. The screening for the transcription factors was performed by measuring an decrease in the expression of immature hepatocyte marker AFP (FIG. 6A), and increase in expression of mature hepatocyte markers CYP1A2 (FIG. 6B), TAT (FIG. 6C) and CYP3A4 (FIG. 6D) after transduction of cells with lentivirus particles comprising the different transcription factor candidates. Transduction of the transcription factors was performed at a multiplicity of infection (MOI) of 10. After transduction, cells were cultured in a culture media comprising 1 μg/ml doxycycline, 1 mM 8-Br-cAMP and 100 nM dexamethasone. Expression of maturation markers was measured 5 days after transduction. Relative expression of maturation markers was normalized to transduction with NFIC, transcript variant 1 (NFIC-1) in the presence of 1 μg/ml doxycycline as a control. Primary human hepatocytes (PHH) expression values correspond to an average of the expression values of lots PHH-AQL and PHH-TLY. PHH cells were obtained from BioIVT and mRNA was extracted from frozen vials. The arrows in FIGS. 6A-D describe the different transcription factors that downregulated the expression levels of immature hepatocyte marker AFP, and upregulated the expression levels of mature hepatocyte markers CYP1A2, TAT and CYP3A4.

### Example 7: Increasing Expression of Transcription Factors NFIC and/or NFIX in Pluripotent Stem Cell Derived Immature Hepatocytes Increases Expression of Mature Hepatocyte Markers

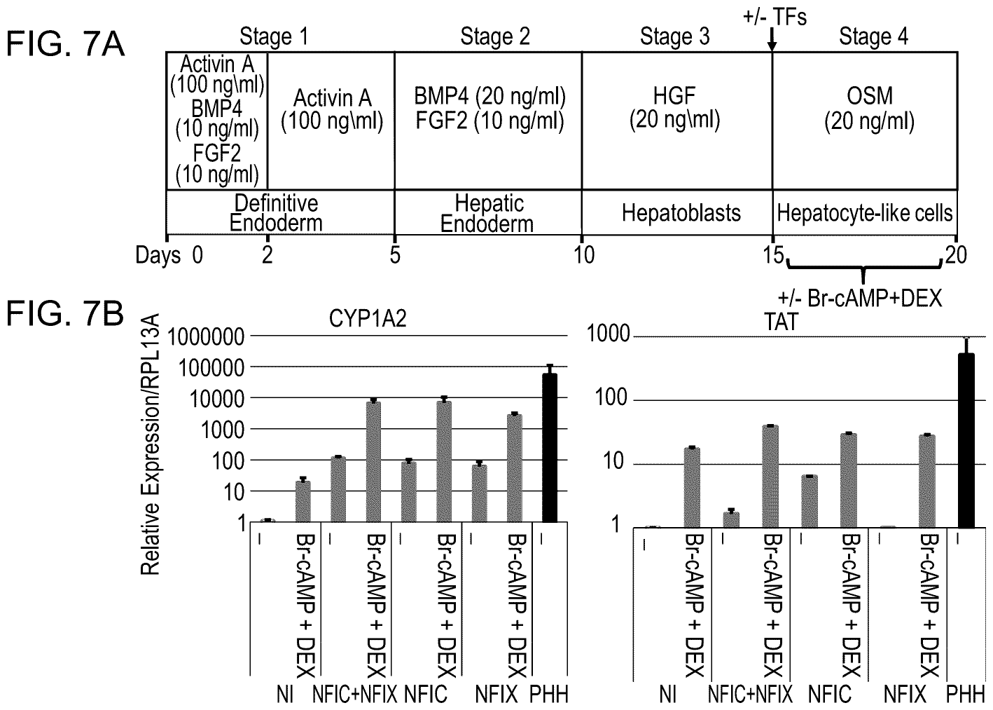

Pluripotent stem cell derived immature hepatocytes were generated using a four stage step-wise differentiation process, as described in detail in Example 1 (FIG. 7A). At the end of stage 3, transductions were performed with lentivirus particles comprising Tet-On3G (MOI of 5) or lentivirus particles comprising Tet-On3G in combination with the transcription factor NFIC, transcript variant 1 (NFIC-1); NFIX; or NFIC, transcript variant 1 (NFIC-1) and NFIX (MOI of 3) at day 15 of differentiation towards the hepatocyte like cells (FIG. 7A). The cells were subsequently cultured for 5 days in stage 4 media, as described above in Example 1, comprising 1 μg/ml of doxycycline in the presence or absence of 1 mM of 8-Br-cAMP and 100 nM of dexamethasone. The expression levels of the mature hepatocyte markers CYP1A2 and TAT were determined relative to the house-keeping gene RPL13A, and were normalized to the non-infected negative control sample (“NI”). Primary human hepatocytes (PHH) expression values correspond to an average of the expression values of lots PHH-AQL and PHH-TLY. PHH cells were obtained from BioIVT and mRNA was extracted from frozen vials. The results of the study, as depicted in FIG. 7B, demonstrate that increasing expression of transcription factors NFIC and/or NFIX in pluripotent stem cell-derived immature hepatocytes increases expression levels of mature hepatocyte markers, and thereby promotes the generation of mature hepatocytes.

### Example 8: Time Course Analysis of Expression of Mature Hepatocyte Markers by Increasing Expression of Transcription Factors NFIC and/or NFIX in Pluripotent Stem Cell Derived Immature Hepatocytes

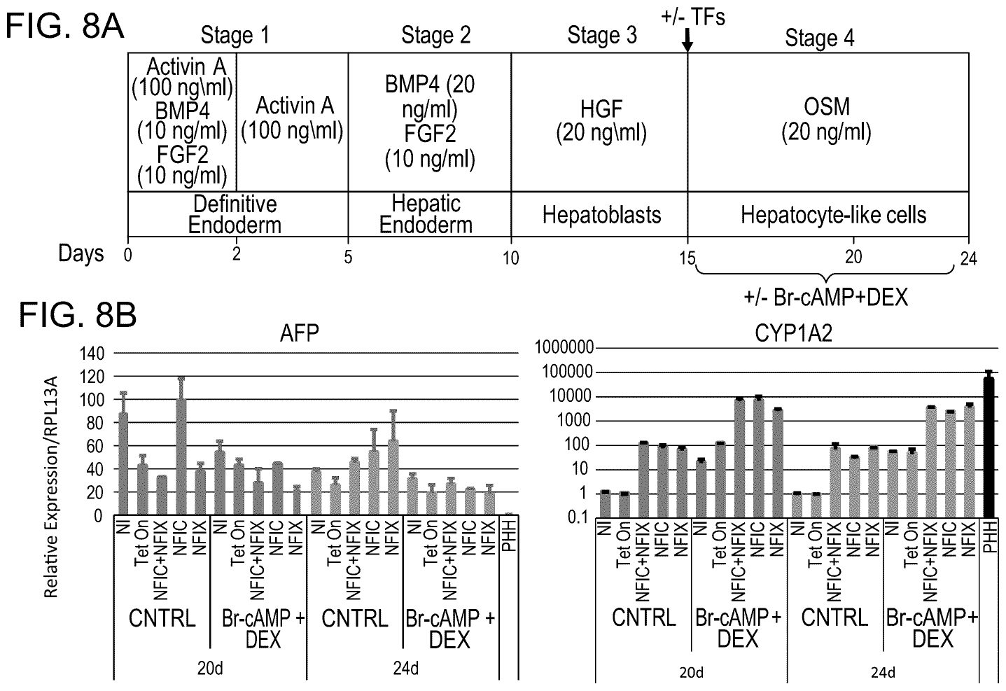

Pluripotent stem cell derived immature hepatocytes were generated using a four stage step-wise differentiation process, as described in detail in Example 1 (FIG. 8A). At the end of stage 3, transductions were performed with lentivirus particles comprising Tet-On3G (MOI of 5) (“TetOn”) or lentivirus particles comprising Tet-On3G in combination with the transcription factor NFIC, transcript variant 1 (NFIC-1); NFIX; or NFIC, transcript variant 1 (NFIC-1) and NFIX (MOI of 3) at day 15 of differentiation towards the hepatocyte like cells (FIG. 8A). The cells were subsequently cultured for 5 days or 9 days in stage 4 media, as described above in Example 1, comprising 1 μg/ml of doxycycline in the presence or absence of 1 mM of 8-Br-cAMP and 100 nM of dexamethasone. The cells were harvested at day 20 and day 24 of cell culture, and the expression levels of the immature hepatocyte marker AFP and mature hepatocyte marker CYP1A2 were determined relative to the house-keeping gene RPL13A, and were normalized to the non-infected (“NI”) negative control sample. Primary human hepatocytes (PHH) expression values correspond to an average of the expression values of lots PHH-AQL and PHH-TLY. PHH cells were obtained from BioIVT and mRNA was extracted from frozen vials. The results of the study, as depicted in FIG. 8B, demonstrate that increasing expression of transcription factors NFIC and/or NFIX in pluripotent stem cell-derived immature hepatocytes decreases expression levels of an immature hepatocyte marker and increases expression levels of a mature hepatocyte marker, and thereby promotes the generation of mature hepatocytes.

### Example 9: Increasing Expression of Transcription Factors NFIC and/or NFIX in Pluripotent Stem Cell Derived Immature Hepatocytes Shifts the Transcriptome Towards the Transcriptome of Mature Hepatocytes

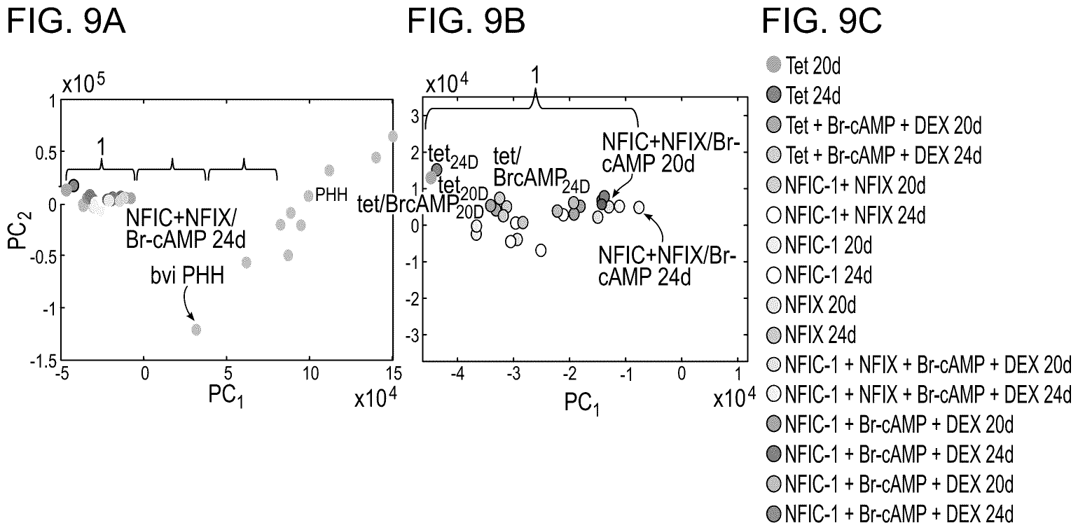

A principal component analysis (PCA) was performed on pluripotent stem cell-derived immature hepatocytes. The pluripotent stem cell derived immature hepatocytes were generated using a four stage step-wise differentiation process, as described in detail in Example 1. At the end of stage 3, transductions were performed with lentivirus particles comprising Tet-On3G (MOI of 5) or lentivirus particles comprising Tet-On3G in combination with the transcription factor NFIC, transcript variant 1 (NFIC-1); NFIX; or NFIC, transcript variant 1 (NFIC-1) and NFIX (MOI of 3) at day 15 of differentiation towards the hepatocyte like cells. The cells were subsequently cultured for 5 days or 9 days in stage 4 media, as described above in Example 1, comprising 1 μg/ml of doxycycline in the presence or absence of 1 mM of 8-Br-cAMP and 100 nM of dexamethasone. The cells were harvested at day 20 and day 24 of cell culture. Ten different primary human hepatocyte (PHH) datasets corresponding to 10 different individuals were used for the PCA analysis. PHH cells were obtained from BioIVT and mRNA was extracted from frozen vials. The results of the study, as depicted in FIG. 9, demonstrate that increasing expression of transcription factors NFIC and/or NFIX in pluripotent stem cell-derived immature hepatocytes results in a shift of 30-34% of the transcriptome towards the transcriptome of primary human hepatocytes.

### Example 10: Functional Assays of Pluripotent Stem Cell Derived Immature Hepatocytes Comprising Increased Expression of Transcription Factors NFIC and/or NFIX

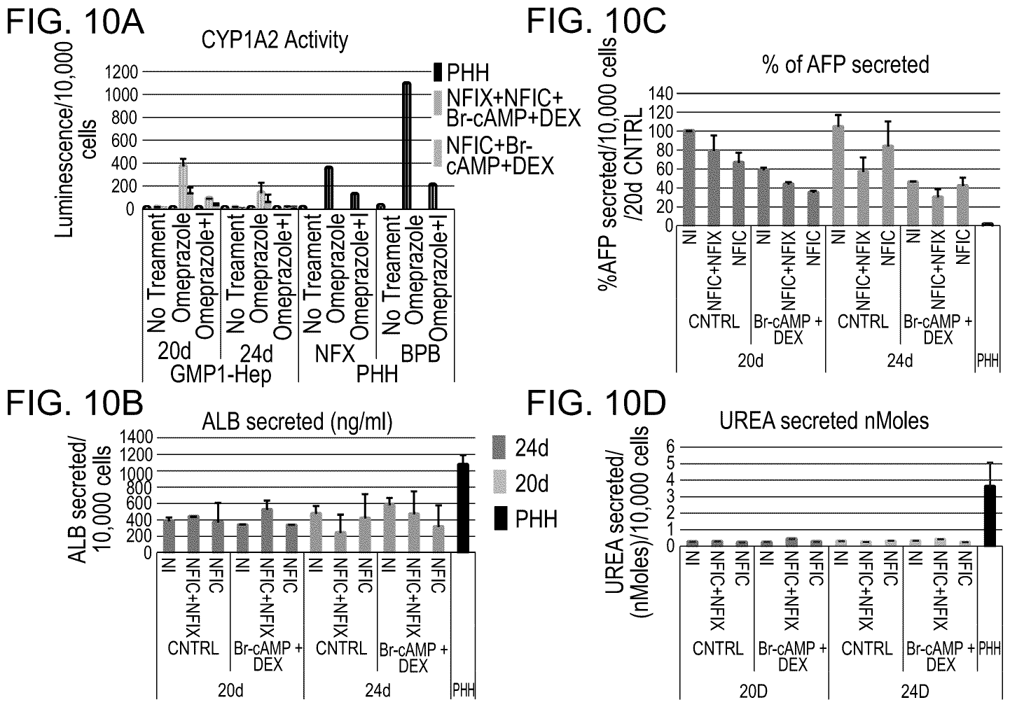

Pluripotent stem cell-derived immature hepatocytes (GMP1-Hep) were generated using a four stage step-wise differentiation process, as described in detail in Example 1. At the end of stage 3, transductions were performed with lentivirus particles comprising Tet-On3G (MOI of 5) or lentivirus particles comprising Tet-On3G in combination with the transcription factor NFIC, transcript variant 1 (NFIC); NFIX; or NFIC, transcript variant 1 (NFIC) and NFIX (MOI of 3) at day 15 of differentiation towards the hepatocyte like cells (FIG. 8A). The cells were subsequently cultured for 5 days or 9 days in stage 4 media, as described above in Example 1, comprising 1 μg/ml of doxycycline in the presence or absence of 1 mM of 8-Br-cAMP and 100 nM of dexamethasone. The cells were harvested at day 20 and day 24 of cell culture. The functional activity assays were performed, as described in detail in Example 1, in order to determine CYP1A2 activity (FIG. 10A), ALB secretion (FIG. 10B), AFP secretion (FIG. 10C) and urea secretion (FIG. 10D). The results of the study, as depicted in FIG. 10, demonstrate that increasing expression of transcription factors NFIC and/or NFIX in pluripotent stem cell derived immature hepatocytes increases CYP1A2 activity, increases secretion of ALB, and decreases secretion of AFP, and thereby promotes the generation of mature hepatocytes.

### Example 11: Increasing Expression of a Combination of Different Transcription Factors in Immature Hepatocytes

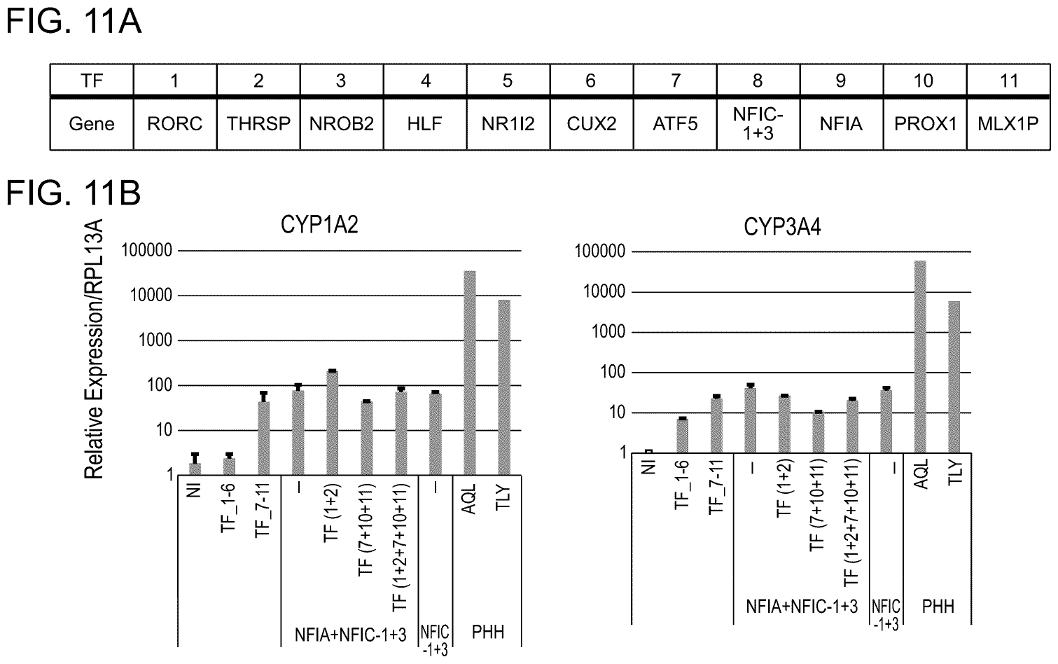

The HuH7-Tet-On3G cells, generated as described above in Example 1, were transduced with lentivirus particles comprising different transcription factors, as described in FIG. 11A, at an MOI of 10. After transduction, the cells were cultured for 5 days in a culture media comprising 1 μg/ml of doxycycline. The expression levels of the mature hepatocyte markers CYP1A2 and CYP3A4 (FIG. 11B) were determined relative to the house-keeping gene RPL13A, and were normalized to the non-infected (“NI”) negative control sample. PHH cells, of lots AQL and TLY, were obtained from BioIVT and the mRNA was extracted from frozen vials. The results of the study, as depicted in FIG. 11B, demonstrate that increasing expression of a combination of different transcription factors do not further increase expression levels of mature hepatocyte markers in immature hepatocytes, relative to the increase observed with increasing expression of NFIC alone.

### Example 12: Time Course Analysis of Expression of Mature Hepatocyte Markers by Increasing Expression of Transcription Factors NFIC and/or NFIX in Pluripotent Stem Cell Derived Immature Hepatocytes

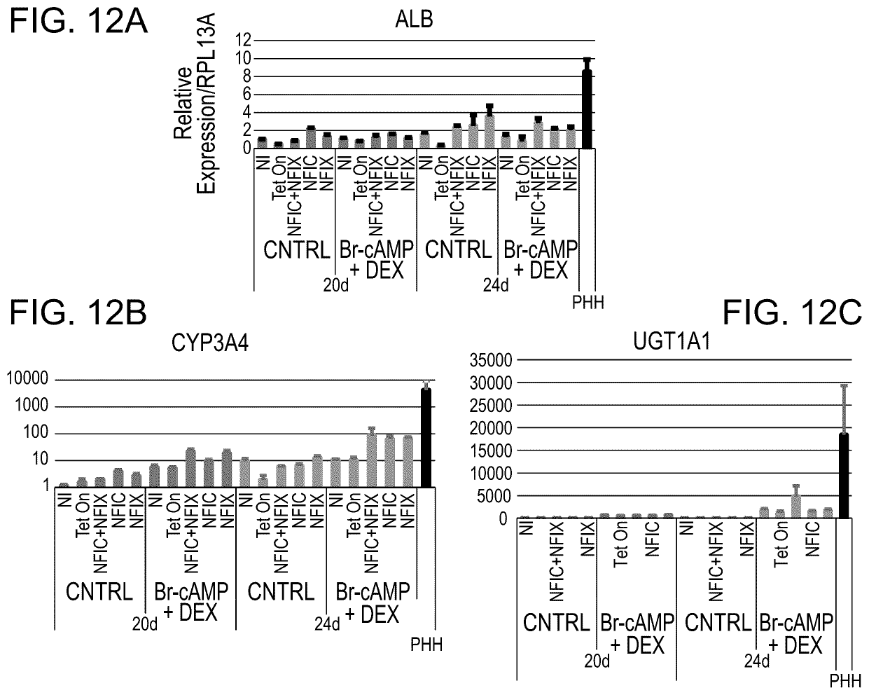

Pluripotent stem cell derived-immature hepatocytes were generated using a four stage step-wise differentiation process, as described in detail in Example 1 (FIG. 8A). At the end of stage 3, transductions were performed with lentivirus particles comprising Tet-On3G (MOI of 5) (“TetOn”) or lentivirus particles comprising Tet-On3G in combination with the transcription factor NFIC, transcript variant 1 (NFIC); NFIX; or NFIC, transcript variant 1 (NFIC) and NFIX (MOI of 3) at day 15 of differentiation towards the hepatocyte like cells (FIG. 8A). The cells were subsequently cultured for 5 days or 9 days in stage 4 media, as described above in Example 1, comprising 1 μg/ml of doxycycline in the presence or absence of 1 mM of 8-Br-cAMP and 100 nM of dexamethasone. The cells were harvested at day 20 and day 24 of cell culture, and the expression levels of mature hepatocyte markers ALB (FIG. 12A), CYP3A4 (FIG. 12B) and UGT1A1 (FIG. 12C) were determined relative to the house-keeping gene RPL13A, and were normalized to the non-infected (“NI”) negative control sample. Primary human hepatocytes (PHH) expression values correspond to an average of the expression values of lots PHH-AQL and PHH-TLY. PHH cells were obtained from BioIVT and mRNA was extracted from frozen vials. The results of the study, as depicted in FIGS. 12A-C, demonstrate that increasing expression of transcription factors NFIC and/or NFIX in pluripotent stem cell derived immature hepatocytes increases expression levels of mature hepatocyte markers, and thereby promotes the generation of mature hepatocytes.

1. A method of generating mature hepatocytes, the method comprising increasing expression of at least one transcription factor selected from the group consisting of Nuclear Factor I X (NFIX) and Nuclear Factor I C (NFIC), in immature hepatocytes, thereby generating mature hepatocytes.

2. The method of claim 1, wherein the transcription factor is NFIX, NFIC, or NFIX and NFIC.

3-4. (canceled)

5. The method of claim 1, wherein the NFIC is at least one alternatively spliced NFIC variant selected from the group consisting of NFIC, transcript variant 1; NFIC, transcript variant 2; NFIC, transcript variant 3; NFIC, transcript variant 4; NFIC, transcript variant 5; and NFIC, transcript variant 1 and NFIC, transcript variant 3.

6-8. (canceled)

9. The method of claim 1, further comprising increasing expression of one or more transcription factors selected from the group consisting of RORC, NR0B2, ESR1, THRSP, TBX15, HLF, ATOH8, NR112, CUX2, ZNF662, TSHZ2, ATF5, NFIA, NFIB, NPAS2, FOS, ONECUT2, PROX1, NR1H4, MLXIPL, ETV1, AR, CEBPB, NR1D1, HEY2, ARID3C, KLF9, and DMRTA1 in the immature hepatocytes.

10. The method of claim 1, further comprising culturing the immature hepatocytes in a culture media comprising dexamethasone, 8-Bromoadenosine 3′,5′-cyclic monophosphate (8-Br-cAMP), or a combination thereof.

11. The method of claim 10, wherein:
(a) the culturing is performed for at least 2, 3, 4, 5, 6, 7, 8 or 9 days;
(b) the concentration of 8-Br-CAMP is at least 0.1 mM, 0.2 mM, 0.4 mM, 0.6 mM, 0.8 nM or 1 mM; and/or
(c) the concentration of dexamethasone is at least 5 nM, 10 nM, 20 nM, 40 nM, 60 nM, 80 nM or 100 nM.

12-13. (canceled)

14. The method of claim 1, wherein increasing the expression of the at least one transcription factor in the immature hepatocytes comprises contacting the immature hepatocytes with the at least one transcription factor.

15. The method of claim 1, wherein the immature hepatocytes comprise an expression vector comprising a nucleic acid encoding the at least one transcription factor.

16. The method of claim 15, wherein the expression vector is:
(a) a viral vector;
(b) a non-viral vector; or
(c) an inducible expression vector.

17-18. (canceled)

19. The method of claim 15, wherein the expression vector comprises:
(a) a promoter operably linked to a nucleic acid encoding the at least one transcription factor; or
(b) a self-cleaving sequence.

20. The method of claim 19, wherein the promoter is an endogenous promoter, an artificial promoter, or an inducible promoter.

21-22. (canceled)

23. The method of claim 1, wherein increasing the expression of the at least one transcription factor in the immature hepatocytes comprises:
(a) transduction of immature hepatocytes with a viral vector encoding the at least one transcription factor; or
(b) transfection of immature hepatocytes with an expression vector encoding the at least one transcription factor.

24. (canceled)

25. The method of claim 1, wherein:
(a) the immature hepatocytes are cultured for at least 2, 3, 4 or 5 days before increasing the expression of the at least one transcription factor; and/or
(b) the immature hepatocytes are cultured for at least 2, 3, 4, 5, 6, 7, 8 or 9 days after increasing the expression of the at least one transcription factor.

26. (canceled)

27. The method of claim 1, wherein;
(a) increasing the expression of NFIX comprises an increase of at least 0.1-fold, 0.2-fold, 0.5-fold, 1-fold, 2-fold, 5-fold, 10-fold, 20-fold, 50-fold, 100-fold, 200-fold, 500-fold, 1,000-fold, or 10,000-fold relative to endogenous expression levels of NFIX in the immature hepatocytes; and/or
(b) increasing the expression of NFIC comprises an increase of at least 0.1-fold, 0.2-fold, 0.5-fold, 1-fold, 2-fold, 5-fold, 10-fold, 20-fold, 50-fold, 100-fold, 200-fold, 500-fold, 1000-fold, or 10,000-fold relative to endogenous expression levels of NFIC in the immature hepatocytes.

28. (canceled)

29. The method of claim 1, wherein the mature hepatocytes exhibit:
(a) an increased expression of albumin (ALB), cytochrome P450 enzyme 1A2 (CYP1A2), cytochrome P450 enzyme 3A4 (CYP3A4), tyrosine aminotransferase (TAT), and/or UDP-glucuronosyltransferase 1A-1 (UGT1A1) relative to immature hepatocytes;
(b) a decreased expression of alpha fetoprotein (AFP) relative to immature hepatocytes; and/or
(c) an increased secretion of albumin (ALB), a decreased secretion of AFP, and/or an increased activity of CYP1A2, relative to immature hepatocytes.

30. The method of claim 29, wherein:
(a) the increased expression of CYP1A2 comprises an increase of at least 2-fold, 5-fold, 10-fold, 50-fold, 100-fold, 200-fold, 500-fold, 1,000-fold, 2,000-fold, 5,000-fold, or 10,000-fold relative to immature hepatocytes;
(b) the increased expression of CYP3A4 comprises an increase of at least 2-fold, 5-fold, 10-fold, 50-fold, 100-fold, 200-fold, 500-fold, 1,000-fold, 2,000-fold, 5,000-fold, or 10,000-fold relative to immature hepatocytes;
(c) the increased expression of TAT comprises an increase of at least 2-fold, 5-fold, 10-fold, 50-fold, 100-fold, 200-fold, 500-fold, 1,000-fold, 2,000-fold, 5,000-fold, or 10,000-fold relative to immature hepatocytes;
(d) the increased expression of UGT1A1 comprises an increase of at least 2-fold, 5-fold, 10-fold, 50-fold, 100-fold, 500-fold, 1,000-fold, 2,000-fold, 5,000-fold, or 10,000-fold relative to immature hepatocytes;
(e) the increased secretion of ALB comprises an increase of at least 5%, 10%, 15%, 20% or 25% relative to immature hepatocytes;
(f) the increased activity of CYP1A2 comprises an increase of at least 2-fold, 5-fold, 10-fold, 50-fold, 100-fold, 200-fold, or 400-fold relative to immature hepatocytes;
(g) the decreased expression of AFP comprises a decrease of at least 0.1-fold, 0.2-fold, 0.5-fold, 1-fold, 2-fold, 3-fold, or 4-fold relative to immature hepatocytes; and/or
(h) the decreased secretion of AFP comprises a decrease of at least 5%, 10%, 20%, 40%, or 60% relative to immature hepatocytes.

31-39. (canceled)

40. The method of claim 1, wherein increasing the expression of the at least one transcription factor in the immature hepatocytes:
(a) shifts the transcriptome of immature hepatocytes towards the transcriptome of mature hepatocytes by at least 1%, 5%, 10%, 20%, 30%, 40%, or 50%; and/or
(b) comprises use of a gene switch construct encoding the at least one transcription factor.

41. The method of claim 1, wherein the immature hepatocytes are derived from pluripotent stem cells; wherein the pluripotent stem cells are embryonic stem cells or induced pluripotent stem cells.

42-43. (canceled)

44. The method of claim 40, wherein the gene switch construct is a transcriptional gene switch construct or a post-transcriptional gene switch construct.

45. (canceled)

46. A method of generating pluripotent stem cell-derived mature hepatocytes, the method comprising:
(a) differentiating pluripotent stem cells to immature hepatocytes, wherein the pluripotent stem cells comprise an expression vector comprising a nucleic acid encoding the at least one transcription factor selected from the group consisting of Nuclear Factor I X (NFIX) and Nuclear Factor I C (NFIC), and
(b) increasing expression of the at least one transcription factor from the expression vector in the immature hepatocytes, thereby generating mature hepatocytes.

47. The method of claim 46, wherein:
(a) the pluripotent stem cells are embryonic stem cells or induced pluripotent stem cells; and/or
(b) the immature hepatocytes comprise hepatoblasts or hepatic stem cells.

48-50. (canceled)

51. The method of claim 46, wherein the transcription factor is NFIX, NFIC, or NFIX and NFIC.

52-53. (canceled)

54. The method of claim 46, wherein the NFIC is at least one alternatively spliced NFIC variant selected from the group consisting of NFIC, transcript variant 1; NFIC, transcript variant 2; NFIC, transcript variant 3; NFIC, transcript variant 4; NFIC, transcript variant 5; and NFIC, transcript variant 1 and NFIC, transcript variant 3.

55-70. (canceled)

71. The method of claim 46, wherein increasing the expression of the at least one transcription factor in the immature hepatocytes comprises inducing expression of the at least one transcription factor in the immature hepatocytes.

72. The method of claim 71, wherein inducing the expression of the at least one transcription factor in the immature hepatocytes comprises use of a gene switch construct encoding the at least one transcription factor.

73-76. (canceled)

77. The method of claim 46, wherein step (a) comprises culturing the pluripotent stem cells in a first differentiation media comprising Activin A, a second differentiation media comprising at least one of BMP4 and FGF2, and a third differentiation media comprising HGF, thereby generating the immature hepatocytes.

78. The method of claim 77, wherein the first differentiation media, the second differentiation media and the third differentiation media are each cultured for at least 5 days.

79. (canceled)

80. The method of claim 46, wherein:
(a) the immature hepatocytes are cultured in a culture media comprising hepatocyte growth factor (HGF) before increasing the expression of the at least one transcription factor; and/or
(b) the immature hepatocytes are cultured in a culture media comprising oncostatin-M (OSM) after increasing the expression of the at least one transcription factor.

81-96. (canceled)

97. A composition comprising a population of mature hepatocytes produced by the method of claim 1.

98. A pharmaceutical composition comprising a population of mature hepatocytes produced by the method of claim 1, and a pharmaceutically acceptable carrier.

99. A composition comprising a population of hepatocytes comprising increased expression levels of at least one transcription factor selected from the group consisting of Nuclear Factor I X (NFIX) and Nuclear Factor I C (NFIC), relative to endogenous expression levels of the transcription factor in the population of hepatocytes.

100-107. (canceled)

108. The composition of claim 99, wherein:
(a) the increased expression comprises exogenous expression of the at least one transcription factor; or
(b) the hepatocytes comprise an expression vector comprising a nucleic acid encoding the at least one transcription factor.

109. (canceled)

110. The composition of claim 108, wherein the expression vector:
(a) is a viral vector;
(b) is a non-viral vector;
(c) is an inducible expression vector;
(d) comprises a promoter operably linked to a nucleic acid encoding the at least one transcription factor;
(e) comprises a gene switch construct encoding the at least one transcription factor; or
(f) further comprises a self-cleaving sequence.

111. The composition of claim 110, wherein:
(a) the viral vector is selected from the group consisting of an adeno-associated virus (AAV) vector, an adenovirus vector, a lentivirus vector, a herpes simplex virus vector, a sendai virus vector, and a retrovirus vector;
(b) the non-viral vector is selected from the group consisting of a plasmid DNA, a linear double-stranded DNA (dsDNA), a linear single-stranded DNA (ssDNA), a nanoplasmid, a minicircle DNA, a single-stranded oligodeoxynucleotides (ssODN), a DDNA oligonucleotide, a single-stranded mRNA (ssRNA), and a double-stranded mRNA (dsRNA);
(c) the non-viral vector comprises a naked nucleic acid, a liposome, a dendrimer, a nanoparticle, a lipid-polymer system, a solid lipid nanoparticle, and/or a liposome protamine/DNA lipoplex (LPD);
(d) the promoter is an endogenous promoter, an artificial promoter, or an inducible promoter;
(e) the gene switch construct is a transcriptional gene switch construct or a post-transcriptional gene switch construct; and/or
(f) the self-cleaving sequence is selected from the group consisting of T2A, P2A, E2A and F2A.

112-125. (canceled)

126. The composition of claim 99, wherein the population of hepatocytes:
(a) is a population of immature hepatocytes or mature hepatocytes;
(b) comprises at least 10⁶ hepatocytes;
(c) further comprises non-hepatocyte cells; and/or
(d) is in the form of organoids.

127-133. (canceled)

134. A composition comprising a population of pluripotent stem cells comprising an expression vector, wherein the expression vector comprises a nucleic acid encoding at least one transcription factor selected from the group consisting of Nuclear Factor I X (NFIX), and Nuclear Factor I C (NFIC).

135-158. (canceled)

159. The composition of claim 134, wherein the population of pluripotent stem cells comprises at least 10⁶ pluripotent stem cells.

160. A method of treating a disease in a subject in need thereof, the method comprising administering to the subject an effective amount of the composition of claim 97, thereby treating the disease in the subject.

161. The method of claim 160, wherein the disease is selected from the group consisting of fulminant hepatic failure due to any cause, viral hepatitis, drug-induced liver injury, cirrhosis, inherited hepatic insufficiency (such as Wilson's disease, Gilbert's syndrome, or al-antitrypsin deficiency), hepatobiliary carcinoma, autoimmune liver disease (such as autoimmune chronic hepatitis or primary biliary cirrhosis), urea cycle disorder, factor VII deficiency, glycogen storage disease type 1, infantile Refsum's disease, phenylketonuria, severe infantile oxalosis, cirrhosis, liver injury, acute liver failure, hepatobiliary carcinoma, hepatocellular carcinoma, genetic cholestasis (PFIC and alagille syndrome), hereditary hemochromatosis, tyrosinemia type 1, argininosuccinic aciduria (ASL), Crigler-Najjar syndrome, familial amyloid polyneuropathy, atypical haemolytic uremic syndrome-1, primary hyperoxaluria type 1, maple syrup urine disease (MSUD), acute intermittent porphyria, coagulation defects, GSD type Ia (in metabolic control), homozygous familial hypercholesterolemia, organic acidurias, and any other condition that results in impaired hepatic function.

162. A kit comprising the composition of claim 97.

163. A kit comprising an expression vector, wherein the expression vector comprises a nucleic acid encoding at least one transcription factor selected from the group consisting of Nuclear Factor I X (NFIX), and Nuclear Factor I C (NFIC).

164-171. (canceled)

172. The method of claim 1, wherein:
(a) NFIX comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by the nucleotide sequence as set forth in SEQ ID NO: 1;
(b) NFIC comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence encoded by any one of the nucleotide sequences of SEQ ID NO: 2 to SEQ ID NO: 6;
(c) NFIX comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to the amino acid sequence set forth in SEQ ID NO: 40; or
(d) NFIC comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to any one of the amino acid sequences set forth in SEQ ID NO: 41-SEQ ID NO: 45.

173-191. (canceled)

192. A composition comprising a population of mature hepatocytes produced by the method of claim 46.

193. A method of treating a disease in a subject in need thereof, the method comprising administering to the subject an effective amount of the composition of claim 99, thereby treating the disease in the subject.
